SEQUENCE LISTING (6 sequences)

SEQ ID NO: 1 - protein, 477 aa, Artificial Sequence
  CBRN
MHHHHHHLVPRGSGLNDIFEAQKIEWHEENLYFQGMAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 2 - protein, 856 aa, Artificial Sequence
  DDB1
MHHHHHHVDEENLYFQGGGRMSYNYVVTAQKPTAVNGCVTGHFTSAEDLNLLIAKNTRLEIYVVTAEGLRPVKEVGMYGKIAVMELFRPKGESKDLLFILTAKYNACILEYKQSGESIDIITRAHGNVQDRIGRPSETGIIGIIDPECRMIGLRLYDGLFKVIPLDRDNKELKAFNIRLEELHVIDVKFLYGCQAPTICFVYQDPQGRHVKTYEVSLREKEFNKGPWKQENVEAEASMVIAVPEPFGGAIIIGQESITYHNGDKYLAIAPPIIKQSTIVCHNRVDPNGSRYLLGDMEGRLFMLLLEKEEQMDGTVTLKDLRVELLGETSIAECLTYLDNGVVFVGSRLGDSQLVKLNVDSNEQGSYVVAMETFTNLGPIVDMCVVDLERQGQGQLVTCSGAFKEGSLRIIRNGIGGNGNSGEIQKLHIRTVPLYESPRKICYQEVSQCFGVLSSRIEVQDTSGGTTALRPSASTQALSSSVSSSKLFSSSTAPHETSFGEEVEVHNLLIIDQHTFEVLHAHQFLQNEYALSLVSCKLGKDPNTYFIVGTAMVYPEEAEPKQGRIVVFQYSDGKLQTVAEKEVKGAVYSMVEFNGKLLASINSTVRLYEWTTEKELRTECNHYNNIMALYLKTKGDFILVGDLMRSVLLLAYKPMEGNFEEIARDFNPNWMSAVEILDDDNFLGAENAFNLFVCQKDSAATTDEERQHLQEVGLFHLGEFVNVFCHGSLVMQNLGETSTPTQGSVLFGTVNGMIGLVTSLSESWYNLLLDMQNRLNKVIKSVGKIEHSFWRSFHTERKTEPATGFIDGDLIESFLDISRPKMQEVVANLQYDDGSGMKREATADDLIKVVEELTRIH

SEQ ID NO: 3 - protein, 11 aa, Artificial Sequence
  HiBiT Tag
VSGWRLFKKIS

SEQ ID NO: 4 - DNA, 21 bp, Artificial Sequence
  gRNA
tgggatcactagcatgtctgc

SEQ ID NO: 5 - DNA, 56 bp, Artificial Sequence
  ssODN
tgggatcactagcatggtgagcggctggcggctgttcaagaagattagctctgcag

SEQ ID NO: 6 - protein, 148 aa, Artificial Sequence
  BRD4
MHHHHHHSSGVDLGTENLYFQSMNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEE

(19) United States
(12) Patent Application Publication
(10) Pub. No.: US 2023/0374007 A1
(43) Pub. Date: Nov. 23, 2023

(54) COMPOUNDS AND THEIR USE IN TREATING CANCER

(71) Applicant: AstraZeneca AB, Södertälje (SE)

(72) Inventors: Iacovos Neal Michaelides, Cambridge (GB); Ulf Börjesson, Södertälje (SE); Thomas George Christopher Hayhow, Cambridge (GB); Jason Grant Kettle, Cambridge (GB); Gavin William Collie, Cambridge (GB); Robert Ian Storer, Cambridge (GB); Christoph Grebner, Södertälje (SE); Matthew William Dampier Perry, Södertälje (SE); Sharanjeet Kaur Bagal, Cambridge (GB); Charlene Fallan, Cambridge (GB)

(21) Appl. No.: 18/247,014

(22) PCT Filed: Sep. 29, 2021
(86) PCT No.: PCT/EP2021/076752

§ 371 Date: Mar. 28, 2023

Related U.S. Application Data
(60) Provisional application No. 63/085,384, filed on Sep. 30, 2020.

(51) Int. Cl. C07D 471/04; C07D 403/04; C07D 401/14; C07D 409/04; C07D 495/14; C07D 487/04; C07D 417/04; C07D 413/04; C07D 491/048; C07D 405/04

(52) U.S. Cl. CPC C07D 471/04; C07D 403/04; C07D 401/14; C07D 409/04; C07D 495/14; C07D 487/04; C07D 417/04; C07D 413/04; C07D 491/048; C07D 405/04

(57) ABSTRACT

The specification generally relates to compounds of Formula (I) and pharmaceutically acceptable salts thereof, where A, Z, Y, RA, Linker and v have any of the meanings defined herein. This specification also relates to the use of such compounds and pharmaceutically acceptable salts thereof in methods of treatment of the human or animal body, for example in the prevention or treatment of cancer. This specification also relates to processes and intermediate compounds involved in the preparation of such compounds and to pharmaceutical compositions containing them.

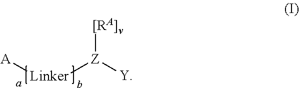

### FIELD

This specification relates to certain E3 ubiqutin ligase binding units which may be incorporated into a proteolysis targeting chimera (PROTAC) compound where such PROTAC compounds in turn may be used for the treatment of certain conditions/diseases in humans, such as cancer. This specification also relates to PROTAC compounds incorporating such beneficial E3 ubiquitin ligase binding ligands and to intermediate compounds that may be useful in the preparation of such PROTACs.

### BACKGROUND

Traditional small molecule drugs reversibly (or sometimes irreversibly) bind to a target protein as a means of modulating a given biological activity. In contrast, PROTACs bind to their target proteins, but then bring about the target protein's degradation. Having achieved this effect, the PROTAC is in theory able to repeat this process with another target protein. Accordingly, unlike with “traditional small molecule” inhibitors, the PROTAC-driven degradation mechanism can in theory operate in a sub-stoichiometric manner—meaning that more modest exposures of a PROTAC compound could still achieve a desired level of efficacy in vivo. In practice this can mean that the degradation power (DC₅₀ and Dₘₐₓ) of a PROTAC can have an improved effect than that reflected only by its binding affinity.

At a simplistic level, PROTAC molecules are often described as having three parts—(1) a part that is capable of binding to the target protein to be degraded, (2) a second part that is capable of binding to an E3 ubiquitin ligase, and finally, a linker that connects (1) and (2) together.

In use, the PROTAC binds to both the target protein and E3 ubiquitin ligase simultaneously to form a ternary complex. The E3 ligase then recruits an E2 conjugating enzyme to the ternary complex, which ubiquitinates the target protein. This has the effect of labelling the target protein for degradation by the cell's proteasome machinery. A PROTAC can then dissociate from the target protein and initiate another cycle of this process in a catalytic manner. Meanwhile, the ubiquitinated target proteins are recognized and degraded by the cell's proteasome machinery.

This PROTAC-mediated approach may be valuable as a method of treating certain diseases where the targeted degradation of specific bodily proteins may be beneficial, for example in the treatment of cancer.

Over recent decades, scientists have built up an understanding of which proteins may be promising targets to inhibit (or degrade) as strategies towards an effective cancer treatment. In turn, this understanding has led scientists to develop potent “traditional small molecule” binders of such target proteins. In more recent years, it has been recognised that such “traditional small molecule” binders/inhibitors may be incorporated into a PROTAC molecule, attaching the linker of the PROTAC to a portion of the binder/inhibitor moiety where it doesn't interfere with the binding that is fundamentally responsible for its potent inhibition.

Incorporating a “traditional small molecule inhibitor” into a PROTAC can lead to both inhibition and a parallel degradation event, via the general PROTAC's special mechanism of action as already described above. Beyond using “traditional small molecule inhibitors” as a part of a PROTAC molecule to affect the degradation of a target, non-functional “small molecule binders” can also be incorporated into a PROTAC molecule to affect the degradation of the target they bind to.

Whichever target protein binding unit (1) is used at one end of a PROTAC's linker unit, a fundamental element that must always be present at the other end of the PROTAC molecule is an E3 ubiquitin ligase binding unit (2) in order to direct the tagging of the target protein for degradation.

Scientific endeavors have already provided a number of potent E3 ubiquitin ligase binding units (2) that have been incorporated into PROTAC molecules. But as with “traditional small molecule” binders and PROTACs alike, there is always the issue of “off-target” activity in vivo which can be important to avoid in the development of safe and effective drug treatments. In other words, a given binding unit may be very potent against the intended target, but if it is inadvertently potent against other unintended biological targets in the human body, it may cause unacceptable toxicities, side effects and so on.

It is therefore an ongoing challenge to develop potent molecules for pharmaceutical use that are also suitably selective—i.e. avoiding inhibition/binding/degradation of unintended biological targets in vivo. The present researchers have noted that some known E3 ubiquitin ligase binding units (2) can also act (unintentionally) as potent degraders of SALL4 and/or Ikaros (IKZF1) amongst others. It is believed that degradation of SALL4 and Ikaros (IKZF1) amongst others, may risk serious unwanted effects in humans, for example developmental toxicities or bone marrow toxicities.

WO2018144649 discloses certain PROTAC compound structures and WO2019140387 discloses compounds that are said to be cereblon binders/ligands.

There is therefore a need to develop E3 ligase binding units (2) suitable for incorporation into PROTACs which are not only potent binders of an E3 ligase, but also have an improved selectivity profile.

The present researchers have also noted that some known E3 ubiquitin binding units (2) also exhibit disadvantageous levels of chemical and metabolic stability.

Therefore, as part of developing current and future PROTAC drug treatments for medicinal use (e.g. cancer), there is still a need to develop E3 ligase binding units (2) that have a combination of beneficial/improved properties that make them more suitable for use as part of a therapeutic PROTAC drug for human use, regardless of which target protein binder unit (1) is attached at the other end of the molecule.

Properties of interest during pharmaceutical discovery and development may relate to selectivity profile, absorption/bioavailability, distribution, metabolism, elimination, toxicity and side-effect profile, stability, manufacturability and so on.

### SUMMARY

The compounds of this specification provide, as a minimum, further potent E3 ubiquitin ligase binding units, specifically cereblon binding units, suitable for incorporation into PROTAC compounds, and therefore to PROTAC compounds containing them. The PROTAC compounds and E3 ubiquitin ligase binders of this specification also have a surprisingly beneficial combination of properties e.g. relating to stability (in human microsomes and to hydrolysis at pH 7.4) and selectivity (e.g. against SALL4 and/or IKZF1—which is expected to help provide a better safety profile for use in vivo).

This specification relates to the above-mentioned E3 ubiquitin ligase binding units and to PROTAC compounds (and pharmaceutically acceptable salts thereof) that incorporate such E3 ubiquitin ligase binding units. This specification also relates to pharmaceutical compositions containing such PROTACs (and pharmaceutically acceptable salts thereof) and their use in methods of treatment in the human or animal body, for example in the treatment or prevention of cancer. This specification also relates to processes and intermediate compounds (and salts thereof) involved in the preparation of said PROTACs.

In the first aspect of this specification there is provided a compound of Formula (I):

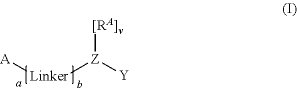

or a pharmaceutically acceptable salt thereof,
wherein:
A is a target protein binder unit;
Z is Z^{A} or Z^{B}:

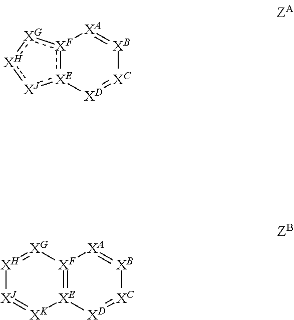

wherein:
[symbol] represents a single covalent bond or a double covalent bond;
1 of X^{A}, X^{B}, X^{C} & X^{D} is CY
0, 1 or 2 of X^{A}, X^{B}, X^{C}, X^{D}, X^{E} & X^{F} is/are N where X^{E} & X^{F} are not both N, and are otherwise C;
and when Z is Z^{A}:
   2 of X^{G}, X^{H} & X^{J} are independently selected from C and N; and
   1 of X^{G}, X^{H} & X^{J} is C, N, S or O;
      where at least one of X^{G}, X^{H} & X^{J} is N, S or O; and
      where any one C of X^{G}, X^{H} & X^{J} is optionally substituted by oxo,
      or when both of X^{G} & X^{J} are C, they both may be optionally substituted by oxo;
and when Z is Z^{B}
   1 of X^{G}, X^{H}, X^{J} & X^{K} is N and are otherwise C; or alternatively
   X^{G} & X^{K} are both N and X^{H} & X^{J} are both C;
Linker is a saturated or a partially or fully unsaturated framework comprising C and H atoms and at least one heteroatom, wherein said framework has end points of attachment ‘a’ and ‘b’ (and where ‘b’ may involve two attachment points ‘b1’ and ‘b2’ in cases where there are two points of attachment to Z at the ‘b’ end of the Linker) and a minimum length of from 6 to 26 atoms between ‘a’ and ‘b’; wherein said framework may include one or more straight and/or branched chains and/or rings and is optionally substituted on any available C atom(s) by one or more F; wherein said Linker is attached either:
once to Z: at any available C or N atom of Z; or
twice to Z: at any two adjacent available C atom(s) and/or N atom(s) at X^{H}, X^{G} & X^{J} (& X^{K} when present) such that a 5 to 7-membered ring is formed by the attachment of the Linker at the two adjacent atoms of Z;
each R^{A} is a substituent on any available C or N atom of Z—in each case independently selected from R^{A1} optionally substituted by one or more R^{A2}; where R^{A} is further selected from R^{A2} when R^{A} is a substituent on an available C atom of Z;
each R^{A1} is independently C₁₋₄alkyl, C₂₋₃alkenyl, C₂₋₃alkynyl, C₁₋₃alkoxyC₁₋₃alkyl, carboxyC₁₋₃alkyl, C₅₋₇carbocyclyl or a 4-6 membered heterocyclyl;
each R^{A2} is independently selected from F, Cl, Br, CN, NH₂, C₁₋₃alkyl, O(C₁₋₃alkyl), NH(C₁₋₃alkyl) and N(C₁₋₃alkyl)₂; wherein said C₁₋₃alkyls are optionally substituted by one or more F;
v is 0, 1, 2 or 3;
Y is:

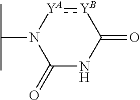

wherein:
Y^{A} & Y^{B} together represent CH—CH or C═C wherein Y^{A} & Y^{B} are each independently substituted by H, F, CN or Me.

This specification also describes, in part, a pharmaceutical composition which comprises a compound of Formula (I), or a pharmaceutically acceptable salt thereof, and at least one pharmaceutically acceptable excipient.

This specification also describes, in part, a compound of Formula (I), or a pharmaceutically acceptable salt thereof, for use in therapy.

This specification also describes, in part, a compound of Formula (I), or a pharmaceutically acceptable salt thereof, for use in the treatment of cancer.

This specification also describes, in part, a method for treating cancer in a warm-blooded animal in need of such treatment, which comprises administering to the warm-blooded animal a therapeutically effective amount of a compound of Formula (I), or a pharmaceutically acceptable salt thereof.

As shown in the experimental section hereinafter, the present researchers have produced a large number of potent and selective E3 ubiquitin ligase binders. They have also gained an understanding of where these selective E3 ubiquitin ligase binders may be linked to the linker of a PROTAC molecule in a way that does not interfere with their potent E3 ubiquitin ligase binding. Accordingly, the present researchers understand that when incorporating an E3 ubiquitin ligase binder into a PROTAC molecule, the linker of said PROTAC should not attach at the Y group of the compound of Formula (I), but may suitably attach at a range of positions on the heterocyclic Z group in the compound of Formula (I).

In a further aspect of this specification there is provided a PROTAC compound containing an E3 ubiquitin ligase binding unit of Formula (Ia):

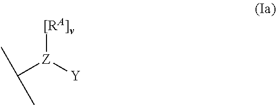

or a pharmaceutically acceptable salt thereof, wherein Z, Y, R^{A} and v may take any of the values defined herein for each of these integers respectively.

In a further aspect of the specification there is provided an E3 ubiquitin ligase binding unit of Formula (Ia), as described herein, for use in a PROTAC compound.

Accordingly, there is provided a unit of of Formula (Ia), as described herein, for use in a PROTAC compound (or pharmaceutically acceptable salt thereof).

Accordingly, there is provided a unit of of Formula (Ia), as described herein, for incorporation into a PROTAC compound (or pharmaceutically acceptable salt thereof).

Therefore, there is provided a E3 ubiquitin ligase binding unit of of Formula (Ia), as described herein, contained within a PROTAC compound (or a pharmaceutically acceptable salt thereof).

### DESCRIPTION OF ILLUSTRATIVE EMBODIMENTS

Many embodiments of this disclosure are detailed throughout the specification and will be apparent to a reader skilled in the art.

A pharmaceutically acceptable salt of a compound of Formula (I) or PROTAC compound described herein may be, for example, an acid-addition salt when said compound contains a basic functional group, such as an amine. An acid-addition salt may be formed using an inorganic acid or an organic acid. A pharmaceutically acceptable salt of said compound may be, for example, a base-addition salt when said compound contains an acidic functional group, such as a carboxylic acid. An acid-addition salt may be formed using an inorganic base or an organic base. “Pharmaceutically acceptable salt” is used to specify that the salt is suitable for use in the human or animal body. An example list of pharmaceutically acceptable salts can be found in the Handbook of Pharmaceutical Salts: Properties, Selection and Use, P. H. Stahl and C. G. Wermuth, editors, Weinheim/Zurich:Wiley-VCH/VHCA, 2002. A pharmaceutically acceptable salt of a compound of Formula (I) or PROTAC compound includes such salts that may be formed within the human or animal body after administration of said compound to said human or animal body.

As used herein the term “alkyl” includes straight chain, branched chain and cyclic alkyl groups and combinations thereof having the specified number of carbon atoms. Therefore, C₁₋₃alkyl includes methyl, ethyl, n-propyl, isopropyl and cyclopropyl; and C₁₋₉alkyl would include (4-isopropylcyclohexyl)methyl. The same principles apply to the term “alkoxy”. Similarly, as used herein the term “alkoxy” includes straight chain, branched chain and/or cyclic alkoxy groups having the specified number of carbon atoms. Therefore, C₁₋₃alkoxy [which may also be written as “O(C₁₋₃alkyl)” ] includes methoxy, ethoxy, n-propoxy, isopropoxy and cyclopropoxy. In a group where two alkyl groups are mentioned, for example, N(C₁₋₃alkyl)₂ each alkyl may be the same or different. Therefore, N(C₁₋₃alkyl)₂ includes for example, (methyl)(cyclopropyl)amine.

In this specification chemical abbreviations familiar to the skilled person may be used including for example “Me”=methyl, “Et”=ethyl, “Pr”=propyl, “Bu”=butyl and “Ph”=phenyl.

In this specification, a group for example such as “A-B-C” where B is defined “a direct bond” equates to “A-C”—i.e. where A and C are directly linked to each other by a single covalent bond.

Where the term “optionally” is used, it is intended that the subsequent feature may occur or may not occur. As such, use of the term “optionally” includes instances where the feature is present, and also instances where the feature is not present. For example, “methyl optionally substituted by one or more F” includes —CH₃, —CH₂F, —CHF₂ and —CF₃.

The term “substituted” means that one or more hydrogens on the designated atom or group is replaced by the indicated substituent(s) provided that any atom(s) bearing such substituent(s) maintains its permitted valency where the skilled person understands that the standard valencies of carbon, nitrogen and oxygen are 4, 3 and 2 respectively. Therefore, “substituted on any available C atom(s)” is to be understood to mean that the substituent(s) is/are limited in their positioning (and/or potentially in their number) according to whether there are any hydrogen atoms remaining on the designated atom or group which could be replaced by said substituent(s).

The dashed bonds included in the Z^{A}, [symbol] , indicate the possibility that the bond may in each case be a single covalent bond or a double covalent bond—in accordance with the atom (or group of atoms) present at each of the X^{E}, X^{F}, X^{G}, X^{H} and X^{J} positions. The skilled person understands that the standard valencies of carbon, nitrogen and oxygen are as mentioned above, and as such they can understand whether each dashed bond should be interpreted as a single bond or a double bond in any given Z^{A} group in this specification.

The term “adjacent” or “adjacent position”—for example in reference to X^{G}, X^{H} and X^{J} of Z refers to the next closest position in the molecular chain/ring system. Accordingly, in the context of Z: X^{G} and X^{H} are adjacent each other, X^{H} and X^{J} are also adjacent each other, but X^{G} is not adjacent X^{J}.

The term “saturated” means that the atoms of the specified framework or group are linked only by single covalent bonds. Accordingly, the term “unsaturated” means that the specified framework or group contains double and/or triple covalent bonds. Examples of unsaturated molecular fragments that may be present within a partly or fully unsaturated group or framework are C═C, C═N, C═O, N═N, C≡C or C≡N in cases where nitrogen and oxygen heteroatoms are permitted/present, and may also include S═O in cases where sulfur heteroatoms are also permitted/present.

It is to be understood that “hereroatom” may represent an oxygen, nitrogen or sulfur atom unless explicitly further limited in a given context.

The term “minimum length of [ . . . ] atoms between ‘a’ and ‘b’” refers to the shortest chain of atoms in the chain between ‘a’ and ‘b’. Therefore, if the chain consisted of —CH₂CH₂CH₂—, the number of atoms in the chain is 3 (the hydrogen atoms are regarded as not being in the chain). Alternatively if the chain consisted of 1,3-phenylene, where the shorter route around the phenyl ring contains 3 C atoms and the long route around the phenyl ring contains 5 C atoms, the minimum length of such a chain would be counted as 3 atoms.

It is to be understood that the points of attachment ‘a’ and ‘b’ each represent single covalent bonds to the relevant adjacent groups/atoms.

It is to be understood that in this specification “rings” may include single rings, fused rings, spirocyclic rings and bridged rings.

In reference to the Linker, as described herein, it is to be understood that the branching, where present may be present on a chain (even a chain of 1 atom length) or on a ring. The skilled person would generally interpret in this manner, but for the avoidance of doubt, it is to be understood that the “branching” that occurs inherently in order to form a ring is not considered “branching” in the context of the Linkers defined herein.

In the examples of this specification, the branching may involve one or more “═O” branches. It is to be understood that said branches may occur on the same or different atoms of the Linker framework. For example it is possible to have two “═O” branches on a sulfur heteroatom in order to form a SO₂ group within the Linker framework. It is further to be understood that ‘branches’ (and definitions for branches provided herein) refer to branches that branch off the main chain of atoms between ‘a’ and ‘b’ (or “b1” and “b2” in relevant cases) leading to a ‘dead end’ in the molecular structure.

In this specification it is to be understood that the point of attachment of a given group to some other group may be represented by a line meeting a bond substantially at right angles to said bond—for example as shown in the left hand side of structure Y hereinabove. In particular, when a “floating” point of attachment is indicated to a Z group (whether the Z groups is depicted as “Z” or shown explicitly as a bicyclic chemical drawing), the bond may be connected to any available carbon or nitrogen atom of said Z group (unless otherwise specified) and this applies irrespective of whether said “floating bond” is drawn over the X^{A}/X^{B}/X^{C}/X^{D}/X^{E}/X^{F} ring of Z or the X^{E}/X^{F}/X^{G}/X^{H}/X^{J}/(X^{K}) ring of Z. Furthermore, in the specific case where a “floating” bond relates to a linkage between Z and the linker of a PROTAC compound (e.g. in the compound of Formula (I)), said floating bond may itself, or in combination with another specified point of connection, provide a double linkage between the Linker and Z, via linkage points ‘b1’ and ‘b2’ in a manner as described herein.

In this specification when “0, 1 or 2 of X^{A}, X^{B}, X^{C}, X^{D}, X^{E} & X^{F} is/are N where X^{E} & X^{F} are not both N, and are otherwise C”—certain of the C atoms are to be understood to implicitly posses a hydrogen atom where necessary in order to satisfy the standard valency (4) for carbon atoms. The skilled person will understand that such H atom cannot be present on a C at X^{E} or X^{F} or on a C at X^{A}, X^{B}, X^{C} or X^{D} when a substituent or Linker is attached to said carbon.

In this specification a saturated heterocyclic group refers to a ring of atoms (including bridged rings, spiro rings, fused rings, and single rings) containing carbon atoms and at least one heteroatom, where the heteroatom(s) is/are each independently selected from N, O and S, and where each atom in the ring is linked to its adjacent atoms by single covalent bonds. Typically, a saturated heterocyclic group will have at least two carbon atoms separating each of the heteroatom(s) present in said group to ensure a suitable level of chemical stability for use in a pharmaceutical context. Where reference is made to a “nitrogen-containing saturated heterocyclic group” this requires the presence of at least one nitrogen heteroatom but does not limit the possibility of one or more non-nitrogen heteroatoms (i.e. S, O) being present in addition. Where reference is made to a cyclic group (e.g. a heterocyclic group) having a specified number of ring atoms, this includes the atoms making up the ring (including atoms involved in the bridge of a bridged ring, and all atoms of a fused or spiro ring) but does not include any hydrogen atoms or other substituent atoms attached to the ring atoms. Therefore, for example, a cyclic group which is 1,4-piperazin-1,4-diyl has 6 ring atoms (4C and 2N).

In this specification a “heterocyclyl” is a cyclic group containing at least one carbon atom and at least one heteroatom (selected from N, S and O unless otherwise stated or the context dictates otherwise). Such heterocyclyl may be fully saturated, partially unsaturated or fully unsaturated. A ‘4-6-membered heterocyclyl’ means that the total number of carbon and heteroatoms is between 4 and 6 within the heterocyclyl.

In this specification an alkylene group (for example a C₁₋₃alkylene) is a straight or branched-chain group having two points of connection made up of the specified number of carbon atoms, hydrogen atoms and single covalent bonds. A C₁alkylene is —CH₂—, a C₂alkylene is —CH₂CH₂— or —CH(Me)-. Accordingly a “cycloalkylene” is an alkylene group that includes a saturated ring of carbon atoms within its structure (including single rings, spiro rings, fused rings and bridged rings) and may be entirely composed of said ring, or may involve a branched ring such that a “C₆cycloalkylene” could represent 2,2-dimethylcyclobut-1,3-diyl.

The term “therapy” is intended to have its normal meaning of dealing with a disease in order to entirely or partially relieve one, some or all of its symptoms, or to correct or compensate for the underlying pathology. The term “therapy” also includes “prophylaxis” or “prophylactic” unless there are specific indications to the contrary. The terms “therapeutic” and “therapeutically” should be interpreted in a corresponding manner.

The term “prophylactic” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly, the term “treat” can be regarded as “applying therapy” where “therapy” is as defined herein.

Some values of variable groups are as follows. One, two or more of such values, may be used in any combination with any other definitions, claims, aspects or embodiments herein (unless the context doesn't permit) to provide further embodiments/claims of the specification.

In one embodiment A is a BRD4 binding unit.
In one embodiment A is a protein binding unit having the formula:

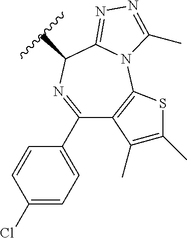 [SMILES: *[C@@H]1N=C(c2ccc(Cl)cc2)c2c(sc(C)c2C)-n2c(C)nnc21]

In one embodiment Z is Z^{A}.
In one embodiment Z is Z^{B}.
In one embodiment X^{A}, X^{B} or X^{C} is CY.
In one embodiment X^{B}, X^{C} or X^{D} is CY.
In one embodiment X^{A} or X^{D} is CY.
In one embodiment X^{B} or X^{C} is CY.
In one embodiment X^{A} or X^{B} is CY.
In one embodiment X^{C} or X^{D} is CY.
In one embodiment X^{A} is CY.
In one embodiment X^{B} is CY.
In one embodiment X^{C} is CY.
In one embodiment X^{D} is CY.
In one embodiment Y and Linker are not attached at adjacent positions of Z (for example Z^{A} or Z^{B}).
In one embodiment 0 or 1 of X^{A}, X^{B}, X^{C}, X^{D}, X^{E} & X^{F} is N, and are otherwise C.
In one embodiment 1 of X^{A}, X^{B}, X^{C}, X^{D}, X^{E} & X^{F} is N, and are otherwise C.
In one embodiment X^{A}, X^{B}, X^{C}, X^{D}, X^{E} & X^{F} are all C.
In one embodiment 0 or 1 of X^{A}, X^{D}, X^{E} & X^{F} is N and X^{A}, X^{B}, X^{C}, X^{D}, X^{E} & X^{F} are otherwise C
In one embodiment 0 or 1 of X^{A} & X^{D} is N and X^{A}, X^{B}, X^{C}, X^{D}, X^{E} & X^{F} are otherwise C.
In one embodiment X^{D} is C or N and X^{A}, X^{B}, X^{C}, X^{E} & X^{F} are all C.
In one embodiment 0 or 1 of X^{E} & X^{F} is N and X^{A}, X^{B}, X^{C}, X^{D}, X^{E} & X^{F} are otherwise C.
In one embodiment when Z is Z^{A}, X^{G} is selected from N, S, O, CH₂ and C(O).
In one embodiment when Z is Z^{A}, at least one of X^{G}, X^{H}& X^{J} is N.
In one embodiment when Z is Z^{A}; X^{G}, X^{H}& X^{J} are collectively selected from (N, C, C), (0, N, C), (N, C, S), (N, N, N), (S, C, C), (N, N, C), (N, C, N), (O, C, C), (O, C, N), (C, N, C) and (N, N, C) respectively.
In one embodiment when Z is Z^{B}, 1 of X^{G}, X^{H}, X^{J} & X^{K} is N and are otherwise C.
In one embodiment when Z is Z^{A}; X^{G} is N, X^{H} is C & X^{J} is C.
In one embodiment when Z is Z^{A}; X^{G} is O, X^{H} is N & X^{J} is C.
In one embodiment when Z is Z^{A}; X^{G} is N, X^{H} is C & X^{J} is S.
In one embodiment when Z is Z^{A}; X^{G} is N, X^{H} is N & X^{J} is N.
In one embodiment when Z is Z^{A}; X^{G} is S, X^{H} is C & X^{J} is C.
In one embodiment when Z is Z^{A}; X^{G} is N, X^{H} is N & X^{J} is C.
In one embodiment when Z is Z^{A}; X^{G} is N, X^{H} is C, & X^{J} is N.
In one embodiment when Z is Z^{A}; X^{G} is O, X^{H} is C & X^{J} is C.
In one embodiment when Z is Z^{A}; X^{G} is O, X^{H} is C & X^{J} is N.
In one embodiment when Z is Z^{A}; X^{G} is C, X^{H} is N & X^{J} is C.
In one embodiment when Z is Z^{A}; X^{G} is N, X^{H} is N & X^{J} is C.
In one embodiment when Z is Z^{B}, X^{G} is N and X^{H}, X^{J} & X^{K} are all C.
In one embodiment when Z is Z^{B}, X^{H} is N and X^{G}, X^{J} & X^{K} are all C.
In one embodiment when Z is Z^{B}, X^{J} is N and X^{G}, X^{H} & X^{K} are all C.
In one embodiment when Z is Z^{B}, X^{K} is N and X^{G}, X^{H} & X^{J} are all C.
In one embodiment when Z is Z^{A}; the (X^{G}—X^{H}-X^{J}) group together is selected from (N—C═C), (N—C—C), (N═C—C), (O—N═C), (N═C—S), (N—N═N), (S—C═C), (N—N═C), (N—C═N), (O—C═C), (O—C═N), (O—C—N), (C—N—C) and (N—N—C).
In one embodiment when Z is Z^{A}; X^{G}, X^{H} & X^{J} are collectively N—C═C.
In one embodiment when Z is Z^{A}; X^{G}, X^{H} & X^{J} are collectively N—C—C.
In one embodiment when Z is Z^{A}; X^{G}, X^{H} & X^{J} are collectively N═C—C.
In one embodiment when Z is Z^{A}; X^{G}, X^{H} & X^{J} are collectively O—N═C.
In one embodiment when Z is Z^{A}; X^{G}, X^{H} & X^{J} are collectively N═C—S.
In one embodiment when Z is Z^{A}; X^{G}, X^{H} & X^{J} are collectively N—N═N.
In one embodiment when Z is Z^{A}; X^{G}, X^{H} & X^{J} are collectively S—C═C.
In one embodiment when Z is Z^{A}; X^{G}, X^{H} & X^{J} are collectively N—N═C.
In one embodiment when Z is Z^{A}; X^{G}, X^{H} & X^{J} are collectively N—C═N.
In one embodiment when Z is Z^{A}; X^{G}, X^{H} & X^{J} are collectively O—C═C.
In one embodiment when Z is Z^{A}; X^{G}, X^{H} & X^{J} are collectively O—C═N.
In one embodiment when Z is Z^{A}; X^{G}, X^{H} & X^{J} are collectively O—C—N.
In one embodiment when Z is Z^{A}; X^{G}, X^{H} & X^{J} are collectively C—N—C.
In one embodiment when Z is Z^{A}; X^{G}, X^{H} & X^{J} are collectively N—N—C.
In one embodiment Z is selected from indole, benzisoxazole, 1H-pyrrolo[2,3-c]pyridine, benzothiazole, 1H-pyrrolo[3,2-b]pyridine, indoline, benzotriazole, indazole, benzothiophene, 2H-indazole, benzimidazole, benzofuran, benzoxazole, 3H-1,3-benzoxazol-2-one, pyrazolo[1,5-a]pyridine, isoindolin-1-one, imidazo[1,2-a]pyridine, isoindoline, isoxazo[4,5-b]pyridine, furo[3,2-b]pyridine, 1H-pyrrolo[2,3-b]pyridine, 1,2,3,4-tetrahydroquinoline and 1,2,3,4-tetrahydroisoquinoline.
In one embodiment Z^{A} is selected from indole, benzisoxazole, 1H-pyrrolo[2,3-c]pyridine, benzothiazole, 1H-pyrrolo[3,2-b]pyridine, indoline, benzotriazole, indazole, benzothiophene, 2H-indazole, benzimidazole, benzofuran, benzoxazole, 3H-1,3-benzoxazol-2-one, pyrazolo[1,5-a]pyridine, isoindolin-1-one, imidazo[1,2-a]pyridine, isoindoline, isoxazo[4,5-b]pyridine, furo[3,2-b]pyridine and 1H-pyrrolo[2,3-b]pyridine.
   The skilled person recognises that while the values of Z and Z^{A} mentioned above and below are presented as neutral heterocycle names for clarity and simplicity, they are in fact radicals. Accordingly, they will have attachments (as defined herein) to a Y group, potential attachments to one or more R^{A} groups, and in the case of Formula (I) and Formula (Ia) for example—there will also be one or two attachments to a Linker.
In one embodiment Z^{A} is indole.
In one embodiment Z^{A} is benzisoxazole.
In one embodiment Z^{A} is 1H-pyrrolo[2,3-c]pyridine.
In one embodiment Z^{A} is benzothiazole.
In one embodiment Z^{A} is 1H-pyrrolo[3,2-b]pyridine
In one embodiment Z^{A} is indoline.
In one embodiment Z^{A} is benzotriazole.
In one embodiment Z^{A} is indazole.
In one embodiment Z^{A} is benzothiophene.
In one embodiment Z^{A} is 2H-indazole.
In one embodiment Z^{A} is benzimidazole.
In one embodiment Z^{A} is benzofuran.
In one embodiment Z^{A} is benzoxazole.
In one embodiment Z^{A} is 3H-1,3-benzoxazol-2-one.
In one embodiment Z^{A} is pyrazolo[1,5-a]pyridine.
In one embodiment Z^{A} is isoindolin-1-one.
In one embodiment Z^{A} is imidazo[1,2-a]pyridine.
In one embodiment Z^{A} is isoindoline.
In one embodiment Z^{A} is isoxazo[4,5-b]pyridine.
In one embodiment Z^{A} is furo[3,2-b]pyridine.
In one embodiment Z^{A} is 1H-pyrrolo[2,3-b]pyridine
In one embodiment Z^{B} is selected from 1,2,3,4-tetrahydroquinoline and 1,2,3,4-tetrahydroisoquinoline.
In one embodiment Z^{B} is 1,2,3,4-tetrahydroquinoline.
In one embodiment Z^{B} is 1,2,3,4-tetrahydroisoquinoline.
In one embodiment the E3 ubiquitin ligase binding unit of Formula (Ia) [or Z, Y, R^{A} and v collectively within the compound of Formula (I)] is selected from any one or more of the following formulae 1 to 54 below:

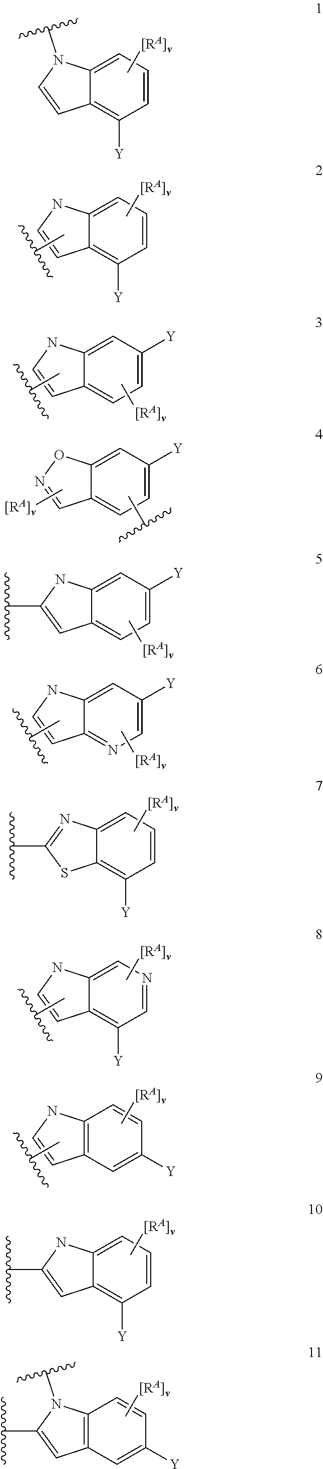

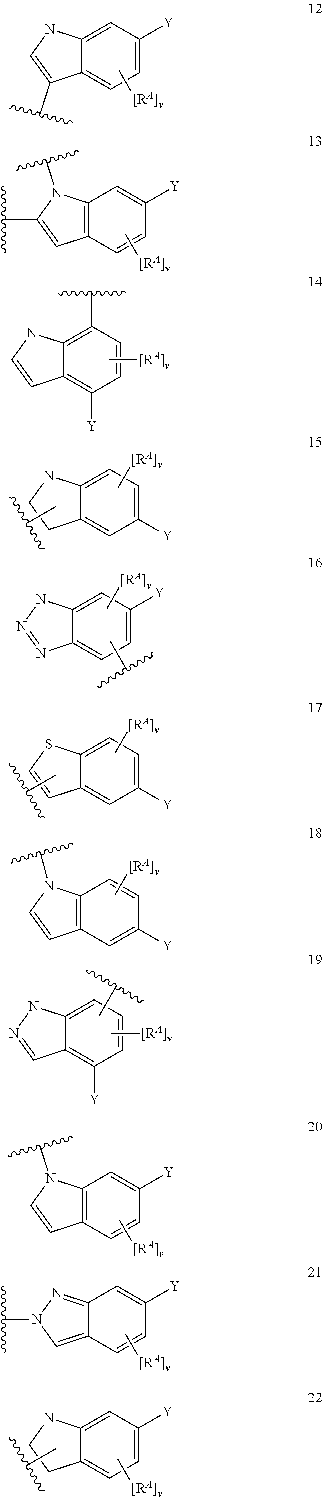

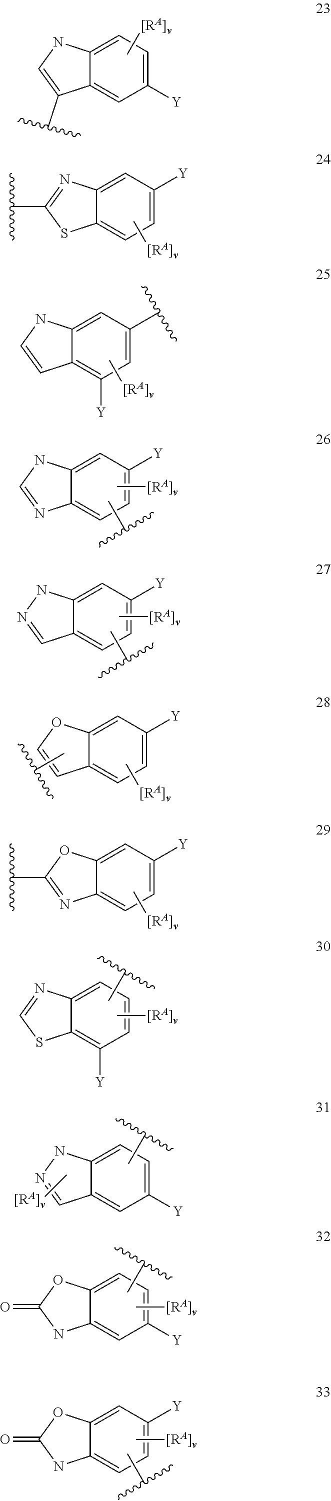

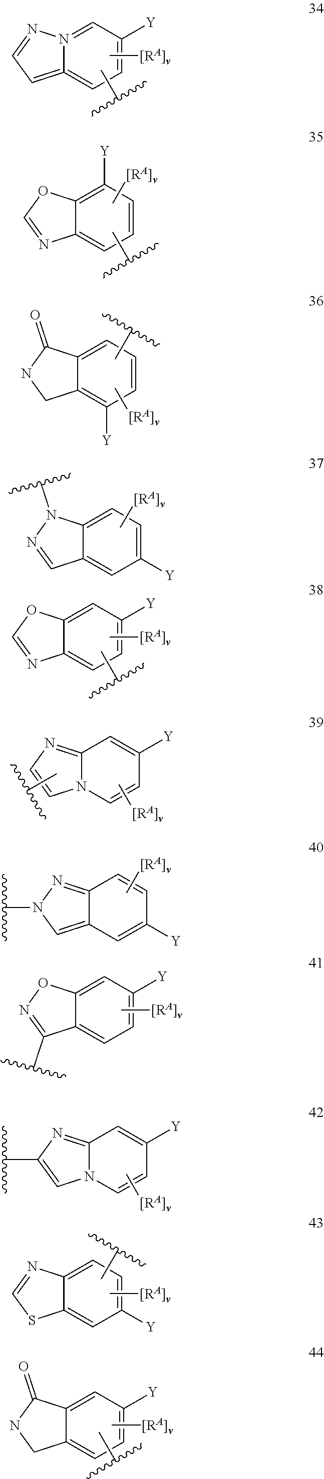

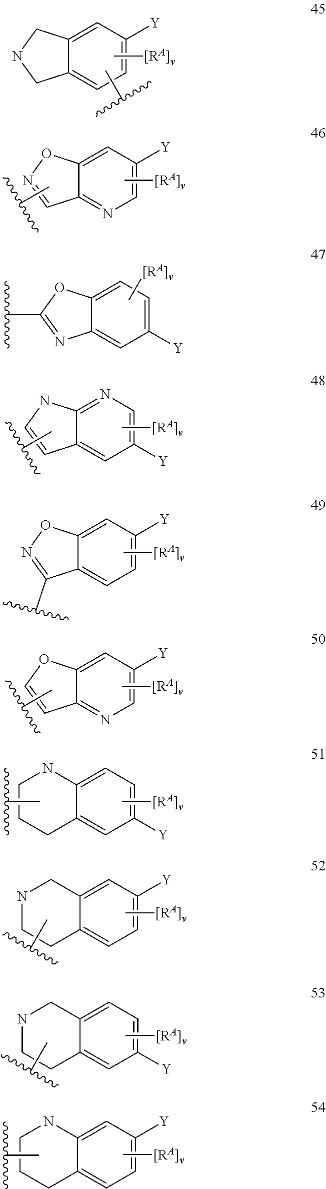

[wherein the floating bonds that are not involved with [R^{A}]ᵥ on each of the above-mentioned formulae 1 to 54 may attach either once at any available C or available N of the bicyclic heterocycle Z, or twice at any two adjacent and available C and/or N atoms of the bicyclic heterocycle Z].
In one embodiment v is 0, 1 or 2.
In one embodiment v is 0 or 1.
In one embodiment v is 0.
In one embodiment v is 1.
In one embodiment v is 2.
In one embodiment v is 3.
In one embodiment v is 1 or 2.
In one embodiment R^{A} is a substituent on any available C or N atom of Z—in each case independently selected from C₁₋₃alkyl optionally substituted by one of more F, C₁₋₃alkenyl, C₁₋₃alkynyl, C₁₋₃alkoxyC₁₋₃alkyl and carboxyC₁₋₃alkyl; and R^{A} is further selected from F, Cl, Br, CN, NH₂, C₁₋₃alkoxy, NH(C₁₋₃alkyl) and N(C₁₋₃alkyl)₂ when said R^{A} is a substituent on an available C of Z.

Therefore, in one embodiment of this specification there is provided a compound of Formula (I):

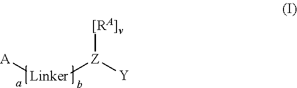

or a pharmaceutically acceptable salt thereof,
wherein:
A is a target protein binder unit;
Z is Z^{A} or Z^{B}

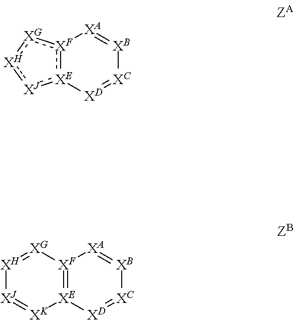

wherein:
[symbol] represents a single covalent bond or a double covalent bond;
1 of X^{A}, X^{B}, X^{C} & X^{D} is CY;
0, 1 or 2 of X^{A}, X^{B}, X^{C}, X^{D}, X^{E} & X^{F} is/are N where X^{E} & X^{F} are not both N, and are otherwise C;
and when Z is Z^{A}:
   2 of X^{G}, X^{H} & X^{J} are independently selected from C and N; and
   1 of X^{G}, X^{H} & X^{J} is C, N, S or O;
      where at least one of X^{G}, X^{H} & X^{J} is N, S or O; and
      where any one C of X^{G}, X^{H}& X^{J} is optionally substituted by oxo,
      or when both of X^{G} & X^{J} are C, they both may be optionally substituted by oxo;
and when Z is Z^{B}
   1 of X^{G}, X^{H}, X^{J} & X^{K} is N and are otherwise C; or alternatively
   X^{G} & X^{K} are both N and X^{H} & X^{J} are both C;
Linker is a saturated or a partially or fully unsaturated framework comprising C and H atoms and at least one heteroatom, wherein said framework has end points of attachment ‘a’ and ‘b’ (and where ‘b’ may involve two attachment points ‘b1’ and ‘b2’ in cases where there are two points of attachment to Z at the ‘b’ end of the Linker) and a minimum length of from 6 to 26 atoms between ‘a’ and ‘b’; wherein said framework may include one or more straight and/or branched chains and/or rings and is optionally substituted on any available C atom(s) by one or more F; wherein said Linker is attached either:
once to Z: at any available C or N atom of Z; or
twice to Z: at any two adjacent available C atom(s) and/or N atom(s) at X^{H}, X^{G} & X^{J} (& X^{K} when present) such that a 5 to 7-membered ring is formed by the attachment of the Linker at the two adjacent atoms of Z;
each R^{A} is a substituent on any available C or N atom of Z—in each case independently selected from C₁₋₃alkyl optionally substituted by one of more F, C₁₋₃alkenyl, C₁₋₃alkynyl, C₁₋₃alkoxyC₁₋₃alkyl and carboxyC₁₋₃alkyl; and R^{A} is further selected from F, Cl, Br, CN, NH₂, C₁₋₃alkoxy, NH(C₁₋₃alkyl) and N(C₁₋₃alkyl)₂ when said R^{A} is a substituent on an available C of Z;
v is 0, 1, 2 or 3;
Y is:

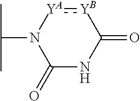

wherein:
Y^{A} & Y^{B} together represent CH—CH or C═C wherein Y^{A} & Y^{B} are each independently substituted by H, F, CN or Me.
In one embodiment, each R^{A} is a substituent on any available C or N atom of Z—in each case independently selected from C₁₋₃alkyl, N(C₁₋₃alkyl)₂ and C₁₋₃alkoxyC₁₋₃alkyl and R^{A} is further selected from F, Cl, CN and C₁₋₃alkoxy when said R^{A} is a substituent on an available C of Z.
In one embodiment, each R^{A} is a substituent on any available C or N atom of Z—in each case independently selected from C₁₋₃alkyl and C₁₋₃alkoxyC₁₋₃alkyl and R^{A} is further selected from F, Cl, and C₁₋₃alkoxy when said R^{A} is a substituent on an available C of Z.
In one embodiment, each R^{A} is a substituent on any available C or N atom of Z—in each case independently selected from methyl, dimethylamino and methoxymethyl and R^{A} is further selected from F, Cl, CN and methoxy when said R^{A} is a substituent on an available C of Z.
In one embodiment, each R^{A} is a substituent on any available C or N atom of Z—in each case independently selected from methyl and methoxymethyl and R^{A} is further selected from F, Cl, and methoxy when said R^{A} is a substituent on an available C of Z.
In one embodiment, each R^{A} is a substituent on any available C or N atom of Z—in each case independently selected from C₁₋₃alkyl (for example Me).
In one embodiment v is 0 or 1 and when v is 1, R^{A} is CN.
In one embodiment v is 0 or 1 and when v is 1, R^{A} is N(C₁₋₃alkyl)₂ [for example: dimethylamino].
In one embodiment v is 0 or 1 and when v is 1, R^{A} is chloro.
In one embodiment v is 0 or 1 and when v is 1, R^{A} is C₁₋₄alkyl [for example: methyl].
In one embodiment v is 0 or 1 and when v is 1, R^{A} is C₁₋₃alkoxy [for example: methoxy].
In one embodiment v is 0 or 1 and when v is 1, R^{A} is fluoro.
In one embodiment v is 0 or 1 and when v is 1, R^{A} is C₁₋₃alkoxyC₁₋₃alkyl [for example: methoxymethyl].
In one embodiment v is 0, 1 or 2, and when v is 1 or 2, the/one R^{A} is fluoro.
In one embodiment v is 0, 1 or 2, and when v is 1 or 2, the/one R^{A} is C₁₋₄alkyl (for example: methyl].
In one embodiment Y^{A} & Y^{B} together represent CH—CH wherein Y^{A} & Y^{B} are each independently substituted by H, F, CN or Me.
In one embodiment Y^{A} & Y^{B} together represent C═C wherein Y^{A} & Y^{B} are each independently substituted by H, F, CN or Me.
In one embodiment Y^{A} & Y^{B} are both substituted by H.
In one embodiment Y^{A} is substituted by H and Y^{B} is substituted by H, F or Me.
In one embodiment Y is selected from 6-fluoro-2,4-dioxohexahydropyrimidin-1-yl, 6-fluoro-2,4-dioxo-pyrimidin-1-yl, 2,4-dioxopyrimidin-1-yl, 6-methyl-2,4-dioxo-pyrimidin-1-yl and 2,6-dioxohexahydropyrimidin-1-yl.
In one embodiment Y is 6-fluoro-2,4-dioxohexahydropyrimidin-1-yl.
In one embodiment Y is 6-fluoro-2,4-dioxo-pyrimidin-1-yl.
In one embodiment Y is 2,4-dioxopyrimidin-1-yl.
In one embodiment Y is 6-methyl-2,4-dioxo-pyrimidin-1-yl.
In one embodiment Y is 2,6-dioxohexahydropyrimidin-1-yl.
In one embodiment, each E3 ubiquitin ligase binding unit of Formula (Ia) [or Z, Y, R^{A} and v collectively within the compound of Formula (I)] is selected from any one or more of the following formulae 1 to 107 below:

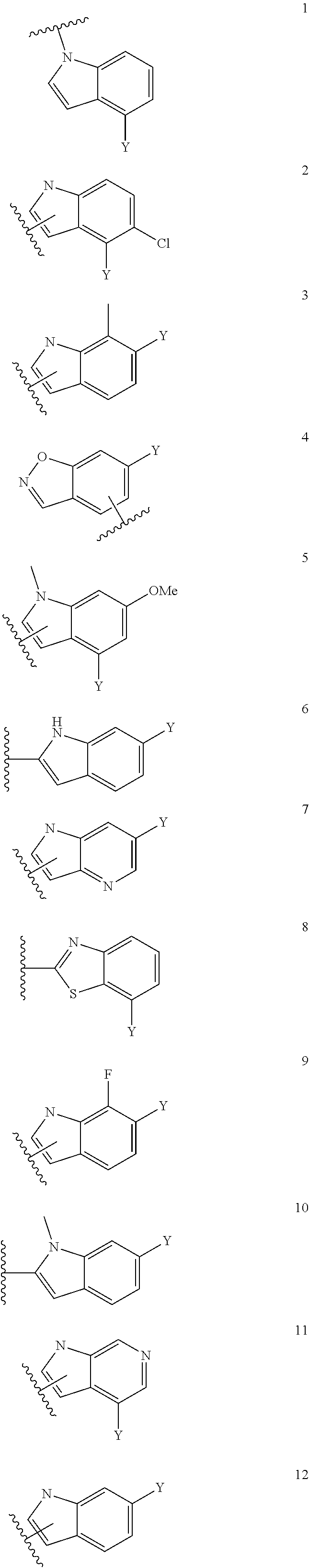

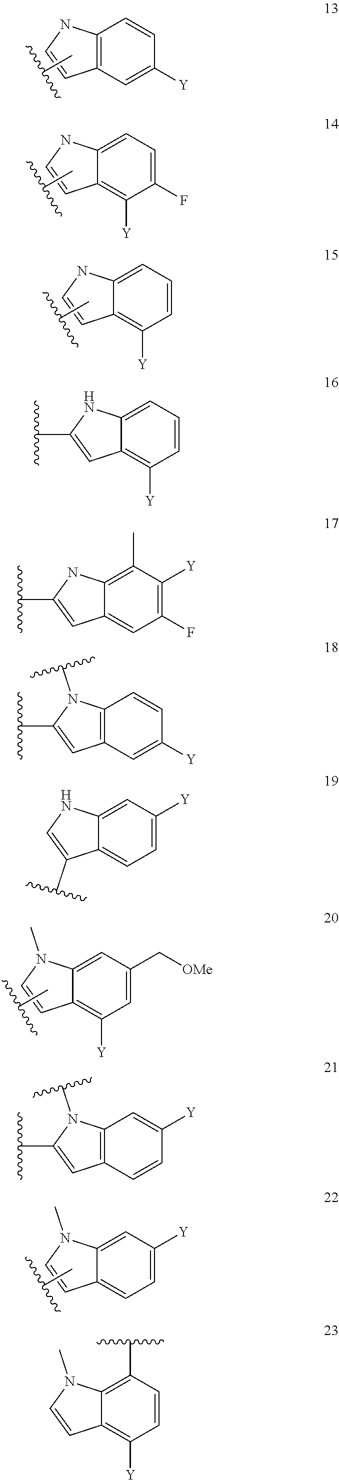

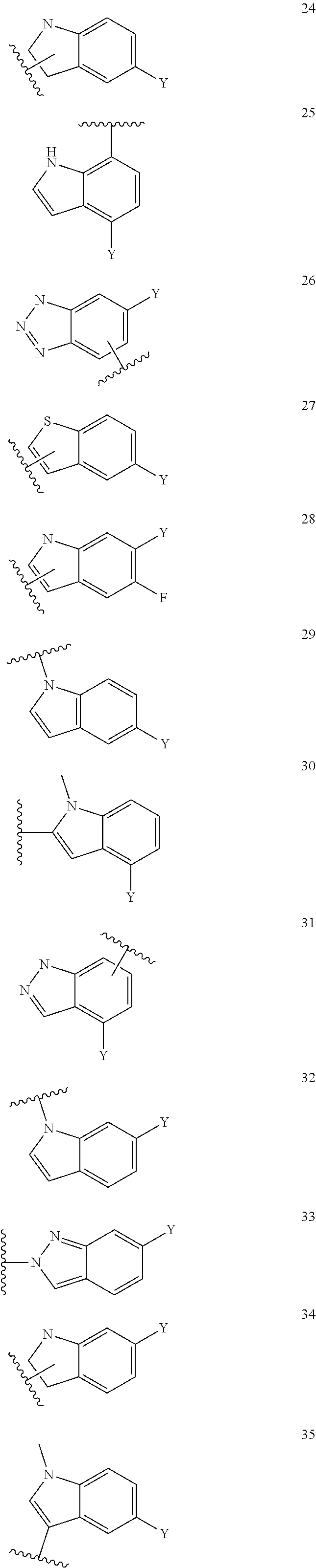

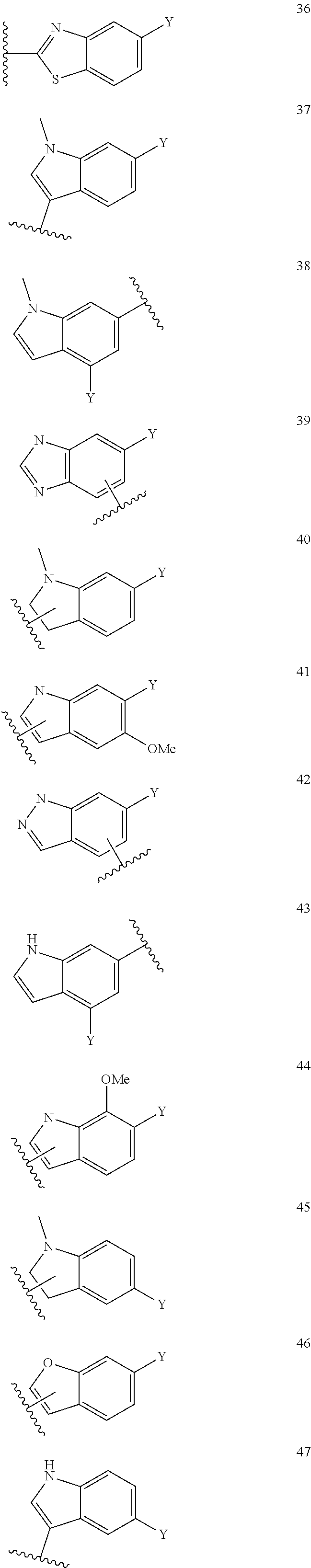

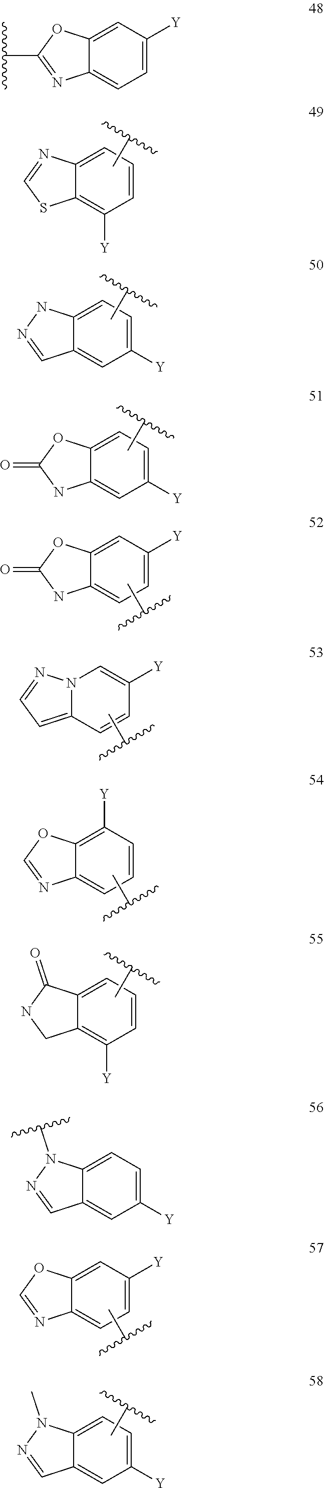

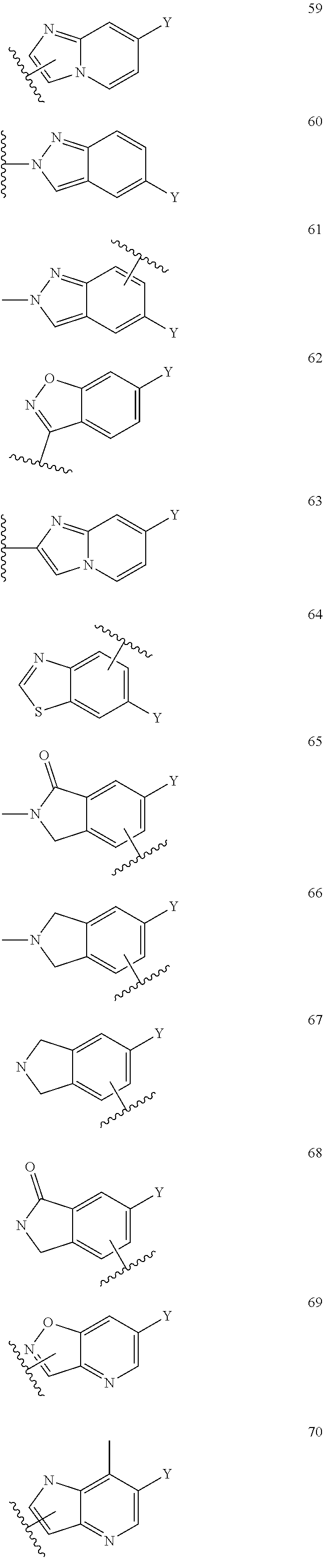

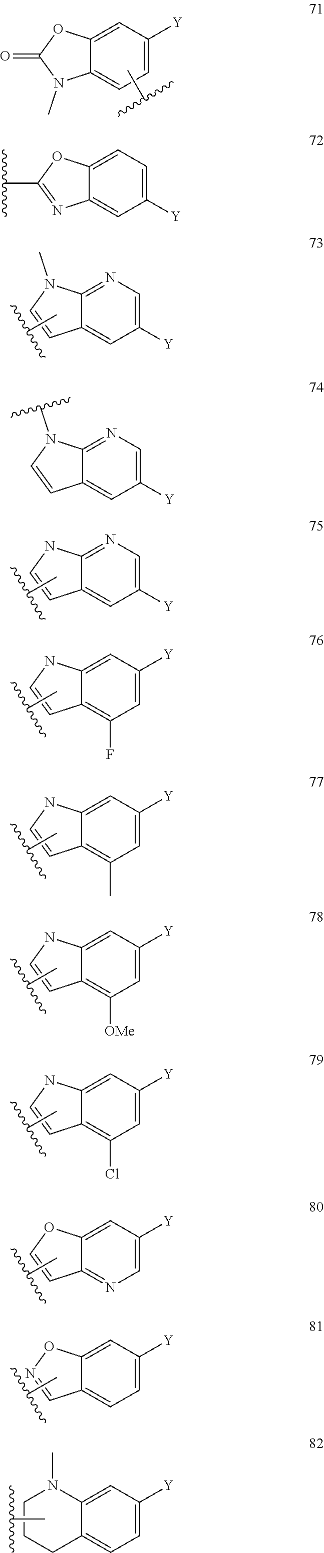

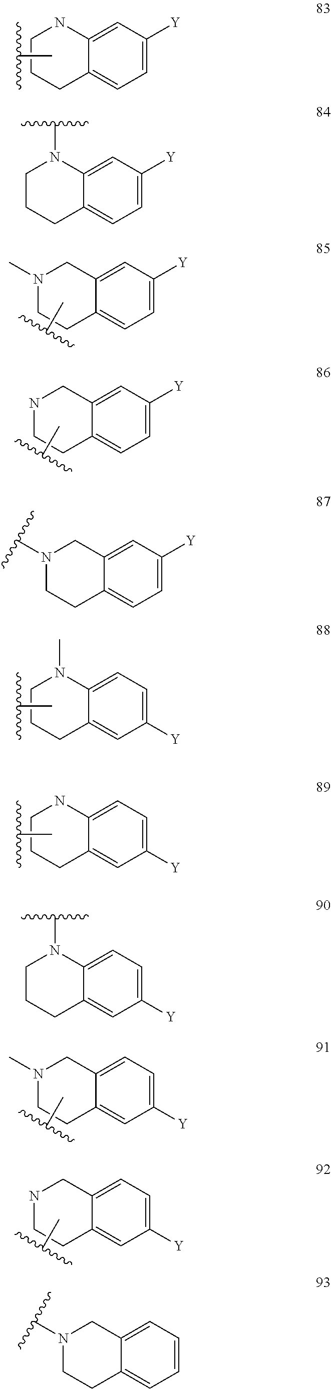

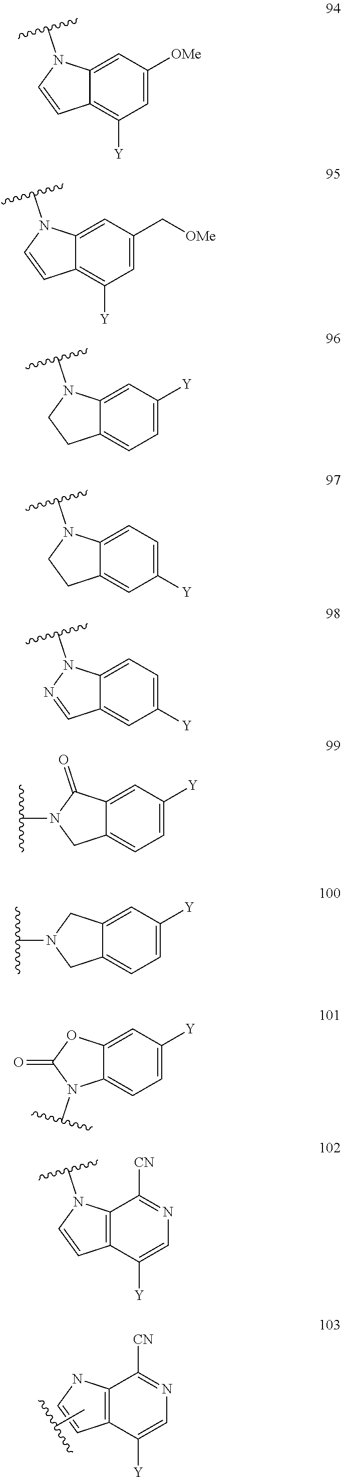

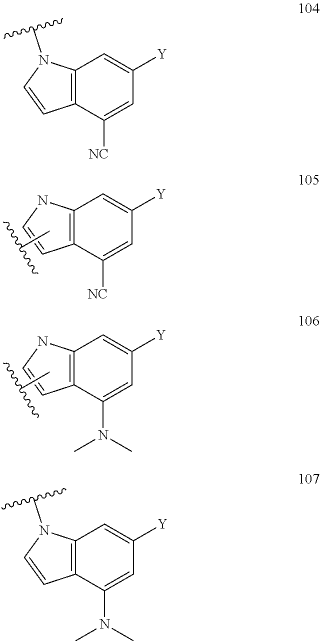

[wherein the floating bond on each of the above-mentioned formulae 1 to 107 may either represent a single point of attachment at any available C or available N of the bicyclic heterocycle Z, or may represent a double attachment via any two adjacent and available C and/or N atoms of the bicyclic heterocycle Z].
In one embodiment the Linker is attached only once to Z.
In one embodiment the framework of the Linker is a saturated or partially unsaturated framework.
In one embodiment the framework of the Linker comprises C and H atoms and at least two heteroatoms.
In one embodiment the framework of the Linker comprises C and H atoms and at least one N heteroatom.
In one embodiment the framework of the Linker comprises C and H atoms and at least two heteroatoms selected from N & O.
In one embodiment the framework of the Linker comprises C and H atoms and at least four heteroatoms.
In one embodiment the framework of the Linker comprises C and H atoms and at least four heteroatoms selected from N and O.
In one embodiment the framework of the Linker comprises C and H atoms and at least two N heteroatoms and at least two O heteroatoms.
In one embodiment the framework of the Linker includes from 1 to 10 heteroatoms.
In one embodiment the framework of the Linker includes from 2 to 10 heteroatoms.
In one embodiment the framework of the Linker includes from 4 to 10 heteroatoms.
In one embodiment the heteroatoms included in the framework of the Linker are selected from N and O only.
In one embodiment the Linker has a minimum length from 8 to 26 atoms between ‘a’ and ‘b’.
In one embodiment the total number of C and hetero atoms in the Linker framework is from 8 to 30.
In one embodiment the total number of C and hetero atoms in the Linker framework is from 10 to 28.
In one embodiment Linker is a saturated or a partially or fully unsaturated framework comprising C and H atoms and at least one heteroatom, wherein said framework has end points of attachment ‘a’ and ‘b’ (and where ‘b’ may involve two attachment points ‘b1’ and ‘b2’ in cases where there are two points of attachment to Z at the ‘b’ end of the Linker) and a minimum length of from 6 to 26 atoms between ‘a’ and ‘b’; wherein said framework consists of one or more straight and/or branched chains and/or rings and is optionally substituted on any available C atom(s) by one or more F; wherein said Linker is attached either:
   once to Z: at any available C or N atom of Z; or
   twice to Z: at any two adjacent available C atom(s) and/or N atom(s) at X^{H}, X^{G} & X^{J} (& X^{K} when present) such that a 5 to 7-membered ring is formed by the attachment of the Linker at the two adjacent atoms of Z;
In one embodiment the framework of the Linker may include (or consist of) one or more straight and/or branched chains and/or rings (wherein the total number of branches is from 0 to 5) that are optionally substituted on any available C atom(s) by one or more F.
In one embodiment the framework of the Linker may include (or consist of) one or more straight and/or branched chains and/or rings (wherein the total number of branches is from 0 to 3) that are optionally substituted on any available C atom(s) by one or more F.
In one embodiment the framework of the Linker may include (or consist of) one or more straight and/or branched chains and/or rings (wherein the total number of branches is 0 to 2) that are optionally substituted on any available C atom(s) by one or more F.
   In one embodiment the total number of branches is 2.
   In one embodiment the total number of branches is 2 and each branch consists of ═O.
In one embodiment any/each branch in the framework of a Linker has from 1 to 5 C and/or hetero atoms.
In one embodiment any/each branch in the framework of a Linker has from 1 to 3 C and/or hetero atoms.
In one embodiment any/each branch in the framework of a Linker has 1 C and/or hetero atom.
In one embodiment the total number of C and/or heteroatoms in the branch(es) (where present) of the framework of the Linker is from 1 to 5.
In one embodiment the total number of C and/or heteroatoms in the branch(es) (where present) of the framework of the Linker is from 1 to 3.
In one embodiment the total number of C and/or heteroatoms in a branch (where present) of the framework of the Linker is 1.
In one embodiment any/each hetero atom(s) present in the branch(es) of a Linker is/are 0 atom(s).
In one embodiment the framework of the Linker is optionally substituted on any available C atom(s) by 1 or 2 F.
In one embodiment the framework of the Linker is optionally substituted on any available C atom(s) by 1 F.
In one embodiment the framework of the Linker is not substituted by any F.
In one embodiment the Linker is a partially saturated framework comprising C and H atoms and at least one heteroatom, wherein said framework has end points of attachment ‘a’ and ‘b’; and
   a minimum length of from 8 to 24 atoms between ‘a’ and ‘b’;
   wherein the total number of C and hetero atoms in the Linker framework is from 10 to 26;
   where said framework comprises one or more straight and/or branched chains and/or rings (wherein the total number of branches is 0 to 2);
   wherein any branch in the framework of the Linker has 1 C and/or hetero atom;
   wherein said Linker is attached once to Z: at any available C or N atom of Z.
In one embodiment the Linker is selected from:

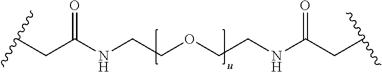

wherein u is 0 to 6.
In one embodiment u is 0.
In one embodiment u is 2.
In one embodiment u is 6.

In one aspect of the specification there is provided a PROTAC compound or a pharmaceutically acceptable salt thereof, containing an E3 ubiquitin ligase binding unit of Formula (Ia), where said PROTAC compound contains a unit of Formula (Ib):

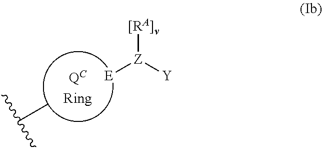

wherein:
Q^{C} Ring is a 4-11 membered saturated heterocyclic group;
E is linked to an available C or available N atom of Z, where
   when E is linked to an available C atom of Z, E is C or N, and
   when E is linked to an available N atom of Z, E is C;
      and where Z, Y, R^{A} and v may take any of the values described herein for each of these integers respectively.
In one embodiment E is N and is linked to an available C of Z.
In one embodiment E is C and is linked to an available C or N of Z.
In one embodiment E is C and is linked to an available N of Z.
In one embodiment E is C and is linked to an available C of Z.
In one embodiment Q^{C} Ring is a 6-membered saturated heterocyclic group.
In one embodiment Q^{C} Ring is piperazine or piperidine.
In one embodiment Q^{C} Ring is 1,4-piperazin-1,4-diyl or piperidin-1,4-diyl.
In one embodiment Q^{C} Ring is piperazine.
In one embodiment E is N and is linked to an available C of Z and Q^{C} Ring is piperazine.
In one embodiment Q^{C} Ring is piperidine.
In one embodiment E is C and Q^{C} Ring is piperidine.

In one aspect of the specification there is provided a PROTAC compound or a pharmaceutically acceptable salt thereof, containing an E3 ubiquitin ligase binding unit of Formula (Ia), where said PROTAC compound contains a unit of Formula (Ic):

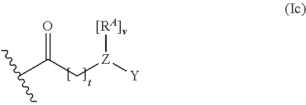

wherein:
t is 1 or 2, and Z, Y, R^{A} and v may take any of the values described herein for each of these variables respectively.
In one embodiment t is 1.
In one embodiment t is 2.

In further embodiments there is/are provided compounds(s) or a pharmaceutically acceptable salt thereof wherein said compound(s) is/are selected from one or more of the “Examples” listed hereinafter. It is to be understood that the Example relates to the title compound name, and is not limited in any way by the method of preparation nor whether a given compound was isolated in the form of a salt rather than as a neutral molecule.

The compounds of Formula (I) and PROTAC compounds containing binding units of Formula (Ia) may have one or more chiral centres and it will be recognised that such compounds may be prepared, isolated and/or supplied with or without the presence of one or more of the other possible enantiomeric and/or diastereomeric isomers of said compounds or that such isomers may be provided in any relative proportions. The preparation of enantioenriched/enantiopure and/or diastereoenriched/diastereopure compounds may be carried out by standard techniques of organic chemistry that are well known in the art, for example by synthesis from enantioenriched or enantiopure starting materials, and/or by use of an appropriately enantioenriched or enantiopure catalyst during synthesis, and/or by resolution of a racemic or partially enriched mixture of stereoisomers, for example via chiral chromatography.

For use in a pharmaceutical context it may be preferable to provide such compounds (or a pharmaceutically acceptable salt thereof) without large amounts of the other stereoisomeric forms being present.

Accordingly, in one embodiment there is provided a composition comprising a compound of Formula (I) [or PROTAC compound containing a unit of Formula (Ia)] or a pharmaceutically acceptable salt thereof, optionally together with one or more of the other stereoisomeric forms of the compound of Formula (I) [or PROTAC compound containing a unit of Formula (Ia)] or pharmaceutically acceptable salt thereof, wherein the compound of Formula (I) [or PROTAC compound containing a unit of Formula (Ia)] or pharmaceutically acceptable salt thereof is present within the composition with a diastereomeric excess (% de) of ≥90%.

In a further embodiment the % de in the above-mentioned composition is ≥95%.

In a further embodiment the % de in the above-mentioned composition is ≥98%.

In a further embodiment the % de in the above-mentioned composition is ≥99%.

In a further embodiment there is provided a composition comprising a compound of Formula (I) [or PROTAC compound containing a unit of Formula (Ia)] or a pharmaceutically acceptable salt thereof, optionally together with one or more of the other stereoisomeric forms of the compound of Formula (I) [or PROTAC compound containing a unit of Formula (Ia)] or pharmaceutically acceptable salt thereof, wherein the compound of Formula (I) [or PROTAC compound containing a unit of Formula (Ia)] or pharmaceutically acceptable salt thereof is present within the composition with an enantiomeric excess (% ee) of ≥90%.

In a further embodiment the % ee in the above-mentioned composition is ≥95%.

In a further embodiment the % ee in the above-mentioned composition is ≥98%.

In a further embodiment the % ee in the above-mentioned composition is ≥99%.

In a further embodiment there is provided a composition comprising a compound of Formula (I) [or PROTAC compound containing a unit of Formula (Ia)] or a pharmaceutically acceptable salt thereof, optionally together with one or more of the other stereoisomeric forms of the compound of Formula (I) [or PROTAC compound containing a unit of Formula (Ia)], or pharmaceutically acceptable salt thereof, wherein the compound of Formula (I) [or PROTAC compound containing a unit of Formula (Ia)], or pharmaceutically acceptable salt thereof is present within the composition with an enantiomeric excess (% ee) of ≥90% and a diastereomeric excess (% de) of ≥90%.

In further embodiments of the above-mentioned composition the % ee and % de may take any combination of values as listed below:
The % ee is ≤5% and the % de is ≥80%.
The % ee is ≤5% and the % de is ≥90%.
The % ee is ≤5% and the % de is ≥95%.
The % ee is ≤5% and the % de is ≥98%.
The % ee is ≤95% and the % de is ≥95%.
The % ee is ≤98% and the % de is ≥98%.
The % ee is ≤99% and the % de is ≥99%.

In a further embodiment there is provided a pharmaceutical composition which comprises a compound of the Formula (I) [or PROTAC compound containing a unit of Formula (Ia)], or a pharmaceutically acceptable salt thereof, in association with a pharmaceutically acceptable excipient.

In one embodiment there is provided a pharmaceutical composition which comprises a compound of the Formula (I) [or PROTAC compound containing a unit of Formula (Ia)], or a pharmaceutically acceptable salt thereof, in association with a pharmaceutically acceptable excipient, optionally further comprising one or more of the other stereoisomeric forms of the compound of Formula (I) [or PROTAC compound containing a unit of Formula (Ia)], or pharmaceutically acceptable salt thereof, wherein the compound of Formula (I) [or PROTAC compound containing a unit of Formula (Ia)], or pharmaceutically acceptable salt thereof is present within the composition with an enantiomeric excess (% ee) of ≥90%.

In a further embodiment the % ee in the above-mentioned composition is ≥95%.

In a further embodiment the % ee in the above-mentioned composition is ≥98%.

In a further embodiment the % ee in the above-mentioned composition is ≥99%.

In one embodiment there is provided a pharmaceutical composition which comprises a compound of the Formula (I) [or PROTAC compound containing a unit of Formula (Ia)], or a pharmaceutically acceptable salt thereof, in association with a pharmaceutically acceptable excipient, optionally further comprising one or more of the other stereoisomeric forms of the compound of Formula (I) [or PROTAC compound containing a unit of Formula (Ia)], or pharmaceutically acceptable salt thereof, wherein the compound of Formula (I) [or PROTAC compound containing a unit of Formula (Ia)], or pharmaceutically acceptable salt thereof is present within the composition with a diastereomeric excess (% de) of ≥90%.

In a further embodiment the % de in the above-mentioned composition is ≥95%.

In a further embodiment the % de in the above-mentioned composition is ≥98%.

In a further embodiment the % de in the above-mentioned composition is ≥99%.

In one embodiment there is provided a pharmaceutical composition which comprises a compound of the Formula (I) [or PROTAC compound containing a unit of Formula (Ia)], or a pharmaceutically acceptable salt thereof, in association with a pharmaceutically acceptable excipient, optionally further comprising one or more of the other stereoisomeric forms of the compound of Formula (I) [or PROTAC compound containing a unit of Formula (Ia)], or pharmaceutically acceptable salt thereof, wherein the compound of Formula (I) [or PROTAC compound containing a unit of Formula (Ia)], or pharmaceutically acceptable salt thereof is present within the composition with an enantiomeric excess (% ee) of ≥90% and a diastereomeric excess (% de) of ≥90%.

In further embodiments of the above-mentioned pharmaceutical composition the % ee and % de may take any combination of values as listed below:
The % ee is ≥95% and the % de is ≥95%.
The % ee is ≥98% and the % de is ≥98%.
The % ee is ≥99% and the % de is ≥99%.

The compounds of Formula (I) [or PROTAC compound containing a unit of Formula (Ia)], and pharmaceutically acceptable salts thereof may be prepared, used or supplied in amorphous form, crystalline form, or semicrystalline form and any given compound of Formula (I) [or PROTAC compound containing a unit of Formula (Ia)], or pharmaceutically acceptable salt thereof may be capable of being formed into more than one crystalline/polymorphic form, including hydrated (e.g. hemi-hydrate, a mono-hydrate, a di-hydrate, a tri-hydrate or other stoichiometry of hydrate) and/or solvated forms. It is to be understood that the present specification encompasses any and all such solid forms of the compound of Formula (I) [or PROTAC compound containing a unit of Formula (Ia)], and pharmaceutically acceptable salts thereof.

In further embodiments there is provided a compound of Formula (I) [or PROTAC compound containing a unit of Formula (Ia)] which is obtainable by the methods described in the ‘Examples’ section hereinafter.

### Intermediate Compounds

A PROTAC compound containing an E3 ubiquitin ligase binding unit of Formula (Ia) may be prepared from certain intermediate compounds, some of which are illustrated in the experimental section hereinafter. For example, such PROTAC compound may be prepared by an alkylation, reductive amination or amide coupling reaction using a compound of Formula (II):

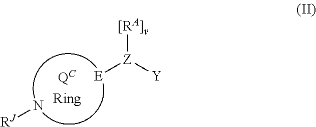

or a salt thereof, wherein:
R^{J} is H;
Q^{C} Ring is a 4-11 membered saturated heterocyclic group;
E is linked to an available C or available N atom of Z, where
   when E is linked to an available C atom of Z, E is C or N, and
   when E is linked to an available N atom of Z, E is C;
      and wherein Z, Y, R^{A} and v may take any of the values described herein for each of these integers respectively.

Such compound of Formula (II) may be coupled with a carboxylic acid using typical amide coupling conditions which are well known to the skilled person. For example, PyBOP or HATU may be used, together with a non-nucleophilic organic base such as DIPEA in a solvent such as DMF at r.t.

Such compound of Formula (II) may alternatively be subject to reductive amination conditions towards forming a PROTAC of this specification.

Such compound of Formula (II) may be alkylated using R-Hal, e.g. R—Cl, or using non-halogen leaving groups e.g. mesylate. Such alkylation coupling may be carried out using conditions well-known to the skilled person, using a non-nucleophilic base in a suitable solvent such as DMA.

Said compound of Formula (II) where R^{J} is H may in turn be prepared by deprotection of a compound of Formula (II) wherein R^{J} is a nitrogen protecting group, for example a tert-butoxycarbonyl (BOC) protecting group. Such a deprotection may be undertaken using acidic conditions which are well known to the skilled person, for example using the conditions exemplified in the experimental section hereinafter for such a deprotection.

Therefore in one aspect of the specification there is provided a compound of Formula (II), as shown above, or a salt thereof, wherein:
R^{J} is H or a N-protecting group;
Q^{C} Ring is a 4-11 membered saturated heterocyclic group;
E is linked to an available C or available N atom of Z, where
   when E is linked to an available C atom of Z, E is C or N, and
   when E is linked to an available N atom of Z, E is C;
      and wherein Z, Y, R^{A} and v may take any of the values described herein for each of these integers respectively.
In one embodiment R^{J} is H or tert-butoxycarbonyl.
In one embodiment R^{J} is H.
In one embodiment R^{J} is tert-butoxycarbonyl.
In one embodiment E is N and is linked to an available C of Z.
In one embodiment E is C and is linked to an available C or N of Z.
In one embodiment E is C and is linked to an available N of Z.
In one embodiment E is C and is linked to an available C of Z.
In one embodiment Q^{C} Ring is a 6-membered saturated heterocyclic group.
In one embodiment Q^{C} Ring is piperazine or piperidine.
In one embodiment E is N and is linked to an available C of Z, and Q^{C} Ring is piperazine.
In one embodiment E is C and is linked to an available C or N of Z, and Q^{C} Ring is piperidine.

Alternatively, for example, such PROTAC compound may be prepared by amide coupling reaction with a compound of Formula (III):

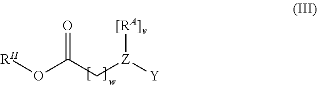

or a salt thereof, wherein w is 1 or 2, R^{H} is H; and where Z, Y, R^{A} and v may take any of the values described herein for each of these integers respectively. Such compound of Formula (III) may be coupled with a primary or secondary amine compound using typical amide coupling conditions which are well known to the skilled person. For example, PyBOP or HATU may be used, together with a non-nucleophilic organic base such as DIPEA in a solvent such as DMF at r.t. The compound of Formula (III) where R^{H} is H may be prepared in turn by hydrolysis of an ester compound of Formula (III), where R^{H} is C₁₋₈hydrocarbyl, for example C₁₋₆alkyl. Such hydrolysis may be carried out using a metal hydroxide salt, for example LiOH in a polar solvent, using conditions shown hereinafter in the experimental section or which are otherwise erll-known to the skilled person.

Therefore, in one aspect of the specification there is provided a compound of Formula (III), as shown above, or a salt thereof, wherein:
w is 1 or 2;
R^{H} is H or C₁₋₈hydrocarbyl; and where
Z, Y, R^{A} and v may take any of the values described herein for each of these integers respectively.
In one embodiment w is 1.
In one embodiment w is 2.
In one embodiment R^{H} is H.
In one embodiment R^{H} is C₁₋₈hydrocarbyl.
In one embodiment R^{H} is H or C₁₋₆alkyl.
In one embodiment R^{H} is C₁-6alkyl.
In one embodiment R^{H} is H or C₁₋₃alkyl.
In one embodiment R^{H} is C₁-3alkyl.
In one embodiment R^{H} is H or methyl.
In one embodiment R^{H} is methyl.
In one embodiment the compound of Formula (III) is other than 3-[6-(2,4-dioxohexahydropyrimidin-1-yl)-2-oxo-1,3-benzoxazol-3-yl]propanoic acid.

In addition to the methods described above, the compounds of Formulae (I), (II) and (III) may be prepared according to the general procedures and chemical transformations demonstrated in the experimental section hereinafter and using standard procedures and knowledge known to the skilled chemist.

In further embodiments of this specification there is/are provided compound(s), or a salt thereof, wherein said compound(s) is/are selected from one or more of the “Intermediates” listed hereinafter in the experimental section.

It is to be understood that the compound of an Intermediate listed hereinafter relates to the title chemical name listed in the experimental section, and is not limited in any way by the method of preparation nor whether a given intermediate compound was isolated in the form of a salt rather than as a neutral molecule.

According to a further aspect of the specification there is provided a pharmaceutical composition, which comprises a compound of Formula (I), or a pharmaceutically acceptable salt thereof, as defined herein, in association with a pharmaceutically acceptable excipient.

According to a further aspect of the specification there is provided a pharmaceutical composition which comprises a PROTAC compound containing an E3 ubiquitin ligase binding unit of Formula (Ia), or a pharmaceutically acceptable salt thereof, as defined herein, in association with a pharmaceutically acceptable excipient.

According to a further aspect of the specification there is provided a pharmaceutical composition, which comprises a compound of Formula (I), or a pharmaceutically acceptable salt thereof, as defined herein, for use in the treatment of cancer.

According to a further aspect of the specification there is provided a pharmaceutical composition, which comprises a PROTAC compound containing an E3 ubiquitin ligase binding unit of Formula (Ia), or a pharmaceutically acceptable salt thereof, as defined herein, for use in the treatment of cancer.

According to a further aspect of the specification there is provided a pharmaceutical composition, which comprises a compound of Formula (I), or a pharmaceutically acceptable salt thereof, as defined herein, for use in the treatment of a solid tumour.

According to a further aspect of the specification there is provided a pharmaceutical composition, which comprises a PROTAC compound containing an E3 ubiquitin ligase binding unit of Formula (Ia), or a pharmaceutically acceptable salt thereof, as defined herein, for use in the treatment of a solid tumour.

According to a further aspect of the specification there is provided a pharmaceutical composition, which comprises a compound of Formula (I), or a pharmaceutically acceptable salt thereof, as defined herein, for use in the treatment of a BRD4-sensitive tumour type.

The compositions may be in a form suitable for oral use (for example as tablets, lozenges, hard or soft capsules, aqueous or oily suspensions, emulsions, dispersible powders or granules, syrups or elixirs) or for parenteral administration (for example as a sterile aqueous or oily solution for intravenous, subcutaneous or intramuscular dosing). The compositions may be obtained by conventional procedures using conventional pharmaceutical excipients that are well known in the art. Thus, compositions intended for oral use may contain, for example, one or more colouring, sweetening, flavouring and/or preservative agents.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host being treated and the particular route of administration.

The size of the dose for therapeutic or prophylactic purposes of compounds of the present specification will naturally vary according to the nature and severity of the disease state, the age and sex of the animal or patient and the route of administration, according to well known principles of medicine.

According to a further aspect of the specification, there is provided a compound of Formula (I), or a pharmaceutically acceptable salt thereof, as defined herein, for use as a medicament.

According to a further aspect of the specification, there is provided a PROTAC compound containing an E3 ubiquitin ligase binding unit of Formula (Ia), or a pharmaceutically acceptable salt thereof, as defined herein, for use as a medicament.

According to a further aspect of the specification, there is provided a compound of Formula (I), or a pharmaceutically acceptable salt thereof, as defined herein, for use in therapy.

According to a further aspect of the specification, there is provided a PROTAC compound containing an E3 ubiquitin ligase binding unit of Formula (Ia), or a pharmaceutically acceptable salt thereof, as defined herein, for use in therapy.

According to a further aspect of the specification, there is provided a compound of Formula (I), or a pharmaceutically acceptable salt thereof, as defined herein, for use in a method of treatment of the human or animal body by therapy.

According to a further aspect of the specification, there is provided a PROTAC compound containing an E3 ubiquitin ligase binding unit of Formula (Ia), or a pharmaceutically acceptable salt thereof, as defined herein, for use in a method of treatment of the human or animal body by therapy.

According to a further aspect of the specification, there is provided a compound of Formula (I), or a pharmaceutically acceptable salt thereof, as defined herein for use in the production of an anti-proliferative effect (for example, in a warm-blooded animal such as man).

According to a further aspect of the specification, there is provided a PROTAC compound containing an E3 ubiquitin ligase binding unit of Formula (Ia), or a pharmaceutically acceptable salt thereof, as defined herein, for use in the production of an anti-proliferative effect (for example, in a warm-blooded animal such as man).

According to a further aspect of the specification, there is provided a compound of Formula (I), or a pharmaceutically acceptable salt thereof, as defined herein for use in the production of a protein degrading effect in a warm-blooded animal such as man.

According to a further aspect of the specification, there is provided a PROTAC compound containing an E3 ubiquitin ligase binding unit of Formula (Ia), or a pharmaceutically acceptable salt thereof, as defined herein, for use in the production of a protein degrading effect in a warm-blooded animal such as man.

According to a further aspect of the specification, there is provided the use of a compound of Formula (I), or a pharmaceutically acceptable salt thereof, as defined herein, for the manufacture of a medicament for the production of an anti-proliferative effect (for example, in a warm-blooded animal such as man).

According to a further aspect of the specification, there is provided the use of a PROTAC compound containing an E3 ubiquitin ligase binding unit of Formula (Ia), or a pharmaceutically acceptable salt thereof, as defined herein, for the manufacture of a medicament for the production of an anti-proliferative effect (for example, in a warm-blooded animal such as man).

According to a further aspect of the specification, there is provided the use of a PROTAC compound containing an E3 ubiquitin ligase binding unit of Formula (Ia), or a pharmaceutically acceptable salt thereof, as defined herein, for the manufacture of a medicament for the production of a protein degrading effect (for example, in a warm-blooded animal such as man).

According to a further aspect of the specification, there is provided a method for producing an anti-proliferative effect in a warm-blooded animal, such as man, in need of such effect, which comprises administering to said animal an effective amount of a compound of Formula (I), or a pharmaceutically acceptable salt thereof, as defined herein.

According to a further aspect of the specification, there is provided a method for producing an anti-proliferative effect in a warm-blooded animal, such as man, in need of such effect, which comprises administering to said animal an effective amount of a PROTAC compound containing an E3 ubiquitin ligase binding unit of Formula (Ia), or a pharmaceutically acceptable salt thereof, as defined herein.

According to a further aspect of the specification, there is provided a method for producing a protein degrading effect in a warm-blooded animal, such as man, in need of such effect, which comprises administering to said animal an effective amount of a PROTAC compound containing an E3 ubiquitin ligase binding unit of Formula (Ia), or a pharmaceutically acceptable salt thereof.

According to a further aspect of the specification, there is provided a compound of Formula (I), or a pharmaceutically acceptable salt thereof, as defined herein, for use as an anti-invasive agent in the containment and/or treatment of solid tumour disease (for example: in a warm-blooded animal such as man).

According to a further aspect of the specification, there is provided a PROTAC compound containing an E3 ubiquitin ligase binding unit of Formula (Ia), or a pharmaceutically acceptable salt thereof, as defined herein, for use as an anti-invasive agent in the containment and/or treatment of solid tumour disease (for example: in a warm-blooded animal such as man).

According to a further aspect of the specification, there is provided the use of a compound of Formula (I), or a pharmaceutically acceptable salt thereof, as defined herein, for the manufacture of a medicament for use as an anti-invasive agent in the containment and/or treatment of solid tumour disease (for example: in a warm-blooded animal such as man).

According to a further aspect of the specification, there is provided the use of a PROTAC compound containing an E3 ubiquitin ligase binding unit of Formula (Ia), or a pharmaceutically acceptable salt thereof, as defined herein, for the manufacture of a medicament for use as an anti-invasive agent in the containment and/or treatment of solid tumour disease (for example: in a warm-blooded animal such as man).

According to a further aspect of the specification, there is provided a method for producing an anti-invasive effect by the containment and/or treatment of solid tumour disease, in a warm-blooded animal, such as man, in need of such effect, which comprises administering to said animal an effective amount of a compound of Formula (I), or a pharmaceutically acceptable salt thereof, as defined herein.

According to a further aspect of the specification, there is provided a method for producing an anti-invasive effect by the containment and/or treatment of solid tumour disease, in a warm-blooded animal, such as man, in need of such effect, which comprises administering to said animal an effective amount of a PROTAC compound containing an E3 ubiquitin ligase binding unit of Formula (Ia), or a pharmaceutically acceptable salt thereof, as defined herein.

According to a further aspect of the specification, there is provided a compound of Formula (I), or a pharmaceutically acceptable salt thereof, as defined herein, for use in the prevention or treatment of cancer (for example: in a warm-blooded animal such as man).

According to a further aspect of the specification, there is provided a PROTAC compound containing an E3 ubiquitin ligase binding unit of Formula (Ia), or a pharmaceutically acceptable salt thereof, as defined herein, for use in the prevention or treatment of cancer (for example: in a warm-blooded animal such as man).

According to a further aspect of the specification there is provided the use of a compound of Formula (I), or a pharmaceutically acceptable salt thereof, as defined herein, for the manufacture of a medicament for the prevention or treatment of cancer (for example: in a warm-blooded animal such as man).

According to a further aspect of the specification there is provided the use of a PROTAC compound containing an E3 ubiquitin ligase binding unit of Formula (Ia), or a pharmaceutically acceptable salt thereof, as defined herein, for the manufacture of a medicament for the prevention or treatment of cancer (for example: in a warm-blooded animal such as man).

According to a further aspect of the specification there is provided a method for the prevention or treatment of cancer in a warm-blooded animal, such as man, in need of such treatment, which comprises administering to said animal an effective amount of a compound of Formula (I), or a pharmaceutically acceptable salt thereof, as defined herein.

According to a further aspect of the specification there is provided a method for the prevention or treatment of cancer in a warm-blooded animal, such as man, in need of such treatment, which comprises administering to said animal an effective amount of a PROTAC compound containing an E3 ubiquitin ligase binding unit of Formula (Ia), or a pharmaceutically acceptable salt thereof, as defined herein.

According to a further aspect of the specification, there is provided a compound of Formula (I), or a pharmaceutically acceptable salt thereof, as defined herein, for use in the prevention or treatment of solid tumour(s) (for example, in a warm-blooded animal such as man).

According to a further aspect of the specification, there is provided a PROTAC compound containing an E3 ubiquitin ligase binding unit of Formula (Ia), or a pharmaceutically acceptable salt thereof, as defined herein, for use in the prevention or treatment of solid tumour(s) (for example, in a warm-blooded animal such as man).

According to a further aspect of the specification, there is provided the use of a compound of Formula (I), or a pharmaceutically acceptable salt thereof, as defined herein, for the manufacture of a medicament for the prevention or treatment of solid tumour(s) (for example, in a warm-blooded animal such as man).

According to a further aspect of the specification, there is provided the use of a PROTAC compound containing an E3 ubiquitin ligase binding unit of Formula (Ia), or a pharmaceutically acceptable salt thereof, as defined herein, for the manufacture of a medicament for the prevention or treatment of solid tumour(s) (for example, in a warm-blooded animal such as man).

According to a further aspect of the specification, there is provided a method for the prevention or treatment of solid tumour(s) in a warm-blooded animal, such as man, in need of such treatment, which comprises administering to said animal an effective amount of a compound of Formula (I), or a pharmaceutically acceptable salt thereof, as defined herein.

According to a further aspect of the specification, there is provided a method for the prevention or treatment of solid tumour(s) in a warm-blooded animal, such as man, in need of such treatment, which comprises administering to said animal an effective amount of a PROTAC compound containing an E3 ubiquitin ligase binding unit of Formula (Ia), or a pharmaceutically acceptable salt thereof, as defined herein.

According to a further aspect of the specification, there is provided a compound of Formula (I), or a pharmaceutically acceptable salt thereof, as defined herein, for use in the prevention or treatment of tumour types that are sensitive to inhibition and/or degradation of BRD4.

According to a further aspect of the specification, there is provided the use of a compound of Formula (I), or a pharmaceutically acceptable salt thereof, as defined herein, for the manufacture of a medicament for the prevention or treatment of those tumour types that are sensitive to inhibition and/or degradation of BRD4.

According to a further aspect of the specification, there is provided a method for the prevention or treatment of those tumour types that are sensitive to inhibition and/or degradation of BRD4, in a warm-blooded animal, such as man, in need of such treatment, which comprises administering to said animal an effective amount of a compound of Formula (I), or a pharmaceutically acceptable salt thereof, as defined herein.

According to a further aspect of the specification, there is provided a compound of Formula (I), or a pharmaceutically acceptable salt thereof, as defined herein, for use in providing an inhibitory and/or degrading effect on BRD4.

According to a further aspect of the specification, there is provided the use of a compound of Formula (I), or a pharmaceutically acceptable salt thereof, as defined herein, for the manufacture of a medicament for providing an inhibitory and/or degrading effect on BRD4.

According to a further aspect of the specification, there is provided a method for providing an inhibitory and/degrading effect on BRD4 in a warm-blooded animal, such as man, in need of such effect, which comprises administering to said animal an effective amount of a compound of Formula (I), or a pharmaceutically acceptable salt thereof, as defined herein.

According to a further aspect of the specification, there is provided a compound of Formula (I), or a pharmaceutically acceptable salt thereof, as defined herein, for use in providing a selective inhibitory and/degrading effect on BRD4.

According to a further aspect of the specification, there is provided the use of a compound of Formula (I), or a pharmaceutically acceptable salt thereof, as defined herein, for the manufacture of a medicament for providing a selective inhibitory and/or degrading effect on BRD4.

According to a further aspect of the specification, there is provided a method for providing a selective inhibitory and/degrading effect on BRD4 in a warm-blooded animal, such as man, in need of such effect, which comprises administering an effective amount of a compound of Formula (I), or a pharmaceutically acceptable salt thereof, as defined herein.

The anti-cancer treatment defined herein may be applied as a sole therapy or may involve, in addition to the compounds of the specification, conventional surgery or radiotherapy or chemotherapy.

Combination therapy as described above may be added on top of standard of care therapy typically carried out according to its usual prescribing schedule.

Although the compounds of Formula (I) are primarily of value as therapeutic agents for use in warm-blooded animals (including man), they are also useful whenever it is required to inhibit and/or degrade BRD4. Thus, they are useful as pharmacological standards for use in the development of new biological tests and in the search for new pharmacological agents.

### Chemical Synthesis and Biological Assay Procedures:

### General Abbreviations

The following abbreviations are used: Ac=acetyl; AcOH=acetic acid; Ac₂O=acetic anhydride; Boc=tert-butyloxycarbonyl; C-18FC=C-18 flash chromatography; CDI=carbonyl diimidazole; dba=dibenzylideneacetone; DBU=1,8-diazabicyclo[5.4.0]undec-7-ene; DCE=1,2-dichloroethane; DCM=dichloromethane; DIAD=diisopropyl azodicarboxylate; DIEA=N,N-Diisopropylethylamine; DMAP=4-(dimethylamino)pyridine; DMF=N,N-dimethylformamide; DMSO=dimethylsulfoxide; EDC=1-ethyl-3-(3-dimethylaminopropyl)carbodiimide; Ephos=dicyclohexyl(3-isopropoxy-2′,4′,6′-triisopropyl-[1,1′-biphenyl]-2-yl)phosphane; “Ephos Pd G4”=dicyclohexyl-[2-propan-2-yloxy-6-[2,4,6-tri(propan-2-yl)phenyl]phenyl]phosphanium; methanesulfonic acid; methyl-(2-phenylphenyl)azanide; palladium(2+) (CAS number: 2132978-44-8); ES=electrospray (in the context of mass spectrometry); EtOAc=ethyl acetate; FA=formic acid; FSC=flash silica chromatography; h=hour(s); HOBt=hydroxybenzotriazole; HPLC=high performance liquid chromatography; m/z=mass-to-charge ratio in connection with mass spectrometry analysis; NCS=N-chlorosuccinimide; NMP=N-methyl-2-pyrrolidone; NMR=nuclear magnetic resonance; Petroleum ether=distilled petroleum of fraction 60-90° C.; Ph=phenyl; PMB=p-methoxybenzyl; PPA=polyphosphoric acid; PyBOP=(benzotriazol-1-yl-oxytripyrrolidinophosphonium hexafluorophosphate; Rochelle's salt=monopotassium monosodium L(+)-tartrate tetrahydrate; r.t.=room temperature (˜18-25° C.); selectfluor=1-chloromethyl-4-fluoro-1,4-diazoniabicyclo[2.2.2]octane bis(tetrafluoroborate); SEM=2-(trimethylethylsilyl)ethoxymethyl; THF=tetrahydrofuran; TFA=trifluoroacetic acid; TMEDA=tetramethylethylenediamine; xantphos=4,5-bis(diphenylphosphino)-9,9-dimethylxanthene.

NMR: Proton NMR (¹H NMR) was carried out at 300 or 400 MHz at a temperature in the range from 15-30° C. in deuterated-DMSO unless otherwise specified. ¹⁹F NMR was carried out in deuterated DMSO unless otherwise stated. Where evident, ¹H NMR (for hydrochloride, formate, 2,2,2-trifluoroacetate salts) and/or ¹⁹F NMR (for 2,2,2-trifluoroacetate salts) peaks of salts were included in the characterisation. In certain instances, exchangeable protons were either too broad or not evident in the spectrum and were therefore not reported in the characterisation. Standard NMR abbreviations are used: s=single, d=doublet, t=triplet, q=quartet, dd=doublet of doublets, dt doublet of triplets, m=multiplet, br=broad.
Preparative HPLC was carried out using one of the following Columns and Eluents, unless otherwise specified:
Column A: Waters XBridge Shield RP18 OBD Column, 30*150 mm, 5 μm
Column B: Waters XBridge Prep OBD C18 Column, 30*150 mm, 5 μm
Column C: Waters XBridge Shield RP18 OBD Column, 19*250 mm, 10 μm
Column D: Waters XSelect CSH Prep C18 OBD Column, 19*250 mm, 5 μm
Column E: Waters Sunfire prep C18 column, 30*150 mm, 5 μm
Column F: Waters SunFire C18 OBD Prep Column, 100 Å, 19*250 mm, 5 μm
Column G: Waters Xselect CSH OBD Column, 30*150 mm, 5 μm
Column H: Waters Sunfire prep C18 OBD column, 19*250 mm, 10 μm
Column J: Waters XBridge prep OBD C18 column, 19*250 mm, 5 μm
Column K: Waters Atlantis prep T3 OBD column 19*250 mm, 10 μm
Column L: Waters XSelect CSH Fluoro Phenyl 30*150 mm, 5 μm
Column X: Waters XSelect CSH F-Phenyl OBD column, 19*250 mm, 5 μm
Column Y: Waters XBridge BEH C18 OBD prep column, 19*250 mm, 5 μm
Column Z: Waters XBridge Phenyl OBD prep column, 19*250 mm, 5 μm.
Eluent A: decreasingly polar mixture of water (with 0.05% TFA) and MeCN
Eluent B: decreasingly polar mixture of water (with 10 mmol/L NH₄HCO₃+0.1% NH₃·H₂O) and MeCN
Eluent C: decreasingly polar mixture of water (with 0.05% TFA) and MeOH
Eluent D: decreasingly polar mixture of water (with 10 mmol/L NH₄HCO₃) and MeCN
Eluent E: decreasingly polar mixture of water (with 0.1% FA) and MeCN
Eluent F: decreasingly polar mixture of water (with 0.05% NH₄OH) and MeCN
Eluent G: decreasingly polar mixture of water (with 10 mmol/L NH₄HCO₃+0.1% NH₃·H₂O) and (MeOH-MeCN 1:2)
   Solvent removal: concentration of solutions (to partly or fully remove solvent) are generally performed under reduced pressure at r.t. or above.
   Chromatography methods: clean-appearing fractions containing the desired product are generally identified and combined together and then concentrated under reduced pressure.

### Example 1: 1-(Benzofuran-6-yl)dihydropyrimidine-2,4 (1H,3H)-dione

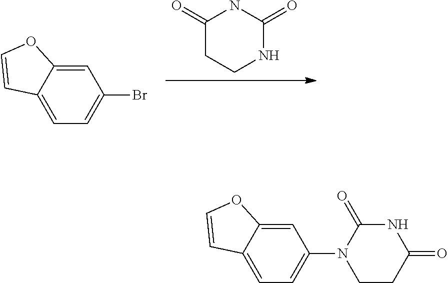 [SMILES: Brc1ccc2ccoc2c1 ; O=C1CCNC(=O)N1 ; O=C1CCN(c2ccc3ccoc3c2)C(=O)N1]

Cs₂CO₃ (471 mg, 1.45 mmol) was added to a degassed mixture of 6-bromobenzofuran (95.0 mg, 0.482 mmol), dihydropyrimidine-2,4 (1H,3H)-dione (165 mg, 1.45 mmol), Ephos (12.9 mg, 0.0241 mmol) and Ephos Pd G4 (22.1 mg, 0.0241 mmol) in 1,4-dioxane (5 mL) at r.t. under N₂. The resulting mixture was stirred at 100° C. for 20 h. The crude product was directly purified by C-18FC (gradient: 25-60% MeCN in water) to give the title compound (53.1 mg, 48%) as a white solid. ¹H NMR: δ 2.74 (2H, t), 3.84 (2H, t), 6.98 (1H, dd), 7.25 (1H, dd), 7.63 (2H, m), 8.03 (1H, d), 10.40 (1H, s). m/z (ES⁺), [M+H]⁺=231.0.

### Example 2: 1-(Benzo[d]thiazol-7-yl)dihydropyrimidine-2,4 (1H,3H)-dione

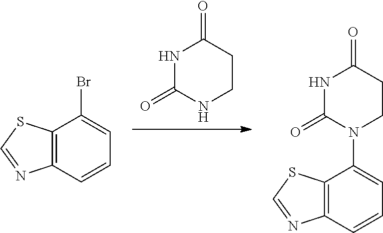

7-Bromobenzo[d]thiazole (100 mg, 0.467 mmol) was added to a degassed mixture of dihydropyrimidine-2,4 (1H,3H)-dione (213 mg, 1.87 mmol), Ephos Pd G4 (42.9 mg, 0.0467 mmol) and Ephos (25.0 mg, 0.0467 mmol) with Cs₂CO₃ (457 mg, 1.40 mmol) in 1,4-dioxane (10 mL) at r.t. under N₂. The resulting mixture was stirred at 100° C. for 16 h. The solvent was then removed under reduced pressure. Purification by C-18FC (gradient: 0-20% MeCN in water) gave material that was further purified by preparative HPLC (Column A, Eluent A, gradient: 4-14%) to give the title compound (29.0 mg, 25%) as a white solid. ¹H NMR: (CD₃OD) δ 2.91 (2H, t), 3.98 (2H, t), 7.51-7.57 (1H, m), 7.67 (1H, t), 8.09 (1H, dd), 9.29 (1H, s). m/z (ES⁺), [M+H]⁺=248.0.

### Example 3: 1-(Pyrazolo[1,5-a]pyridin-6-yl)dihydropyrimidine-2,4 (1H,3H)-dione

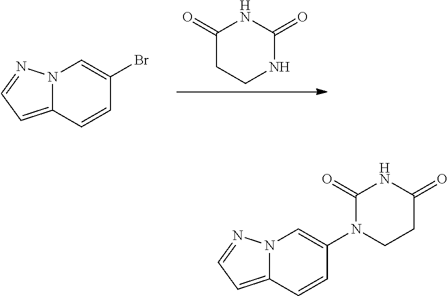 [SMILES: Brc1ccc2ccnn2c1 ; O=C1CCNC(=O)N1 ; O=C1CCN(c2ccc3ccnn3c2)C(=O)N1]

A degassed mixture of Cs₂CO₃ (496 mg, 1.52 mmol), 6-bromopyrazolo[1,5-a]pyridine (100 mg, 0.508 mmol), dihydropyrimidine-2,4 (1H,3H)-dione (174 mg, 1.52 mmol), Ephos (13.6 mg, 0.0254 mmol) and Ephos Pd G4 (23.3 mg, 0.025 mmol) in 1,4-dioxane (10 mL) was stirred at 120° C. under N₂ for 20 h. The resulting mixture was filtered and the solid was washed with 1,4-dioxane. The solvents of the filtrate were removed under reduced pressure. The residue was purified by C-18FC (gradient: 0-30% MeCN in water) and then further purified by preparative HPLC (Column B, Eluent B, gradient: 2-25%) to give the title compound (12.4 mg, 11%) as a white solid. ¹H NMR: δ 2.75 (t, 2H), 3.82 (t, 2H), 6.64 (dd, 1H), 7.26 (dd, 1H), 7.70 (dd, 1H), 8.02 (d, 1H), 8.80 (dt, 1H), 10.49 (s, 1H). m/z (ES⁺), [M+H]⁺=231.0.

### Example 4: 1-(Benzo[d]oxazol-7-yl)dihydropyrimidine-2,4 (1H,3H)-dione

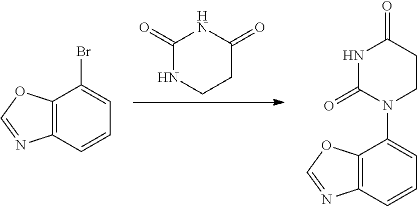 [SMILES: Brc1cccc2ncoc12 ; O=C1CCNC(=O)N1 ; O=C1CCN(c2cccc3ncoc23)C(=O)N1]

Ephos Pd G4 (46.4 mg, 0.0505 mmol) was added to a degassed mixture of Ephos (27.0 mg, 0.0505 mmol), Cs₂CO₃ (494 mg, 1.52 mmol), 7-bromobenzo[d]oxazole (100 mg, 0.505 mmol) and dihydropyrimidine-2,4 (1H,3H)-dione (230 mg, 2.02 mmol) in 1,4-dioxane (12 mL) at r.t. under N₂. The resulting mixture was stirred at 100° C. for 16 h. The solvent was then removed under reduced pressure. Purification by FSC (gradient 0-4% MeOH in DCM) gave the title compound (40.0 mg, 34%) as a pale yellow solid. ¹H NMR: δ 10.60 (s, 1H), 8.79 (s, 1H), 7.78-7.63 (m, 1H), 7.44 (d, 2H), 3.91 (t, 2H), 2.79 (t, 2H). m/z (ES⁺), [M+H]⁺=232.1.

### Example 5: 1-(Benzo[d]oxazol-6-yl)dihydropyrimidine-2,4 (1H,3H)-dione

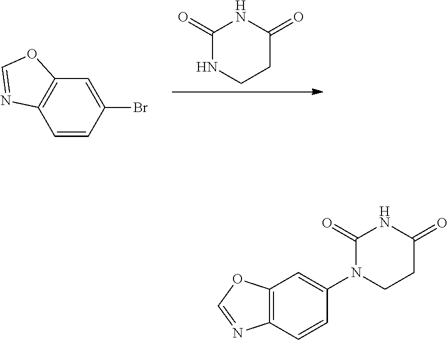 [SMILES: Brc1ccc2ncoc2c1 ; O=C1CCNC(=O)N1 ; O=C1CCN(c2ccc3ncoc3c2)C(=O)N1]

Cs₂CO₃ (494 mg, 1.52 mmol) was added to a degassed mixture of Ephos Pd G4 (46.4 mg, 0.0505 mmol), Ephos (27.0 mg, 0.0505 mmol), dihydropyrimidine-2,4 (1H,3H)-dione (173 mg, 1.52 mmol) and 6-bromobenzo-[d]oxazole (100 mg, 0.505 mmol) in 1,4-dioxane (8 mL) at r.t. under N₂. The resulting mixture was stirred at 100° C. for 17 h. The solvent was then removed under reduced pressure. Purification by FSC (gradient 0-70% EtOAc in petroleum ether) gave material that was further purified by FSC (gradient 0-10% MeOH in DCM) to give the title compound (26.0 mg, 22%) as a yellow solid. ¹H NMR: δ 2.75 (2H, t), 3.85 (2H, t), 7.39 (1H, dd), 7.76-7.85 (2H, m), 8.77 (1H, s), 10.45 (1H, s). m/z (ES⁺), [M+H]⁺=232.1.

### Example 6: 1-(1-Methyl-1H-indazol-5-yl)dihydropyrimidine-2,4 (1H,3H)-dione

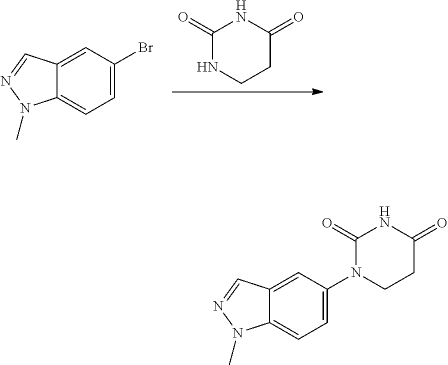 [SMILES: Cn1ncc2cc(Br)ccc21 ; O=C1CCNC(=O)N1 ; Cn1ncc2cc(N3CCC(=O)NC3=O)ccc21]

A degassed mixture of Cs₂CO₃ (509 mg, 1.56 mmol), 5-bromo-1-methyl-1H-indazole (110 mg, 0.52 mmol), dihydropyrimidine-2,4 (1H,3H)-dione (178 mg, 1.56 mmol), Ephos (13.9 mg, 0.026 mmol) and Ephos Pd G4 (23.9 mg, 0.026 mmol) in 1,4-dioxane (10 mL) was stirred at 100° C. for 14 h. The resulting mixture was filtered, washed with THF and the solvents of the filtrate were removed under reduced pressure. Purification by C-18FC (gradient: 0-100% MeCN in water) gave the title compound (75.0 mg, 59%) as a white solid. ¹H NMR: 8.05 (d, 1H), 7.71-7.60 (m, 2H), 7.37 (dd, 1H), 4.05 (s, 3H), 3.80 (t, 2H), 2.73 (t, 2H). m/z (ES⁺), [M+H]⁺=245.2.

### Example 7: 1-(Imidazo[1,2-a]pyridin-7-yl)dihydropyrimidine-2,4 (1H,3H)-dione

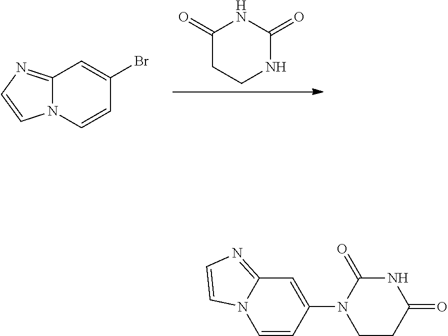 [SMILES: Brc1ccn2ccnc2c1 ; O=C1CCNC(=O)N1 ; O=C1CCN(c2ccn3ccnc3c2)C(=O)N1]

A degassed mixture of Ephos (13.6 mg, 0.0254 mmol), Ephos Pd G4 (23.3 mg, 0.0254 mmol), Cs₂CO₃ (496 mg, 1.52 mmol), 7-bromoimidazo[1,2-a]pyridine (100 mg, 0.51 mmol) and dihydropyrimidine-2,4 (1H,3H)-dione (174 mg, 1.52 mmol) in 1,4-dioxane (10 mL) was stirred at 120° C. for 15 h. The resulting mixture was filtered, washed with 1,4-dioxane and the solvents of the filtrate were removed under reduced pressure. The residue was purified by C-18FC (gradient: 0-45% MeCN in water) and further purified by preparative HPLC (Column B, Eluent B, gradient: 2-25%) to give the title compound (27.2 mg, 23%) as a white solid. ¹H NMR: δ 8.50 (dd, 1H), 7.91 (t, 1H), 7.55 (d, 1H), 7.48-7.43 (m, 1H), 6.99 (dd, 1H), 6.05 (s, 1H), 3.88 (t, 2H), 2.73 (t, 2H). m/z (ES⁺), [M+H]⁺=231.2.

### Example 8: 1-(2-Methyl-2H-indazol-5-yl)dihydropyrimidine-2,4 (1H,3H)-dione

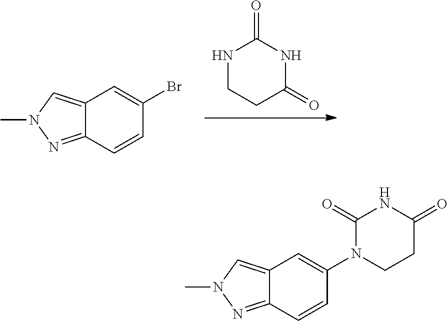 [SMILES: Cn1cc2cc(Br)ccc2n1 ; O=C1CCNC(=O)N1 ; Cn1cc2cc(N3CCC(=O)NC3=O)ccc2n1]

Ephos Pd G4 (43.5 mg, 0.0474 mmol) was added to a degassed mixture of 5-bromo-2-methyl-2H-indazole (100 mg, 0.474 mmol), dihydropyrimidine-2,4 (1H,3H)-dione (81.0 mg, 0.710 mmol), Ephos (25.3 mg, 0.0473 mmol) and Cs₂CO₃ (463 mg, 1.42 mmol) in 1,4-dioxane (5 mL) at r.t. under N₂. The resulting mixture was stirred at 120° C. for 16 h. The solvent was then removed under reduced pressure. Purification by C-18FC (gradient: 5-60% MeOH in water (containing 0.1% FA) gave the title compound (16.0 mg, 14%) as a white solid. ¹H NMR: δ 10.32 (s, 1H), 8.34 (s, 1H), 7.63-7.55 (m, 2H), 7.20 (dd, 1H), 4.17 (s, 3H), 3.80 (t, 2H), 2.73 (t, 2H). m/z (ES⁺), [M+H]⁺=245.2.

### Example 9: 1-(Benzo[d]thiazol-6-yl)dihydropyrimidine-2,4 (1H,3H)-dione

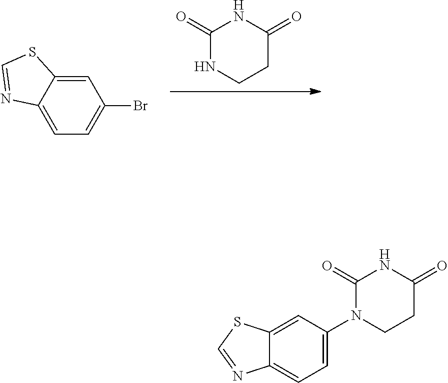 [SMILES: Brc1ccc2ncsc2c1 ; O=C1CCNC(=O)N1 ; O=C1CCN(c2ccc3ncsc3c2)C(=O)N1]

Cs₂CO₃ (470 mg, 1.44 mmol) was added to a degassed mixture of 6-bromobenzo[d]thiazole (103 mg, 0.48 mmol), dihydropyrimidine-2,4 (1H,3H)-dione (165 mg, 1.44 mmol), Ephos (12.9 mg, 0.0241 mmol) and Ephos Pd G4 (22.1 mg, 0.0241 mmol) in 1,4-dioxane (5 mL) at r.t. under N₂. The resulting mixture was stirred at 100° C. for 20 h. The solvent was then removed under reduced pressure. Purification by C-18FC (gradient: 10-30% MeCN in water) gave the title compound (50.0 mg, 42%) as a white solid. ¹H NMR: 2.76 (2H, t), 3.88 (2H, t), 7.54 (1H, dd), 8.12 (2H, m), 9.40 (1H, s), 10.47 (1H, s). m/z (ES⁺), [M+H]⁺=248.0.

### Example 10: 1-(2-Methyl-3-oxoisoindolin-5-yl)dihydropyrimidine-2,4 (1H,3H)-dione

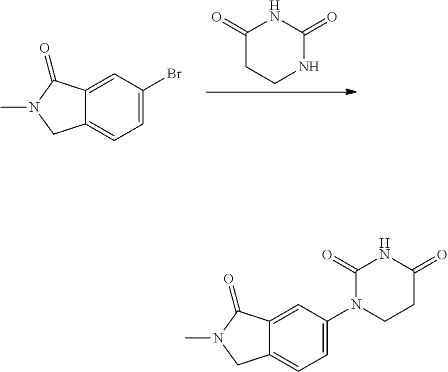 [SMILES: CN1Cc2ccc(Br)cc2C1=O ; O=C1CCNC(=O)N1 ; CN1Cc2ccc(N3CCC(=O)NC3=O)cc2C1=O]

Cs₂CO₃ (216 mg, 0.663 mmol) was added to a degassed mixture of dihydropyrimidine-2,4 (1H,3H)-dione (76.0 mg, 0.666 mmol), 6-bromo-2-methylisoindolin-1-one (50.0 mg, 0.221 mmol), Ephos (11.8 mg, 0.0221 mmol) and Ephos Pd G4 (20.3 mg, 0.0221 mmol) in 1,4-dioxane (5 mL) at r.t. under N₂. The resulting suspension was stirred at 100° C. for 16 h. The solid was filtered and washed with DMF. The filtrate was concentrated under reduced pressure. Purification by C-18FC (gradient: 0-80% MeCN in water) gave the title compound (31.0 mg, 54%) as a white solid. ¹H NMR: δ 10.43 (s, 1H), 7.65-7.49 (m, 3H), 4.46 (s, 2H), 3.85 (t, 2H), 3.09 (s, 3H), 2.74 (t, 2H). m/z (ES⁺), [M+H]⁺=260.2.

### Example 11: 1-(1-Methyl-1H-pyrrolo[2,3-b]pyridin-5-yl)dihydropyrimidine-2,4 (1H,3H)-dione

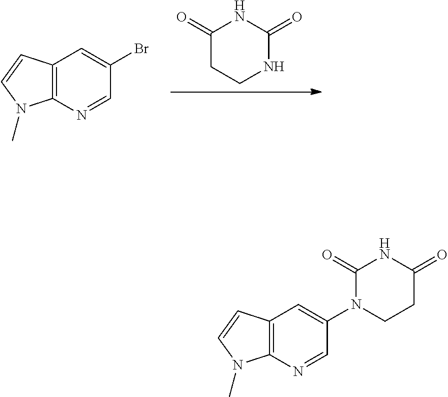 [SMILES: Cn1ccc2cc(Br)cnc21 ; O=C1CCNC(=O)N1 ; Cn1ccc2cc(N3CCC(=O)NC3=O)cnc21]

Cs₂CO₃ (232 mg, 0.712 mmol) was added to a degassed mixture of dihydropyrimidine-2,4 (1H,3H)-dione (81 mg, 0.710 mmol), 5-bromo-1-methyl-1H-pyrrolo[2,3-b]pyridine (50.0 mg, 0.237 mmol), Ephos (12.7 mg, 0.0237 mmol) and Ephos Pd G4 (21.8 mg, 0.0237 mmol) in 1,4-dioxane (5 mL) at r.t. under N₂. The resulting solution was stirred at 100° C. for 16 h. The solid was filtered and washed with DMF. The filtrate was concentrated and purified by C-18FC (gradient: 0-80% MeCN in water) to give the title compound (30.0 mg, 52%) as a white solid. ¹H NMR: δ 10.39 (s, 1H), 8.23 (d, 1H), 7.92 (d, 1H), 7.57 (d, 1H), 6.48 (d, 1H), 3.82 (d, 5H), 2.76 (t, 2H). m/z (ES⁺), [M+H]⁺=245.1.

### Example 12: 1-(3-Methyl-2-oxo-2,3-dihydrobenzo[d]oxazol-6-yl)dihydropyrimidine-2,4 (1H,3H)-dione

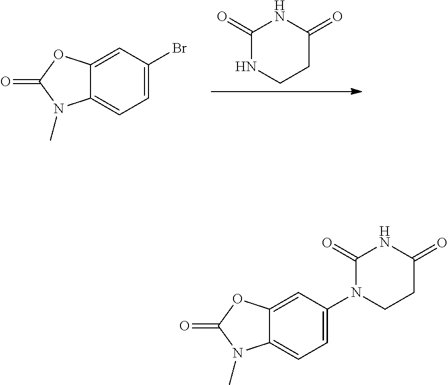 [SMILES: Cn1c(=O)oc2cc(Br)ccc21 ; O=C1CCNC(=O)N1 ; Cn1c(=O)oc2cc(N3CCC(=O)NC3=O)ccc21]

Cs₂CO₃ (286 mg, 0.878 mmol) was added to a degassed mixture of 6-bromo-3-methylbenzo[d]oxazol-2 (3H)-one (100 mg, 0.44 mmol), dihydropyrimidine-2,4 (1H,3H)-dione (50.0 mg, 0.439 mmol), Ephos (11.7 mg, 0.0219 mmol) and Ephos Pd G4 (20.1 mg, 0.0219 mmol) in 1,4-dioxane (10 mL) at r.t. under N₂. The resulting solution was stirred at 100° C. for 16 h. The solvent was then removed under reduced pressure. Purification by C-18FC (gradient 0-100% MeCN in water) gave the title compound (60.0 mg, 52%) as a yellow solid. ¹H NMR: δ 2.72 (t, 2H), 3.36 (s, 3H), 3.76 (t, 2H), 7.20 (dd, 1H), 7.27 (d, 1H), 7.39 (d, 1H), 10.35 (s, 1H). m/z (ES⁺), [M+H]⁺=262.2.

### Example 13: 1-(1H-Indol-6-yl)pyrimidine-2,4 (1H,3H)-dione

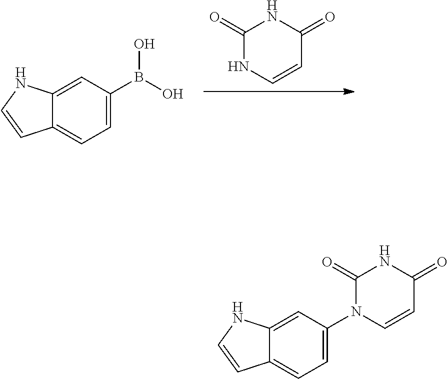 [SMILES: OB(O)c1ccc2cc[nH]c2c1 ; O=c1cc[nH]c(=O)[nH]1 ; O=c1ccn(-c2ccc3cc[nH]c3c2)c(=O)[nH]1]

(1H-Indol-6-yl)boronic acid (100 mg, 0.621 mmol) and pyrimidine-2,4 (1H,3H)-dione (80.0 mg, 0.714 mmol) were added to a mixture of diacetoxycopper (113 mg, 0.622 mmol) and TMEDA (72.2 mg, 0.621 mmol) in MeOH (4 mL) and water (1.00 ml) at r.t. under air. The resulting mixture was stirred at r.t. for 24 h. The solvent was then removed under reduced pressure. Purification C-18FC (gradient: 5-60% MeCN in water (containing 0.05% TFA)) gave the title compound (5.00 mg, 4%) as a yellow solid. ¹H NMR: δ 5.64 (1H, dd), 6.50 (1H, s), 6.98 (1H, dd), 7.43 (1H, s), 7.47 (1H, t), 7.61 (1H, d), 7.72 (1H, d), 11.34 (1H, s), 11.37 (1H, s). m/z (ES⁺), [M+H]⁺=228.2.

### Example 14: 5-Fluoro-1-(1H-indol-6-yl)pyrimidine-2,4 (1H,3H)-dione

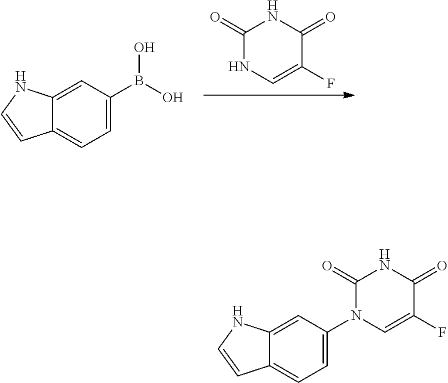 [SMILES: OB(O)c1ccc2cc[nH]c2c1 ; O=c1[nH]cc(F)c(=O)[nH]1 ; O=c1[nH]c(=O)n(-c2ccc3cc[nH]c3c2)cc1F]

(1H-Indol-6-yl)boronic acid (200 mg, 1.24 mmol) and 5-fluoropyrimidine-2,4 (1H,3H)-dione (162 mg, 1.24 mmol) were added to a mixture of diacetoxycopper (226 mg, 1.24 mmol) and pyridine (201 μL, 2.48 mmol) in DMF (8 mL) at r.t. under O₂. The resulting mixture was stirred at 60° C. for 16 h. The solvent was then removed under reduced pressure. Purification by C-18FC (gradient: 5-60% MeCN in water (containing 0.1% FA)) gave the title compound (70.0 mg, 23%) as a yellow solid. ¹H NMR: δ 6.46-6.54 (1H, m), 7.00 (1H, dd), 7.43-7.50 (2H, m), 7.60 (1H, d), 8.20 (1H, d), 11.36 (1H, s), 11.90 (1H, br s). ¹⁹F NMR (282 MHz) δ −170.46. m/z (ES⁺), [M+H]⁺=246.2.

### Example 15: 1-(1H-Indol-6-yl)-5-methylpyrimidine-2,4 (1H,3H)-dione

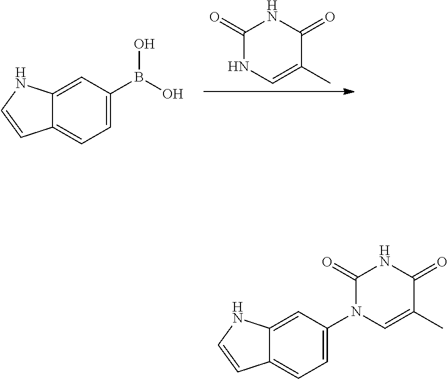

Pyridine (201 μL, 2.48 mmol) was added to a mixture of diacetoxycopper (226 mg, 1.24 mmol), 5-methylpyrimidine-2,4 (1H,3H)-dione (157 mg, 1.24 mmol) and (1H-indol-6-yl)boronic acid (200 mg, 1.24 mmol) in DMF (10 mL) at r.t. under O₂. The resulting mixture was stirred at 60° C. for 17 h. The solvent was then removed under reduced pressure. Purification by C-18FC (gradient: 0-30% MeCN in water (containing 0.1% FA)) gave the title compound (0.139 g, 45%) as a brown solid. ¹H NMR: δ 1.82 (3H, d), 6.50 (1H, t), 6.97 (1H, dd), 7.42 (1H, s), 7.46 (1H, t), 7.58-7.65 (2H, m), 11.21-11.52 (2H, m). m/z (ES⁺), [M+H]⁺=242.2.

### Intermediate 16a: 4-Bromo-6-methoxy-1-methyl-1H-indole

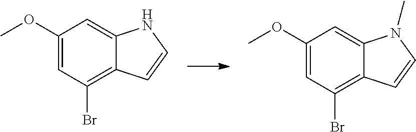

NaH (60% dispersion in mineral oil, 80.0 mg, 1.99 mmol) was added to a solution of 4-bromo-6-methoxy-1H-indole (300 mg, 1.33 mmol) in THF (10 mL) at 0° C. under N₂. The resulting mixture was stirred at r.t. for 20 minutes before the addition of Mel (83.0 μL, 1.33 mmol). The resulting mixture was stirred at r.t. for 1 h. The reaction was quenched with saturated NH₄Cl (20 mL) and extracted with EtOAc (3×20 mL). The combined organic extracts were dried (Na₂SO₄) and concentrated to give a yellow solid. Purification by FSC (gradient: 0-20% EtOAc in petroleum ether) gave the title compound (0.210 g, 66%) as a yellow solid. ¹H NMR: δ 3.76 (3H, s), 3.82 (3H, s), 6.28 (1H, dd), 6.93 (1H, d), 7.04 (1H, dd), 7.30 (1H, d). m/z (ES⁺), [M+H]⁺=240.0.

### Example 16: 1-(6-Methoxy-1-methyl-1H-indol-4-yl)dihydropyrimidine-2,4 (1H,3H)-dione

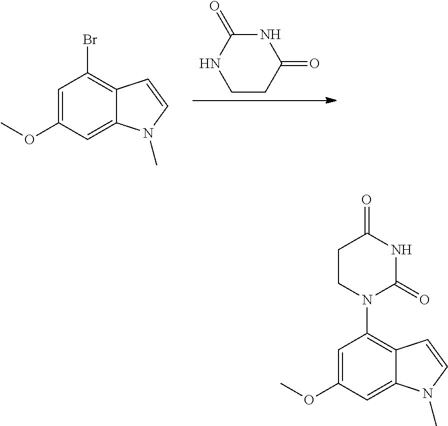 [SMILES: COc1cc(Br)c2ccn(C)c2c1 ; O=C1CCNC(=O)N1 ; COc1cc(N2CCC(=O)NC2=O)c2ccn(C)c2c1]

Ephos (33.4 mg, 0.0625 mmol) and Ephos Pd G4 (57.4 mg, 0.0625 mmol) were added to a degassed mixture of Cs₂CO₃ (814 mg, 2.50 mmol), 4-bromo-6-methoxy-1-methyl-1H-indole (300 mg, 1.25 mmol) and dihydropyrimidine-2,4 (1H,3H)-dione (428 mg, 3.75 mmol) in DMF (20 mL) at r.t. under N₂. The resulting mixture was stirred at 100° C. for 16 h. The solvent was then removed under reduced pressure. Purification by C-18FC (gradient: 5-40% MeCN in water) provided material that was further purified by preparative HPLC (Column C, Eluent C, gradient: 24-49%) to give the title compound (118 mg, 35%) as a white solid. ¹H NMR: δ 2.75 (2H, t), 3.76-3.79 (5H, m), 3.82 (3H, s), 6.29 (1H, d), 6.65 (1H, d), 6.95 (1H, s), 7.18 (1H, d), 10.31 (1H, s). m/z (ES⁺), [M+H]⁺=274.2.

### Intermediate 17a: tert-Butyl 4-(2,4-dioxotetrahydropyrimidin-1 (2H)-yl)-1H-pyrrolo[2,3-c]pyridine-1-carboxylate

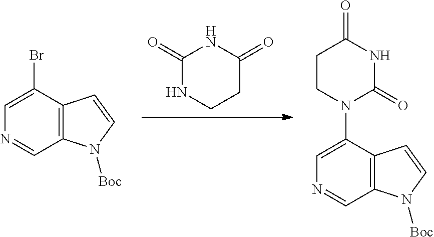 [SMILES: CC(C)(C)OC(=O)n1ccc2c(Br)cncc21 ; O=C1CCNC(=O)N1 ; CC(C)(C)OC(=O)n1ccc2c(N3CCC(=O)NC3=O)cncc21]

Ephos Pd G4 (309 mg, 0.336 mmol) was added to a degassed mixture of Ephos (180 mg, 0.337 mmol), Cs₂CO₃ (2.19 g, 6.72 mmol), dihydropyrimidine-2,4 (1H,3H)-dione (1.536 g, 13.46 mmol) and tert-butyl 4-bromo-1H-pyrrolo[2,3-c]pyridine-1-carboxylate (1.00 g, 3.37 mmol) in 1,4-dioxane (40 mL) at r.t. under N₂. The resulting mixture was stirred at 100° C. for 16 h. The solvent was then removed under reduced pressure. Purification by C-18FC (gradient 5-80% MeCN in water) gave the title compound (150 mg, 13%) as a pale yellow solid. m/z (ES⁺), [M+H]⁺=331.2.

### Example 17: 1-(1H-Pyrrolo[2,3-c]pyridin-4-yl)dihydropyrimidine-2,4 (1H,3H)-dione

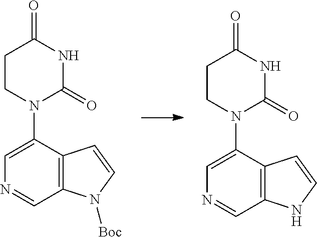 [SMILES: CC(C)(C)OC(=O)n1ccc2c(N3CCC(=O)NC3=O)cncc21 ; O=C1CCN(c2cncc3[nH]ccc23)C(=O)N1]

tert-Butyl 4-(2,4-dioxotetrahydropyrimidin-1 (2H)-yl)-1H-pyrrolo[2,3-c]pyridine-1-carboxylate (150 mg, 0.454 mmol) was dissolved in 2,2,2-trifluoroethanol (10 mL) and sealed into a microwave tube. The reaction was heated to 120° C. for 1 h in a microwave reactor and then cooled to r.t. The solvent was removed under reduced pressure. Purification by preparative HPLC (Column B, Eluent D, gradient: 3-24%) gave the title compound (80.0 mg, 77%) as a white solid. ¹H NMR: δ 2.79 (2H, t), 3.84 (2H, t), 6.50 (1H, d), 7.63 (1H, t), 8.05 (1H, s), 8.68 (1H, s), 10.42 (1H, s), 11.75 (1H, s). m/z (ES⁺), [M+H]⁺=231.3.

### Intermediate 18a: tert-Butyl 4-(2,4-dioxotetrahydropyrimidin-1 (2H)-yl)-1H-indole-1-carboxylate

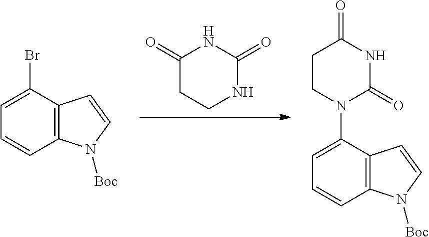 [SMILES: CC(C)(C)OC(=O)n1ccc2c(Br)cccc21 ; O=C1CCNC(=O)N1 ; CC(C)(C)OC(=O)n1ccc2c(N3CCC(=O)NC3=O)cccc21]

Cs₂CO₃ (1.65 g, 5.06 mmol) was added to a degassed mixture of Ephos Pd G4 (155 mg, 0.169 mmol), Ephos (90.0 mg, 0.168 mmol), dihydropyrimidine-2,4 (1H,3H)-dione (578 mg, 5.06 mmol) and tert-butyl 4-bromo-1H-indole-1-carboxylate (500 mg, 1.69 mmol) in 1,4-dioxane (20 mL) at r.t. under N₂. The resulting mixture was stirred at 100° C. for 17 h. The solvent was then removed under reduced pressure. Purification by C-18FC (gradient 0-50% MeCN in water (containing 0.1% conc. HCl)) gave the title compound (100 mg, 18%) as a pale yellow solid. ¹H NMR: δ 1.64 (9H, s), 2.79 (2H, t), 3.80 (2H, t), 6.69 (1H, d), 7.20 (1H, d), 7.36 (1H, t), 7.69 (1H, d), 8.02 (1H, d), 10.42 (1H, s). m/z (ES⁺), [M+H]⁺=330.1.

### Example 18: 1-(1H-Indol-4-yl)dihydropyrimidine-2,4 (1H,3H)-dione

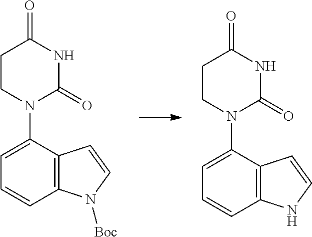 [SMILES: CC(C)(C)OC(=O)n1ccc2c(N3CCC(=O)NC3=O)cccc21 ; O=C1CCN(c2cccc3[nH]ccc23)C(=O)N1]

tert-Butyldimethylsilyl trifluoromethanesulfonate (126 μL, 0.549 mmol) was added to a solution of tert-butyl 4-(2,4-dioxotetrahydropyrimidin-1 (2H)-yl)-1H-indole-1-carboxylate (90.0 mg, 0.273 mmol) in DCM (1 mL) at r.t. under air. The resulting solution was stirred at r.t. for 16 h. The solvent was then removed under reduced pressure. Purification by C-18FC (gradient: 0-30% MeCN in water (containing 0.1% conc. HCl)) provided material that was further purified by preparative HPLC (Column D, Eluent E, gradient: 20-30%) to give the title compound (15.0 mg, 24%) as a pale yellow solid. ¹H NMR: δ 2.76 (2H, t), 3.78 (2H, t), 6.39 (1H, ddd), 6.93 (1H, dd), 7.09 (1H, t), 7.31-7.39 (2H, m), 10.32 (1H, s), 11.24 (1H, s). m/z (ES⁺), [M+H]⁺=230.0.

### Intermediate 19a: (4-Bromo-1H-indol-6-yl)methanol

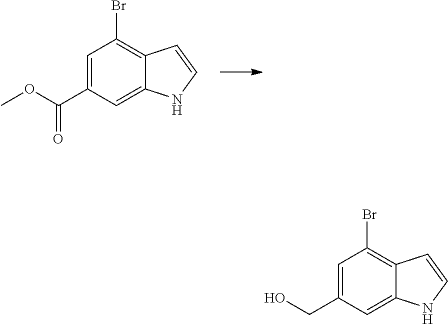

A solution of LiAlH₄ in THF (2.5M, 6.30 ml, 15.7 mmol) was added dropwise to a solution of methyl 4-bromo-1H-indole-6-carboxylate (1.00 g, 3.94 mmol) in THF (20 mL) at 0° C. under N₂. The resulting mixture was stirred at r.t. for 2 h. The reaction mixture was quenched dropwise with water (0.5 mL) and then 15% NaOH (1.5 mL) and water (0.5 mL). The mixture was then filtered through celite and concentrated to dryness to afford the title compound (600 mg, 67%) as a yellow solid. ¹H NMR: δ 4.56 (2H, s), 5.20 (1H, br s), 6.33-6.37 (1H, m), 7.18 (1H, d), 7.36 (1H, t), 7.42 (1H, t), 11.40 (1H, s). m/z (ES⁺), [M+H]⁺=226.0

### Intermediate 19b: 4-Bromo-6-(methoxymethyl)-1-methyl-1H-indole

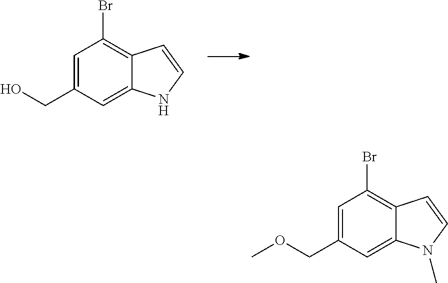

NaH (60% dispersion in mineral oil, 389 mg, 9.73 mmol) was added to a solution of (4-bromo-1H-indol-6-yl)methanol (550 mg, 2.43 mmol) in DMF (20 mL) at 0° C. under N₂. The resulting mixture was stirred at r.t. for 0.5 h before the addition of Mel (456 μL, 7.30 mmol). The resulting mixture was stirred at r.t. for 3 h. The reaction was quenched with saturated NH₄Cl (50 mL) and extracted with EtOAc (3×50 mL). The combined organic extracts were dried (Na₂SO₄) and concentrated to give a yellow liquid. Purification by FSC (gradient: 0-30% EtOAc in petroleum ether) gave the title compound (410 mg, 66%) as a yellow oil. ¹H NMR: δ 3.30 (3H, s), 3.81 (3H, s), 4.51 (2H, s), 6.37 (1H, dd), 7.22 (1H, d), 7.41-7.49 (2H, m). m/z (ES⁺), [M+H]⁺=256.1.

### Example 19: 1-(6-(Methoxymethyl)-1-methyl-1H-indol-4-yl)dihydropyrimidine-2,4 (1H,3H)-dione

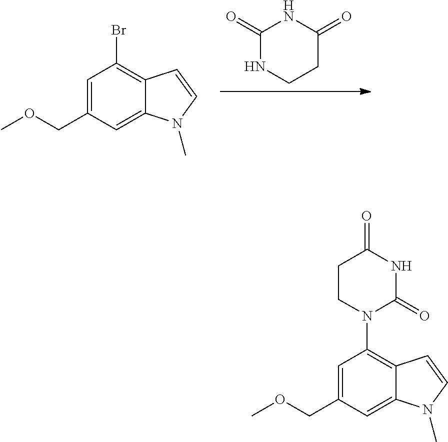 [SMILES: COCc1cc(Br)c2ccn(C)c2c1 ; O=C1CCNC(=O)N1 ; COCc1cc(N2CCC(=O)NC2=O)c2ccn(C)c2c1]

Ephos (42.1 mg, 0.0787 mmol) and Ephos Pd G4 (72.3 mg, 0.0787 mmol) were added to a degassed mixture of Cs₂CO₃ (1.03 g, 3.16 mmol), 4-bromo-6-(methoxymethyl)-1-methyl-1H-indole (400 mg, 1.57 mmol) and dihydropyrimidine-2,4 (1H,3H)-dione (539 mg, 4.72 mmol) in 1,4-dioxane (10 mL) at r.t. under N₂. The resulting mixture was stirred at 100° C. for 16 h. The mixture was cooled to r.t. and silica was added. The solvent was removed under reduced pressure. The crude product was dry-loaded and purified by C-18FC (gradient: 5-40% MeCN in water (containing 0.1% conc. HCl)) gave the title compound (0.305 g, 67%) as a white solid. ¹H NMR: δ 2.76 (2H, t), 3.32 (3H, s) 3.74-3.82 (5H, m), 4.51 (2H, s), 6.36 (1H, d), 6.95 (1H, s), 7.30-7.37 (2H, m), 10.31 (1H, s). m/z (ES⁺), [M+H]⁺=288.1.

### Intermediate 20a: tert-Butyl 5-(2,4-dioxotetrahydropyrimidin-1 (2H)-yl)indoline-1-carboxylate

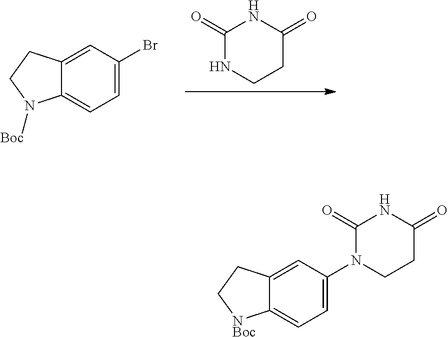 [SMILES: CC(C)(C)OC(=O)N1CCc2cc(Br)ccc21 ; O=C1CCNC(=O)N1 ; CC(C)(C)OC(=O)N1CCc2cc(N3CCC(=O)NC3=O)ccc21]

Ephos (44.8 mg, 0.0838 mmol) and Ephos Pd G4 (77.0 mg, 0.0838 mmol) were added to a degassed mixture of Cs₂CO₃ (1.09 g, 3.35 mmol), tert-butyl 5-bromoindoline-1-carboxylate (500 mg, 1.68 mmol) and dihydropyrimidine-2,4 (1H,3H)-dione (574 mg, 5.03 mmol) in 1,4-dioxane (30 mL) at r.t. under N₂. The resulting mixture was stirred at 100° C. for 16 h. The mixture was cooled to r.t. and silica was added. The solvent was removed under reduced pressure. The crude product was dry-loaded and purified by FSC (gradient: 0-98% EtOAc in petroleum ether) to give the title compound (140 mg, 25%) as a white solid. ¹H NMR: δ 1.49 (9H, s), 2.68 (2H, t), 3.04 (2H, t), 3.69 (2H, t), 3.90 (2H, t), 7.06 (1H, dd), 7.14 (1H, d), 7.61-7.77 (1H, m), 10.30 (1H, s). m/z (ES⁺), [M-tBu+2H]⁺=276.1.

### Example 20: 1-(Indolin-5-yl)dihydropyrimidine-2,4 (1H,3H)-dione

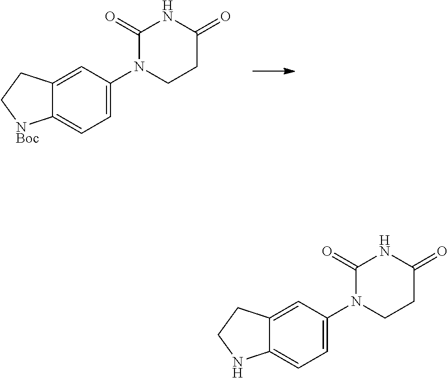 [SMILES: CC(C)(C)OC(=O)N1CCc2cc(N3CCC(=O)NC3=O)ccc21 ; O=C1CCN(c2ccc3c(c2)CCN3)C(=O)N1]

TFA (1 mL) was added to a solution of tert-butyl 5-(2,4-dioxotetrahydropyrimidin-1 (2H)-yl)indoline-1-carboxylate (140 mg, 0.422 mmol) in DCM (4 mL). The resulting mixture was stirred at r.t. for 2 h. The solvent was then removed under reduced pressure. The reaction mixture was diluted with DCM and the solvent was removed under reduced pressure. Purification by C-18FC (gradient: 0-20% MeCN in water) gave the title compound (56.0 mg, 57%) as a white solid. ¹H NMR: δ 2.70 (2H, t), 3.10 (2H, t), 3.64 (2H, dt), 3.73 (2H, t), 7.12 (2H, q), 7.26 (1H, d), 10.33 (1H, s). m/z (ES⁺), [M+H]⁺=232.2.

### Example 21: 1-(1-Methylindolin-5-yl)dihydropyrimidine-2,4 (1H,3H)-dione

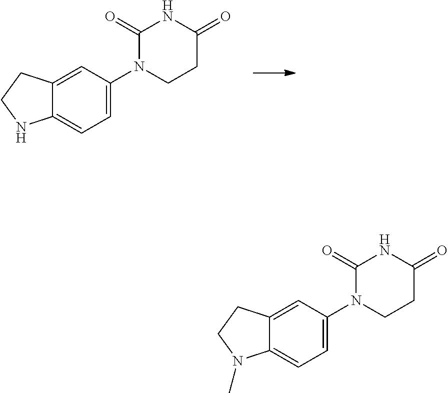 [SMILES: O=C1CCN(c2ccc3c(c2)CCN3)C(=O)N1 ; CN1CCc2cc(N3CCC(=O)NC3=O)ccc21]

NaOAc (142 mg, 1.73 mmol) was added to a mixture of 1-(indolin-5-yl)dihydropyrimidine-2,4 (1H,3H)-dione (100 mg, 0.43 mmol) and paraformaldehyde (104 mg, 3.46 mmol) in DCM (10 mL) at r.t. The resulting mixture was stirred at r.t. for 1 h before the addition of sodium triacetoxyborohydride (229 mg, 1.08 mmol). The resulting mixture was stirred at r.t. for 3 h. The reaction mixture was poured into water (20 mL) and extracted with DCM (3×20 mL). The combined organic extracts were dried (Na₂SO₄) and concentrated to give a brown solid. Purification by preparative HPLC (Column E, Eluent E, gradient: 9-19%) gave the title compound (22.0 mg, 21%) as a brown solid. ¹H NMR: δ 2.67 (2H, t), 2.70 (3H, s), 2.86 (2H, t), 3.26 (2H, t), 3.65 (2H, t), 6.49 (1H, d), 6.93 (1H, dd), 6.98 (1H, s), 10.23 (1H, s). m/z (ES⁺), [M+H]⁺=246.1.

### Intermediate 22a: tert-Butyl 6-(2,4-dioxotetrahydropyrimidin-1 (2H)-yl)indoline-1-carboxylate

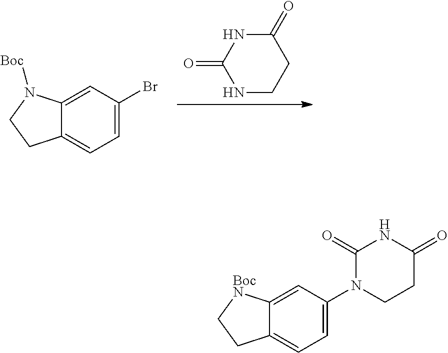 [SMILES: CC(C)(C)OC(=O)N1CCc2ccc(Br)cc21 ; O=C1CCNC(=O)N1 ; CC(C)(C)OC(=O)N1CCc2ccc(N3CCC(=O)NC3=O)cc21]

Ephos (90.0 mg, 0.168 mmol) and Ephos Pd G4 (154 mg, 0.168 mmol) were added to a degassed mixture of Cs₂CO₃ (2.19 g, 6.72 mmol), tert-butyl 6-bromoindoline-1-carboxylate (1.00 g, 3.35 mmol) and dihydropyrimidine-2,4 (1H,3H)-dione (1.148 g, 10.06 mmol) in 1,4-dioxane (40 mL) at r.t. under N₂. The resulting mixture was stirred at 100° C. for 16 h. The mixture was cooled to r.t. and silica was added. The solvent was removed under reduced pressure. The crude product was dry-loaded and purified by FSC (gradient 0-99% EtOAc in petroleum ether) to give the title compound (420 mg, 38%) as a white solid. ¹H NMR: δ 1.50 (9H, s), 2.70 (2H, t), 3.05 (2H, t), 3.74 (2H, t), 3.94 (2H, t), 6.87 (1H, dd), 7.19 (1H, d), 7.63 (1H, s), 10.33 (1H, s). m/z (ES⁺), [M-tBu+2H]⁺=276.2.

### Example 22: 1-(Indolin-6-yl)dihydropyrimidine-2,4 (1H,3H)-dione

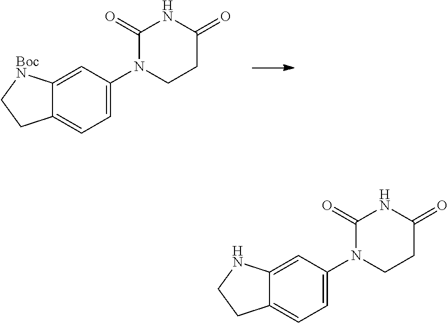 [SMILES: CC(C)(C)OC(=O)N1CCc2ccc(N3CCC(=O)NC3=O)cc21 ; O=C1CCN(c2ccc3c(c2)NCC3)C(=O)N1]

TFA (2 mL) was added to a solution of tert-butyl 6-(2,4-dioxotetrahydropyrimidin-1 (2H)-yl)indoline-1-carboxylate (470 mg, 1.42 mmol) in DCM (8 mL). The resulting mixture was stirred at r.t. for 3 h. The solvent was then removed under reduced pressure. The reaction mixture was diluted with DCM and the solvent removed under reduced pressure. Purification C-18FC (gradient 0-20% MeCN in water) gave the title compound (0.233 g, 71%) as a white solid. ¹H NMR: δ 2.70 (2H, t), 3.06 (2H, t), 3.63 (2H, t), 3.75 (2H, t), 6.96 (1H, d), 7.01 (1H, d), 7.28 (1H, d), 10.34 (1H, s). m/z (ES⁺), [M+H]⁺=232.1.

### Example 23: 1-(1-Methylindolin-6-yl)dihydropyrimidine-2,4 (1H,3H)-dione

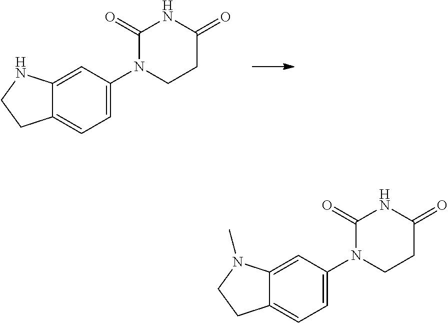 [SMILES: O=C1CCN(c2ccc3c(c2)NCC3)C(=O)N1 ; CN1CCc2ccc(N3CCC(=O)NC3=O)cc21]

NaOAc (142 mg, 1.73 mmol) was added to a mixture of 1-(indolin-6-yl)dihydropyrimidine-2,4 (1H,3H)-dione (100 mg, 0.432 mmol) and paraformaldehyde (104 mg, 3.46 mmol) in DCM (5 mL) at r.t. The resulting mixture was stirred at r.t. for 1 h before the addition of sodium triacetoxyborohydride (229 mg, 1.08 mmol). The resulting mixture was stirred at r.t. for 3 h. The reaction mixture was poured into water (20 mL) and extracted with DCM (3×20 mL). The combined organic extracts were dried (Na₂SO₄) and concentrated to afford a yellow solid. Purification by preparative HPLC (Column B, Eluent D, gradient: 20-40%) gave the title compound (15.1 mg, 14%) as a white solid. ¹H NMR: δ 2.47 (3H, s), 2.69 (2H, t), 3.69-3.82 (6H, m), 7.12 (1H, dd), 7.15-7.27 (2H, m), 10.33 (1H, s). m/z (ES⁺), [M+H]⁺=246.0.

### Intermediate 24a: tert-Butyl 5-(2,4-dioxotetrahydropyrimidin-1 (2H)-yl)isoindoline-2-carboxylate

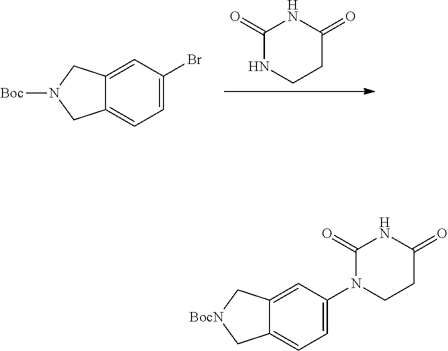 [SMILES: CC(C)(C)OC(=O)N1Cc2ccc(Br)cc2C1 ; O=C1CCNC(=O)N1 ; CC(C)(C)OC(=O)N1Cc2ccc(N3CCC(=O)NC3=O)cc2C1]

Ephos (0.179 g, 0.335 mmol) and Ephos Pd G4 (0.308 g, 0.335 mmol) were added to a degassed mixture of Cs₂CO₃ (2.19 g, 6.72 mmol), tert-butyl 5-bromoisoindoline-2-carboxylate (1.00 g, 3.35 mmol) and dihydropyrimidine-2,4 (1H,3H)-dione (1.15 g, 10.1 mmol) in 1,4-dioxane (30 mL) at r.t. under N₂. The resulting mixture was stirred at 100° C. for 16 h. The mixture was cooled to r.t. and silica was added. The solvent was removed under reduced pressure. The crude product was dry-loaded and purified by FSC (gradient: 0-98% EtOAc in petroleum ether) to give the title compound (0.250 g, 23%) as a white solid. ¹H NMR: δ 1.47 (9H, s), 2.71 (2H, t), 3.77 (2H, t), 4.59 (4H, br s), 7.19-7.39 (3H, m), 10.38 (1H, s). m/z (ES⁺), [M-tBu+2H]⁺=276.2.

### Example 24: 1-(Isoindolin-5-yl)dihydropyrimidine-2,4 (1H,3H)-dione

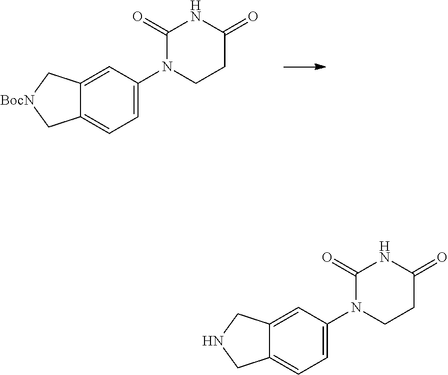 [SMILES: CC(C)(C)OC(=O)N1Cc2ccc(N3CCC(=O)NC3=O)cc2C1 ; O=C1CCN(c2ccc3c(c2)CNC3)C(=O)N1]

TFA (2 mL) was added to a solution of tert-butyl 5-(2,4-dioxotetrahydropyrimidin-1 (2H)-yl)isoindoline-2-carboxylate (280 mg, 0.845 mmol) in DCM (8 mL). The resulting mixture was stirred at r.t. for 3 h. The solvent was then removed under reduced pressure. The reaction mixture was diluted with DCM and the solvent removed under reduced pressure. Purification by C-18FC (gradient 0-10% MeCN in water) gave the title compound in the form of a trifluoroacetate salt (61.0 mg, 21%) as a white solid. ¹H NMR: δ 2.72 (2H, t), 3.78 (2H, t), 4.52 (4H, d), 7.32 (1H, d), 7.36-7.45 (2H, m), 9.56 (2H, s), 10.40 (1H, s). m/z (ES⁺), [M+H]⁺=232.2.

### Example 25: 1-(2-Methylisoindolin-5-yl)dihydropyrimidine-2,4 (1H,3H)-dione

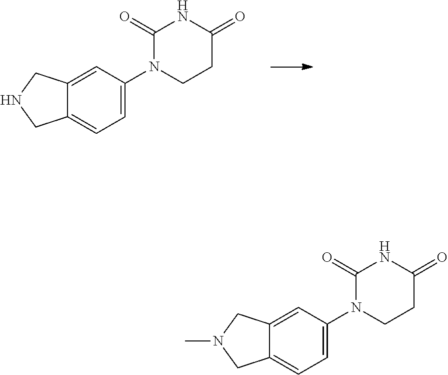 [SMILES: O=C1CCN(c2ccc3c(c2)CNC3)C(=O)N1 ; CN1Cc2ccc(N3CCC(=O)NC3=O)cc2C1]

NaOAc (95.0 mg, 1.16 mmol) was added to a mixture of 1-(isoindolin-5-yl)dihydropyrimidine-2,4 (1H,3H)-dione 2,2,2-trifluoroacetate (100 mg, 0.290 mmol), paraformaldehyde (69.6 mg, 2.32 mmol) in DCM (10 mL). The resulting mixture was stirred at r.t. for 1 h before the addition of sodium triacetoxyborohydride (153 mg, 0.722 mmol). The resulting mixture was stirred at r.t. for 3 h. The reaction mixture was poured into water (20 mL) and extracted with DCM (3×20 mL). The combined organic extracts were dried (Na₂SO₄) and concentrated to give a brown solid. Purification by preparative HPLC (Column B, Eluent B, gradient: 5-30%) gave the title compound (19.5 mg, 27%) as a white powder. ¹H NMR: δ 2.67 (2H, t), 2.68 (3H, s), 2.85 (2H, t), 3.27 (2H, t), 3.70 (2H, t), 6.45 (1H, d), 6.50 (1H, dd), 7.01 (1H, d), 10.26 (1H, s). m/z (ES⁺), [M+H]⁺=246.0.

### Intermediate 26a: tert-Butyl 6-(2,4-dioxotetrahydropyrimidin-1 (2H)-yl)-3,4-dihydroquinoline-1 (2H)-carboxylate

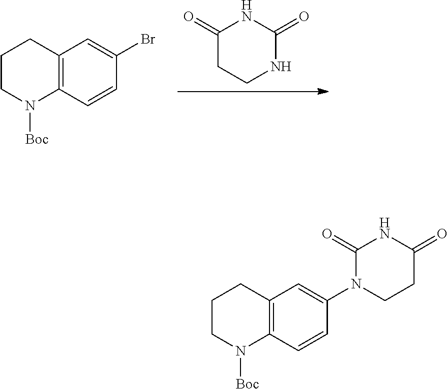 [SMILES: CC(C)(C)OC(=O)N1CCCc2cc(Br)ccc21 ; O=C1CCNC(=O)N1 ; CC(C)(C)OC(=O)N1CCCc2cc(N3CCC(=O)NC3=O)ccc21]

Cs₂CO₃ (1.94 g, 5.95 mmol) was added to a degassed mixture of tert-butyl 6-bromo-3,4-dihydroquinoline-1 (2H)-carboxylate (620 mg, 1.99 mmol), dihydropyrimidine-2,4 (1H,3H)-dione (680 mg, 5.96 mmol), Ephos (53.1 mg, 0.0993 mmol) and Ephos Pd G4 (91.0 mg, 0.0991 mmol) in 1,4-dioxane (20 mL) at r.t. under N₂. The resulting mixture was stirred at 100° C. for 20 h. The solids were filtered off and washed with 1,4-dioxane (20 mL). The filtrate was concentrated to dryness. Purification by C-18FC (gradient 30-70% MeCN in water) gave the title compound (650 mg, 95%) as a white solid. ¹H NMR: (CD₃OD) δ 1.54 (9H, s), 1.91-1.97 (2H, m), 2.79-2.84 (m, 4H), 3.70-3.74 (m, 2H), 3.85 (2H, t), 7.09-7.16 (2H, m), 7.65 (1H, d). m/z (ES⁺), [M−tBu+2H]⁺=290.1.

### Example 26: 1-(1,2,3,4-Tetrahydroquinolin-6-yl)dihydropyrimidine-2,4 (1H,3H)-dione

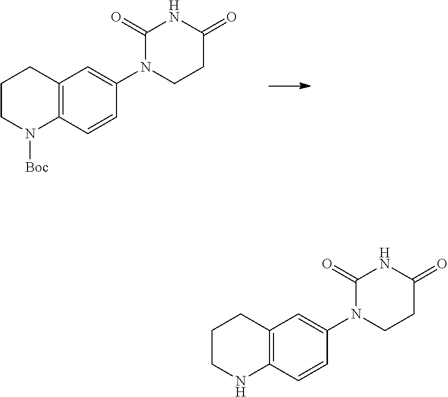 [SMILES: CC(C)(C)OC(=O)N1CCCc2cc(N3CCC(=O)NC3=O)ccc21 ; O=C1CCN(c2ccc3c(c2)CCCN3)C(=O)N1]

tert-Butyl 6-(2,4-dioxotetrahydropyrimidin-1 (2H)-yl)-3,4-dihydroquinoline-1 (2H)-carboxylate (600 mg, 1.74 mmol) was added to a solution of HCl in 1,4-dioxane (4M, 35.0 mL, 140 mmol) to give a white suspension. The resulting mixture was stirred at r.t. for 3 h. The precipitate was collected by filtration, washed with EtOAc and dried under vacuum to give the title compound in the form of a hydrochloride salt (390 mg, 80%) as a white solid. ¹H NMR: δ 1.94 (2H, p), 2.69 (2H, t), 2.78 (2H, t), 3.25-3.35 (2H, m), 3.72 (2H, t), 4.06 (2H, s), 7.02-7.09 (1H, m), 7.13-7.20 (2H, m). m/z (ES⁺), [M+H]⁺=246.1.

### Example 27: 1-(1-Methyl-1,2,3,4-tetrahydroquinolin-6-yl)dihydropyrimidine-2,4 (1H,3H)-dione

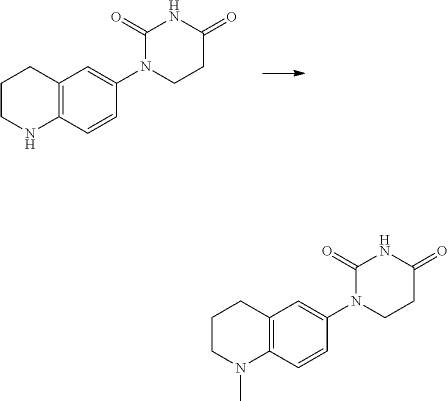 [SMILES: O=C1CCN(c2ccc3c(c2)CCCN3)C(=O)N1 ; CN1CCCc2cc(N3CCC(=O)NC3=O)ccc21]

Paraformaldehyde (29.4 mg, 0.979 mmol) was added to a mixture of 1-(1,2,3,4-tetrahydroquinolin-6-yl)dihydropyrimidine-2,4 (1H,3H)-dione hydrochloride (80.0 mg, 0.284 mmol) in MeOH (6 mL) to give a white suspension. The resulting mixture was stirred at r.t. for 0.5 h before the addition of NaBH₃CN (61.5 mg, 0.979 mmol). The resulting mixture was stirred at r.t. for 16 h and then purified directly by C-18FC (gradient: 10-50% MeCN in water) to give the title compound (70.0 mg, 95%) as a white solid. ¹H NMR: δ 1.88 (2H, m), 2.66 (4H, dt), 2.82 (3H, s), 3.15-3.18 (2H, m), 3.64 (2H, t), 6.54 (1H, d), 6.83 (1H, d), 6.91 (1H, dd), 10.20 (1H, s). m/z (ES⁺), [M+H]⁺=260.0.

### Intermediate 28a: tert-Butyl 7-(2,4-dioxotetrahydropyrimidin-1 (2H)-yl)-3,4-dihydroisoquinoline-2 (1H)-carboxylate

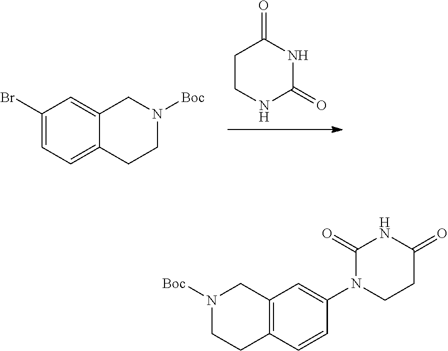 [SMILES: CC(C)(C)OC(=O)N1CCc2ccc(Br)cc2C1 ; O=C1CCNC(=O)N1 ; CC(C)(C)OC(=O)N1CCc2ccc(N3CCC(=O)NC3=O)cc2C1]

Cs₂CO₃ (1.88 g, 5.77 mmol) was added to a mixture of tert-butyl 7-bromo-3,4-dihydroisoquinoline-2 (1H)-carboxylate (600 mg, 1.92 mmol), dihydropyrimidine-2,4 (1H,3H)-dione (658 mg, 5.77 mmol), Ephos (51.4 mg, 0.0961 mmol) and Ephos Pd G4 (88.3 mg, 0.0961 mmol) in 1,4-dioxane (30 mL) under N₂. The resulting mixture was stirred at 100° C. for 18 h. The resulting mixture was filtered and washed with 1,4-dioxane. The filtrate was concentrated and purification by C-18FC (gradient: 0-70% MeCN in water) gave the title compound (580 mg, 87%) as a pale yellow solid. ¹H NMR: δ 1.41 (s, 9H), 2.74 (t, 2H), 2.67 (t, 2H), 3.53 (t, 2H), 3.73 (t, 2H), 4.47 (s, 2H), 7.10-7.17 (m, 3H), 10.34 (s, 1H). m/z (ES⁺), [M+Na]=368.2.

### Example 28: 1-(1,2,3,4-Tetrahydroisoquinolin-7-yl)dihydropyrimidine-2,4 (1H,3H)-dione

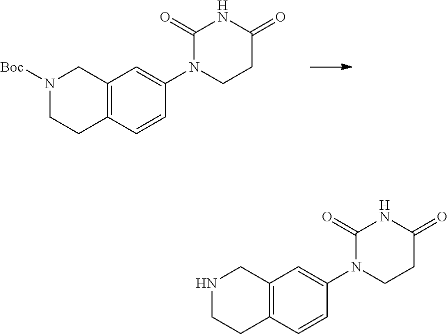 [SMILES: CC(C)(C)OC(=O)N1CCc2ccc(N3CCC(=O)NC3=O)cc2C1 ; O=C1CCN(c2ccc3c(c2)CNCC3)C(=O)N1]

tert-Butyl 7-(2,4-dioxotetrahydropyrimidin-1 (2H)-yl)-3,4-dihydroisoquinoline-2 (1H)-carboxylate (560 mg, 1.62 mmol) was added to a solution of HCl in 1,4-dioxane (4M, 40.0 mL, 160 mmol). The resulting mixture was stirred at r.t. for 2 h. The resulting reaction mixture was filtered, the precipitate was washed with 1,4-dioxane (3 mL) and DCM (3 mL) to give the title compound in the form of a hydrochloride salt (400 mg, 88%) as a pale yellow solid. ¹H NMR: δ 2.71 (t, 2H), 3.00 (t, 2H), 3.30-3.34 (m, 2H), 3.76 (t, 2H), 4.24 (s, 2H), 7.17-7.28 (m, 3H), 9.50-9.70 (2H, m), 10.40 (s, 1H). m/z (ES⁺), [M+H]⁺=246.1.

### Example 29: 1-(2-Methyl-1,2,3,4-tetrahydroisoquinolin-7-yl)dihydropyrimidine-2,4 (1H,3H)-dione

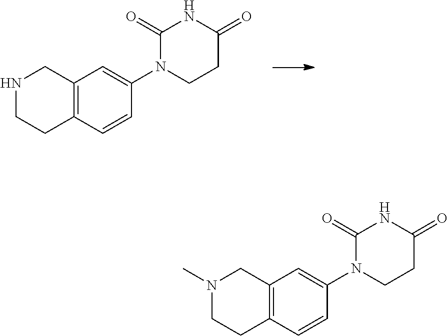 [SMILES: O=C1CCN(c2ccc3c(c2)CNCC3)C(=O)N1 ; CN1CCc2ccc(N3CCC(=O)NC3=O)cc2C1]

Paraformaldehyde (32.0 mg, 1.07 mmol) was added to a mixture of 1-(1,2,3,4-tetrahydroisoquinolin-7-yl)dihydropyrimidine-2,4 (1H,3H1)-dione hydrochloride (60.0 mg, 0.213 mmol) in MeOH (5 mL). The resulting suspension was stirred at r.t. for 4 h before the addition of NaBH₃CN (40.1 mg, 0.638 mmol). The resulting mixture was stirred at r.t. overnight and then purified directly by preparative HPLC (Column B, Eluent B, gradient: 10-25%) to give the title compound (37.7 mg, 68%) as a white solid. ¹H NMR: δ 2.33 (s, 3H), 2.58 (t, 2H), 2.68 (t, 2H), 2.79 (t, 2H), 3.45 (s, 2H), 3.73 (t, 2H), 6.99 (d, 1H), 7.03-7.14 (m, 2H) 10.32 (s, 1H). m/z (ES⁺), [M+H]⁺=260.1.

### Intermediate 30a: tert-Butyl 6-(2,4-dioxotetrahydropyrimidin-1 (2H)-yl)-3,4-dihydroisoquinoline-2 (1H)-carboxylate

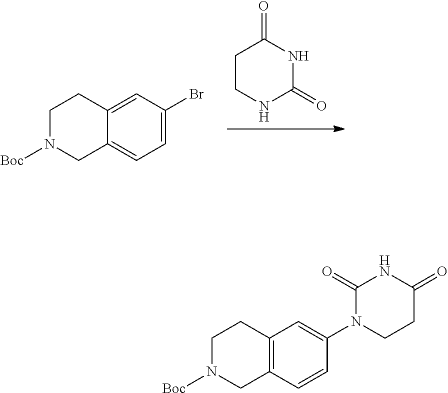

Cs₂CO₃ (1.88 g, 5.77 mmol) was added to a degassed mixture of tert-butyl 6-bromo-3,4-dihydroisoquinoline-2 (1H)-carboxylate (600 mg, 1.92 mmol), dihydropyrimidine-2,4 (1H,3H)-dione (658 mg, 5.77 mmol), Ephos (51.4 mg, 0.0961 mmol) and Ephos Pd G4 (88.3 mg, 0.0961 mmol) in 1,4-dioxane (30 mL) at r.t. under N₂. The resulting mixture was stirred at 100° C. for 18 h. The resulting mixture was filtered, washed with 1,4-dioxane and the filtrate was concentrated. Purification by C-18FC (gradient: 0-70% MeCN in water) gave the title compound (423 mg, 64%) as a pale yellow solid. ¹H NMR: δ 10.34 (s, 1H), 7.26-7.02 (m, 3H), 4.47 (s, 2H), 3.73 (t, 2H), 3.53 (t, 2H), 2.75 (t, 2H), 2.67 (t, 2H), 1.41 (s, 9H). m/z (ES⁺), [M+Na]=368.1.

### Example 30: 1-(1,2,3,4-Tetrahydroisoquinolin-6-yl)dihydropyrimidine-2,4 (1H,3H)-dione

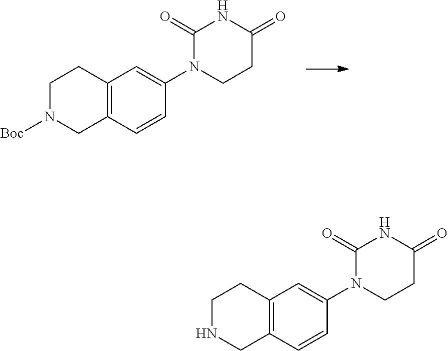 [SMILES: CC(C)(C)OC(=O)N1CCc2cc(N3CCC(=O)NC3=O)ccc2C1 ; O=C1CCN(c2ccc3c(c2)CCNC3)C(=O)N1]

tert-Butyl 6-(2,4-dioxotetrahydropyrimidin-1 (2H)-yl)-3,4-dihydroisoquinoline-2 (1H)-carboxylate (400 mg, 1.16 mmol) was added to a solution of HCl in 1,4-dioxane (4M, 40.0 mL, 160 mmol). The resulting mixture was stirred at r.t. for 2 h. The resulting reaction mixture was filtered, the precipitate was washed with 1,4-dioxane (2×2.5 mL) to give the title compound in the form of a hydrochloride salt (0.287 g, 88%) as a yellow solid. ¹H NMR: δ 2.71 (t, 2H), 3.01 (t, 2H), 3.30-3.42 (m, 2H), 3.77 (t, 2H), 4.24 (s, 2H), 7.17-7.29 (m, 3H), 9.45-9.70 (m, 2H), 10.39 (s, 1H). m/z (ES⁺), [M+H]⁺=246.2.

### Example 31: 1-(2-Methyl-1,2,3,4-tetrahydroisoquinolin-6-yl)dihydropyrimidine-2,4 (1H,3H)-dione

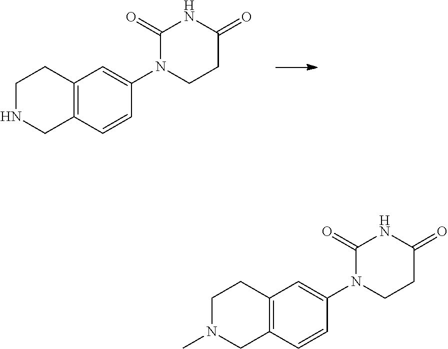 [SMILES: O=C1CCN(c2ccc3c(c2)CCNC3)C(=O)N1 ; CN1CCc2cc(N3CCC(=O)NC3=O)ccc2C1]

Paraformaldehyde (32.0 mg, 1.06 mmol) was added to a mixture of 1-(1,2,3,4-tetrahydroisoquinolin-6-yl)dihydropyrimidine-2,4 (1H,3H)-dione hydrochloride (60.0 mg, 0.213 mmol) in MeOH (5 mL). The resulting mixture was stirred at r.t. for 2 h before the addition of NaBH₃CN (40.1 mg, 0.638 mmol). The resulting mixture was stirred at r.t. overnight and then purified directly by preparative HPLC (Column A, Eluent F, gradient: 12-22%) to give the title compound (39.6 mg, 72%) as a white solid. ¹H NMR: δ 10.33 (s, 1H), 7.17-7.04 (m, 2H), 7.00 (d, 1H), 3.74 (t, 2H), 3.46 (s, 2H), 2.80 (t, 2H), 2.69 (t, 2H), 2.59 (t, 2H), 2.34 (s, 3H). m/z (ES⁺), [M+H]⁺=260.0.

### Intermediate 32a: tert-Butyl 7-(2,4-dioxotetrahydropyrimidin-1 (2H)-yl)-3,4-dihydroquinoline-1 (2H)-carboxylate

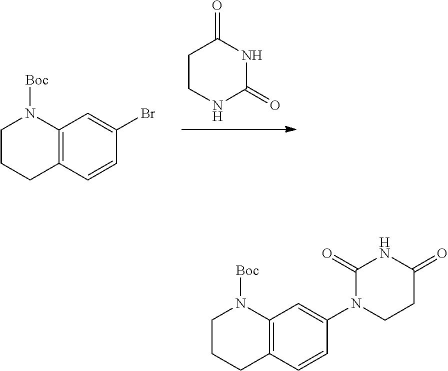 [SMILES: CC(C)(C)OC(=O)N1CCCc2ccc(Br)cc21 ; O=C1CCNC(=O)N1 ; CC(C)(C)OC(=O)N1CCCc2ccc(N3CCC(=O)NC3=O)cc21]

Cs₂CO₃ (1.88 mg, 5.77 mmol) was added to a degassed mixture of tert-butyl 7-bromo-3,4-dihydroquinoline-1 (2H)-carboxylate (600 mg, 1.92 mmol), dihydropyrimidine-2,4 (1H,3H)-dione (658 mg, 5.77 mmol), Ephos (51.4 mg, 0.0961 mmol) and Ephos Pd G4 (88.3 mg, 0.0961 mmol) in 1,4-dioxane (20 mL) at r.t. under N₂. The resulting mixture was stirred at 100° C. for 20 h. The solvent was then removed under reduced pressure. Purification by C-18FC (gradient: 30-70% MeCN in water) gave the title compound (520 mg, 78%) as a white solid. ¹H NMR: (CD₃OD) δ 1.54 (s, 9H), 1.90-1.97 (m, 2H), 2.76-2.85 (m, 4H), 3.69-3.76 (m, 2H), 3.86 (t, 2H), 7.01 (dd, 1H), 7.16 (d, 1H), 7.64 (d, 1H). m/z (ES⁺), [M-tBu+2H]⁺=290.1.

### Example 32: 1-(1,2,3,4-Tetrahydroquinolin-7-yl)dihydropyrimidine-2,4 (1H,3H)-dione

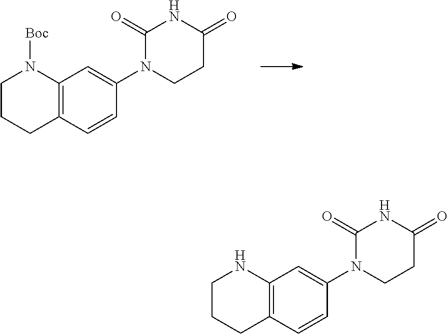 [SMILES: CC(C)(C)OC(=O)N1CCCc2ccc(N3CCC(=O)NC3=O)cc21 ; O=C1CCN(c2ccc3c(c2)NCCC3)C(=O)N1]

tert-Butyl 7-(2,4-dioxotetrahydropyrimidin-1 (2H)-yl)-3,4-dihydroquinoline-1 (2H)-carboxylate (500 mg, 1.45 mmol) was added to a solution of HCl in 1,4-dioxane (4M, 30 mL, 120.00 mmol) to give a colourless solution. The resulting mixture was stirred at r.t. for 16 h. The solvent was then removed under reduced pressure. The residue was resuspended in EtOAc. The precipitate was collected by filtration, washed with EtOAc and dried under vacuum to give the title compound in the form of a hydrochloride salt (300 mg, 74%) as a yellow solid. ¹H NMR: δ 1.91-2.01 (2H, m), 2.70-2.80 (4H, m), 3.18-3.34 (2H, m), 3.74 (2H, t), 7.03-7.07 (2H, m), 7.20 (1H, d), 10.39 (1H, s). m/z (ES⁺), [M+H]⁺=246.1.

### Example 33: 1-(1-Methyl-1,2,3,4-tetrahydroquinolin-7-yl)dihydropyrimidine-2,4 (1H,3H)-dione

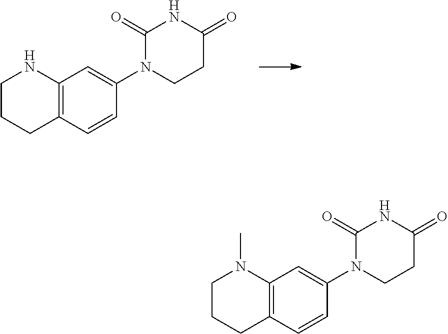 [SMILES: O=C1CCN(c2ccc3c(c2)NCCC3)C(=O)N1 ; CN1CCCc2ccc(N3CCC(=O)NC3=O)cc21]

Formaldehyde (25.6 mg, 0.853 mmol) was added to a mixture of 1-(1,2,3,4-tetrahydroquinolin-7-yl)dihydropyrimidine-2,4 (1H,3H)-dione hydrochloride (80.0 mg, 0.284 mmol) in MeOH (4 mL) to give a white suspension. The resulting mixture was stirred at r.t. for 0.5 h before the addition of NaBH₃CN (53.5 mg, 0.851 mmol). The resulting mixture was stirred at r.t. for 16 h and then purified directly by C-18FC (gradient: 25-50% MeCN in water) to give the title compound (60.0 mg, 81%) as a white solid. ¹H NMR: δ 1.88 (2H, m), 2.67 (4H, t), 2.80 (3H, s), 3.18 (2H, m), 3.69 (2H, t), 6.43 (1H, dd), 6.49 (1H, d), 6.87 (1H, d), 10.24 (1H, s). m/z (ES⁺), [M+H]⁺=260.2.

### Intermediate 34a: 1-(1-(Tetrahydro-2H-pyran-2-yl)-1H-indazol-4-yl)dihydropyrimidine-2,4 (1H,3H)-dione

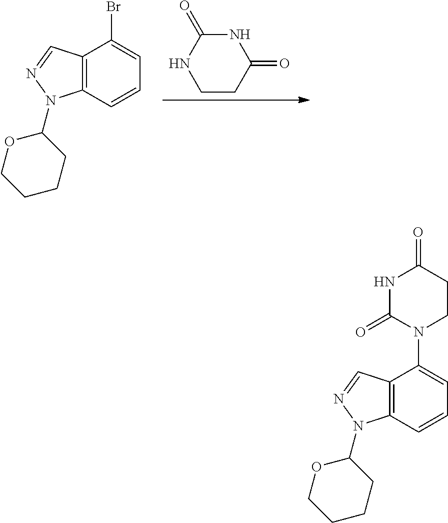

Ephos (19.0 mg, 0.0355 mmol) and Ephos Pd G4 (32.7 mg, 0.0356 mmol) were added to a degassed mixture of Cs₂CO₃ (464 mg, 1.42 mmol), 4-bromo-1-(tetrahydro-2H-pyran-2-yl)-1H-indazole (200 mg, 0.711 mmol) and dihydropyrimidine-2,4 (1H,3H)-dione (244 mg, 2.14 mmol) in 1,4-dioxane (10 mL) at r.t. under N₂. The resulting mixture was stirred at 100° C. for 16 h. The solvent was then removed under reduced pressure. The crude product was purified by FSC (gradient: 0-7% MeOH in DCM) to give the title compound (180 mg, 81%) as a yellow solid. m/z (ES⁺), [M+H]⁺=315.2.

### Example 34: 1-(1H-Indazol-4-yl)dihydropyrimidine-2,4 (1H,3H)-dione

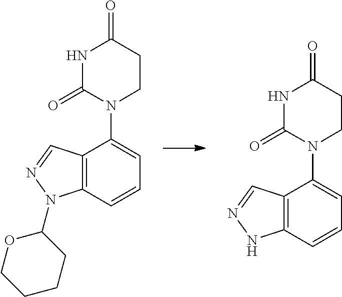 [SMILES: O=C1CCN(c2cccc3c2cnn3C2CCCCO2)C(=O)N1 ; O=C1CCN(c2cccc3[nH]ncc23)C(=O)N1]

HCl in 1,4-dioxane (4M, 1.27 mL, 5.08 mmol) was added to a solution of 1-(1-(tetrahydro-2H-pyran-2-yl)-1H-indazol-4-yl)dihydropyrimidine-2,4 (1H, 3H)-dione (160 mg, 0.509 mmol) in DCM (10 mL). The resulting solution was stirred at r.t. for 4 h. The solvent was then removed under reduced pressure. Purification by C-18FC (gradient: 5-23% MeCN in water (containing 0.1% FA)) gave the title compound (52.0 mg, 44%) as a white solid. ¹H NMR: δ 2.79 (2H, t), 3.88 (2H, t), 7.02 (1H, d), 7.36 (1H, t), 7.46 (1H, d), 8.02 (1H, s), 10.43 (1H, s). m/z (ES⁺), [M+H]⁺=231.0.

### Intermediate 35a: tert-Butyl 5-(2,4-dioxotetrahydropyrimidin-1 (2H)-yl)-1H-indazole-1-carboxylate

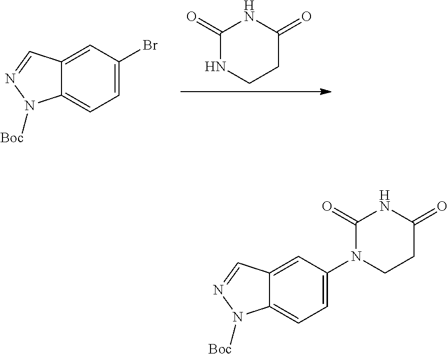 [SMILES: CC(C)(C)OC(=O)n1ncc2cc(Br)ccc21 ; O=C1CCNC(=O)N1 ; CC(C)(C)OC(=O)n1ncc2cc(N3CCC(=O)NC3=O)ccc21]

Ephos (18.0 mg, 0.0337 mmol) and Ephos Pd G4 (30.9 mg, 0.0336 mmol) were added to a degassed mixture of Cs₂CO₃ (658 mg, 2.02 mmol), dihydropyrimidine-2,4 (1H,3H)-dione (230 mg, 2.02 mmol) and tert-butyl 5-bromo-1H-indazole-1-carboxylate (200 mg, 0.673 mmol) in 1,4-dioxane (12 mL) at r.t. under N₂. The resulting mixture was stirred at 100° C. for 15 h. The resulting reaction mixture was filtered, washed with THF and the solvents of the filtrate were removed under reduced pressure. Purification by C-18FC (gradient 0-100% MeCN in water) gave the title compound (60.0 mg, 27%) as a white solid. ¹H NMR: δ 10.42 (s, 1H), 8.43 (d, 1H), 8.07 (d, 1H), 7.83 (d, 1H), 7.65-7.50 (m, 1H), 3.83 (dt, 2H), 2.76 (t, 2H), 1.66 (s, 9H). m/z (ES⁺), [M+H]⁺=331.2.

### Example 35: 1-(1H-Indazol-5-yl)dihydropyrimidine-2,4 (1H,3H)-dione

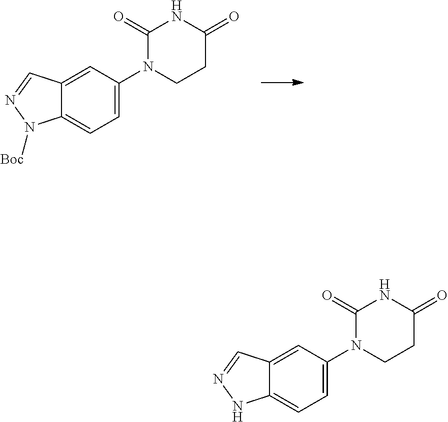 [SMILES: CC(C)(C)OC(=O)n1ncc2cc(N3CCC(=O)NC3=O)ccc21 ; O=C1CCN(c2ccc3[nH]ncc3c2)C(=O)N1]

A suspension of tert-butyl 5-(2,4-dioxotetrahydropyrimidin-1 (2H)-yl)-1H-indazole-1-carboxylate (18 mg, 0.054 mmol) in water (10 mL) was stirred at 100° C. for 6 h. Water was removed under reduced pressure. Purification by preparative HPLC (Column C, Eluent D, gradient: 5-20%) gave the title compound (2.1 mg, 17%) as a white solid. ¹H NMR: δ 8.08 (s, 1H), 7.68 (d, 1H), 7.54 (d, 1H), 7.32 (dd, 1H), 3.80 (t, 2H), 2.73 (t, 2H). m/z (ES⁺), [M+H]⁺=231.0.

### Intermediate 36a: 5-(2,4-Dioxotetrahydropyrimidin-1 (2H)-yl)-3-hydroxypicolinaldehyde

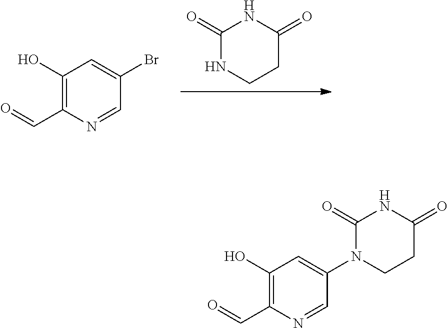 [SMILES: O=Cc1ncc(Br)cc1O ; O=C1CCNC(=O)N1 ; O=Cc1ncc(N2CCC(=O)NC2=O)cc1O]

Ephos Pd G4 (59.1 mg, 0.0643 mmol) was added to a degassed mixture of 5-bromo-3-hydroxypicolinaldehyde (260 mg, 1.29 mmol), dihydropyrimidine-2,4 (1H,3H)-dione (441 mg, 3.86 mmol), Ephos (34.4 mg, 0.0643 mmol) and Cs₂CO₃ (839 mg, 2.58 mmol) in 1,4-dioxane (20 mL) at r.t. under N₂. The resulting mixture was stirred at 100° C. for 16 h. The solvent was then removed under reduced pressure. Purification by C-18FC (gradient: 0-10% MeCN in water (containing 0.1% FA)) gave the title compound (0.100 g, 33%) as a yellow solid. ¹H NMR: δ 2.74 (2H, t), 3.93 (2H, t), 7.43 (1H, d), 8.37 (1H, d), 10.04 (1H, s), 10.65 (1H, s), 10.92 (1H, s). m/z (ES⁺), [M+H]⁺=236.2.

### Intermediate 36b: (E)-5-(2,4-Dioxotetrahydropyrimidin-1 (2H)-yl)-3-hydroxypicolinaldehyde oxime

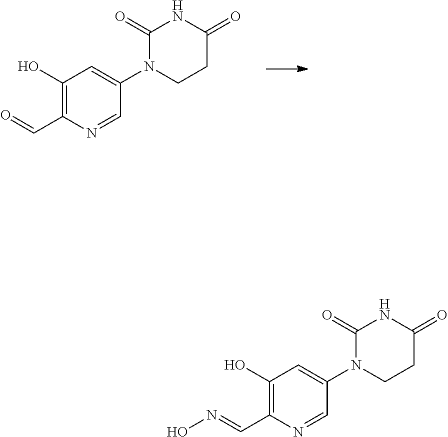 [SMILES: O=Cc1ncc(N2CCC(=O)NC2=O)cc1O ; O=C1CCN(c2cnc(/C=N/O)c(O)c2)C(=O)N1]

NaOAc (94.0 mg, 1.15 mmol) was added to a mixture of 5-(2,4-dioxotetrahydropyrimidin-1 (2H)-yl)-3-hydroxypicolinaldehyde (90.0 mg, 0.383 mmol) and hydroxylamine hydrochloride (53.2 mg, 0.766 mmol) in MeOH (15 mL) at r.t. under air. The resulting mixture was stirred at r.t. for 1 h. The solvent was then removed under reduced pressure. Purification by FSC (gradient: 0-8% MeOH in DCM) gave the title compound (70.0 mg, 73%) as a white solid. ¹H NMR: δ 2.72 (2H, t), 3.86 (2H, t), 7.35 (1H, d), 8.19 (1H, d), 8.30 (1H, s), 10.41 (1H, s), 10.53 (1H, s), 11.83 (1H, s). m/z (ES⁺), [M+H]⁺=251.2.

### Example 36: 1-(Isoxazolo[4,5-b]pyridin-6-yl)dihydropyrimidine-2,4 (1H,3H)-dione

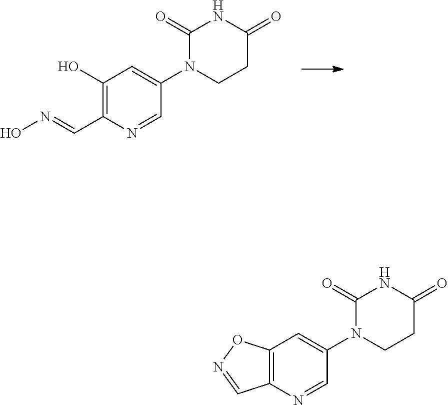 [SMILES: O=C1CCN(c2cnc(/C=N/O)c(O)c2)C(=O)N1 ; O=C1CCN(c2cnc3cnoc3c2)C(=O)N1]

DIAD (69.9 μL, 0.360 mmol) was added to a mixture of (E)-5-(2,4-dioxotetrahydropyrimidin-1 (2H)-yl)-3-hydroxypicolinaldehyde oxime (60 mg, 0.240 mmol), PPh₃ (94 mg, 0.358 mmol) in THF (3 mL) at r.t. under N₂. The resulting mixture was stirred at r.t. for 16 h. The solvent was then removed under reduced pressure. Purification by C-18FC (gradient: 5-30% MeCN in water (containing 0.05% TFA)) gave the title compound (40 mg, 72%) as a white solid. ¹H NMR: δ 2.73 (2H, t), 3.89 (2H, t), 7.45 (1H, d), 8.23 (1H, d), 10.64 (1H, s), 11.76 (1H, s). m/z (ES⁺), [M+H]⁺=233.2.

### Intermediate 37a: 6-Bromo-2-(trimethylsilyl)furo[3,2-b]pyridine

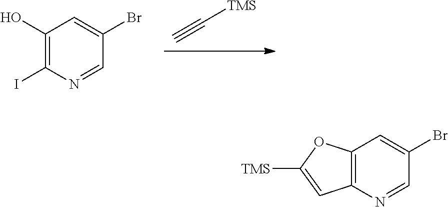

Ethynyltrimethylsilane (246 mg, 2.50 mmol) was added to a mixture of copper(I) iodide (31.8 mg, 0.167 mmol), bis(triphenylphosphine)palladium chloride (117 mg, 0.167 mmol) and 5-bromo-2-iodopyridin-3-ol (500 mg, 1.67 mmol) in triethylamine (10 mL) at r.t. under N₂. The resulting mixture was stirred at r.t. for 17 h. The solvent was then removed under reduced pressure. Purification by FSC (gradient 0-10% EtOAc in petroleum ether) gave the title compound (340 mg, 75%) as a brown solid. ¹H NMR: δ 0.36 (9H, s), 7.41 (1H, d), 8.44 (1H, dd), 8.61 (1H, d). m/z (ES⁺), [M+H]⁺=272.1.

### Intermediate 37b: 1-(2-(Trimethylsilyl)furo[3,2-b]pyridin-6-yl)dihydropyrimidine-2,4 (1H,3H)-dione

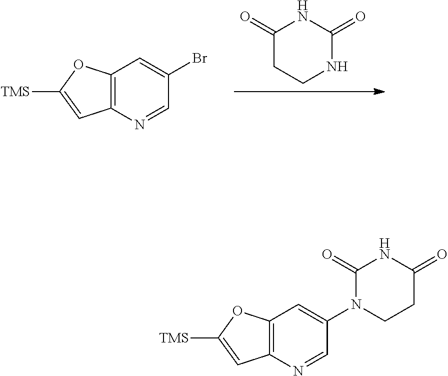 [SMILES: C[Si](C)(C)c1cc2ncc(Br)cc2o1 ; O=C1CCNC(=O)N1 ; C[Si](C)(C)c1cc2ncc(N3CCC(=O)NC3=O)cc2o1]

Ephos (59.4 mg, 0.111 mmol) and Ephos Pd G4 (102 mg, 0.111 mmol) were added to a degassed mixture of Cs₂CO₃ (724 mg, 2.22 mmol), dihydropyrimidine-2,4 (1H,3H)-dione (380 mg, 3.33 mmol) and 6-bromo-2-(trimethylsilyl)furo[3,2-b]pyridine (300 mg, 1.11 mmol) in 1,4-dioxane (20 mL) at r.t. under N₂. The resulting mixture was stirred at 100° C. for 17 h. The solvent was then removed under reduced pressure. Purification by C-18FC (gradient: 0-50% MeCN in water (containing 0.1% FA)) gave the title compound (120 mg, 36%) as a pale yellow solid. ¹H NMR: δ 0.35 (9H, s), 2.75 (2H, t), 3.85 (2H, t), 7.36 (1H, d), 7.99-8.06 (1H, m), 8.51 (1H, d), 10.51 (1H, s). m/z (ES⁺), [M+H]⁺=304.2.

### Example 37: 1-(Furo[3,2-b]pyridin-6-yl)dihydropyrimidine-2,4 (1H,3H)-dione

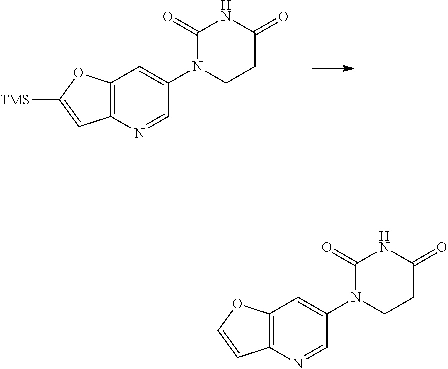 [SMILES: C[Si](C)(C)c1cc2ncc(N3CCC(=O)NC3=O)cc2o1 ; O=C1CCN(c2cnc3ccoc3c2)C(=O)N1]

A solution of tetra-n-butylammonium fluoride in THF (1M, 330 μL, 0.330 mmol) was added to a solution of 1-(2-(trimethylsilyl)furo[3,2-b]pyridin-6-yl)dihydropyrimidine-2,4 (1H,3H)-dione (100 mg, 0.330 mmol) in THF (10 mL) at r.t. under air. The resulting mixture was stirred at r.t. for 20 minutes. The solvent was then removed under reduced pressure. Purification by C-18FC (gradient: 0-40% MeCN in water (containing 0.1% NH₄HCO₃)) gave the title compound (23.0 mg, 30%) as a pale yellow solid. ¹H NMR: δ 2.75 (2H, t), 3.86 (2H, t), 7.14 (1H, dd), 8.07 (1H, dd), 8.32 (1H, d), 8.53 (1H, d), 10.51 (1H, br s). m/z (ES⁺), [M+H]⁺=232.2.

### Intermediate 38a: 6-Bromo-1-((2-(trimethylsilyl)ethoxy)methyl)-1H-benzo[d][1,2,3]triazole

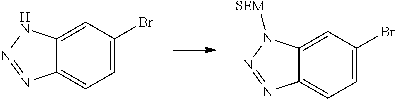 [SMILES: Brc1ccc2nn[nH]c2c1 ; C[Si](C)(C)CCOCn1nnc2ccc(Br)cc21]

NaH (60% dispersion in mineral oil, 99.0 mg, 2.47 mmol) was added to 6-bromo-1H-benzo[d][1,2,3]triazole (245 mg, 1.24 mmol) in DMF (6 mL) at r.t. under N₂. The resulting mixture was stirred at r.t. for 0.5 h before the addition of (2-(chloromethoxy)ethyl)trimethylsilane (268 mg, 1.61 mmol). The resulting solution was stirred at r.t. for 16 h. The reaction was quenched with NH₄C1 (1 mL) and then directly purified by C-18FC (gradient: 50-90% MeCN in water) to give the title compound (265 mg, 65%) as a brown oil which was used in the next step without further purification (this material contains the title compound and a 2^{nd} unidentified regioisomer in a 1:1 ratio). m/z (ES⁺), [M+H]⁺=328.0.

### Intermediate 38b: 1-(1-((2-(Trimethylsilyl)ethoxy)methyl)-1H-benzo[d][1,2,3]triazol-6-yl)dihydro-pyrimidine-2,4 (1H,3H)-dione

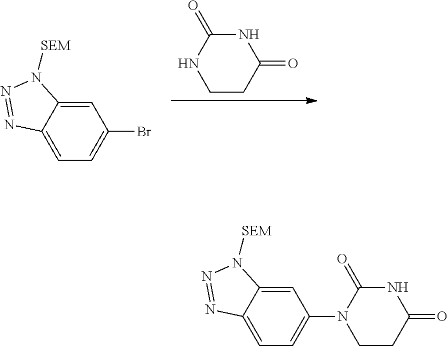 [SMILES: C[Si](C)(C)CCOCn1nnc2ccc(Br)cc21 ; O=C1CCNC(=O)N1 ; C[Si](C)(C)CCOCn1nnc2ccc(N3CCC(=O)NC3=O)cc21]

Cs₂CO₃ (595 mg, 1.83 mmol) was added to a degassed mixture of 6-bromo-1-((2-(trimethylsilyl)ethoxy)-methyl)-1H-benzo[d][1,2,3]triazole (This material contains the stated compound and a 2^{nd} unidentified regioisomer in a 1:1 ratio) (200 mg, 0.609 mmol), dihydropyrimidine-2,4 (1H,3H)-dione (209 mg, 1.83 mmol) Ephos (16.3 mg, 0.0305 mmol) and Ephos Pd G4 (28.0 mg, 0.0305 mmol) in 1,4-dioxane (10 mL) at r.t. under N₂. The resulting mixture was stirred at 100° C. for 20 h. The solvent was then removed under reduced pressure. Purification by C-18FC (gradient: 30-60% MeCN in water) gave the title compound (200 mg, 91%) as a white solid (this material contains the title compound and a 2^{nd} unidentified regioisomer in a 1:1 ratio) which was used in the next step without further purification. m/z (ES⁺), [M+H]⁺=362.2.

### Example 38: 1-(1H-Benzo[d][1,2,3]triazol-6-yl)dihydropyrimidine-2,4 (1H,3H)-dione

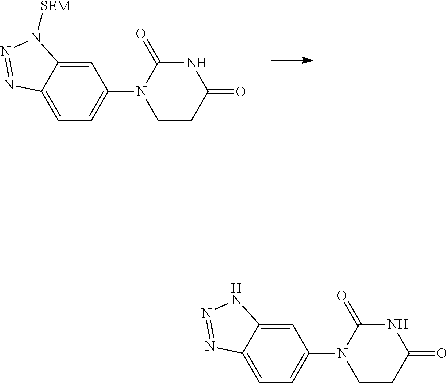 [SMILES: C[Si](C)(C)CCOCn1nnc2ccc(N3CCC(=O)NC3=O)cc21 ; O=C1CCN(c2ccc3nn[nH]c3c2)C(=O)N1]

1-(1-((2-(Trimethylsilyl)ethoxy)methyl)-1H-benzo[d][1,2,3]triazol-6-yl)dihydropyrimidine-2,4 (1H,3H)-dione (this material contains the stated compound and a 2^{nd} unidentified regioisomer in a 1:1 ratio) (70 mg, 0.194 mmol) was added to a solution of HCl in EtOAc (4M, 5.00 mL, 20.0 mmol) to give a white suspension. The resulting mixture was stirred at r.t. for 2 h. The solvent was then removed under reduced pressure. The crude product was purified by preparative HPLC (Column A, Eluent F, gradient: 0-2%) to give the title compound (35.0 mg, 78%) as a white solid. ¹H NMR: δ 2.75 (2H, t), 3.87 (2H, t), 7.41 (1H, dd), 7.81 (1H, d), 7.90 (1H, d), 10.44 (1H, s). m/z (ES⁺), [M+H]⁺=232.2.

### Intermediate 39a: 4-(2,4-Dioxotetrahydropyrimidin-1 (2H)-yl)-2-hydroxybenzaldehyde

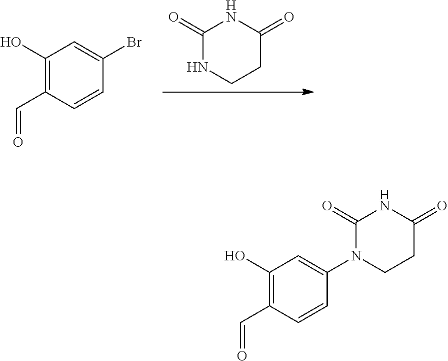 [SMILES: O=Cc1ccc(Br)cc1O ; O=C1CCNC(=O)N1 ; O=Cc1ccc(N2CCC(=O)NC2=O)cc1O]

A degassed mixture of Cs₂CO₃ (584 mg, 1.79 mmol), 4-bromo-2-hydroxybenzaldehyde (120 mg, 0.597 mmol), dihydropyrimidine-2,4 (1H,3H)-dione (204 mg, 1.79 mmol), Ephos (16.0 mg, 0.0299 mmol) and Ephos Pd G4 (27.4 mg, 0.0298 mmol) in 1,4-dioxane (10 mL) was stirred at 100° C. under N, for 15 h. The reaction mixture was directly purified by C-18FC (gradient: 0-100% MeCN in water (containing 0.1% FA)) to give the title compound (50.0 mg, 36%) as a yellow solid. ¹H NMR: δ 10.85 (s, 1H), 10.52 (s, 1H), 10.19 (s, 1H), 7.66 (d, 1H), 7.09-6.88 (m, 2H), 3.86 (t, 2H), 2.72 (t, 2H). m/z (ES⁺), [M+H]⁺=235.1.

### Intermediate 39b: 4-(2,4-Dioxotetrahydropyrimidin-1 (2H)-yl)-2-hydroxybenzaldehyde oxime

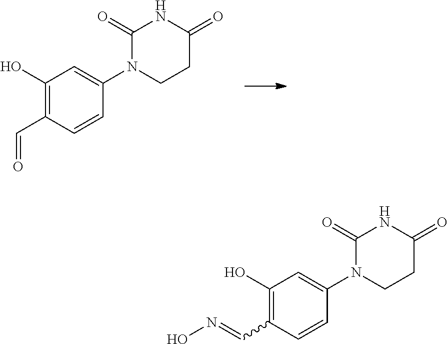 [SMILES: O=Cc1ccc(N2CCC(=O)NC2=O)cc1O ; O=C1CCN(c2ccc(C=NO)c(O)c2)C(=O)N1]

Hydroxylamine-O-sulfonic acid (77.0 mg, 0.681 mmol) was added to a stirred suspension of 4-(2,4-dioxotetra-hydropyrimidin-1 (2H)-yl)-2-hydroxybenzaldehyde (80.0 mg, 0.342 mmol) in MeOH (10 mL). The resulting mixture was stirred at r.t. for 0.5 h, then NaHCO₃ (57.4 mg, 0.683 mmol) was added to the mixture. Water (1 mL) was then added and the resulting mixture was stirred for 0.5 h before the addition of 0.5 mL HCl (1M). The solvent was removed under reduced pressure and the residue was dissolved in MeOH (10 mL) and the mixture filtered. The filtrate was concentrated and purified by C-18FC (gradient: 0-100% MeCN in water (containing 0.1% TFA)) to give the title compound as an inseparable mixture of isomers (E:Z=4:1, 65.0 mg, 76%) as a yellow solid which was used in the next step without further purification. m/z (ES⁺), [M+H]⁺=250.0.

### Example 39: 1-(Benzo[d]isoxazol-6-yl)dihydropyrimidine-2,4 (1H,3H)-dione

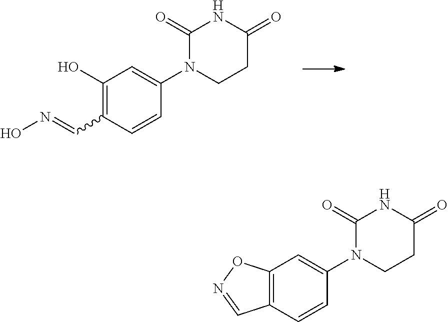 [SMILES: O=C1CCN(c2ccc(C=NO)c(O)c2)C(=O)N1 ; O=C1CCN(c2ccc3cnoc3c2)C(=O)N1]

DIAD (88.0 μL, 0.453 mmol) was added dropwise to a stirred suspension of PPh₃ (95.0 mg, 0.362 mmol) and an inseparable mixture of isomers of 4-(2,4-dioxotetrahydropyrimidin-1 (2H)-yl)-2-hydroxybenzaldehyde oxime (E:Z=4:1, 45.0 mg, 0.181 mmol) in DCM (10 mL) at r.t. The resulting mixture was stirred at r.t. for 2 h and then at 50° C. for 4 h. The solvent was removed under reduced pressure. Purification by C-18FC (gradient: 0-100% MeCN in water) gave material that was further purified by preparative HPLC (Column A, Eluent F, gradient: 3-10%) to give the title compound (10.0 mg, 24%) as a white solid. ¹H NMR: δ 7.33 (d, 1H), 6.65 (d, 1H), 6.50 (dd, 1H), 6.01 (s, 1H), 3.72 (t, 2H), 2.66 (t, 2H). m/z (ES⁻), [M−H]⁻=230.0

### Intermediate 40a: tert-Butyl 6-bromo-1H-pyrrolo[3,2-b]pyridine-1-carboxylate

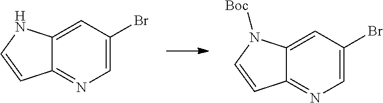 [SMILES: Brc1cnc2cc[nH]c2c1 ; CC(C)(C)OC(=O)n1ccc2ncc(Br)cc21]

Di-tert-butyl dicarbonate (884 μL 3.81 mmol) was added to a mixture of 6-bromo-1H-pyrrolo[3,2-b]pyridine (500 mg, 2.54 mmol), triethylamine (707 μL, 5.08 mmol) and DMAP (31.0 mg, 0.254 mmol) in DCM (20 mL) at r.t. under N₂. The resulting mixture was stirred at r.t. for 2 h. The solvent was then removed under reduced pressure. Purification by FSC (gradient: 0-30% EtOAc in petroleum ether) to give the title compound (0.700 g, 93%) as a white solid. ¹H NMR: (CDCl₃) δ 1.68 (9H, s), 6.75 (1H, d), 7.79 (1H, d), 8.57 (2H, d). m/z (ES⁺), [M+H]⁺=297.1.

### Intermediate 40b: tert-Butyl 6-(2,4-dioxotetrahydropyrimidin-1 (2H)-yl)-1H-pyrrolo[3,2-b]pyridine-1-carboxylate

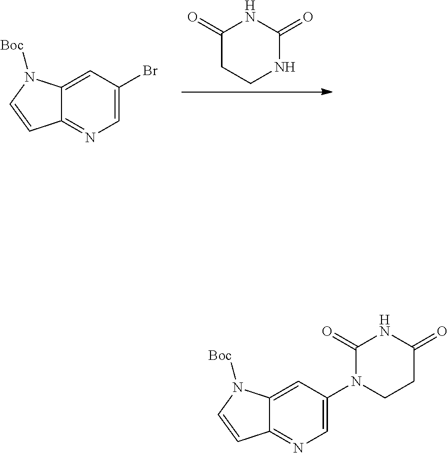 [SMILES: CC(C)(C)OC(=O)n1ccc2ncc(Br)cc21 ; O=C1CCNC(=O)N1 ; CC(C)(C)OC(=O)n1ccc2ncc(N3CCC(=O)NC3=O)cc21]

Ephos (45.0 mg, 0.0841 mmol) and Ephos Pd G4 (77.0 mg, 0.0838 mmol) were added to a mixture of Cs₂CO₃ (548 mg, 1.68 mmol), dihydropyrimidine-2,4 (1H,3H)-dione (288 mg, 2.52 mmol) and tert-butyl 6-bromo-1H-pyrrolo[3,2-b]pyridine-1-carboxylate (250 mg, 0.84 mmol) in 1,4-dioxane (20 mL) at r.t. under N₂. The resulting mixture was stirred at 100° C. for 16 h. The solvent was then removed under reduced pressure. Purification by C-18FC (gradient: 5-50% MeCN in water (containing 0.1% FA)) gave the title compound (150 mg, 54%) as a white solid. ¹H NMR: δ 1.64 (9H, s), 2.77 (2H, t), 3.90 (2H, t), 6.84 (1H, d), 7.99 (1H, d), 8.31 (1H, d), 8.50 (1H, d), 10.48 (1H, s). m/z (ES⁺), [M+H]⁺=331.1.

### Example 40: 1-(1H-Pyrrolo[3,2-b]pyridin-6-yl)dihydropyrimidine-2,4 (1H,3H)-dione

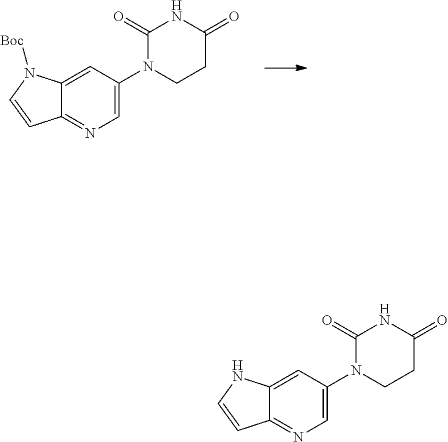 [SMILES: CC(C)(C)OC(=O)n1ccc2ncc(N3CCC(=O)NC3=O)cc21 ; O=C1CCN(c2cnc3cc[nH]c3c2)C(=O)N1]

tert-Butyl 6-(2,4-dioxotetrahydropyrimidin-1 (2H)-yl)-1H-pyrrolo[3,2-b]pyridine-1-carboxylate (150 mg, 0.454 mmol) was dissolved in 2,2,2-trifluoroethanol (10 mL) and sealed into a microwave tube. The reaction was heated to 120° C. for 1 h in a microwave reactor and then cooled to r.t. The solvent was then removed under reduced pressure. Purification by C-18FC (gradient: 0-10% MeCN in water (containing 0.1% FA)) to give the title compound (78.0 mg, 75%) as a white solid. ¹H NMR: δ 2.76 (2H, t), 3.84 (2H, t), 6.54-6.60 (1H, m), 7.68 (1H, t), 7.72-7.77 (1H, m), 8.30 (1H, d), 10.40 (1H, s), 11.40 (1H, s). m/z (ES⁺), [M+H]⁺=231.0.

### Intermediate 41a: 6-Bromo-1-((2-(trimethylsilyl)ethoxy)methyl)-1H-benzo[d]imidazole

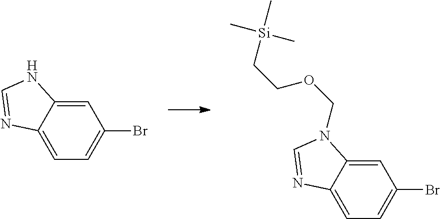 [SMILES: Brc1ccc2nc[nH]c2c1 ; C[Si](C)(C)CCOCn1cnc2ccc(Br)cc21]

NaH (60% dispersion in mineral oil, 0.264 g, 6.60 mmol) was added to a solution of 6-bromo-1H-benzo[d]imidazole (1.00 g, 5.08 mmol) in DMF (15 mL) at 0° C. under N₂. The resulting suspension was stirred at r.t for 15 minutes before the addition of (2-(chloromethoxy)ethyl)trimethylsilane (1.10 g, 6.60 mmol) and the mixture was then stirred at r.t. for 2 h. The reaction mixture was quenched with water (10 mL) and extracted with EtOAc (3×50 mL). The combined organic extracts were dried (Na₂SO₄) and concentrated to give a brown residue. Purification by FSC (gradient: 0-10% EtOAc in petroleum ether) gave the title compound (1.40 g, 84%) as a yellow liquid (this material contains the title compound and the regioisomer: 5-bromo-1-((2-(trimethylsilyl)ethoxy)methyl)-1H-benzo[d]imidazole in a 94:6 ratio) which was used in the next step without further purification. m/z (ES⁺), [M+H]⁺=329.0.

### Intermediate 41b: 1-(1-((2-(Trimethylsilyl)ethoxy)methyl)-1H-benzo[d]imidazol-6-yl)dihydropyrimidine-2,4 (1H,3H)-dione

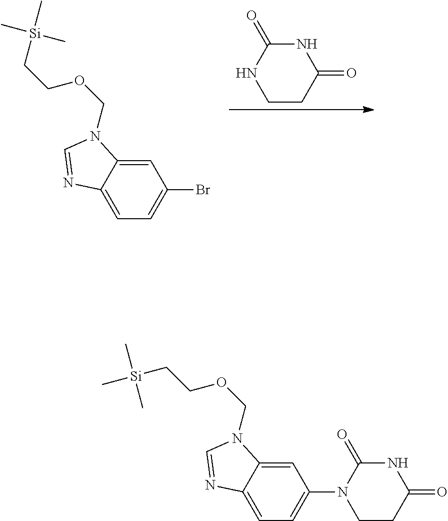 [SMILES: C[Si](C)(C)CCOCn1cnc2ccc(Br)cc21 ; O=C1CCNC(=O)N1 ; C[Si](C)(C)CCOCn1cnc2ccc(N3CCC(=O)NC3=O)cc21]

Cs₂CO₃ (398 mg, 1.22 mmol) was added to a mixture of 6-bromo-1-((2-(trimethylsilyl)ethoxy)methyl)-1H-benzo[d]imidazole (this material contains the stated compound and the regioisomer: 5-bromo-1-((2-(trimethylsilyl)ethoxy)methyl)-1H-benzo[d]imidazole in a 94:6 ratio) (200 mg, 0.611 mmol), Ephos Pd G4 (31.6 mg, 0.0344 mmol), Ephos (16.4 mg, 0.0307 mmol) and dihydropyrimidine-2,4 (1H,3H)-dione (139 mg, 1.22 mmol) in 1,4-dioxane (10 mL) under N₂. The resulting solution was stirred at 100° C. for 16 h. The reaction was filtered and the filtrate concentrated. Purification by C-18FC (gradient: 0-80% MeCN in water) gave the title compound (100 mg, 45%) as a white solid (this material was also contaminated with an indistinguishable quantity of its regioisomer) which was used in the next step without further purification. m/z (ES⁺), [M+H]⁺=361.2.

### Example 41: 1-(1H-Benzo[d]imidazol-6-yl)dihydropyrimidine-2,4 (1H,311)-dione

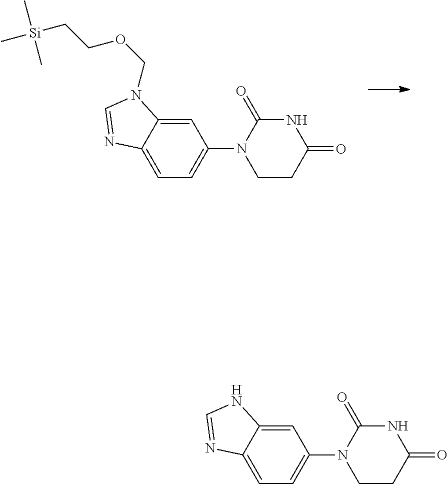 [SMILES: C[Si](C)(C)CCOCn1cnc2ccc(N3CCC(=O)NC3=O)cc21 ; O=C1CCN(c2ccc3nc[nH]c3c2)C(=O)N1]

1-(1-((2-(Trimethylsilyl)ethoxy)methyl)-1H-benzo[d]imidazol-6-yl)dihydropyrimidine-2,4 (1H,3H)-dione (this material was contaminated with an indistinguishable quantity of its regioisomer) (50.0 mg, 0.139 mmol) was added to a solution of HCl in 1,4-dioxane (4M, 4.00 mL, 16.0 mmol) and stirred at 50° C. for 16 h. The solvent was removed under reduced pressure and the crude product purified by preparative HPLC (Column B, Eluent B, gradient: 2-50%) to give the title compound (11.0 mg, 34%) as a white solid. ¹H NMR: δ 8.25 (s, 1H), 7.55 (m, 2H), 7.16 (d, 1H), 3.81 (t. 2H), 2.74 (t. 2H). m/z (ES⁺), [M+H]⁺=231.2.

### Intermediate 42a: 3-(4-Methoxybenzyl)dihydropyrimidine-2,4 (1H,3H)-dione

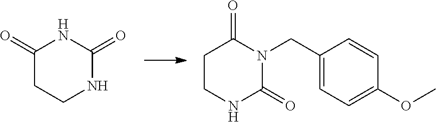 [SMILES: O=C1CCNC(=O)N1 ; COc1ccc(CN2C(=O)CCNC2=O)cc1]

Cs₂CO₃ (5.71 g, 17.5 mmol) was added to a solution of 1-(chloromethyl)-4-methoxybenzene (915 mg, 5.84 mmol) and dihydropyrimidine-2,4 (1H,3H)-dione (1.00 g, 8.76 mmol) in DMF (30 mL) at r.t. under air. The resulting mixture was stirred at r.t. for 20 h. The reaction mixture was poured into water (100 mL), extracted with EtOAc (100 mL) and the organic layer washed with brine (3×100 ml). The organic layer was dried (Na₂SO₄) and concentrated to give a pale yellow solid. The crude solid was triturated with EtOAc to give a solid which was collected by filtration and dried under vacuum to give the title compound (1.20 g, 88%) as a white solid. ¹H NMR: δ 2.62 (2H, t), 3.15-3.27 (2H, m), 3.71 (3H, s), 4.71 (2H, s), 6.79-6.89 (2H, m), 7.12-7.22 (2H, m), 7.79 (1H, s). m/z (ES⁺), [M+H]⁺=235.1.

### Intermediate 42b: tert-Butyl 6-(3-(4-methoxybenzyl)-2,4-dioxotetrahydropyrimidin-1 (2H)-yl)-1H-indazole-1-carboxylate

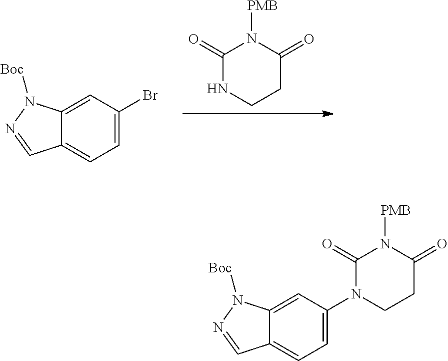

Pd₂(dba)₃ (231 mg, 0.252 mmol) was added to a degassed mixture of xantphos (292 mg, 0.505 mmol), Cs₂CO₃ (1.10 g, 3.38 mmol), tert-butyl 6-bromo-1H-indazole-1-carboxylate (500 mg, 1.68 mmol) and 3-(4-methoxybenzyl)dihydropyrimidine-2,4 (1H,3H)-dione (464 mg, 1.68 mmol) in 1,4-dioxane (30 mL) at r.t. under N₂. The resulting mixture was stirred at 100° C. for 16 h. The solvent was then removed under reduced pressure. Purification by C-18FC (gradient: 0-70% MeCN in water (containing 0.1% NH₄HCO₃)) gave the title compound (200 mg, 26%) as a white solid. ¹H NMR: δ 1.64 (9H, d), 2.94 (2H, t), 3.72 (3H, s), 3.92 (2H, t), 4.83 (2H, s), 6.81-6.93 (2H, m), 7.15-7.29 (2H, m), 7.38 (1H, dd), 7.88 (1H, d), 8.04-8.10 (1H, m), 8.41 (1H, s). m/z (ES⁺), [M-Boc+2H]⁺=351.1.

### Example 42: 1-(1H-Indazol-6-yl)dihydropyrimidine-2,4 (1H,3H)-dione

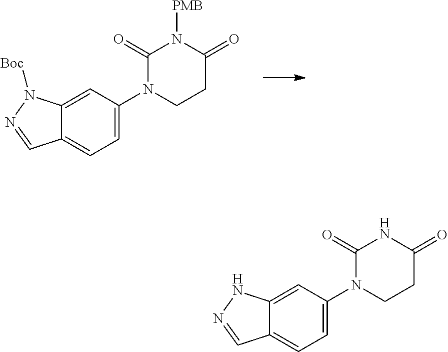

Ceric ammonium nitrate (2.56 g, 4.67 mmol) was added to tert-butyl 6-(3-(4-methoxybenzyl)-2,4-dioxotetra-hydropyrimidin-1 (2H)-yl)-1H-indazole-1-carboxylate (700 mg, 1.55 mmol) in MeCN (10 mL) and water (10 mL) at r.t. under air. The resulting solution was stirred at r.t. for 4 h. The solvent was removed under reduced pressure. Purification by C-18FC (gradient: 0-40% MeCN in water (containing 0.1% conc. HCl)) provided material that was further purified by preparative HPLC (Column F, Eluent A, gradient: 15-18%) to give the title compound (40.0 mg, 11%) as a white solid. ¹H NMR: δ 2.75 (2H, t), 3.86 (2H, t), 7.10 (1H, dd), 7.44-7.50 (1H, m), 7.75 (1H, d), 8.07 (1H, s), 10.38 (1H, s), 13.09 (1H, s). m/z (ES⁺), [M+H]⁺=231.1.

### Intermediate 43a: 4-bromo-2-(4-methoxybenzyl)isoindolin-1-one

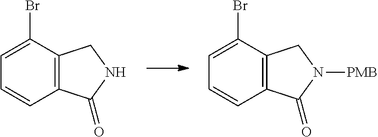

NaH (60% dispersion in mineral oil, 28.5 mg, 1.19 mmol) was added to a solution of 4-bromoisoindolin-1-one (229 mg, 1.08 mmol) in DMF (10 mL) at 0° C. under N₂. The mixture was stirred at r.t. for 3 h before the addition of 1-(chloromethyl)-4-methoxybenzene (169 mg, 1.08 mmol) at 0° C. The resulting mixture was stirred at r.t. overnight before the addition of MeOH (1 mL). Then the mixture was purified directly by C-18FC (gradient: 0-80% MeCN in water) to give the title compound (300 mg, 84%) as a pale yellow gum. ¹H NMR: δ 7.81 (dd, 1H), 7.75 (dd, 1H), 7.49 (t, 1H), 7.30-7.21 (m, 2H), 6.96-6.88 (m, 2H), 4.68 (s, 2H), 4.26 (s, 2H), 3.74 (s, 3H). m/z (ES⁺), [M+H]⁺=334.1.

### Intermediate 43b: 1-(2-(4-Methoxybenzyl)-1-oxoisoindolin-4-yl)dihydropyrimidine-2,4 (1H,3H)-dione

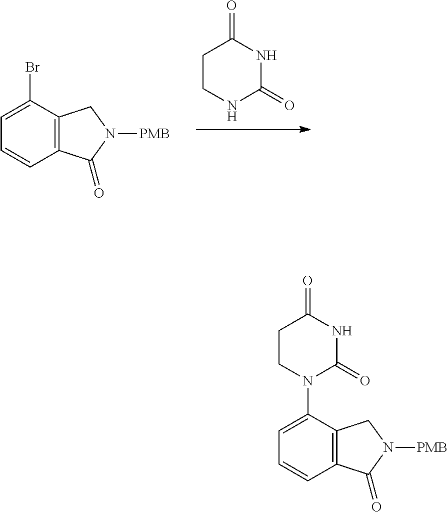

1^{st} experiment: Cs₂CO₃ (294 mg, 0.902 mmol) was added to a degassed mixture of 4-bromo-2-(4-methoxybenzyl)isoindolin-1-one (100 mg, 0.301 mmol), dihydropyrimidine-2,4 (1H,3H)-dione (103 mg, 0.903 mmol), Ephos (16.1 mg, 0.0301 mmol) and Ephos Pd G4 (27.7 mg, 0.0302 mmol) in 1,4-dioxane (8 mL) under N₂. The resulting suspension was stirred at 100° C. for 16 h. The solvent was removed under reduced pressure to give the crude product.

2^{nd} experiment: Cs₂CO₃ (441 mg, 1.35 mmol) was added to a degassed mixture of 4-bromo-2-(4-methoxy-benzyl)isoindolin-1-one (150 mg, 0.452 mmol), dihydropyrimidine-2,4 (1H,3H)-dione (155 mg, 1.36 mmol), Ephos (24.2 mg, 0.0453 mmol) and Ephos Pd G4 (41.5 mg, 0.0452 mmol) in 1,4-dioxane (8 mL) under N₂. The resulting suspension was stirred at 100° C. for 16 h. The reaction was combined with the crude product of the 1^{st} experiment and the solvent was removed under reduced pressure. Purification by C-18FC (gradient: 0-60% MeCN in water) gave the title compound (50 mg, average yield 18%) as a white solid. ¹H NMR: (CDCl₃) δ 7.89 (dd, 1H), 7.58 (t, 1H), 7.44 (s, 1H), 7.38 (dd, 1H), 7.27 (d, 2H), 6.92-6.86 (m, 2H), 4.76 (s, 2H), 4.25 (s, 2H), 3.88 (t, 2H), 3.82 (s, 3H), 2.84 (t, 2H). m/z (ES⁺), [M+H]⁺=366.1.

### Example 43: 1-(1-Oxoisoindolin-4-yl)dihydropyrimidine-2,4 (1H,3H)-dione

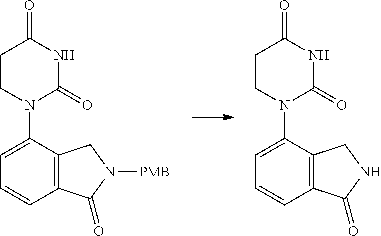

A solution of 1-(2-(4-methoxybenzyl)-1-oxoisoindolin-4-yl)dihydropyrimidine-2,4 (1H,3H)-dione (80.0 mg, 0.219 mmol) in TFA (10 mL) was stirred at 90° C. for 5 h. The solvent was removed under reduced pressure. Purification by preparative HPLC (Column X, Eluent E, gradient: 5-30%) to give the title compound (34.7 mg, 65%) as a white solid. ¹H NMR: δ 10.48 (s, 1H), 8.59 (s, 1H), 7.61 (t, 1H), 7.59-7.52 (m, 2H), 4.31 (s, 2H), 3.82 (t, 2H), 2.74 (t, 2H). m/z (ES⁺), [M+H]⁺=246.1.

### Intermediate 44a: 6-Bromo-2-(4-methoxybenzyl)isoindolin-1-one

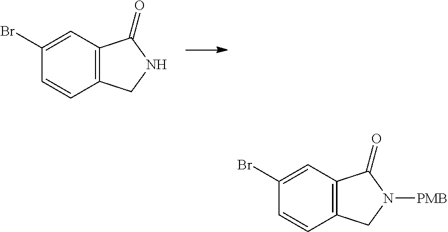

NaH (60% dispersion in mineral oil, 153 mg, 3.82 mmol) was added to a solution of 6-bromoisoindolin-1-one (540 mg, 2.55 mmol) in DMF (15 mL) at 0° C. under N₂. The resulting solution was stirred at r.t. for 3 h before the addition of 1-(chloromethyl)-4-methoxybenzene (512 mg, 3.27 mmol) at 0° C. The resulting mixture was stirred overnight at r.t. Then EtOAc (40 mL) and water (30 mL) were added to the resulting mixture. The organic layer was dried (Na₂SO₄) and concentrated to give the crude product. Purification by C-18FC (gradient: 0-100% MeCN in water) gave the title compound (320 mg, 38%) as a yellow solid. ¹H NMR: δ 7.84 (d, 1H), 7.77 (dd, 1H), 7.53 (d, 1H), 7.26-7.18 (m, 2H), 6.95-6.83 (m, 2H), 4.65 (s, 2H), 4.31 (s, 2H), 3.73 (s, 3H). m/z (ES⁺), [M+H]⁺=332/334.

### Intermediate 44b: 1-(2-(4-Methoxybenzyl)-3-oxoisoindolin-5-yl)dihydropyrimidine-2,4 (1H,3H)-dione

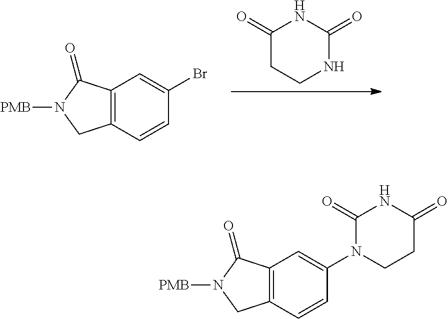

Cs₂CO₃ (353 mg, 1.08 mmol) was added to a degassed mixture of dihydropyrimidine-2,4 (1H,3H)-dione (124 mg, 1.08 mmol), 6-bromo-2-(4-methoxybenzyl)isoindolin-1-one (120 mg, 0.361 mmol), Ephos (19.3 mg, 0.0361 mmol) and Ephos Pd G4 (33.2 mg, 0.0361 mmol) in 1,4-dioxane (10 mL) under N₂. The resulting solution was stirred at 100° C. for 16 h and then purified directly by C-18FC (gradient: 0-100% MeCN in water) to give the title compound (100 mg, 76%) as a white solid. ¹H NMR: δ 10.44 (s, 1H), 7.67 (d, 1H), 7.59-7.50 (m, 2H), 7.22 (d, 2H), 6.95-6.88 (m, 2H), 4.67 (s, 2H), 4.33 (s, 2H), 3.85 (t, 2H), 3.73 (s, 3H), 2.74 (t, 2H). m/z (ES⁺), [M+H]⁺=366.2.

### Example 44: 1-(3-Oxoisoindolin-5-yl)dihydropyrimidine-2,4 (1H,3H)-dione

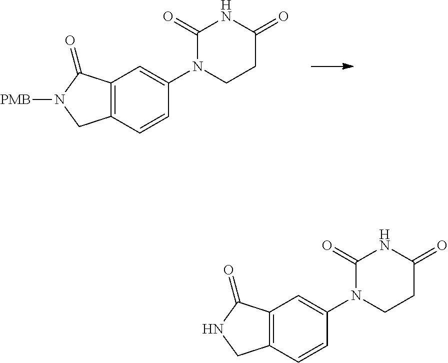

1^{st} experiment: A solution of 1-(2-(4-methoxybenzyl)-3-oxoisoindolin-5-yl)dihydropyrimidine-2,4 (1H,3H)-dione (20.0 mg, 0.0547 mmol) in TFA (1 mL) was stirred at 90° C. for 5 h before being cooled to r.t.

2^{nd} experiment: A solution of 1-(2-(4-methoxybenzyl)-3-oxoisoindolin-5-yl)dihydropyrimidine-2,4 (1H,3H)-dione (100 mg, 0.274 mmol) in TFA (2 mL) was stirred at 80° C. for 16 h. The reaction solution was combined with the 1^{st} experiment's solution and the mixture directly purified by preparative HPLC (Column B, Eluent B, gradient: 2-10%) to give the title compound (60.8 mg, average yield 75%) as a white solid. ¹H NMR: δ 8.60 (s, 1H), 7.64-7.51 (m, 3H), 4.37 (s, 2H), 3.84 (t, 2H), 2.74 (t, 2H). m/z (ES⁺), [M+H]⁺=246.1.

### Intermediate 45a: 5-Fluorodihydropyrimidine-2,4 (1H,3H)-dione

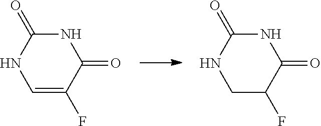 [SMILES: O=c1[nH]cc(F)c(=O)[nH]1 ; O=C1NCC(F)C(=O)N1]

Pd/C (10% on activated carbon, 245 mg, 0.230 mmol) was added to a solution of 5-fluoropyrimidine-2,4 (1H,3H)-dione (300 mg, 2.31 mmol) in MeOH (40 mL). The resulting mixture was stirred under H₂ (1 atm) at r.t. for 36 h. The reaction mixture was filtered on a celite pad. The solvent of the filtrate was removed under reduced pressure to give the title compound (250 mg, 82%) as a white solid. ¹H NMR: δ 10.43 (s, 1H), 7.69 (s, 1H), 5.15 (ddd, 1H), 3.56 (ddd, 1H), 3.40 (td, 1H).

### Intermediate 45b: tert-Butyl 6-(5-fluoro-2,4-dioxotetrahydropyrimidin-1 (2H)-yl)-1H-indole-1-carboxylate

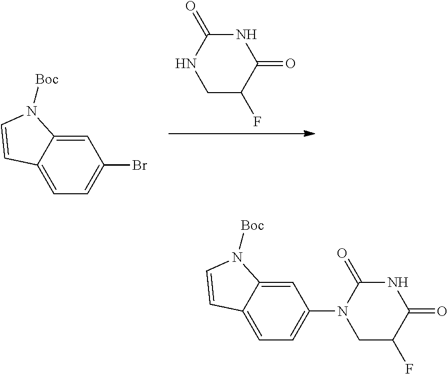 [SMILES: CC(C)(C)OC(=O)n1ccc2ccc(Br)cc21 ; O=C1NCC(F)C(=O)N1 ; CC(C)(C)OC(=O)n1ccc2ccc(N3CC(F)C(=O)NC3=O)cc21]

Ephos (8.1 mg, 0.015 mmol) and Ephos Pd G4 (13.9 mg, 0.0151 mmol) were added to a degassed mixture of Cs₂CO₃ (296 mg, 0.908 mmol), 5-fluorodihydropyrimidine-2,4 (1H,3H)-dione (120 mg, 0.908 mmol) and tert-butyl 6-bromo-1H-indole-1-carboxylate (90.0 mg, 0.304 mmol) in 1,4-dioxane (10 mL) at r.t. under N₂. The resulting mixture was stirred at 100° C. for 14 h. The reaction mixture was filtered, the solvent removed under reduced pressure to give a residue. Purification by C-18FC (gradient: 0-100% MeCN in water) gave the title compound (50 mg, 48%) as a white solid. ¹H NMR: δ 10.90 (s, 1H), 8.06 (d, 1H), 7.72 (d, 1H), 7.65 (d, 1H), 7.22 (dd, 1H), 6.74 (d, 1H), 5.43 (ddd, 1H), 4.31-4.02 (m, 2H), 1.63 (s, 9H). m/z (ES⁺), [M-tBu+2H]⁺=292.2.

### Example 45: 5-Fluoro-1-(1H-indol-6-yl)dihydropyrimidine-2,4 (1H,3H)-dione

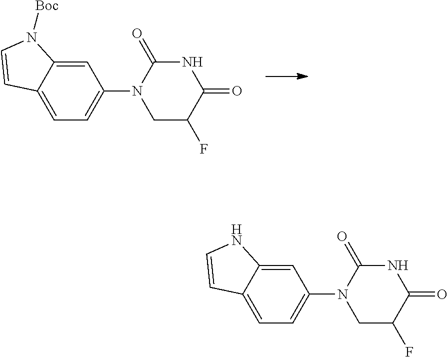 [SMILES: CC(C)(C)OC(=O)n1ccc2ccc(N3CC(F)C(=O)NC3=O)cc21 ; O=C1NC(=O)N(c2ccc3cc[nH]c3c2)CC1F]

A suspension of tert-butyl 6-(5-fluoro-2,4-dioxotetrahydropyrimidin-1 (2H)-yl)-1H-indole-1-carboxylate (25 mg, 0.072 mmol) in water (20 mL) was stirred at 100° C. for 5 h. The water was removed under reduced pressure to give a residue. Purification by preparative HPLC (Column B, Eluent B, gradient: 19-28%) gave the title compound (11 mg, 62%) as a white solid. ¹H NMR: δ 11.18 (s, 1H), 10.43 (s, 1H), 7.54 (d, 1H), 7.39 (d, 1H), 7.34 (s, 1H), 6.94 (dd, 1H), 6.44 (s, 1H), 5.41 (dt, 1H), 3.96-4.26 (m, 2H). ¹⁹F NMR (376 MHz) δ −198.07. m/z (ES⁺), [M+H]⁺=248.0.

### Intermediate 46a: 6-Bromo-3-((2-(trimethylsilyl)ethoxy)methyl)benzo[d]oxazol-2 (3H)-one

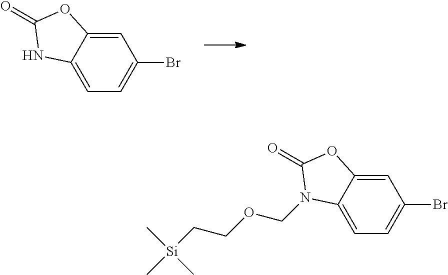 [SMILES: O=c1[nH]c2ccc(Br)cc2o1 ; C[Si](C)(C)CCOCn1c(=O)oc2cc(Br)ccc21]

NaH (60% dispersion in mineral oil, 90.0 mg, 2.25 mmol) was added to a solution of 6-bromobenzo[d]oxazol-2 (3H)-one (400 mg, 1.87 mmol) in DMF (10 mL) at 0° C. under N₂. The resulting solution was stirred at 0° C. for 15 minutes before the addition of (2-(chloromethoxy)ethyl)trimethylsilane (374 mg, 2.24 mmol). The mixture was stirred for 16 h at r.t. The reaction mixture was quenched with saturated NH₄Cl (10 mL) and extracted with EtOAc (2×25 mL). The combined organic extracts were dried (Na₂SO₄) and concentrated to give a brown residue. Purification by FSC (gradient: 0-30% EtOAc in petroleum ether) gave the title compound (0.520 g, 81%) as a colourless gum. ¹H NMR: (CDCl₃) δ 7.41 (d, 1H), 7.37 (dd, 1H), 7.06 (d, 1H), 5.28 (s, 2H), 3.70-3.60 (m, 2H), 0.98-0.92 (m, 2H), 0.00 (s, 9H).

### Intermediate 46b: 1-(2-Oxo-3-((2-(trimethylsilyl)ethoxy)methyl)-2,3-dihydrobenzo[d]oxazol-6-yl)dihydropyrimidine-2,4 (1H,3H)-dione

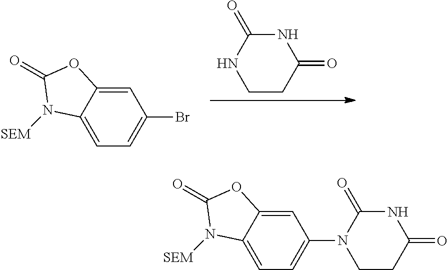 [SMILES: C[Si](C)(C)CCOCn1c(=O)oc2cc(Br)ccc21 ; O=C1CCNC(=O)N1 ; C[Si](C)(C)CCOCn1c(=O)oc2cc(N3CCC(=O)NC3=O)ccc21]

Cs₂CO₃ (284 mg, 0.872 mmol) was added to a degassed mixture of 6-bromo-3-((2-(trimethylsilyl)ethoxy)-methyl)benzo[d]oxazol-2 (3H)-one (100 mg, 0.290 mmol), dihydropyrimidine-2,4 (1H,3H)-dione (66.3 mg, 0.581 mmol), Ephos Pd G4 (15.0 mg, 0.0163 mmol) and Ephos (7.8 mg, 0.015 mmol) in 1,4-dioxane (5 mL) at r.t. under N₂. The resulting suspension was stirred at 100° C. for 16 h. The reaction was then filtered and the filtrate concentrated. Purification by C-18FC (gradient: 0-80% MeCN in water) gave the title compound (60.0 mg, 55%) as a yellow solid. ¹H NMR: δ 10.38 (s, 1H), 7.41 (d, 1H), 7.32 (d, 1H), 7.19 (dd, 1H), 5.24 (s, 2H), 3.75 (t, 2H), 3.65-3.54 (m, 2H), 2.70 (t, 2H), 0.92-0.81 (m, 2H), −0.06 (s, 9H). m/z (ES⁺), [M+H]⁺=378.2.

### Example 46: 1-(2-Oxo-2,3-dihydrobenzo[d]oxazol-6-yl)dihydropyrimidine-2,4 (1H,3H)-dione

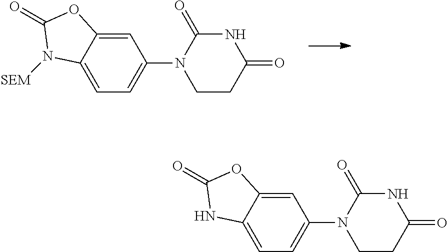 [SMILES: C[Si](C)(C)CCOCn1c(=O)oc2cc(N3CCC(=O)NC3=O)ccc21 ; O=C1CCN(c2ccc3[nH]c(=O)oc3c2)C(=O)N1]

1-(2-Oxo-3-((2-(trimethylsilyl)ethoxy)methyl)-2,3-dihydrobenzo[d]oxazol-6-yl)dihydropyrimidine-2,4 (1H,3H)-dione (60.0 mg, 0.159 mmol) was added to a solution of DCM (2 mL) and TFA (2 mL) at r.t. under N₂. The resulting solution was stirred at r.t. for 16 h. The solvent was removed and the residue was dissolved in DMF (2 mL). K₂CO₃ (220 mg, 1.59 mmol) was then added and the mixture was stirred at 60° C. for 2 h. The reaction was filtered and the filtrate concentrated. Purification by C-18FC (gradient: 0-80% MeCN in water) gave the title compound (15.0 mg, 38%) as a yellow solid. ¹H NMR: δ 11.67 (s, 1H), 10.35 (s, 1H), 7.31 (d, 1H), 7.08 (d, 2H), 3.73 (t, 2H), 2.69 (t, 2H). m/z (ES⁺), [M+H]⁺=248.1.

### Intermediate 47a: 5-Bromo-3-((2-(trimethylsilyl)ethoxy)methyl)benzo[d]oxazol-2 (3H)-one

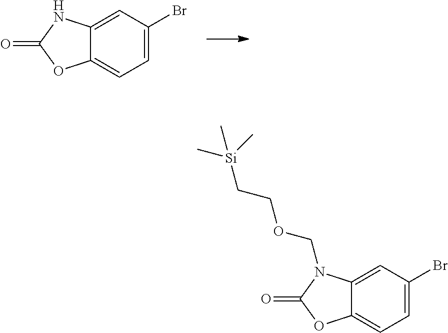 [SMILES: O=c1[nH]c2cc(Br)ccc2o1 ; C[Si](C)(C)CCOCn1c(=O)oc2ccc(Br)cc21]

NaH (60% dispersion in mineral oil, 70.6 mg, 1.77 mmol) was added to a solution of 5-bromobenzo[d]oxazol-2 (3H)-one (315 mg, 1.47 mmol) in DMF (10 mL) at 0° C. under N₂. The resulting solution was stirred at r.t. for 15 minutes before the addition of (2-(chloromethoxy)ethyl)trimethylsilane (294 mg, 1.77 mmol) at r.t. The mixture was stirred for 2 h at r.t. The reaction mixture was quenched with saturated NH₄Cl (25 mL) and extracted with EtOAc (2×50 mL). The combined organic extracts were dried (Na₂SO₄) and concentrated to give a yellow solid. Purification by C-18FC (gradient: 30-80% MeCN in water) gave the title compound (380 mg, 75%) as a white solid. ¹H NMR: δ 0.05 (9H, s), 0.88 (2H, m), 3.61 (2H, m), 5.26 (2H, s), 7.36 (2H, d), 7.61 (1H, d).

### Intermediate 47b: 1-(2-Oxo-3-((2-(trimethylsilyl)ethoxy)methyl)-2,3-dihydrobenzo[d]oxazol-5-yl)dihydropyrimidine-2,4 (1H,3H)-dione

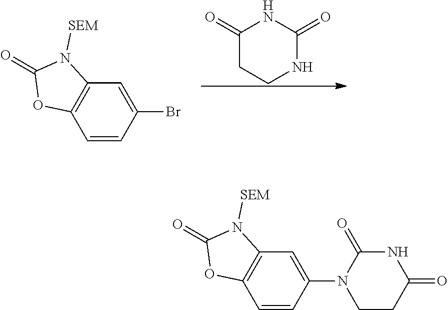 [SMILES: C[Si](C)(C)CCOCn1c(=O)oc2ccc(Br)cc21 ; O=C1CCNC(=O)N1 ; C[Si](C)(C)CCOCn1c(=O)oc2ccc(N3CCC(=O)NC3=O)cc21]

Cs₂CO₃ (710 mg, 2.18 mmol) was added to a degassed mixture of 5-bromo-3-((2-(trimethylsilyl)ethoxy)-methyl)benzo[d]oxazol-2 (3H)-one (250 mg, 0.726 mmol), dihydropyrimidine-2,4 (1H,3H)-dione (249 mg, 2.18 mmol), Ephos (19.4 mg, 0.0363 mmol) and Ephos Pd G4 (33.3 mg, 0.0363 mmol) in 1,4-dioxane (15 mL). The resulting suspension was stirred at 100° C. overnight. The solvent was then removed under reduced pressure. Purification by C-18FC (gradient: 50-80% MeCN in water) gave the title compound (180 mg, 66%) as a yellow solid. ¹H NMR: δ −0.06 (9H, s), 0.87 (2H, dd), 2.71 (2H, t), 3.60 (2H, dd), 3.75 (2H, t), 5.22 (2H, s), 7.11 (1H, dd), 7.36 (2H, m), 10.38 (1H, s). m/z (ES⁻), [M−H]⁻=376.

### Example 47: 1-(2-Oxo-2,3-dihydrobenzo[d]oxazol-5-yl)dihydropyrimidine-2,4 (1H,3H)-dione

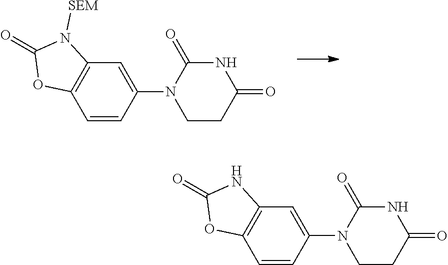 [SMILES: C[Si](C)(C)CCOCn1c(=O)oc2ccc(N3CCC(=O)NC3=O)cc21 ; O=C1CCN(c2ccc3oc(=O)[nH]c3c2)C(=O)N1]

1-(2-Oxo-3-((2-(trimethylsilyl)ethoxy)methyl)-2,3-dihydrobenzo[d]oxazol-5-yl)dihydropyrimidine-2,4 (1H,3H)-dione (80.0 mg, 0.212 mmol) was added to a solution of HCl in 1,4-dioxane (4M, 10.0 mL, 40.0 mmol). The resulting mixture was stirred at r.t. for 16 h. The solvent was removed under reduced pressure and the residue was dissolved in DMF (5 mL). K₂CO₃ (80 mg, 0.58 mmol) was then added and the mixture was stirred at 50° C. for 3 h. The reaction mixture was filtered and the filtrate concentrated. Purification by preparative HPLC (Column E, Eluent E, gradient: 5-28%) gave the title compound (40.0 mg, 76%) as a white solid. ¹H NMR: δ 2.71 (2H, t), 3.76 (2H, t), 7.02 (1H, dd), 7.09 (1H, d), 7.30 (1H, d), 10.36 (1H, s). m/z (ES⁺), [M+H]⁺=248.1.

### Intermediate 48a: 6-Bromo-7-methyl-1-((2-(trimethylsilyl)ethoxy)methyl)-1H-pyrrolo[3,2-b]pyridine

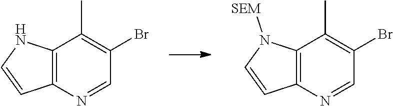 [SMILES: Cc1c(Br)cnc2cc[nH]c12 ; Cc1c(Br)cnc2ccn(COCC[Si](C)(C)C)c12]

NaH (60% dispersion in mineral oil, 284 mg, 7.11 mmol) was added to 6-bromo-7-methyl-1H-pyrrolo[3,2-b]pyridine (500 mg, 2.37 mmol) in DMF (10 mL) at 0° C. under N₂. The mixture was stirred for 20 minutes before the addition of (2-(chloromethoxy)ethyl)trimethylsilane (462 μL, 2.61 mmol). The resulting mixture was stirred at r.t. for 2 h. The reaction mixture was quenched with water (50 mL), extracted with EtOAc (4×50 mL). The combined organic extracts were dried (Na₂SO₄) and concentrated to give the title compound (800 mg, 99%) as a yellow oil. ¹H NMR: δ −0.11 (9H, s), 0.81 (2H, t), 2.78 (3H, s), 3.45 (2H, t), 5.63 (2H, s), 6.56 (1H, d), 7.79 (1H, d), 8.43 (1H, d). m/z (ES⁺), [M+H]⁺=343.0.

### Intermediate 48b: tert-Butyl (7-methyl-1-((2-(trimethylsilyl)ethoxy)methyl)-1H-pyrrolo[3,2-b]pyridin-6-yl)carbamate

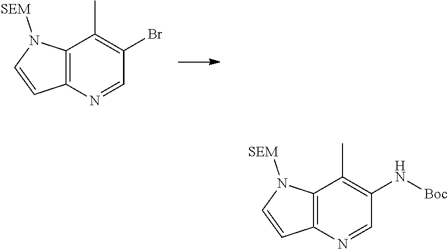 [SMILES: Cc1c(Br)cnc2ccn(COCC[Si](C)(C)C)c12 ; Cc1c(NC(=O)OC(C)(C)C)cnc2ccn(COCC[Si](C)(C)C)c12]

Ephos Pd G4 (80.0 mg, 0.0871 mmol) was added to a degassed mixture of 6-bromo-7-methyl-1-((2-(trimethylsilyl)-ethoxy)methyl)-1H-pyrrolo[3,2-b]pyridine (598 mg, 1.75 mmol), tert-butyl carbamate (410 mg, 3.50 mmol), Ephos (46.8 mg, 0.0875 mmol) and Cs₂CO₃ (1.14 g, 3.50 mmol) in 1,4-dioxane (15 mL) at r.t. under N₂. The resulting mixture was stirred at 100° C. for 16 h. The solvent was then removed under reduced pressure. Purification by FSC (gradient: 0-5% MeOH in DCM) gave the title compound (260 mg, 39%) as a yellow solid. ¹H NMR: δ −0.08 (9H, s), 0.82 (2H, t), 1.45 (9H, s), 2.52 (3H, s), 3.45 (2H, t), 5.60 (2H, s), 6.50 (1H, d), 7.70 (1H, d), 8.11 (1H, s), 8.77 (1H, s). m/z (ES⁺), [M+H]⁺=378.2.

### Intermediate 48c: 7-Methyl-1-((2-(trimethylsilyl)ethoxy)methyl)-1H-pyrrolo[3,2-b]pyridin-6-amine

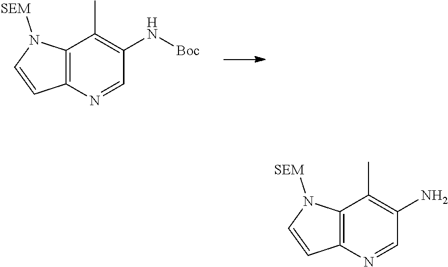 [SMILES: Cc1c(NC(=O)OC(C)(C)C)cnc2ccn(COCC[Si](C)(C)C)c12 ; Cc1c(N)cnc2ccn(COCC[Si](C)(C)C)c12]

tert-Butyl (7-methyl-1-((2-(trimethylsilyl)ethoxy)methyl)-1H-pyrrolo[3,2-b]pyridin-6-yl)carbamate (400 mg, 1.06 mmol) was dissolved in 2,2,2-trifluoroethanol (10 mL) and sealed into a microwave tube. The reaction was heated to 140° C. for 3 h in a microwave reactor and then cooled to r.t. The solvent was removed under reduced pressure to give the title compound (260 mg, 88%) as a yellow oil. ¹H NMR: δ 0.02 (9H, s), 0.92 (2H, t), 2.49 (3H, s), 3.47 (2H, brs), 3.55 (2H, t), 5.61 (2H, s), 6.42 (1H, d), 7.46 (1H, d), 7.99 (1H, s). m/z (ES⁺), [M+H]⁺=278.2.

### Example 48: 1-(7-Methyl-1H-pyrrolo[3,2-b]pyridin-6-yl)pyrimidine-2,4 (1H,3H)-dione

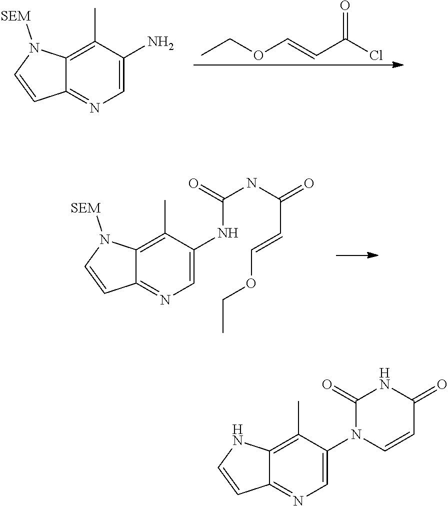

(E)-3-Ethoxyacryloyl chloride (364 mg, 2.71 mmol) was added to silver cyanate (675 mg, 4.50 mmol) in toluene (5 mL) at r.t. under N₂. The resulting mixture was stirred at 120° C. for 1 h. The reaction was cooled to 0° C. before the addition of the supernatant to a solution of 7-methyl-1-((2-(trimethylsilyl)ethoxy)methyl)-1H-pyrrolo[3,2-b]pyridin-6-amine (250 mg, 0.901 mmol) in DMF (5 mL) at 0° C. under air. The resulting mixture was stirred at 0° C. for 1 h. The reaction mixture was diluted with EtOAc (100 mL), and washed sequentially with water (3×50 mL) and saturated brine (50 mL). The organic layer was dried (Na₂SO₄) and concentrated to give (E)-3-ethoxy-N-((7-methyl-1-((2-(trimethylsilyl)ethoxy)methyl)-1H-pyrrolo[3,2-b]pyridin-6-yl)carbamoyl)-acrylamide as a yellow oil which was used directly in the next step without any further purification. m/z (ES⁺), [M+H]⁺=419.2. The material was then dissolved in DCM (8 mL) before the addition of TFA (4 mL, 51.92 mmol) at r.t. under air. The resulting mixture was stirred at r.t. for 2 days. The solvent was then removed under reduced pressure. The reaction mixture was diluted with 7M ammonia in MeOH (5 mL) and stirred at r.t. for 0.5 h. The solvent was then removed under reduced pressure. Purification by C-18FC (gradient: 5-10% MeCN in water (containing 0.05% TFA)) gave the title compound (70.0 mg, 32% over two-steps) as a white solid. ¹H NMR: δ 2.51 (3H, s), 5.79 (1H, dd), 6.81 (1H, dd), 7.70 (1H, d), 8.14 (1H, d), 8.64 (1H, d), 11.63 (1H, s), 12.62 (1H, s). m/z (ES⁺), [M+H]⁺=243.2.

### Example 49: 1-(7-Methyl-1H-pyrrolo[3,2-b]pyridin-6-yl)dihydropyrimidine-2,4 (1H,3H)-dione

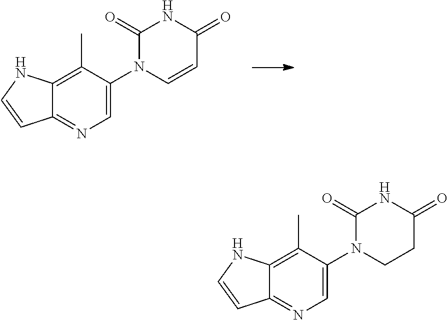 [SMILES: Cc1c(-n2ccc(=O)[nH]c2=O)cnc2cc[nH]c12 ; Cc1c(N2CCC(=O)NC2=O)cnc2cc[nH]c12]

A mixture of 1-(7-methyl-1H-pyrrolo[3,2-b]pyridin-6-yl)pyrimidine-2,4 (1H,3H)-dione (50.0 mg, 0.206 mmol) and Pd/C (10% on activated carbon, 110 mg, 0.103 mmol) in MeOH (30 mL) was stirred under an atmosphere of H₂ (1 atm) at r.t. for 8 h. The reaction mixture was filtered through a pad of celite. The solvent of the filtrate was then removed under reduced pressure. Purification by preparative HPLC (Column G, Eluent A, gradient: 3-13%) gave the title compound (25.0 mg, 50%) as a white solid. ¹H NMR: δ 2.60 (3H, s), 2.71-2.94 (2H, m), 3.64-3.72 (1H, m), 3.81-3.91 (1H, m), 6.75-6.86 (1H, m), 8.16 (1H, t), 8.68 (1H, s), 10.59 (1H, s), 12.71 (1H, s). m/z (ES⁺), [M+H]⁺=245.1

### Intermediate 50a: tert-Butyl 6-bromo-4-fluoro-1H-indole-1-carboxylate

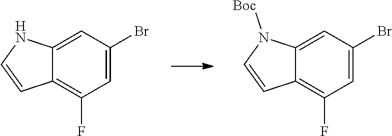 [SMILES: Fc1cc(Br)cc2[nH]ccc12 ; CC(C)(C)OC(=O)n1ccc2c(F)cc(Br)cc21]

DMAP (0.080 g, 0.655 mmol) was added to a solution of DIEA (2.29 mL, 13.1 mmol), di-tert-butyl dicarbonate (2.28 mL, 9.82 mmol) and 6-bromo-4-fluoro-1H-indole (1.40 g, 6.54 mmol) in DCM (20 mL) at r.t. under air. The resulting solution was stirred at r.t. for 2 h. The solvent was removed under reduced pressure. Purification by FSC (gradient: 0-5% EtOAc in petroleum ether) gave the title compound (2.00 g, 97%) as a white solid. ¹H NMR: (CDCl₃) δ 1.67 (9H, s), 6.62 (1H, dd), 7.09 (1H, dd), 7.52 (1H, d), 8.17 (1H, s).

### Intermediate 50b: tert-Butyl 6-(2,4-dioxotetrahydropyrimidin-1 (2H)-yl)-4-fluoro-1H-indole-1-carboxylate

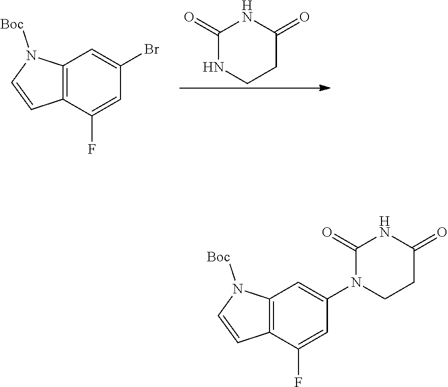 [SMILES: CC(C)(C)OC(=O)n1ccc2c(F)cc(Br)cc21 ; O=C1CCNC(=O)N1 ; CC(C)(C)OC(=O)n1ccc2c(F)cc(N3CCC(=O)NC3=O)cc21]

Ephos (34.0 mg, 0.0636 mmol) and Ephos Pd G4 (58.5 mg, 0.0637 mmol) were added to a degassed mixture of Cs₂CO₃ (415 mg, 1.27 mmol), dihydropyrimidine-2,4 (1H,3H)-dione (218 mg, 1.91 mmol) and tert-butyl 6-bromo-4-fluoro-1H-indole-1-carboxylate (200 mg, 0.64 mmol) in 1,4-dioxane (10 mL) at r.t. under N₂. The resulting mixture was stirred at 100° C. for 17 h. The solvent was then removed under reduced pressure. Purification by FSC (gradient: 0-8% MeOH in DCM) gave the title compound (210 mg, 95%) as a pale yellow solid. ¹H NMR: (CDCl₃) δ 1.68 (9H, s), 2.88 (2H, t), 3.95 (2H, t), 6.68 (1H, d), 6.91-7.01 (1H, m), 7.59 (1H, d), 8.00 (1H, s). m/z (ES⁺), [M+H]⁺=348.2.

### Example 50: 1-(4-Fluoro-1H-indol-6-yl)dihydropyrimidine-2,4 (1H,3H)-dione

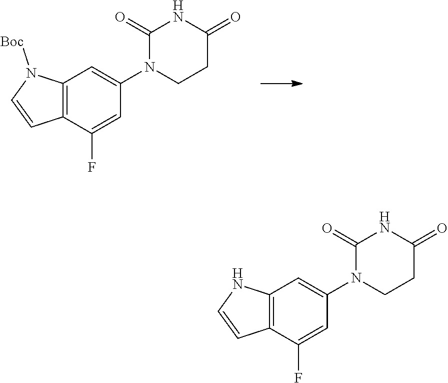 [SMILES: CC(C)(C)OC(=O)n1ccc2c(F)cc(N3CCC(=O)NC3=O)cc21 ; O=C1CCN(c2cc(F)c3cc[nH]c3c2)C(=O)N1]

tert-Butyldimethylsilyl trifluoromethanesulfonate (205 mg, 0.776 mmol) was added to a solution of tert-butyl 6-(2,4-dioxotetrahydropyrimidin-1 (2H)-yl)-4-fluoro-1H-indole-1-carboxylate (180 mg, 0.518 mmol) in MeCN (10 mL) at r.t. under air. The resulting solution was stirred at r.t. for 1 h. The solvent was then removed under reduced pressure. Purification by C-18FC (gradient: 0-30% MeCN in water (containing 0.1% FA)) provided material that was further purified by preparative HPLC (Column H, Eluent E, gradient: 25-37%) to give the title compound (32.0 mg, 25%) as a pale yellow solid. ¹H NMR: δ 2.73 (2H, t), 3.81 (2H, t), 6.45-6.53 (1H, m), 6.83 (1H, dd), 7.21 (1H, t), 7.39-7.47 (1H, m), 10.36 (1H, s), 11.49 (1H, s). m/z (ES⁺), [M+H]⁺=248.2.

### Intermediate 51a: tert-Butyl 6-bromo-4-methyl-1H-indole-1-carboxylate

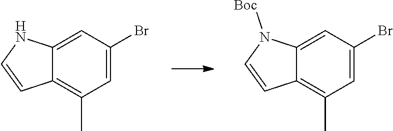 [SMILES: Cc1cc(Br)cc2[nH]ccc12 ; Cc1cc(Br)cc2c1ccn2C(=O)OC(C)(C)C]

DMAP (17.5 mg, 0.143 mmol) was added to a solution of DIEA (499 μL, 2.86 mmol), di-tert-butyl dicarbonate (497 μL, 2.14 mmol) and 6-bromo-4-methyl-1H-indole (300 mg, 1.43 mmol) in DCM (20 mL). The resulting solution was stirred at r.t. for 2 h. The solvent was then removed under reduced pressure. Purification by FSC (gradient 0-4% EtOAc in petroleum ether) gave the title compound (440 mg, 99%) as a brown oil. ¹H NMR: δ 1.60 (9H, s), 2.46 (3H, s), 6.72-6.79 (1H, m), 7.20-7.27 (1H, m), 7.65 (1H, d), 8.00-8.05 (1H, m).

### Intermediate 51b: tert-Butyl 6-(2,4-dioxotetrahydropyrimidin-1 (2H)-yl)-4-methyl-1H-indole-1-carboxylate

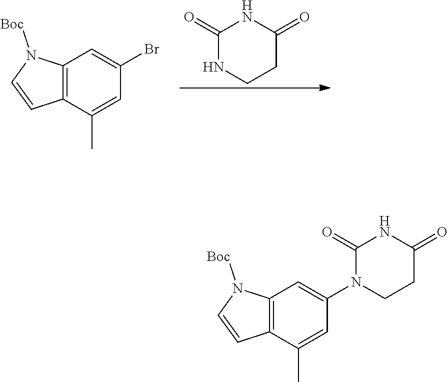 [SMILES: Cc1cc(Br)cc2c1ccn2C(=O)OC(C)(C)C ; O=C1CCNC(=O)N1 ; Cc1cc(N2CCC(=O)NC2=O)cc2c1ccn2C(=O)OC(C)(C)C]

Ephos (69.0 mg, 0.129 mmol) and Ephos Pd G4 (118 mg, 0.128 mmol) were added to a degassed mixture of Cs₂CO₃ (840 mg, 2.58 mmol), dihydropyrimidine-2,4 (1H,3H)-dione (441 mg, 3.87 mmol) and tert-butyl 6-bromo-4-methyl-1H-indole-1-carboxylate (400 mg, 1.29 mmol) in 1,4-dioxane (16 mL) at r.t. under N₂. The resulting mixture was stirred at 100° C. for 17 h. The solvent was then removed under reduced pressure. Purification by FSC (gradient: 0-9% MeOH in DCM) gave the title compound (240 mg, 54%) as a brown solid. ¹H NMR: δ 1.60 (9H, s), 2.46 (3H, s), 2.71 (2H, t), 3.79 (2H, t), 6.71-6.78 (1H, m), 6.99-7.06 (1H, m), 7.66 (1H, d), 7.82-7.88 (1H, m), 10.33 (1H, s). m/z (ES⁺), [M+H]⁺=366.1.

### Example 51: 1-(4-Methyl-1H-indol-6-yl)dihydropyrimidine-2,4 (1H,3H)-dione

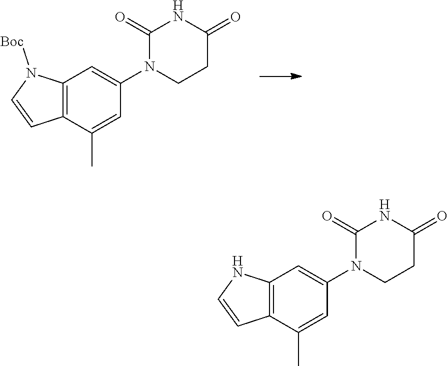 [SMILES: Cc1cc(N2CCC(=O)NC2=O)cc2c1ccn2C(=O)OC(C)(C)C ; Cc1cc(N2CCC(=O)NC2=O)cc2[nH]ccc12]

tert-Butyl 6-(2,4-dioxotetrahydropyrimidin-1 (2H)-yl)-4-methyl-1H-indole-1-carboxylate (260 mg, 0.757 mmol) was dissolved in 2,2,2-trifluoroethanol (3 mL) and sealed into a microwave tube. The reaction was heated to 120° C. for 1 h in a microwave reactor and then cooled to r.t. The solvent was then removed under reduced pressure. Purification by C-18FC (gradient: 0-23% MeCN in water (containing 0.1% FA)) gave the title compound (176 mg, 96%) as a brown solid. ¹H NMR: δ 2.46 (3H, s), 2.71 (2H, t), 3.76 (2H, t), 6.41-6.47 (1H, m), 6.72-6.78 (1H, m), 7.15 (1H, s), 7.34 (1H, t), 10.26 (1H, s), 11.12 (1H, s). m/z (ES⁺), [M+H]⁺=244.3.

### Intermediate 52a: tert-Butyl 6-bromo-4-methoxy-1H-indole-1-carboxylate

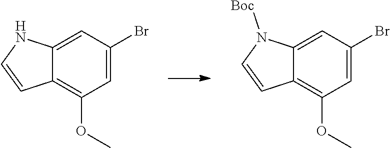 [SMILES: COc1cc(Br)cc2[nH]ccc12 ; COc1cc(Br)cc2c1ccn2C(=O)OC(C)(C)C]

DMAP (16.2 mg, 0.133 mmol) was added to a solution of DIEA (464 μL, 2.65 mmol), di-tert-butyl dicarbonate (462 μL, 1.99 mmol) and 6-bromo-4-methoxy-1H-indole (300 mg, 1.33 mmol) in DCM (20 mL). The resulting solution was stirred at r.t. for 2 h. The solvent was removed under reduced pressure. Purification by FSC (gradient: 0-7% EtOAc in petroleum ether) gave the title compound (0.430 g, 99%) as a brown solid. ¹H NMR: δ 1.60 (9H, s), 3.89 (3H, s), 6.62-6.69 (1H, m), 6.95 (1H, d), 7.55 (1H, d), 7.79-7.86 (1H, m).

### Intermediate 52b: tert-Butyl 6-(2,4-dioxotetrahydropyrimidin-1 (2H)-yl)-4-methoxy-1H-indole-1-carboxylate

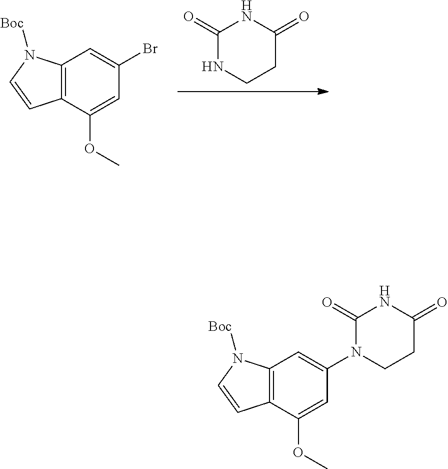 [SMILES: COc1cc(Br)cc2c1ccn2C(=O)OC(C)(C)C ; O=C1CCNC(=O)N1 ; COc1cc(N2CCC(=O)NC2=O)cc2c1ccn2C(=O)OC(C)(C)C]

Ephos (65.6 mg, 0.123 mmol) and Ephos Pd G4 (113 mg, 0.123 mmol) were added to a degassed mixture of Cs₂CO₃ (799 mg, 2.45 mmol), dihydropyrimidine-2,4 (1H,3H)-dione (420 mg, 3.68 mmol) and tert-butyl 6-bromo-4-methoxy-1H-indole-1-carboxylate (400 mg, 1.23 mmol) in 1,4-dioxane (16 mL) at r.t. under N₂. The resulting mixture was stirred at 100° C. for 17 h. The solvent was then removed under reduced pressure. Purification by FSC (gradient: 0-7% MeOH in DCM) gave the title compound (260 mg, 59%) as a brown solid. ¹H NMR: δ 1.60 (9H, s), 2.72 (2H, t), 3.81 (2H, t), 3.87 (3H, s), 6.62-6.72 (1H, m), 6.79 (1H, d), 7.56 (1H, d), 7.60-7.67 (1H, m), 10.35 (1H, s). m/z (ES⁺), [M+H]⁺=360.1.

### Example 52: 1-(4-Methoxy-1H-indol-6-yl)dihydropyrimidine-2,4 (1H,3H)-dione

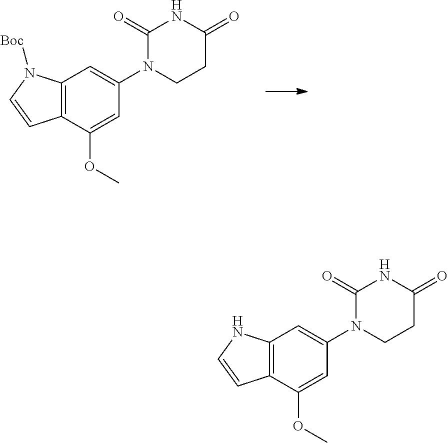 [SMILES: COc1cc(N2CCC(=O)NC2=O)cc2c1ccn2C(=O)OC(C)(C)C ; COc1cc(N2CCC(=O)NC2=O)cc2[nH]ccc12]

tert-Butyl 6-(2,4-dioxotetrahydropyrimidin-1 (2H)-yl)-4-methoxy-1H-indole-1-carboxylate (250 mg, 0.696 mmol) was dissolved in 2,2,2-trifluoroethanol (3 mL) and sealed into a microwave tube. The reaction was heated to 120° C. for 1 h in a microwave reactor and then cooled to r.t. The solvent was then removed under reduced pressure. Purification by C-18FC (gradient: 0-25% MeCN in water (containing 0.1% FA)) gave the title compound (155 mg, 86%) as a brown solid. ¹H NMR: δ 2.72 (2H, t), 3.78 (2H, t), 3.85 (3H, s), 6.38-6.44 (1H, m), 6.48 (1H, d), 6.94 (1H, s), 7.25 (1H, t), 10.27 (1H, s), 11.15 (1H, s). m/z (ES⁺), [M+H]⁺=260.2.

### Intermediate 53a: tert-Butyl 6-bromo-4-chloro-1H-indole-1-carboxylate

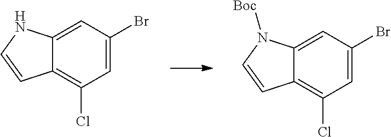 [SMILES: Clc1cc(Br)cc2[nH]ccc12 ; CC(C)(C)OC(=O)n1ccc2c(Cl)cc(Br)cc21]

DMAP (21.2 mg, 0.174 mmol) was added to a solution of DIEA (606 μL, 3.47 mmol), di-tert-butyl dicarbonate (604 μL, 2.60 mmol) and 6-bromo-4-chloro-1H-indole (400 mg, 1.74 mmol) in DCM (10 mL). The resulting solution was stirred at r.t. for 2 h. The solvent was then removed under reduced pressure. Purification by FSC (gradient: 0-5% EtOAc in petroleum ether) gave the title compound (458 mg, 80%) as a white solid. ¹H NMR: δ 1.64 (9H, s), 6.75 (1H, dt), 7.59 (1H, t), 7.81 (1H, d), 8.14-8.23 (1H, m). m/z (ES⁺), [M+Na]⁺=352.3.

### Intermediate 53b: tert-Butyl 4-chloro-6-(2,4-dioxotetrahydropyrimidin-1 (2H)-yl)-1H-indole-1-carboxylate

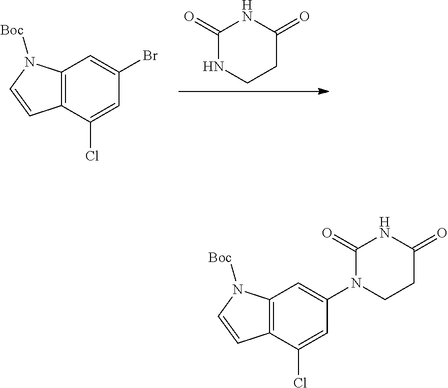 [SMILES: CC(C)(C)OC(=O)n1ccc2c(Cl)cc(Br)cc21 ; O=C1CCNC(=O)N1 ; CC(C)(C)OC(=O)n1ccc2c(Cl)cc(N3CCC(=O)NC3=O)cc21]

Ephos (72.5 mg, 0.136 mmol) and Ephos Pd G4 (124 mg, 0.135 mmol) were added to a degassed mixture of Cs₂CO₃ (883 mg, 2.71 mmol), dihydropyrimidine-2,4 (1H,3H)-dione (464 mg, 4.07 mmol) and tert-butyl 6-bromo-4-chloro-1H-indole-1-carboxylate (448 mg, 1.36 mmol) in 1,4-dioxane (10 mL) at r.t. under N₂. The resulting mixture was stirred at 100° C. for 17 h. The solvent was then removed under reduced pressure. Purification by C-18FC (gradient: 0-50% MeCN in water (containing 0.1% FA)) gave the title compound (190 mg, 39%) as a brown solid. ¹H NMR: δ 1.61 (9H, s), 2.72 (2H, t), 3.84 (2H, t), 6.73 (1H, dd), 7.38 (1H, d), 7.79 (1H, d), 8.01 (1H, dd), 10.42 (1H, s). m/z (ES⁺), [M+H]⁺=364.0.

### Example 53: 1-(4-Chloro-1H-indol-6-yl)dihydropyrimidine-2,4 (1H,3H)-dione

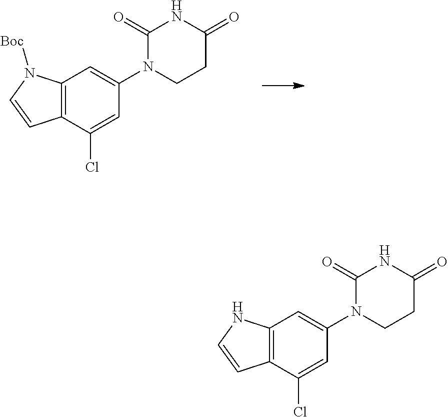 [SMILES: CC(C)(C)OC(=O)n1ccc2c(Cl)cc(N3CCC(=O)NC3=O)cc21 ; O=C1CCN(c2cc(Cl)c3cc[nH]c3c2)C(=O)N1]

tert-Butyl 4-chloro-6-(2,4-dioxotetrahydropyrimidin-1 (2H)-yl)-1H-indole-1-carboxylate (180 mg, 0.495 mmol) was added in 2,2,2-trifluoroethanol (3 mL) and sealed into a microwave tube. The reaction was heated to 120° C. for 2 h in a microwave reactor and then cooled to r.t. The solvent was then removed under reduced pressure. Purification by C-18FC (gradient: 0-50% MeCN in water (containing 0.1% FA)) gave the title compound (81.0 mg, 62%) as a white solid. ¹H NMR: δ 2.73 (2H, t), 3.80 (2H, t), 6.46 (1H, ddd), 7.10 (1H, d), 7.30-7.37 (1H, m), 7.50 (1H, t), 10.35 (1H, s), 11.53 (1H, s). m/z (ES⁺), [M+H]⁺=264.2.

### Intermediate 54a: tert-Butyl 6-bromo-7-fluoro-1H-indole-1-carboxylate

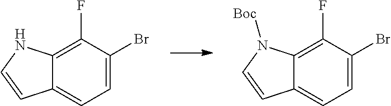 [SMILES: Fc1c(Br)ccc2cc[nH]c12 ; CC(C)(C)OC(=O)n1ccc2ccc(Br)c(F)c21]

DMAP (0.114 g, 0.933 mmol) was added to a solution of DIEA (2.45 mL, 14.0 mmol), di-tert-butyl dicarbonate (2.17 mL, 9.34 mmol) and 6-bromo-7-fluoro-1H-indole (1.00 g, 4.67 mmol) in DCM (30 mL) at r.t. under N₂. The resulting mixture was stirred at r.t. for 1 h. The solvent was then removed under reduced pressure. Purification by FSC (gradient: 0-10% EtOAc in petroleum ether) gave the title compound (1.50 g, 102%) as a pale yellow oil which solidified on standing. ¹H NMR: (CDCl₃) δ 1.66 (9H, s), 6.55 (1H, dd), 7.20 (1H, d), 7.37 (1H, dd), 7.62 (1H, d). m/z (ES⁺), [M-Boc+2H]⁺=214.0.

### Intermediate 54b: tert-Butyl 6-((tert-butoxycarbonyl)amino)-7-fluoro-1H-indole-1-carboxylate

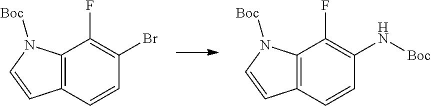 [SMILES: CC(C)(C)OC(=O)n1ccc2ccc(Br)c(F)c21 ; CC(C)(C)OC(=O)Nc1ccc2ccn(C(=O)OC(C)(C)C)c2c1F]

Ephos Pd G4 (0.175 g, 0.191 mmol) was added to a degassed mixture of tert-butyl 6-bromo-7-fluoro-1H-indole-1-carboxylate (1.00 g, 3.18 mmol), tert-butyl carbamate (0.746 g, 6.37 mmol), Ephos (0.170 g, 0.318 mmol) and Cs₂CO₃ (2.07 g, 6.35 mmol) in 1,4-dioxane (20 mL) at r.t. under N₂. The resulting mixture was stirred at 100° C. for 16 h. The solvent was then removed under reduced pressure. Purification by FSC (gradient: 0-5% EtOAc in petroleum ether) gave the title compound (1.00 g, 90%) as a colourless gum. ¹H NMR: (CDCl₃) δ 1.54 (9H, s), 1.65 (9H, s), 6.51 (1H, dd), 6.72 (1H, s), 7.22-7.31 (1H, m), 7.54 (1H, d), 7.93 (1H, br s). m/z (ES⁺), [M+Na]⁺=373.

### Intermediate 54c: 3-((7-Fluoro-1H-indol-6-yl)amino)propanoic acid

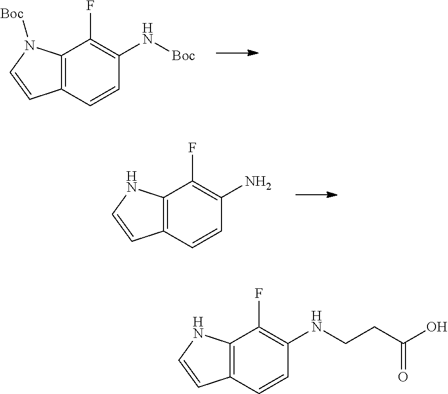 [SMILES: CC(C)(C)OC(=O)Nc1ccc2ccn(C(=O)OC(C)(C)C)c2c1F ; Nc1ccc2cc[nH]c2c1F ; O=C(O)CCNc1ccc2cc[nH]c2c1F]

TFA (8.00 mL, 104 mmol) was added to a solution of tert-butyl 6-((tert-butoxycarbonyl)amino)-7-fluoro-1H-indole-1-carboxylate (900 mg, 2.57 mmol) in DCM (20 mL) at r.t. The resulting mixture was stirred at r.t. for 1 h. The solvent was then removed under reduced pressure. To the residue was added toluene (20 mL) and to the mixture was added acrylic acid (229 mg, 3.18 mmol) at r.t. The resulting mixture was stirred at 110° C. for 2 h. The solvent was then removed under reduced pressure. Purification by C-18FC (gradient: 0-15% MeCN in water (containing 0.5% TFA)) gave the title compound in the form of a trifluoroacetate salt (0.310 g, 36%) as a yellow foam. ¹H NMR: δ 2.58 (2H, t), 3.42 (2H, t), 6.41 (1H, td), 6.75 (1H, t), 7.19-7.32 (2H, m), 10.27 (1H, br s), 11.30 (1H, s). m/z (ES⁺), [M+H]⁺=223.1.

### Example 54: 1-(7-Fluoro-1H-indol-6-yl)dihydropyrimidine-2,4 (1H,3H)-dione

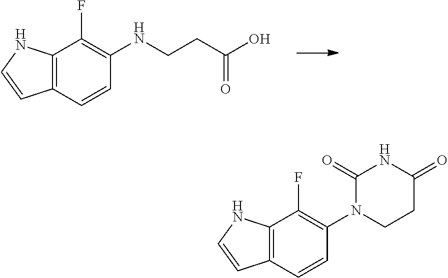 [SMILES: O=C(O)CCNc1ccc2cc[nH]c2c1F ; O=C1CCN(c2ccc3cc[nH]c3c2F)C(=O)N1]

Urea (232 mg, 3.87 mmol) was added to the trifluoroacetate salt of 3-((7-fluoro-1H-indol-6-yl)amino)propanoic acid (260 mg, 0.773 mmol) in AcOH (6 mL) at r.t. The resulting mixture was stirred at 100° C. for 16 h. The solvent was then removed under reduced pressure. Purification by C-18FC (gradient: 0-25% MeCN in water (containing 0.05% TFA)) gave the title compound (75 mg, 39%) as a brown solid. ¹H NMR: δ 2.75 (2H, t), 3.75 (2H, t), 6.53 (1H, td), 6.98 (1H, dd), 7.37 (1H, d), 7.46 (1H, t), 10.43 (1H, s), 11.71 (1H, s). m/z (ES⁺), [M+H]⁺=248.2.

### Example 55: 1-(1H-Indol-6-yl)dihydropyrimidine-2,4 (1H,3H)-dione

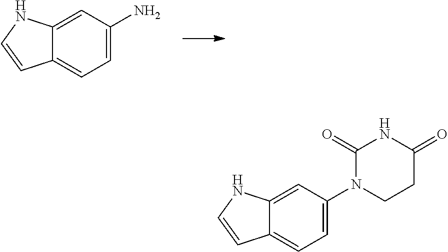 [SMILES: Nc1ccc2cc[nH]c2c1 ; O=C1CCN(c2ccc3cc[nH]c3c2)C(=O)N1]

Acrylic acid (131 mg, 1.82 mmol) was added dropwise to a mixture of 1H-indol-6-amine (200 mg, 1.51 mmol) in toluene (2 mL) at r.t. The resulting solution was stirred at 80° C. for 12 h. The solvent was then removed under reduced pressure. The residue was dissolved in AcOH (2 mL) and to the solution was added urea (176 mg, 2.94 mmol) at r.t. under air. The resulting solution was stirred at 120° C. for 12 h. The solvent was then removed under reduced pressure. Purification by preparative TLC (DCM:MeOH=10:1) gave the title compound (56.0 mg, 16%) as a white solid. ¹H NMR: δ 11.16 (s, 1H), 10.29 (s, 1H), 7.52 (d, 1H), 7.40-7.30 (m, 2H), 6.94 (dd, 1H), 6.45-6.39 (m, 1H), 3.80 (t, 2H), 2.73 (t, 2H). m/z (ES⁺), [M+H]⁺=230.2.

### Example 56: 1-(1H-Indol-5-yl)dihydropyrimidine-2,4 (1H,3H)-dione

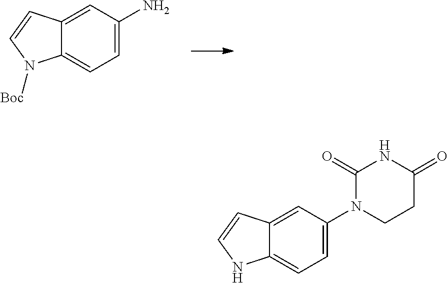 [SMILES: CC(C)(C)OC(=O)n1ccc2cc(N)ccc21 ; O=C1CCN(c2ccc3[nH]ccc3c2)C(=O)N1]

Acrylic acid (465 mg, 6.46 mmol) was added to a solution of tert-butyl 5-amino-1H-indole-1-carboxylate (500 mg, 2.15 mmol) in toluene (5 mL) at r.t. under N₂. The resulting solution was stirred at 110° C. for 12 h. The solvent was removed under reduced pressure and to the crude mixture was added AcOH (5 mL) and urea (388 mg, 6.46 mmol) at r.t. under air. The resulting solution was stirred at 120° C. for 12 h. The solvent was then removed under reduced pressure. Purification by C-18FC (gradient: 0-30% MeCN in water (containing 0.1% FA)) provided a pale yellow solid material that was further purified by preparative HPLC (Column E, Eluent E, gradient: 20-28%) to give the title compound (160 mg, 32%) as a white solid. ¹H NMR: δ 2.72 (2H, t), 3.76 (2H, t), 6.43 (1H, d), 7.02 (1H, dd), 7.34-7.42 (2H, m), 7.45 (1H, d), 10.23 (1H, s), 11.14 (1H, s). m/z (ES⁺), [M+H]⁺=230.0.

### Intermediate 57a: 7-Methyl-6-nitro-1H-indole

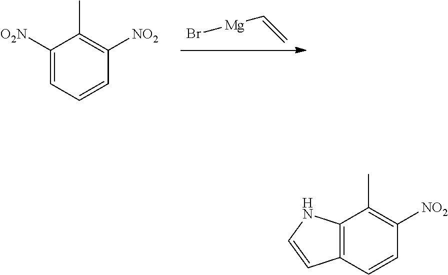

Vinylmagnesium bromide (1M solution in THF, 31.3 mL, 31.3 mmol) was added dropwise to a solution of 2-methyl-1,3-dinitrobenzene (1.90 g, 10.4 mmol) in THF (50 mL) at −78° C. under N₂ over a period of 5 minutes. The resulting mixture was stirred at −78° C. for 4 h. The reaction mixture was quenched with saturated NH₄Cl (20 mL), diluted with EtOAc (100 mL) and washed sequentially with water (50 mL) and saturated brine (100 mL). The organic layer was dried (Na₂SO₄) and concentrated under reduced pressure to give the crude product. Purification by FSC (gradient: 0-25% EtOAc in petroleum ether) gave the title compound as a brown solid (290 mg, 16%). ¹H NMR: δ 2.74 (3H, s), 6.61 (1H, dd), 7.53 (1H, d), 7.64-7.75 (2H, m), 11.86 (1H, s). m/z (ES⁺), [M+H]⁺=177.3.

### Intermediate 57b: tert-Butyl 7-methyl-6-nitro-1H-indole-1-carboxylate

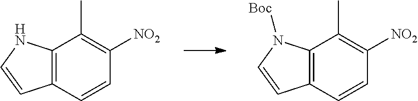 [SMILES: Cc1c([N+](=O)[O-])ccc2cc[nH]c12 ; Cc1c([N+](=O)[O-])ccc2ccn(C(=O)OC(C)(C)C)c12]

DMAP (19.4 mg, 0.159 mmol) was added to a solution of 7-methyl-6-nitro-1H-indole (280 mg, 1.59 mmol), di-tert-butyl dicarbonate (443 μL, 1.91 mmol) and triethylamine (443 μL, 3.18 mmol) in DCM (30 mL) at r.t. under air. The resulting mixture was stirred at r.t. for 2 h. The solvent was then removed under reduced pressure. Purification by FSC (gradient: 0-8% EtOAc in petroleum ether) gave the title compound (326 mg, 74%) as a yellow solid. ¹H NMR: δ 1.62 (9H, s), 2.51 (3H, s), 6.85 (1H, d), 7.67 (1H, d), 7.84 (1H, d), 7.95 (1H, d). m/z (ES⁺), [M+H]⁺=277.1.

### Intermediate 57c: tert-Butyl 6-amino-7-methyl-1H-indole-1-carboxylate

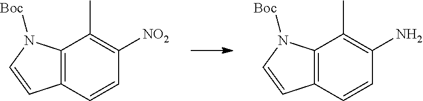 [SMILES: Cc1c([N+](=O)[O-])ccc2ccn(C(=O)OC(C)(C)C)c12 ; Cc1c(N)ccc2ccn(C(=O)OC(C)(C)C)c12]

Zinc (297 mg, 4.54 mmol) was added to a mixture of tert-butyl 7-methyl-6-nitro-1H-indole-1-carboxylate (251 mg, 0.908 mmol) in EtOH (12 mL) and saturated NH₄Cl (3 mL) at r.t. The resulting solution was stirred at r.t. for 2 h. The reaction mixture was filtered through a pad of celite and the solvent was removed under reduced pressure. Purification by FSC (gradient: 0-20% EtOAc in petroleum ether) gave the title compound (66.7 mg, 30%) as a pale yellow oil. ¹H NMR: δ 1.57 (9H, s), 2.13 (3H, s), 4.86 (2H, s), 6.44 (1H, d), 6.67 (1H, d), 7.11 (1H, d), 7.28 (1H, d). m/z (ES⁺), [M+H]⁺=247.2.

### Example 57: 1-(7-Methyl-1H-indol-6-yl)dihydropyrimidine-2,4 (1H,3H)-dione

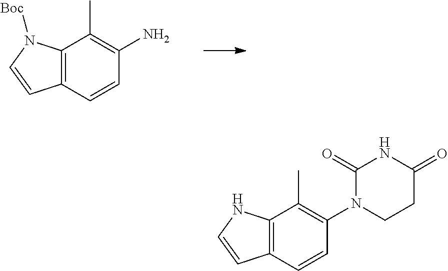 [SMILES: Cc1c(N)ccc2ccn(C(=O)OC(C)(C)C)c12 ; Cc1c(N2CCC(=O)NC2=O)ccc2cc[nH]c12]

Acrylic acid (70.2 mg, 0.974 mmol) was added to a solution of tert-butyl 6-amino-7-methyl-1H-indole-1-carboxylate (40.0 mg, 0.162 mmol) in toluene (2 mL). The resulting mixture was stirred at 100° C. for 10 h. The solvent was then removed under reduced pressure. The residue was dissolved in AcOH (2 mL), urea (9.8 mg, 0.16 mmol) was added and the resulting solution was stirred at 120° C. for 3 h. The solvent was then removed under reduced pressure. Purification by C-18FC (gradient: 0-30% MeCN in water) gave the title compound (3.00 mg, 8%) as a white solid. ¹H NMR: δ 2.33 (3H, s), 2.68-2.86 (2H, m), 3.55 (1H, m), 3.77 (1H, m), 6.44 (1H, m), 6.89 (1H, d), 7.39 (2H, m), 10.28 (1H, s), 11.17 (1H, s). m/z (ES⁺), [M+H]⁺=244.3.

### Intermediate 58a: 6-Bromo-5-fluoro-7-methyl-1H-indole

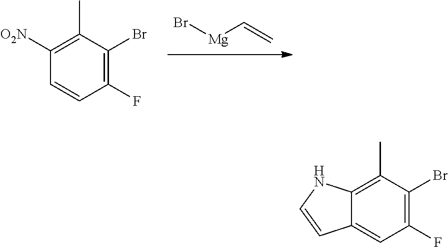

Vinylmagnesium bromide (1M solution in THF, 19.2 mL, 19.2 mmol) was added to a solution of 2-bromo-1-fluoro-3-methyl-4-nitrobenzene (1.50 g, 6.41 mmol) in THF (30 mL) at −78° C. under N₂. The resulting mixture was stirred at −78° C. for 2 h. The reaction was quenched with saturated NH₄Cl (200 mL) and extracted with EtOAc (3×200 mL). The combined organic extracts were dried (Na₂SO₄) and concentrated under reduced pressure to give a brown residue. Purification by FSC (gradient: 0-19% EtOAc in petroleum ether) gave the title compound (0.500 g, 34%) as a yellow solid. ¹H NMR: (CDCl₃) δ 2.60 (3H, s), 6.54 (1H, dd), 7.24-7.29 (1H, m), 8.10 (1H, s). m/z (ES⁺), [M+H]⁺=228.0.

### Intermediate 58b: tert-Butyl 6-bromo-5-fluoro-7-methyl-1H-indole-1-carboxylate

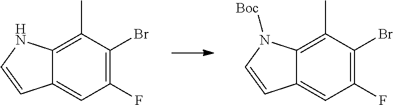 [SMILES: Cc1c(Br)c(F)cc2cc[nH]c12 ; Cc1c(Br)c(F)cc2ccn(C(=O)OC(C)(C)C)c12]

Di-tert-butyl dicarbonate (611 μL, 2.63 mmol) was added to a mixture of 6-bromo-5-fluoro-7-methyl-1H-indole (500 mg, 2.19 mmol), triethylamine (917 μL, 6.58 mmol) and DMAP (26.8 mg, 0.219 mmol) in DCM (20 mL) at r.t. under air. The resulting mixture was stirred at r.t. for 2 h. The solvent was then removed under reduced pressure. Purification by FSC (gradient: 0-5% EtOAc in petroleum ether) gave the title compound (540 mg, 75%) as a yellow oil. ¹H NMR: (CDCl₃) δ 1.63 (9H, s), 2.63 (3H, s), 6.48 (1H, d), 7.14 (1H, d), 7.54 (1H, d).

### Intermediate 58c: tert-Butyl 6-((tert-butoxycarbonyl)amino)-5-fluoro-7-methyl-1H-indole-1-carboxylate

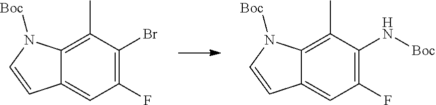 [SMILES: Cc1c(Br)c(F)cc2ccn(C(=O)OC(C)(C)C)c12 ; Cc1c(NC(=O)OC(C)(C)C)c(F)cc2ccn(C(=O)OC(C)(C)C)c12]

Cs₂CO₃ (1.49 g, 4.57 mmol) was added to a degassed mixture of Ephos Pd G4 (70.0 mg, 0.0762 mmol), Ephos (40.8 mg, 0.0763 mmol), tert-butyl carbamate (357 mg, 3.05 mmol) and tert-butyl 6-bromo-5-fluoro-7-methyl-1H-indole-1-carboxylate (500 mg, 1.52 mmol) in 1,4-dioxane (10 mL) at r.t. under N₂. The resulting mixture was stirred at 100° C. for 17 h. The solvent was then removed under reduced pressure. Purification by FSC (gradient: 0-100% EtOAc in petroleum ether) gave the title compound (280 mg, 50%) as a white solid. ¹H NMR: (CDCl₃) δ 1.50 (9H, s), 1.63 (9H, s), 2.47 (3H, s), 6.01 (1H, br s), 6.46 (1H, d), 7.11 (1H, d), 7.53 (1H, d). m/z (ES⁺), [M+Na]⁺=387.2.

### Intermediate 58d: 3-((5-Fluoro-7-methyl-1H-indol-6-yl)amino)propanoic acid

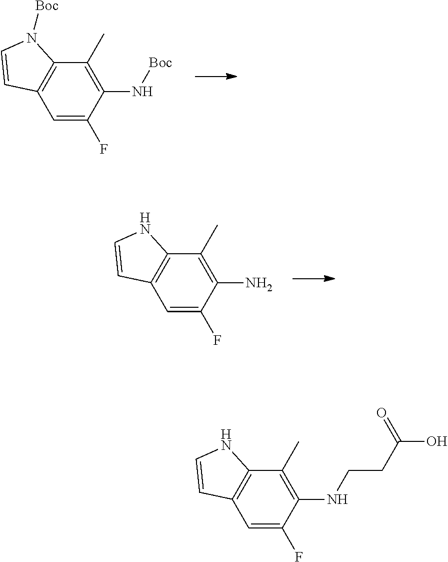 [SMILES: Cc1c(NC(=O)OC(C)(C)C)c(F)cc2ccn(C(=O)OC(C)(C)C)c12 ; Cc1c(N)c(F)cc2cc[nH]c12 ; Cc1c(NCCC(=O)O)c(F)cc2cc[nH]c12]

tert-Butyl 6-((tert-butoxycarbonyl)amino)-5-fluoro-7-methyl-1H-indole-1-carboxylate (280 mg, 0.768 mmol) was dissolved in 2,2,2-trifluoroethanol (8 mL) and sealed into a microwave tube. The reaction was heated to 150° C. for 1 h in a microwave reactor and then cooled to r.t. The solvent was removed and to the residue was added toluene (5 mL) followed by acrylic acid (84.0 mg, 1.17 mmol). The resulting mixture was stirred at 110° C. for 16 h. The solvent was then removed under reduced pressure. Purification by C-18FC (gradient: 5-30% MeCN in water (containing 0.05% FA)) gave the title compound in impure form (100 mg) as a yellow solid which was used in the next step without further purification. m/z (ES⁺), [M+H]⁺=237.3.

### Example 58: 1-(5-Fluoro-7-methyl-1H-indol-6-yl)dihydropyrimidine-2,4 (1H,3H)-dione

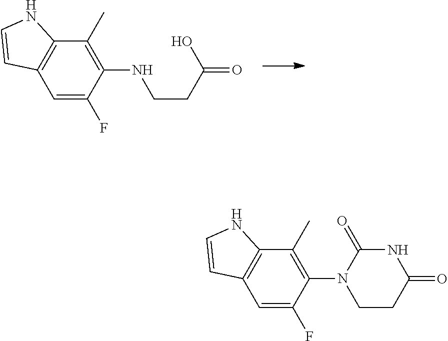 [SMILES: Cc1c(NCCC(=O)O)c(F)cc2cc[nH]c12 ; Cc1c(N2CCC(=O)NC2=O)c(F)cc2cc[nH]c12]

Urea (127 mg, 2.11 mmol) was added to a solution of impure 3-((5-fluoro-7-methyl-1H-indol-6-yl)amino)-propanoic acid (100 mg) in AcOH (3 mL) at r.t. under air. The resulting mixture was stirred at 100° C. for 4 h. The crude product was directly purified by C-18FC (gradient: 5-30% MeCN in water (containing 0.05% TFA)) and further purified by preparative HPLC (Column X, Eluent A, gradient: 35-45%) to give the title compound (15.0 mg, 7% over 3 steps) as a white solid. ¹H NMR: δ 2.38 (3H, s), 2.56-2.72 (1H, m), 2.78-2.95 (1H, m), 3.48-3.61 (1H, m), 3.61-3.75 (1H, m), 6.45 (1H, dd), 7.25 (1H, d), 7.46 (1H, t), 10.42 (1H, s), 11.29 (1H, s). ¹⁹F NMR (282 MHz) δ −133.32. m/z (ES⁺), [M+H]⁺=262.2.

### Example 59: 1-(1-Methyl-1H-indol-6-yl)dihydropyrimidine-2,4 (1H,3H)-dione

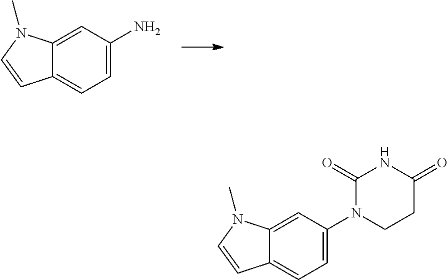 [SMILES: Cn1ccc2ccc(N)cc21 ; Cn1ccc2ccc(N3CCC(=O)NC3=O)cc21]

Acrylic acid (444 mg, 6.16 mmol) was added to a solution of 1-methyl-1H-indol-6-amine (300 mg, 2.05 mmol) in toluene (5 mL) at r.t. under N₂. The resulting solution was stirred at 110° C. for 12 h. The solvent was then removed under reduced pressure. The residue was dissolved in AcOH (5 mL) and to the solution was added urea (370 mg, 6.16 mmol) at r.t. under air. The resulting solution was stirred at 120° C. for 12 h. The solvent was then removed under reduced pressure. Purification by C-18FC (gradient: 0-30% MeCN in water (containing 0.1% FA)) gave a yellow material which was further purified by preparative HPLC (Column E, Eluent E, gradient: 42-60%) to give the title compound (90.0 mg, 18%) as a white solid. ¹H NMR: δ 2.74 (2H, t), 3.78 (3H, s), 3.81 (2H, t), 6.43 (1H, d), 6.99 (1H, dd), 7.36 (1H, d), 7.41 (1H, t), 7.53 (1H, d), 10.31 (1H, s). m/z (ES⁺), [M+H]⁺=244.1.

### Intermediate 60a: tert-Butyl 6-bromo-5-fluoro-1H-indole-1-carboxylate

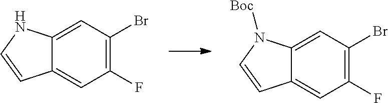 [SMILES: Fc1cc2cc[nH]c2cc1Br ; CC(C)(C)OC(=O)n1ccc2cc(F)c(Br)cc21]

DMAP (228 mg, 1.87 mmol) was added to a solution of DIEA (4.90 mL, 28.1 mmol), di-tert-butyl dicarbonate (4.34 mL, 18.7 mmol) and 6-bromo-5-fluoro-1H-indole (2.00 g, 9.34 mmol) in DCM (10 mL) at r.t. under N₂. The resulting mixture was stirred at r.t. for 2 h. The solvent was then removed under reduced pressure. Purification by FSC (gradient: 0-30% EtOAc in petroleum ether) gave the title compound (2.30 g, 78%) as a white solid. ¹H NMR: δ 1.62 (9H, s), 6.71 (1H, dd), 7.62 (1H, d), 7.75 (1H, d), 8.27 (1H, d).

### Intermediate 60b: tert-Butyl 6-amino-5-fluoro-1H-indole-1-carboxylate

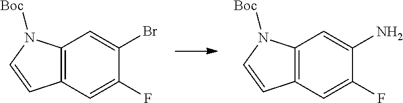 [SMILES: CC(C)(C)OC(=O)n1ccc2cc(F)c(Br)cc21 ; CC(C)(C)OC(=O)n1ccc2cc(F)c(N)cc21]

Copper(I) iodide (133 mg, 0.698 mmol) was added to a mixture of tert-butyl 6-bromo-5-fluoro-1H-indole-1-carboxylate (1.10 g, 3.50 mmol), aqueous NH₄OH (28%, 2.44 mL, 17.5 mmol) and K₃PO₄ (2.23 g, 10.5 mmol), L-proline (81.0 mg, 0.704 mmol) in DMF (15 mL) at r.t. under N₂. The resulting mixture was stirred at 90° C. for 16 h. The reaction mixture was quenched with water (100 mL), extracted with EtOAc (2×100 mL) and the combined organic extracts were washed with saturated brine (3×50 mL). The organic layer was dried (Na₂SO₄) and concentrated. Purification FSC (gradient 0-20% EtOAc in petroleum ether) gave the impure title compound (600 mg, 68% [10% purity based on ¹H NMR]) as a pale yellow solid. This was an inseparable mixture with 6-bromo-5-fluoro-1H-indole in a 1:10 ratio by (i.e. ˜10% pure) which was used in the next step without further purification. ¹H NMR: (desired compound only, CDCl₃) δ 1.67 (9H, s), 6.42 (1H, d), 7.16 (1H, d), 7.25 (2H, s), 7.42 (1H, d), 7.69 (1H, d). m/z (ES⁺), [M+H]⁺=251.1.

### Example 60: 1-(5-Fluoro-1H-indol-6-yl)dihydropyrimidine-2,4 (1H,3H)-dione

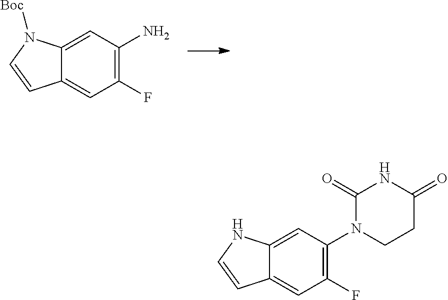 [SMILES: CC(C)(C)OC(=O)n1ccc2cc(F)c(N)cc21 ; O=C1CCN(c2cc3[nH]ccc3cc2F)C(=O)N1]

Acrylic acid (84 mg, 1.17 mmol) was added to a solution of tert-butyl 6-amino-5-fluoro-1H-indole-1-carboxylate (10% purity based on ¹H NMR, 580 mg, 0.232 mmol) in toluene (10 mL) at r.t. The resulting mixture was stirred at 110° C. for 16 h. The solvent was then removed under reduced pressure. The residue was dissolved in AcOH (10 mL) and to the solution was added urea (69.6 mg, 1.16 mmol) at r.t. under air. The resulting mixture was stirred at 120° C. for 4 h. The solvent was then removed under reduced pressure. Purification by preparative HPLC (Column J, Eluent A, gradient: 15-18%) gave the title compound (28.0 mg, 49%) as a white solid. ¹H NMR: δ 2.74 (2H, t), 3.73 (2H, t), 6.41-6.49 (1H, m), 7.39 (1H, d), 7.42 (1H, d), 7.46 (1H, t), 10.40 (1H, s), 11.29 (1H, s). ¹⁹F NMR (282 MHz) δ −133.61. m/z (ES⁺), [M+H]⁺=248.2.

### Intermediate 61a: tert-Butyl 5-methoxy-6-nitro-1H-indole-1-carboxylate

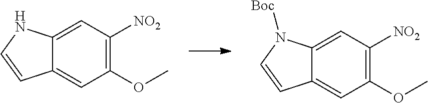 [SMILES: COc1cc2cc[nH]c2cc1[N+](=O)[O-] ; COc1cc2ccn(C(=O)OC(C)(C)C)c2cc1[N+](=O)[O-]]

DMAP (140 mg, 1.15 mmol) was added to DIEA (3.00 mL, 17.2 mmol), di-tert-butyl dicarbonate (2.66 mL, 11.46 mmol) and 5-methoxy-6-nitro-1H-indole (1.10 g, 5.72 mmol) in DCM (20 mL) at r.t. under air. The resulting solution was stirred at r.t. for 2 h. The solvent was then removed under reduced pressure. Purification by FSC (gradient: 0-20% EtOAc in petroleum ether) gave the title compound (1.50 g, 90%) as a pale yellow solid. ¹H NMR: (CDCl₃) δ 1.69 (9H, s), 3.99 (3H, s), 6.57 (1H, dd), 7.17 (1H, s), 7.78 (1H, d), 8.69 (1H, s). m/z (ES⁺), [M+H]⁺=293.1.

### Intermediate 61b: tert-Butyl 6-amino-5-methoxy-1H-indole-1-carboxylate

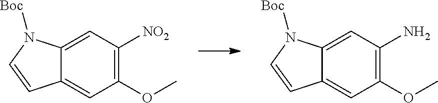 [SMILES: COc1cc2ccn(C(=O)OC(C)(C)C)c2cc1[N+](=O)[O-] ; COc1cc2ccn(C(=O)OC(C)(C)C)c2cc1N]

Trichlorosilane (1.78 g, 13.1 mmol) was added to a mixture of tert-butyl 5-methoxy-6-nitro-1H-indole-1-carboxylate (1.10 g, 3.76 mmol) and DIEA (3.29 mL, 18.8 mmol) in MeCN (15 mL) at 0° C. under N₂. The resulting solution was stirred at r.t. for 16 h. 20 mL of a saturated solution of NaHCO₃ was added dropwise and the biphasic mixture was allowed to stir for 0.5 h. The reaction mixture was poured into water (100 mL) and extracted with EtOAc (3×100 mL). The combined organic extracts were dried (Na₂SO₄) and concentrated to give the title compound (800 mg, 81%) as a black solid. ¹H NMR: (CDCl₃) δ 1.67 (9H, s), 3.91 (3H, s), 6.43 (1H, dd), 6.94 (1H, s), 7.37 (1H, d), 7.61 (1H, s). m/z (ES⁺), [M+H]⁺=263.1.

### Intermediate 61c: 3-((1-(tert-Butoxycarbonyl)-5-methoxy-1H-indol-6-yl)amino)propanoic acid

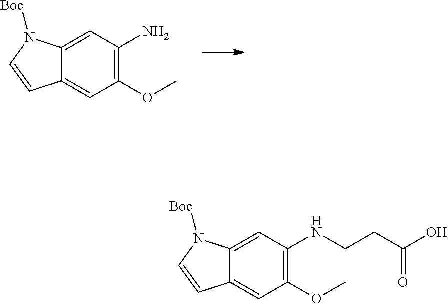 [SMILES: COc1cc2ccn(C(=O)OC(C)(C)C)c2cc1N ; COc1cc2ccn(C(=O)OC(C)(C)C)c2cc1NCCC(=O)O]

Acrylic acid (412 mg, 5.72 mmol) was added to a solution of tert-butyl 6-amino-5-methoxy-1H-indole-1-carboxylate (500 mg, 1.91 mmol) in toluene (5 mL) at r.t. under N₂. The resulting mixture was stirred at 110° C. for 16 h. The solvent was then removed under reduced pressure. Purification by FSC (gradient: 0-10% MeOH in DCM) gave the title compound in an impure form (610 mg) as a yellow oil which was used in the next step without further purification. m/z (ES⁺), [M+H]⁺=335.2

### Example 61: 1-(5-Methoxy-1H-indol-6-yl)dihydropyrimidine-2,4 (1H,3H)-dione

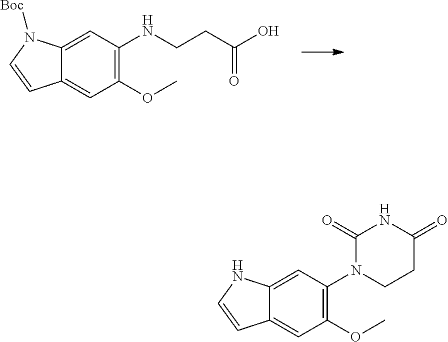 [SMILES: COc1cc2ccn(C(=O)OC(C)(C)C)c2cc1NCCC(=O)O ; COc1cc2cc[nH]c2cc1N1CCC(=O)NC1=O]

Urea (329 mg, 5.47 mmol) was added to a solution of impure 3-((1-(tert-butoxycarbonyl)-5-methoxy-1H-indol-6-yl)amino)propanoic acid (610 mg) in AcOH (10 mL) at r.t. under air. The resulting mixture was stirred at 110° C. for 3 h. The solvent was then removed under reduced pressure. Purification by C-18FC (gradient: 0-50% water (containing 0.05% conc. HCl) in MeCN) gave the title compound (65.0 mg, 13% over two-steps) as a white solid. ¹H NMR: δ 2.70 (2H, t), 3.32 (2H, t), 3.79 (3H, s), 6.38 (1H, d), 7.16 (1H, s), 7.27 (1H, d), 7.34 (1H, t), 10.22 (1H, s), 11.01 (1H, s). m/z (ES⁺), [M+H]⁺=260.1.

### Intermediate 62a: 7-Methoxy-6-nitro-1H-indole

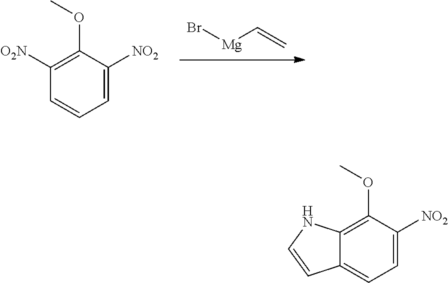

Vinylmagnesium bromide (1M in THF, 115 mL, 115 mmol) was added to a solution of 2-methoxy-1,3-dinitrobenzene (7.60 g, 38.4 mmol) in THF (150 mL) at −78° C. under N₂. The resulting mixture was stirred at −78° C. for 2 h. The reaction mixture was quenched with saturated NH₄Cl (200 mL) and extracted with EtOAc (3×200 mL). The combined organic extracts were dried (Na₂SO₄) and concentrated to give a brown residue. Purification by FSC (gradient: 0-19% EtOAc in petroleum ether) gave the title compound (3.00 g, 41%) as a yellow solid. ¹H NMR: (CDCl₃) δ 4.12 (3H, s), 6.66 (1H, dd), 7.41 (1H, dd), 7.48 (1H, dd), 7.79 (1H, d), 8.83 (1H, br s). m/z (ES⁺), [M+H]⁺=193.1.

### Intermediate 62b: tert-Butyl 7-methoxy-6-nitro-1H-indole-1-carboxylate

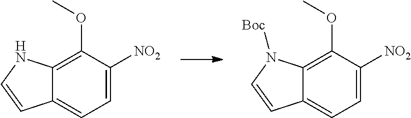 [SMILES: COc1c([N+](=O)[O-])ccc2cc[nH]c12 ; COc1c([N+](=O)[O-])ccc2ccn(C(=O)OC(C)(C)C)c12]

Di-tert-butyl dicarbonate (7.25 mL, 31.2 mmol) was added to a mixture of 7-methoxy-6-nitro-1H-indole (3.00 g, 15.6 mmol), DIEA (8.18 mL, 46.8 mmol) and DMAP (191 mg, 1.56 mmol) in DCM (100 mL) at r.t. The resulting mixture was stirred at r.t. for 1 h. The solvent was then removed under reduced pressure. Purification by FSC (gradient: 0-9% EtOAc in petroleum ether) gave the title compound (3.78 g, 83%) as a pale yellow crystalline solid. ¹H NMR: (CDCl₃) δ 1.68 (9H, s), 3.95 (3H, s), 6.62 (1H, d), 7.35 (1H, d), 7.70 (1H, d), 7.75 (1H, d). m/z (ES⁺), [M-tBu+2H]⁺=237.0.

### Intermediate 62c: tert-Butyl 6-amino-7-methoxy-1H-indole-1-carboxylate

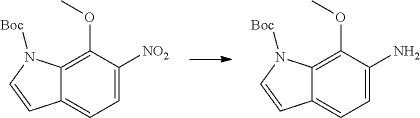 [SMILES: COc1c([N+](=O)[O-])ccc2ccn(C(=O)OC(C)(C)C)c12 ; COc1c(N)ccc2ccn(C(=O)OC(C)(C)C)c12]

tert-Butyl 7-methoxy-6-nitro-1H-indole-1-carboxylate (3.60 g, 12.3 mmol) and Pd/C (10% on activated carbon, 2.62 g, 2.46 mmol) in MeOH (100 mL) and DCM (10 mL) were stirred under H₂ (1 atm) at r.t. for 1 h. The reaction mixture was filtered through a celite pad. The solvent was then removed under reduced pressure. Purification by by FSC (gradient: 0-17% EtOAc in petroleum ether) gave the title compound (650 mg, 20%) as a pale yellow oil. ¹H NMR: (CDCl₃) δ 1.66 (9H, s), 3.77 (3H, s), 6.45 (1H, d), 6.77 (1H, d), 7.12 (1H, d), 7.38 (1H, d). m/z (ES⁺), [M+H]⁺=263.1.

### Example 62: 1-(7-Methoxy-1H-indol-6-yl)dihydropyrimidine-2,4 (1H,3H)-dione

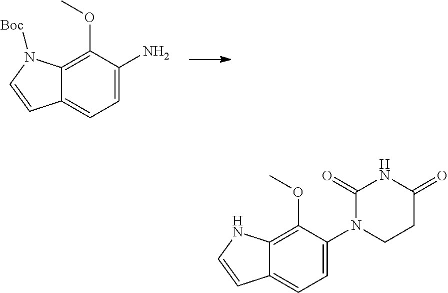 [SMILES: COc1c(N)ccc2ccn(C(=O)OC(C)(C)C)c12 ; COc1c(N2CCC(=O)NC2=O)ccc2cc[nH]c12]

Acrylic acid (495 mg, 6.86 mmol) was added to tert-butyl 6-amino-7-methoxy-1H-indole-1-carboxylate (600 mg, 2.29 mmol) in toluene (15 mL) at r.t. under N₂. The resulting mixture was stirred at 110° C. for 16 h. The solvent was removed under reduced pressure. The residue was dissolved in AcOH (15 mL) and urea (412 mg, 6.86 mmol) was added to the mixture. The resulting mixture was stirred at 110° C. for 3 h. The solvent was then removed under reduced pressure. Purification by C-18FC (gradient: 0-20% MeCN in water (containing 0.1% FA)) gave the title compound (240 mg, 41%) as a white solid. ¹H NMR: δ 2.73 (2H, t), 3.67 (2H, t), 3.90 (3H, s), 6.47 (1H, dd), 6.87 (1H, d), 7.28 (1H, d), 7.37 (1H, t), 10.32 (1H, s), 11.34 (1H, s). m/z (ES⁺), [M+H]⁺=260.0.

### Intermediate 63a: tert-Butyl 4-amino-5-fluoro-1H-indole-1-carboxylate

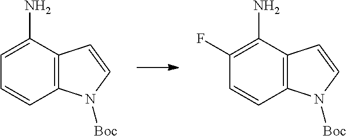 [SMILES: CC(C)(C)OC(=O)n1ccc2c(N)cccc21 ; CC(C)(C)OC(=O)n1ccc2c(N)c(F)ccc21]

Selectfluor (839 mg, 2.37 mmol) was added to a solution of tert-butyl 4-amino-1H-indole-1-carboxylate (550 mg, 2.37 mmol) in MeCN (40 mL) at r.t. The resulting solution was stirred at r.t. for 3 h. The solvent was then removed under reduced pressure. Purification by FSC (gradient: 0-20% EtOAc in petroleum ether) gave the title compound (78.0 mg, 13%) as a brown oil. ¹H NMR: δ 1.61 (9H, s), 5.49 (2H, s), 6.91 (1H, d), 6.96 (1H, dd), 7.20 (1H, d), 7.50 (1H, d). m/z (ES⁺), [M+H]⁺=251.1.

### Example 63: 1-(5-Fluoro-1H-indol-4-yl)dihydropyrimidine-2,4 (1H,3H)-dione

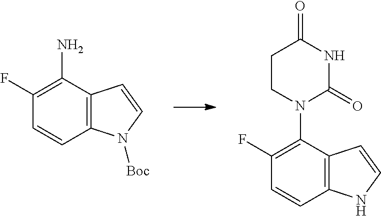 [SMILES: CC(C)(C)OC(=O)n1ccc2c(N)c(F)ccc21 ; O=C1CCN(c2c(F)ccc3[nH]ccc23)C(=O)N1]

Acrylic acid (51.8 mg, 0.719 mmol) was added to a solution of tert-butyl 4-amino-5-fluoro-1H-indole-1-carboxylate (45.0 mg, 0.180 mmol) in toluene (5 mL). The resulting mixture was stirred at 120° C. for 16 h. The solvent was removed under reduced pressure. The residue was dissolved in AcOH (5 mL), urea (10.8 mg, 0.180 mmol) was added and the resulting solution was stirred at 120° C. for 5 h. The solvent was then removed under reduced pressure. Purification by preparative HPLC (Column B, Eluent B, gradient: 10-30%) gave the title compound (3.6 mg, 8%) as a white solid. ¹H NMR: δ 2.61-2.73 (1H, m), 2.89-2.95 (1H, m), 3.69-3.74 (1H, m), 3.79-3.86 (1H, m), 6.49 (1H, d), 7.02-7.09 (1H, m), 7.34-7.42 (1H, m), 7.46 (1H, d), 10.48 (1H, s), 11.36 (1H, s). ¹⁹F NMR (376 MHz) δ −133.89. m/z (ES⁺), [M+H]⁺=248.0.

### Intermediate 64a: tert-Butyl 4-amino-5-chloro-1H-indole-1-carboxylate

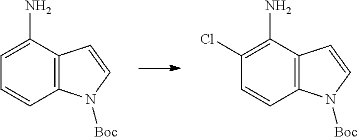 [SMILES: CC(C)(C)OC(=O)n1ccc2c(N)cccc21 ; CC(C)(C)OC(=O)n1ccc2c(N)c(Cl)ccc21]

NCS (172 mg, 1.29 mmol) was added to a solution of tert-butyl 4-amino-1H-indole-1-carboxylate (300 mg, 1.29 mmol) in DCM (10 mL) at 0° C. under air. The resulting mixture was stirred at 0° C. for 2 h. The solvent was then removed under reduced pressure. Purification by FSC (gradient: 0-30% EtOAc in petroleum ether) gave the title compound (50.0 mg, 15%) as a yellow oil. ¹H NMR: δ 1.60 (9H, s), 5.71 (2H, s), 6.95 (1H, d), 7.11 (1H, d), 7.27 (1H, d), 7.50 (1H, d). m/z (ES⁺), [M+H]⁺=267.2.

### Example 64: 1-(5-Chloro-1H-indol-4-yl)dihydropyrimidine-2,4 (1H,3H)-dione

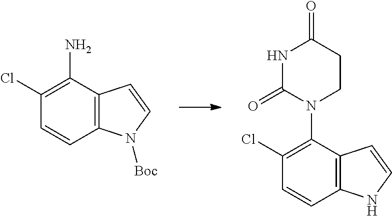 [SMILES: CC(C)(C)OC(=O)n1ccc2c(N)c(Cl)ccc21 ; O=C1CCN(c2c(Cl)ccc3[nH]ccc23)C(=O)N1]

Acrylic acid (97.0 mg, 1.35 mmol) was added to a solution of tert-butyl 4-amino-5-chloro-1H-indole-1-carboxylate (90.0 mg, 0.337 mmol) in toluene (6 mL) at r.t. under air. The resulting mixture was stirred at 120° C. for 50 h. The solvent was then removed under reduced pressure. The residue was dissolved in AcOH (5 mL), urea (35.5 mg, 0.591 mmol) was added and the resulting mixture was stirred at 120° C. for 4 h. The solvent was then removed under reduced pressure. Purification by C-18FC (gradient 5-50% MeCN in water (containing 0.1% FA)) gave the title compound (16.0 mg, 18%) as a yellow solid. ¹H NMR: δ 2.64-2.80 (1H, m), 2.80-2.96 (1H, m), 3.57-3.80 (2H, m), 6.45-6.53 (1H, m), 7.19 (1H, d), 7.37-7.50 (2H, m), 10.45 (1H, s), 11.46 (1H, s). m/z (ES⁺), [M+H]⁺=264.1.

### Intermediate 65a: tert-Butyl 6-bromo-3-(2-methoxy-2-oxoethyl)-1H-indole-1-carboxylate

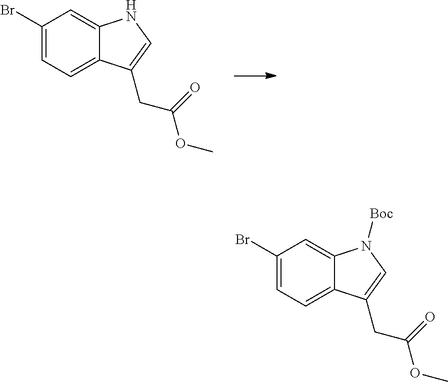 [SMILES: COC(=O)Cc1c[nH]c2cc(Br)ccc12 ; COC(=O)Cc1cn(C(=O)OC(C)(C)C)c2cc(Br)ccc12]

DMAP (91.0 mg, 0.745 mmol) was added to a solution of di-tert-butyl dicarbonate (2.60 mL, 11.2 mmol), DIEA (3.91 mL, 22.4 mmol) and methyl 2-(6-bromo-1H-indol-3-yl)acetate (2.00 g, 7.46 mmol) in DCM (20 mL) at r.t. under air. The resulting solution was stirred at r.t. for 2 h. The solvent was then removed under reduced pressure. Purification by FSC (gradient: 0-20% EtOAc in petroleum ether) gave the title compound (2.70 g, 98%) as a white solid. ¹H NMR: δ 1.63 (9H, s), 3.63 (3H, s), 3.83 (2H, d), 7.44 (1H, dd), 7.54 (1H, d), 7.66 (1H, s), 8.22 (1H, d).

### Intermediate 65b: tert-Butyl 6-(2,4-dioxotetrahydropyrimidin-1 (2H)-yl)-3-(2-methoxy-2-oxoethyl)-1H-indole-1-carboxylate

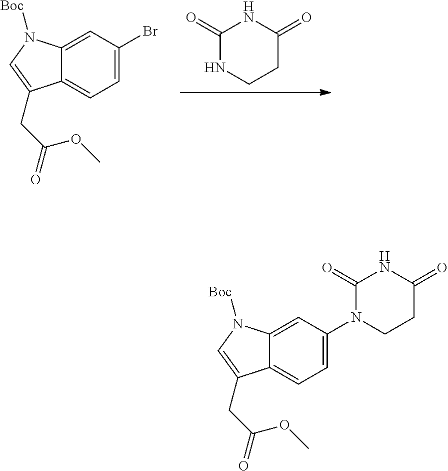

Ephos Pd G4 (337 mg, 0.367 mmol) was added to degassed mixture of Ephos (196 mg, 0.366 mmol), Cs₂CO₃ (7.17 g, 22.0 mmol), dihydropyrimidine-2,4 (1H,3H)-dione (2.51 g, 22.0 mmol) and tert-butyl 6-bromo-3-(2-methoxy-2-oxoethyl)-1H-indole-1-carboxylate (2.70 g, 7.33 mmol) in 1,4-dioxane (20 mL) at r.t. The resulting mixture was stirred at 100° C. for 17 h. The solvent was then removed under reduced pressure. Purification by FSC (gradient: 0-80% EtOAc in petroleum ether) gave the title compound (2.80 g, 95%) as a pale yellow solid. ¹H NMR: δ 1.63 (9H, s), 2.75 (2H, t), 3.64 (3H, s), 3.80-3.88 (4H, m), 7.24 (1H, dd), 7.56 (1H, d), 7.66 (1H, s), 8.04 (1H, d), 10.38 (1H, s). m/z (ES⁺), [M+H]⁺=402.1.

### Intermediate 65c: 2-(1-(tert-Butoxycarbonyl)-6-(2,4-dioxotetrahydropyrimidin-1 (2H)-yl)-1H-indol-3-yl)-acetic acid

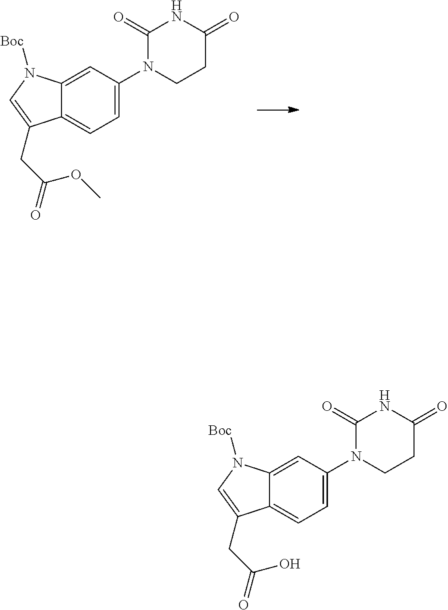 [SMILES: COC(=O)Cc1cn(C(=O)OC(C)(C)C)c2cc(N3CCC(=O)NC3=O)ccc12 ; CC(C)(C)OC(=O)n1cc(CC(=O)O)c2ccc(N3CCC(=O)NC3=O)cc21]

Trimethylstannanol (6.08 g, 33.6 mmol) was added to a solution of tert-butyl 6-(2,4-dioxotetrahydropyrimidin-1 (2H)-yl)-3-(2-methoxy-2-oxoethyl)-1H-indole-1-carboxylate (2.70 g, 6.73 mmol) in DCE (50 mL) at r.t. under air. The resulting solution was stirred at 80° C. for 17 h. The solvent was then removed under reduced pressure. Purification by FSC (gradient: 0-20% MeOH in DCM) gave the title compound (2.20 g, 84%) as a white solid. ¹H NMR: δ 1.63 (9H, s), 2.75 (2H, t), 3.71 (2H, s), 3.84 (2H, t), 7.23 (1H, dd), 7.56 (1H, d), 7.63 (1H, s), 8.03 (1H, d), 10.38 (1H, s), 12.43 (1H, s). m/z (ES⁺), [M-tBu+2H]⁺=332.1.

### Example 65: 2-(6-(2,4-Dioxotetrahydropyrimidin-1 (2H)-yl)-1H-indol-3-yl)acetic acid

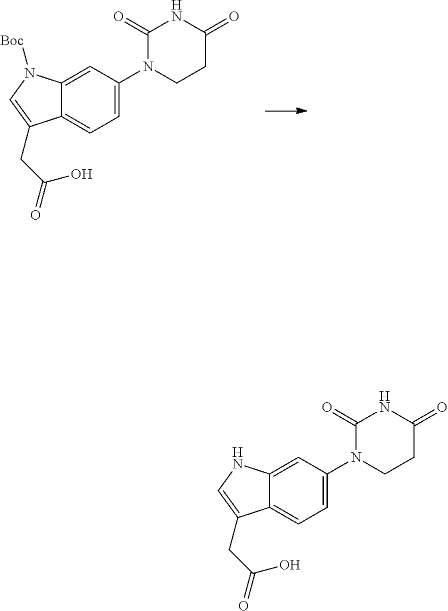 [SMILES: CC(C)(C)OC(=O)n1cc(CC(=O)O)c2ccc(N3CCC(=O)NC3=O)cc21 ; O=C(O)Cc1c[nH]c2cc(N3CCC(=O)NC3=O)ccc12]

TFA (20.0 ml, 260 mmol) was added to a solution of 2-(1-(tert-butoxycarbonyl)-6-(2,4-dioxotetrahydro-pyrimidin-1 (2H)-yl)-1H-indol-3-yl)acetic acid (2.16 g, 5.58 mmol) in DCM (60 mL) at r.t. under air. The resulting solution was stirred at r.t. for 2 h and then stirred at 60° C. for 7 days. The solvent was then removed under reduced pressure. Purification by C-18FC (gradient: 0-20% MeCN in water (containing 0.1% conc. HCl)) gave the title compound (1.00 g, 62%) as a pale yellow solid. ¹H NMR: δ 2.72 (2H, t), 3.64 (2H, s), 3.78 (2H, t), 6.94 (1H, dd), 7.28 (2H, dd), 7.47 (1H, d), 10.28 (1H, s), 10.98 (1H, d), 12.16 (1H, br s). m/z (ES⁺), [M+H]⁺=288.1.

### Example 66: 2-(6-(2,4-Dioxotetrahydropyrimidin-1 (2H)-yl)-1H-indol-3-yl)-N-methylacetamide

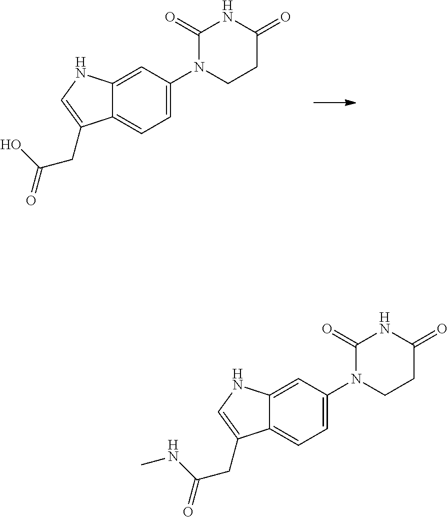 [SMILES: O=C(O)Cc1c[nH]c2cc(N3CCC(=O)NC3=O)ccc12 ; CNC(=O)Cc1c[nH]c2cc(N3CCC(=O)NC3=O)ccc12]

PyBOP (136 mg, 0.261 mmol) was added to a solution of DIEA (61.0 μL, 0.35 mmol), methylamine (4M in THF, 218 μL, 0.872 mmol) and 2-(6-(2,4-dioxotetrahydropyrimidin-1 (2H)-yl)-1H-indol-3-yl)acetic acid (50.0 mg, 0.174 mmol) in DMF (2 mL) at r.t. under air. The resulting solution was stirred at r.t. for 2 h. The crude product was purified by C-18FC (gradient: 0-40% MeCN in water (containing 0.1% FA)) to give the title compound (22.0 mg, 42%) as a pale yellow solid. ¹H NMR: δ 2.57 (3H, d), 2.73 (2H, t), 3.48 (2H, s), 3.78 (2H, t), 6.93 (1H, dd), 7.22 (1H, d), 7.28 (1H, d), 7.52 (1H, d), 7.77 (1H, d), 10.28 (1H, s), 10.96 (1H, d). m/z (ES⁺), [M+H]⁺=301.1.

### Intermediate 67a: Ethyl 4-(4-bromo-2-nitrophenyl)-3-oxobutanoate

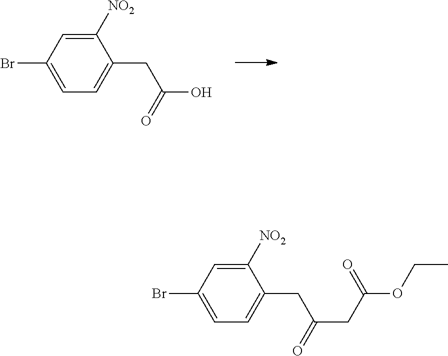 [SMILES: O=C(O)Cc1ccc(Br)cc1[N+](=O)[O-] ; CCOC(=O)CC(=O)Cc1ccc(Br)cc1[N+](=O)[O-]]

Step 1: CDI (2.06 g, 12.7 mmol) was added to a solution of 2-(4-bromo-2-nitrophenyl)acetic acid (3.00 g, 11.5 mmol) in THF (30 mL) at r.t. under N₂. The resulting solution was stirred at r.t. for 4 h.

Step 2 (performed in parallel): Magnesium ethoxide (2.64 g, 23.1 mmol) was added to a solution of 3-ethoxy-3-oxopropanoic acid (6.10 g, 46.2 mmol) in THF (30 mL) at r.t. under N₂. The resulting solution was stirred at r.t. for 1 h. The solvent was then removed under reduced pressure.

Step 3: The solution from Step 1 was added to the crude mixture of Step 2. The resulting solution was stirred at r.t. for 18 h. The solvent was then removed under reduced pressure. Purification by FSC (gradient: 0-32% EtOAc in petroleum ether) gave the title compound (2.40 g, 63%) as a white solid. ¹H NMR: δ 1.19 (3H, t), 3.73 (2H, s), 4.10 (2H, q), 4.31 (2H, s), 7.43 (1H, d), 7.93-7.96 (1H, m), 8.26 (1H, d). m/z (ES⁺), [M+H]⁺=332.0.

### Intermediate 67b: Ethyl 2-(6-bromo-1H-indol-2-yl)acetate

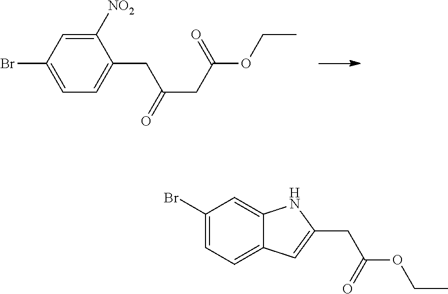 [SMILES: CCOC(=O)CC(=O)Cc1ccc(Br)cc1[N+](=O)[O-] ; CCOC(=O)Cc1cc2ccc(Br)cc2[nH]1]

A 15% solution of titanium trichloride in 2 N HCl (72 mL) was added to a stirred solution of ethyl 4-(4-bromo-2-nitrophenyl)-3-oxobutanoate (2.40 g, 7.27 mmol), ammonium acetate (15M in water, 13.0 mL, 195 mmol) in acetone (24 mL) at r.t. The resulting solution was stirred at r.t. for 2 h. The reaction mixture was diluted with EtOAc (50 mL) and washed sequentially with water (2×30 mL), saturated brine (2×25 mL). The organic layer was dried (Na₂SO₄) and concentrated to give the title compound (1.65 g, 80%) as a brown solid. ¹H NMR: δ 1.19 (3H, t), 3.82 (2H, s), 4.10 (2H, q), 6.29 (1H, s), 7.06 (1H, dd), 7.39 (1H, d), 7.49 (1H, d), 11.18 (1H, s). m/z (ES⁺), [M+H]⁺=284.0.

### Intermediate 67c: tert-Butyl 6-bromo-2-(2-ethoxy-2-oxoethyl)-1H-indole-1-carboxylate

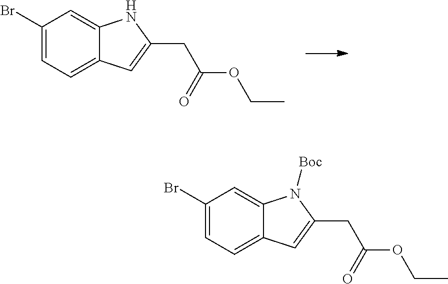 [SMILES: CCOC(=O)Cc1cc2ccc(Br)cc2[nH]1 ; CCOC(=O)Cc1cc2ccc(Br)cc2n1C(=O)OC(C)(C)C]

DMAP (143 mg, 1.17 mmol) was added to a solution of ethyl 2-(6-bromo-1H-indol-2-yl)acetate (1.65 g, 5.85 mmol), di-tert-butyl dicarbonate (2.72 mL, 11.7 mmol) and DIEA (2.04 mL, 11.7 mmol) in DCM (20 mL) at r.t. under air. The resulting solution was stirred at r.t. for 2 h. The solvent was then removed under reduced pressure. Purification by FSC (gradient: 0-15% EtOAc in petroleum ether) gave the title compound (1.70 g, 76%) as a white solid. ¹H NMR: δ 1.18-1.24 (3H, m), 1.59 (9H, s), 4.03-4.16 (4H, m), 6.66 (1H, s), 7.39-7.41 (1H, m), 7.53 (1H, d), 8.23 (1H, d). m/z (ES⁺), [M-tBu+2H]⁺=327.9.

### Intermediate 67d: 2-(6-Bromo-1-(tert-butoxycarbonyl)-1H-indol-2-yl)acetic acid

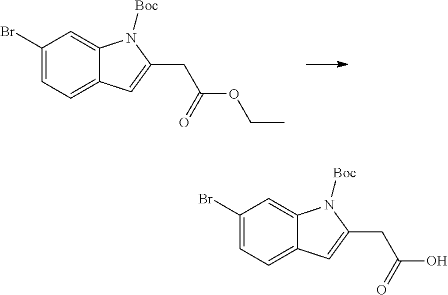 [SMILES: CCOC(=O)Cc1cc2ccc(Br)cc2n1C(=O)OC(C)(C)C ; CC(C)(C)OC(=O)n1c(CC(=O)O)cc2ccc(Br)cc21]

A mixture of LiOH (160 mg, 6.67 mmol) in water (10 mL) was added to a solution of tert-butyl 6-bromo-2-(2-ethoxy-2-oxoethyl)-1H-indole-1-carboxylate (1.70 g, 4.45 mmol) in THF (20 mL) at r.t. The resulting mixture was stirred at r.t. for 16 h. The reaction mixture was neutralised using 2M HCl. The reaction mixture was then diluted with EtOAc (100 mL), and washed sequentially with water (2×50 mL). The organic layer was dried (Na₂SO₄) and concentrated. Purification by C-18FC (gradient: 0-33% MeOH in water) gave the title compound (1.10 g, 70%) as a white solid. ¹H NMR: δ 1.57 (9H, s), 3.55 (2H, s), 6.27 (1H, s), 7.26-7.28 (1H, m), 7.38 (1H, d), 8.16 (1H, d). m/z (ES⁺), [M-tBu+2H]⁺=299.9.

### Intermediate 67e: tert-Butyl 6-bromo-2-(2-(methylamino)-2-oxoethyl)-1H-indole-1-carboxylate

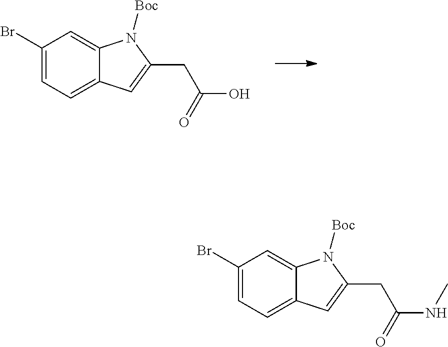 [SMILES: CC(C)(C)OC(=O)n1c(CC(=O)O)cc2ccc(Br)cc21 ; CNC(=O)Cc1cc2ccc(Br)cc2n1C(=O)OC(C)(C)C]

PyBOP (1.10 g, 2.11 mmol) was added to a mixture of 2-(6-bromo-1-(tert-butoxycarbonyl)-1H-indol-2-yl)acetic acid (500 mg, 1.41 mmol), DIEA (370 μL, 2.12 mmol) and methylamine (2M solution in THF, 1.41 mL, 2.82 mmol) in DCM (20 mL) at r.t. The resulting solution was stirred at r.t. for 16 h. The solvent was then removed under reduced pressure. Purification by C-18FC (gradient: 0-60% MeOH in water) gave the title compound (500 mg, 96%) as a white solid. ¹H NMR: δ 1.55 (9H, s), 2.54-2.61 (3H, m), 3.83 (2H, s), 6.55 (1H, s), 7.33-7.36 (1H, m), 7.48 (1H, d), 7.78 (1H, d), 8.21 (1H, d). m/z (ES⁺), [M+H]⁺=369.0.

### Example 67: 2-(6-(2,4-Dioxotetrahydropyrimidin-1 (2H)-yl)-1H-indol-2-yl)-N-methylacetamide

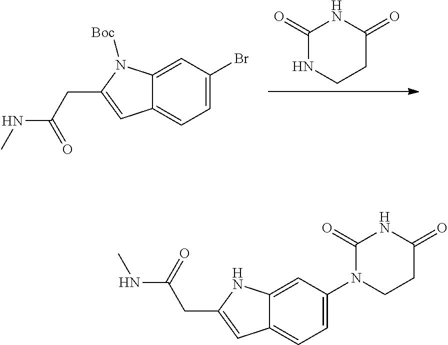 [SMILES: CNC(=O)Cc1cc2ccc(Br)cc2n1C(=O)OC(C)(C)C ; O=C1CCNC(=O)N1 ; CNC(=O)Cc1cc2ccc(N3CCC(=O)NC3=O)cc2[nH]1]

Ephos (29.1 mg, 0.0544 mmol) and Ephos Pd G4 (50.0 mg, 0.0544 mmol) were added to a degassed mixture of Cs₂CO₃ (710 mg, 2.18 mmol), tert-butyl 6-bromo-2-(2-(methylamino)-2-oxoethyl)-1H-indole-1-carboxylate (400 mg, 1.09 mmol) and dihydropyrimidine-2,4 (1H,3H)-dione (373 mg, 3.27 mmol) in 1,4-dioxane (15 mL) at r.t. under N₂. The resulting mixture was stirred at 100° C. for 16 h. The solvent was then removed under reduced pressure. Purification by C-18FC (gradient: 0-22% MeCN in water) gave material that was further purified by preparative HPLC (Column C, Eluent C, gradient: 15-35%) to give the title compound (13.7 mg, 4%) as a pink solid. ¹H NMR: δ 2.52-2.64 (3H, m), 2.72-2.74 (2H, m), 3.57 (2H, s), 3.77-3.79 (2H, m), 6.21 (1H, d), 6.87-6.91 (1H, m), 7.25 (1H, s), 7.41 (1H, d), 7.93 (1H, s), 10.27 (1H, s), 11.03 (1H, s). m/z (ES⁺), [M+H]⁺=301.2

### Intermediate 68a: tert-Butyl 2-(6-bromo-1H-indol-1-yl)acetate

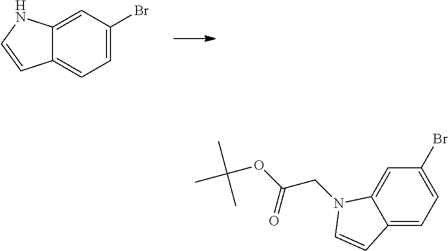 [SMILES: Brc1ccc2cc[nH]c2c1 ; CC(C)(C)OC(=O)Cn1ccc2ccc(Br)cc21]

K₂CO₃ (2.82 g, 20.4 mmol) was added to a solution of 6-bromo-1H-indole (2.00 g, 10.2 mmol) and tert-butyl 2-bromoacetate (3.98 g, 20.4 mmol) in MeCN (20 mL) at r.t. under N₂. The resulting mixture was stirred at 80° C. for 16 h. The solvent was then removed under reduced pressure. Purification by FSC (gradient: 0-20% EtOAc in petroleum ether) gave the title compound (3.00 g, 95%) as a yellow solid. ¹H NMR: δ 1.40 (9H, s), 5.01 (2H, s), 6.46-6.47 (1H, m), 7.14-7.16 (1H, m), 7.33 (1H, d), 7.49 (1H, d), 7.61-7.68 (1H, m). m/z (ES⁺), [M+H]⁺=310.0.

### Intermediate 68b: tert-Butyl 2-(6-(2,4-dioxotetrahydropyrimidin-1 (2H)-yl)-1H-indol-1-yl)acetate

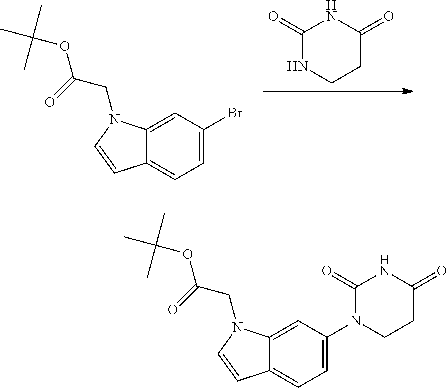

Ephos (172 mg, 0.322 mmol) and Ephos Pd G4 (296 mg, 0.322 mmol) were added to a degassed mixture of Cs₂CO₃ (4.20 g, 12.9 mmol), tert-butyl 2-(6-bromo-1H-indol-1-yl)acetate (2.00 g, 6.45 mmol) and dihydropyrimidine-2,4 (1H,3H)-dione (2.21 g, 19.4 mmol) in 1,4-dioxane (60 mL) at r.t. under N₂. The resulting mixture was stirred at 100° C. for 16 h. The solvent was then removed under reduced pressure. Purification by FSC (gradient: 0-9% MeOH in DCM) gave the title compound (1.50 g, 68%) as a yellow solid. ¹H NMR: δ 1.42 (9H, s), 2.73 (2H, t), 3.79 (2H, t), 4.98 (2H, s), 6.46 (1H, d), 7.01 (1H, dd), 7.32-7.39 (2H, m), 7.53 (1H, d), 10.32 (1H, s). m/z (ES⁺), [M+Na]⁺=366.1.

### Intermediate 68c: 2-(6-(2,4-Dioxotetrahydropyrimidin-1 (2H)-yl)-1H-indol-1-yl)acetic acid

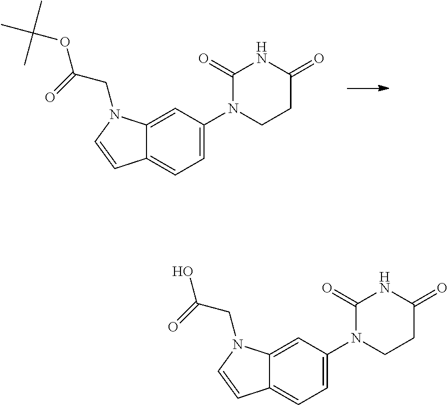 [SMILES: CC(C)(C)OC(=O)Cn1ccc2ccc(N3CCC(=O)NC3=O)cc21 ; O=C(O)Cn1ccc2ccc(N3CCC(=O)NC3=O)cc21]

tert-Butyl 2-(6-(2,4-dioxotetrahydropyrimidin-1 (2H)-yl)-1H-indol-1-yl)acetate (1.50 g, 4.37 mmol) was added to formic acid (20 mL) at r.t. The resulting solution was stirred at r.t. for 1 h. The solvent was removed under reduced pressure. Purification by C-18FC (gradient: 0-26% MeOH in water) gave the title compound (750 mg, 60%) as a pink solid. ¹H NMR: δ 2.72 (2H, t), 3.78 (2H, t), 4.99 (2H, s), 6.45 (1H, d), 7.00 (1H, dd), 7.36 (1H, d), 7.39 (1H, s), 7.52 (1H, d), 10.30 (1H, s), 12.87 (1H, br s). m/z (ES⁺), [M+H]⁺=288.1.

### Example 68: 2-(6-(2,4-Dioxotetrahydropyrimidin-1 (2H)-yl)-1H-indol-1-yl)-N-methylacetamide

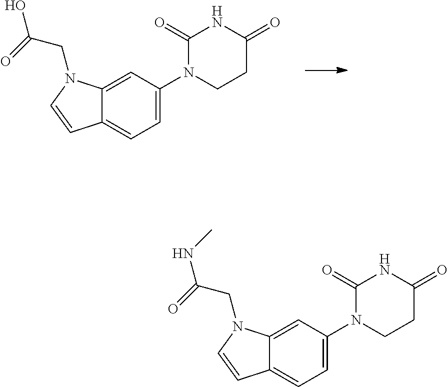 [SMILES: O=C(O)Cn1ccc2ccc(N3CCC(=O)NC3=O)cc21 ; CNC(=O)Cn1ccc2ccc(N3CCC(=O)NC3=O)cc21]

PyBOP (353 mg, 0.678 mmol) was added to a solution of 2-(6-(2,4-dioxotetrahydropyrimidin-1 (2H)-yl)-1H-indol-1-yl)acetic acid (130 mg, 0.453 mmol), DIEA (119 μL, 0.681 mmol) and methylamine (2M solution in THF, 1.13 mL, 2.26 mmol) in DCM (3 mL) at r.t. The resulting solution was stirred at r.t. for 3 h. The solvent was then removed under reduced pressure. Purification by C-18FC (gradient: 0-18% MeCN in water) gave the title compound (62.9 mg, 46%) as a white solid. ¹H NMR: δ 2.62 (3H, d), 2.73 (2H, t), 3.79 (2H, t), 4.78 (2H, s), 6.46 (1H, dd), 7.00 (1H, dd), 7.31-7.39 (2H, m), 7.53 (1H, d), 8.04 (1H, q), 10.32 (1H, s). m/z (ES⁺), [M+H]⁺=301.3.

### Intermediate 69a: tert-Butyl 3-(6-bromo-1H-indol-1-yl)propanoate

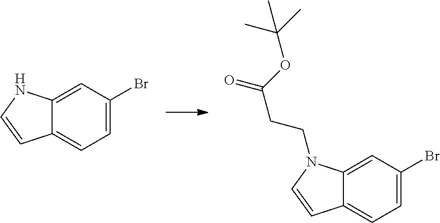 [SMILES: Brc1ccc2cc[nH]c2c1 ; CC(C)(C)OC(=O)CCn1ccc2ccc(Br)cc21]

DBU (1.15 mL, 7.63 mmol) was added to a solution of 6-bromo-1H-indole (3.00 g, 15.3 mmol) and tert-butyl acrylate (2.94 g, 22.9 mmol) in MeCN (30 mL) at r.t. under N₂. The resulting mixture was stirred at 60° C. for 16 h. The solvent was then removed under reduced pressure. Purification by FSC (gradient: 0-25% EtOAc in petroleum ether) gave the title compound (4.20 g, 85%) as a yellow solid. ¹H NMR: δ 1.28 (9H, s), 2.70 (2H, t), 4.37 (2H, t), 6.43 (1H, dd), 7.12 (1H, dd), 7.36 (1H, d), 7.47 (1H, d), 7.75 (1H, s). m/z (ES⁺), [M+H]⁺=326.0.

### Intermediate 69b: tert-Butyl 3-(6-(2,4-dioxotetrahydropyrimidin-1 (2H)-yl)-1H-indol-1-yl)propanoate

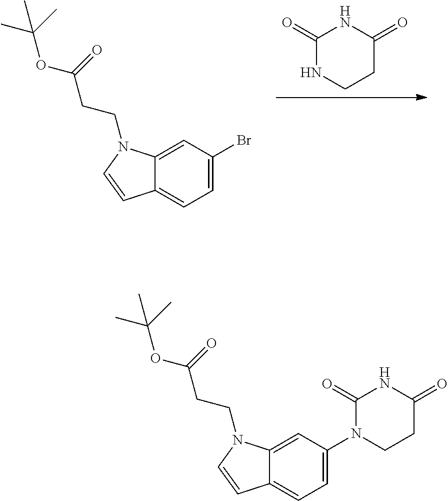 [SMILES: CC(C)(C)OC(=O)CCn1ccc2ccc(Br)cc21 ; O=C1CCNC(=O)N1 ; CC(C)(C)OC(=O)CCn1ccc2ccc(N3CCC(=O)NC3=O)cc21]

Ephos (165 mg, 0.309 mmol) and Ephos Pd G4 (283 mg, 0.308 mmol) were added to a degassed mixture of Cs₂CO₃ (4.02 g, 12.3 mmol), tert-butyl 3-(6-bromo-1H-indol-1-yl)propanoate (2.00 g, 6.17 mmol) and dihydropyrimidine-2,4 (1H,3H)-dione (1.41 g, 12.4 mmol) in 1,4-dioxane (60 mL) at r.t. under N₂. The resulting mixture was stirred at 100° C. for 16 h. The solvent was then removed under reduced pressure. Purification by FSC (gradient: 0-9% MeOH in DCM) gave the title compound (1.20 g, 54%) as a yellow solid. ¹H NMR: δ 1.32 (9H, s), 2.72 (4H, t), 3.79 (2H, t), 4.34 (2H, t), 6.41 (1H, d), 6.96 (1H, dd), 7.35 (1H, d), 7.45 (1H, s), 7.49 (1H, d), 10.29 (1H, s). m/z (ES⁺), [M+Na]⁺=380.1.

### Intermediate 69c: 3-(6-(2,4-Dioxotetrahydropyrimidin-1 (2H)-yl)-1H-indol-1-yl)propanoic acid

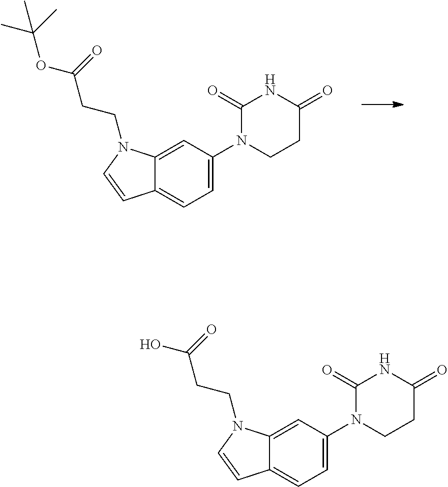 [SMILES: CC(C)(C)OC(=O)CCn1ccc2ccc(N3CCC(=O)NC3=O)cc21 ; O=C(O)CCn1ccc2ccc(N3CCC(=O)NC3=O)cc21]

tert-Butyl 3-(6-(2,4-dioxotetrahydropyrimidin-1 (2H)-yl)-1H-indol-1-yl)propanoate (1.20 g, 3.36 mmol) was added to formic acid (10 mL) at r.t. under air. The resulting solution was stirred at r.t. for 2 h. The solvent was removed under reduced pressure. Purification by C-18FC (gradient: 0-15% MeCN in water (containing 0.5% conc. HCl)) gave the title compound (620 mg, 61%) as a white solid. ¹H NMR: δ 2.64-2.79 (4H, m), 3.80-3.82 (2H, m), 4.36-4.38 (2H, m), 6.40 (1H, d), 6.97-6.98 (1H, m), 7.37 (1H, d), 7.43-7.54 (2H, m), 10.29 (1H, s). m/z (ES⁺), [M+H]⁺=302.1.

### Example 69: 3-(6-(2,4-Dioxotetrahydropyrimidin-1 (2H)-yl)-1H-indol-1-yl)-N-methylpropanamide

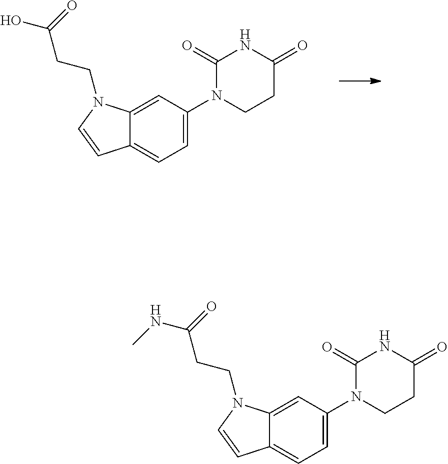 [SMILES: O=C(O)CCn1ccc2ccc(N3CCC(=O)NC3=O)cc21 ; CNC(=O)CCn1ccc2ccc(N3CCC(=O)NC3=O)cc21]

PyBOP (337 mg, 0.648 mmol) was added to a solution of 3-(6-(2,4-dioxotetrahydropyrimidin-1 (2H)-yl)-1H-indol-1-yl)propanoic acid (130 mg, 0.431 mmol), DIEA (113 μL, 0.647 mmol) and methylamine (2M solution in THF, 1.08 mL, 2.16 mmol) in DCM (3 mL) at r.t. The resulting solution was stirred at r.t. for 3 h. The solvent was then removed under reduced pressure. Purification C-18FC (gradient: 0-18% MeCN in water) gave the title compound (50.2 mg, 37%) as a pink solid. ¹H NMR: δ 2.50-2.60 (5H, m), 2.74 (2H, t), 3.80 (2H, t), 4.36 (2H, t), 6.41 (1H, d), 6.97 (1H, dd), 7.32 (1H, d), 7.45 (1H, s), 7.50 (1H, d), 7.85 (1H, q), 10.32 (1H, s). m/z (ES⁺), [M+H]⁺=315.2.

### Intermediate 70a: tert-Butyl 2-(4-bromo-1H-indol-1-yl)acetate

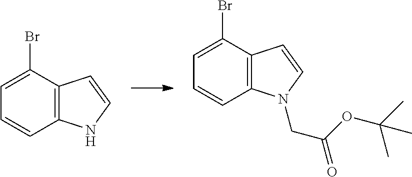 [SMILES: Brc1cccc2[nH]ccc12 ; CC(C)(C)OC(=O)Cn1ccc2c(Br)cccc21]

K₂CO₃ (4.23 g, 30.6 mmol) was added to a solution of 4-bromo-1H-indole (2.00 g, 10.2 mmol) and tert-butyl 2-bromoacetate (2.98 g, 15.3 mmol) in DMF (20 mL) at r.t. under air. The resulting mixture was stirred at 80° C. for 2 h. The reaction mixture was poured into water (50 mL), extracted with EtOAc (3×50 mL) then the combined organic extracts were dried (Na₂SO₄) and concentrated to give a pale yellow oil. Purification by FSC (gradient: 0-20% EtOAc in petroleum ether) gave the title compound (2.80 g, 88%) as a colourless oil. ¹H NMR: δ 1.42 (9H, s), 5.06 (2H, s), 6.43 (1H, dd), 7.08 (1H, t), 7.27 (1H, dd), 7.42 (1H, dt), 7.47 (1H, d). m/z (ES⁺), [M+H]⁺=310.1.

### Intermediate 70b: tert-Butyl 2-(4-(2,4-dioxotetrahydropyrimidin-1 (2H)-yl)-1H-indol-1-yl)acetate

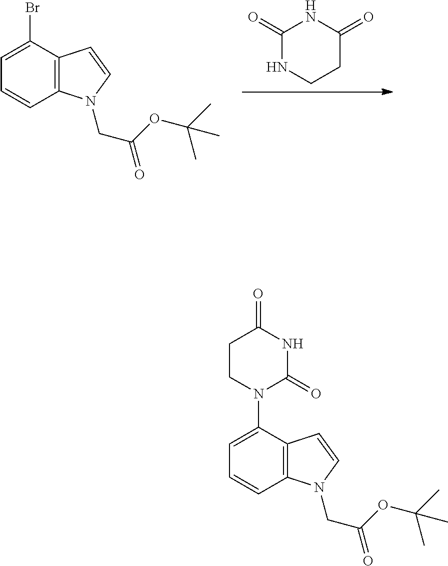 [SMILES: CC(C)(C)OC(=O)Cn1ccc2c(Br)cccc21 ; O=C1CCNC(=O)N1 ; CC(C)(C)OC(=O)Cn1ccc2c(N3CCC(=O)NC3=O)cccc21]

Ephos (86.0 mg, 0.161 mmol) was added to a degassed mixture of Ephos Pd G4 (148 mg, 0.161 mmol), Cs₂CO₃ (3.15 g, 9.67 mmol), dihydropyrimidine-2,4 (1H,3H)-dione (1.10 g, 9.64 mmol) and tert-butyl 2-(4-bromo-1H-indol-1-yl)acetate (1.00 g, 3.22 mmol) in 1,4-dioxane (50 mL) at r.t. under N₂. The resulting mixture was stirred at 90° C. for 17 h. The solvent was then removed under reduced pressure. Purification by C-18FC (gradient: 0-60% MeCN in water (containing 0.1% conc. HCl)) gave the title compound (400 mg, 36%) as a pale yellow solid. ¹H NMR: δ 1.42 (9H, s), 2.77 (2H, t), 3.78 (2H, t), 5.02 (2H, s), 6.42 (1H, dd), 6.98 (1H, dd), 7.10-7.22 (1H, m), 7.30 (1H, dt), 7.35 (1H, d), 10.35 (1H, s). m/z (ES⁺), [M-tBu+2H]⁺=288.1.

### Intermediate 70c: 2-(4-(2,4-Dioxotetrahydropyrimidin-1 (2H)-yl)-1H-indol-1-yl)acetic acid

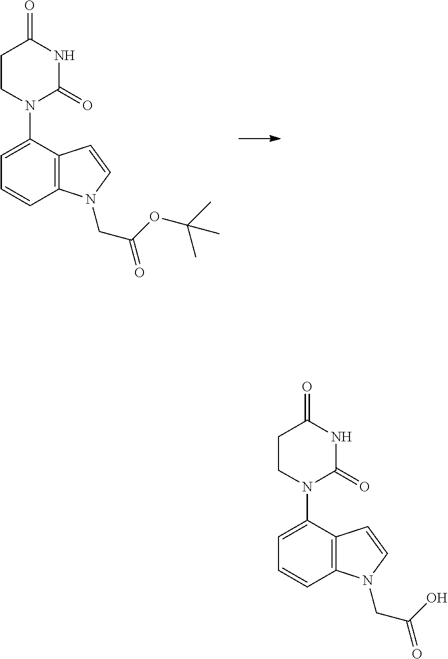 [SMILES: CC(C)(C)OC(=O)Cn1ccc2c(N3CCC(=O)NC3=O)cccc21 ; O=C(O)Cn1ccc2c(N3CCC(=O)NC3=O)cccc21]

TFA (5.00 mL, 64.9 mmol) was added to a solution of tert-butyl 2-(4-(2,4-dioxotetrahydropyrimidin-1 (2H)-yl)-1H-indol-1-yl)acetate (350 mg, 1.02 mmol) in DCM (5 mL) at r.t. under air. The resulting solution was stirred at r.t. for 6 h. The solvent was removed under reduced pressure. Purification by C-18FC (gradient: 0-30% MeCN in water (containing 0.1% conc. HCl)) gave the title compound (170 mg, 58%) as a pale yellow solid. ¹H NMR: δ 2.77 (2H, t), 3.79 (2H, t), 5.04 (2H, s), 6.42 (1H, dd), 6.98 (1H, dd), 7.14 (1H, t), 7.31-7.38 (2H, m), 10.34 (1H, s), 12.97 (1H, s). m/z (ES⁺), [M+H]⁺=288.2.

### Example 70: 2-(4-(2,4-Dioxotetrahydropyrimidin-1 (2H)-yl)-1H-indol-1-yl)-N-methylacetamide

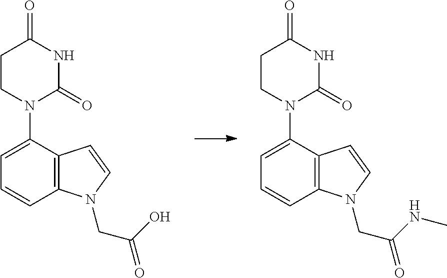 [SMILES: O=C(O)Cn1ccc2c(N3CCC(=O)NC3=O)cccc21 ; CNC(=O)Cn1ccc2c(N3CCC(=O)NC3=O)cccc21]

DIEA (73.0 μL, 0.418 mmol) was added to a mixture of PyBOP (145 mg, 0.279 mmol), methylamine (4M solution in THF, 70.0 μL, 0.280 mmol) and 2-(4-(2,4-dioxotetrahydropyrimidin-1 (2H)-yl)-1H-indol-1-yl)acetic acid (40.0 mg, 0.139 mmol) in DMF (1 mL) at r.t. under air. The resulting solution was stirred at r.t. for 2 h and then purified directly by C-18FC (gradient: 0-20% MeCN in water (containing 0.1% conc. HCl)) gave the title compound (35.0 mg, 84%) as a pale yellow solid. ¹H NMR: δ 2.62 (3H, d), 2.77 (2H, t), 3.78 (2H, t), 4.81 (2H, s), 6.41 (1H, dd), 6.97 (1H, dd), 7.14 (1H, t), 7.28-7.37 (2H, m), 8.12 (1H, d), 10.34 (1H, s). m/z (ES⁺), [M+H]⁺=301.2

### Intermediate 71a: 1-Acetyl-6-bromo-1H-indol-3-yl acetate

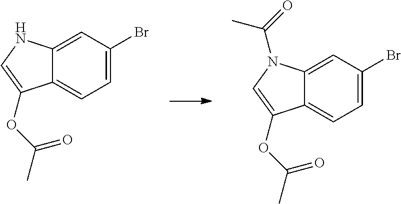 [SMILES: CC(=O)Oc1c[nH]c2cc(Br)ccc12 ; CC(=O)Oc1cn(C(C)=O)c2cc(Br)ccc12]

DMAP (385 mg, 3.15 mmol) was added to a mixture of 6-bromo-1H-indol-3-yl acetate (4.00 g, 15.7 mmol), Ac₂O (14.9 mL, 158 mmol) and triethylamine (4.39 mL, 31.5 mmol) in THF (60 mL) at r.t. The resulting mixture was stirred at 80° C. for 2 h. The solvent was removed under reduced pressure. Purification by FSC (gradient: 0-20% EtOAc in petroleum ether) gave the title compound (2.34 g, 50%) as a purple solid. ¹H NMR: δ 2.36 (3H, s), 2.60 (3H, s), 7.48 (2H, t), 7.92 (1H, s), 8.48-8.55 (1H, m). m/z (ES⁺), [M+H]⁺=296.0.

### Intermediate 71b: tert-Butyl 4-(6-bromo-1H-indol-3-yl)piperazine-1-carboxylate

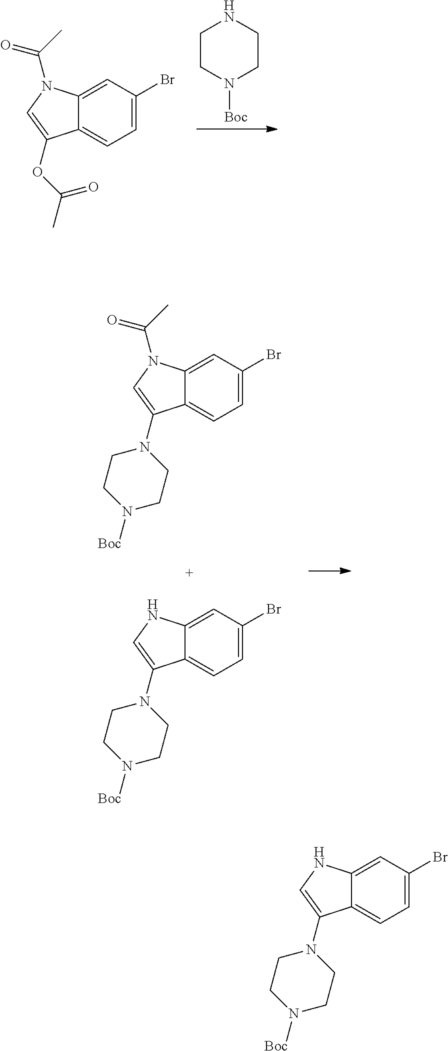

4-Methylbenzenesulfonic acid (256 mg, 1.49 mmol) was added to a mixture of 1-acetyl-6-bromo-1H-indol-3-yl acetate (2.20 g, 7.43 mmol) and tert-butyl piperazine-1-carboxylate (6.92 g, 37.2 mmol) in toluene (50 mL) at r.t. The resulting mixture was stirred at 120° C. for 24 h. The solvent was then removed under reduced pressure. Purification by FSC (gradient: 0-30% EtOAc in petroleum ether) gave a 2:3 mixture of tert-butyl 4-(1-acetyl-6-bromo-1H-indol-3-yl)piperazine-1-carboxylate and tert-butyl 4-(6-bromo-1H-indol-3-yl)piperazine-1-carboxylate (1.09 g) as a purple solid which was used directly in the next step without any further purification. m/z (ES⁺), [M+H]⁺=422.1 & 380.1. The product mixture was then dissolved in MeOH (40 mL) and to this solution was added triethylamine (1.80 mL, 12.9 mmol) at r.t. The resulting mixture was stirred at 60° C. for 2 h. The solvent was removed under reduced pressure to give the title compound (1.00 g, 35% over two steps) as a purple solid. ¹H NMR: δ 1.43 (9H, s), 2.89 (4H, t), 3.51 (4H, t), 6.92 (1H, d), 7.07 (1H, dd), 7.48 (2H, dd), 10.74 (1H, s). m/z (ES⁺), [M+H]⁺=380.1.

### Intermediate 71c: tert-Butyl 6-bromo-3-(4-(tert-butoxycarbonyl)piperazin-1-yl)-1H-indole-1-carboxylate

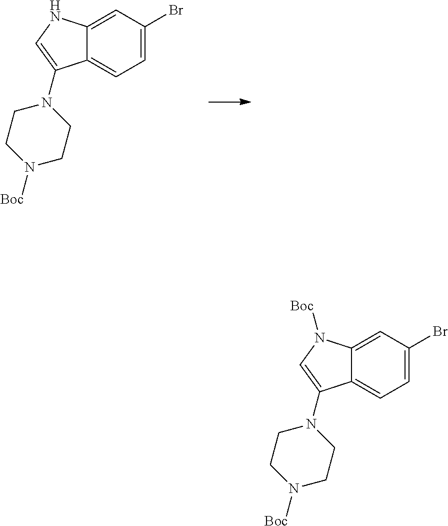 [SMILES: CC(C)(C)OC(=O)N1CCN(c2c[nH]c3cc(Br)ccc23)CC1 ; CC(C)(C)OC(=O)N1CCN(c2cn(C(=O)OC(C)(C)C)c3cc(Br)ccc23)CC1]

Di-tert-butyl dicarbonate (1.22 mL, 5.25 mmol) was added to a mixture of tert-butyl 4-(6-bromo-1H-indol-3-yl)piperazine-1-carboxylate (1.00 g, 2.63 mmol), triethylamine (1.10 mL, 7.89 mmol) and DMAP (32.0 mg, 0.262 mmol) in DCM (30 mL) at r.t. The resulting mixture was stirred at r.t. for 1 h. The solvent was removed under reduced pressure. Purification by FSC (gradient: 0-15% EtOAc in petroleum ether) gave the title compound (1.20 g, 95%) as a pale yellow solid. ¹H NMR: δ 1.42 (9H, s), 1.61 (9H, s), 2.95 (4H, t), 3.51 (4H, t), 7.07 (1H, s), 7.40 (1H, dd), 7.62 (1H, d), 8.24 (1H, s). m/z (ES⁺), [M+H]⁺=482.2.

### Intermediate 71d: tert-Butyl 3-(4-(tert-butoxycarbonyl)piperazin-1-yl)-6-(2,4-dioxotetrahydropyrimidin-1 (2H)-yl)-1H-indole-1-carboxylate

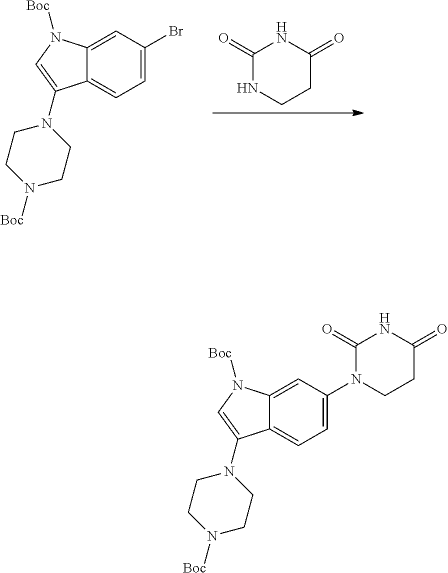 [SMILES: CC(C)(C)OC(=O)N1CCN(c2cn(C(=O)OC(C)(C)C)c3cc(Br)ccc23)CC1 ; O=C1CCNC(=O)N1 ; CC(C)(C)OC(=O)N1CCN(c2cn(C(=O)OC(C)(C)C)c3cc(N4CCC(=O)NC4=O)ccc23)CC1]

Ephos Pd G4 (115 mg, 0.125 mmol) was added to a degassed mixture of tert-butyl 6-bromo-3-(4-(tert-butoxycarbonyl)piperazin-1-yl)-1H-indole-1-carboxylate (1.20 g, 2.50 mmol), dihydropyrimidine-2,4 (1H,3H)-dione (1.14 g, 9.99 mmol), Cs₂CO₃ (1.63 g, 5.00 mmol) and Ephos (67.0 mg, 0.125 mmol) in 1,4-dioxane (30 mL) at r.t. under argon. The resulting mixture was stirred at 100° C. for 16 h. The solvent was removed under reduced pressure. Purification by FSC (gradient: 0-70% EtOAc in petroleum ether) gave the title compound (1.00 g, 78%) as an off-white solid. ¹H NMR: δ 1.43 (9H, s), 1.61 (9H, s), 2.73 (2H, t), 2.94-3.01 (4H, m), 3.49-3.55 (4H, m), 3.83 (2H, t), 7.07 (1H, s), 7.21 (1H, dd), 7.64 (1H, d), 8.05 (1H, s), 10.37 (1H, s). m/z (ES⁺), [M+H]⁺=514.3.

### Example 71: 1-(3-(Piperazin-1-yl)-1H-indol-6-yl)dihydropyrimidine-2,4 (1H,3H)-dione

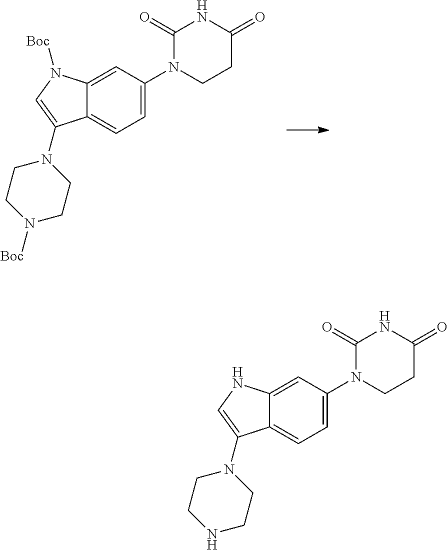 [SMILES: CC(C)(C)OC(=O)N1CCN(c2cn(C(=O)OC(C)(C)C)c3cc(N4CCC(=O)NC4=O)ccc23)CC1 ; O=C1CCN(c2ccc3c(N4CCNCC4)c[nH]c3c2)C(=O)N1]

tert-Butyldimethylsilyl trifluoromethanesulfonate (1.47 g, 5.56 mmol) was added to a mixture of tert-butyl 3-(4-(tert-butoxycarbonyl)piperazin-1-yl)-6-(2,4-dioxotetrahydropyrimidin-1 (2H)-yl)-1H-indole-1-carboxylate (950 mg, 1.85 mmol) in DCM (30 mL) at r.t. The resulting mixture was stirred at r.t. for 16 h. A second portion of tert-butyldimethylsilyl trifluoromethanesulfonate (1.47 g, 5.56 mmol) was added and the reaction was stirred for a further 5 h. The solvent was removed under reduced pressure. Purification by C-18FC (gradient: 0-20% MeCN in water (10 mmol NH₄HCO₃)) gave the title compound (490 mg, 85%) as a pale yellow solid. ¹H NMR: δ 2.72 (2H, t), 2.90-3.01 (8H, m), 3.78 (2H, t), 6.85-6.92 (2H, m), 7.22 (1H, d), 7.49 (1H, d), 10.28 (1H, s), 10.60 (1H, d). m/z (ES⁺), [M+H]⁺=314.1.

### Example 72: 1-(3-(4-Methylpiperazin-1-yl)-1H-indol-6-yl)dihydropyrimidine-2,4 (1H,3H)-dione

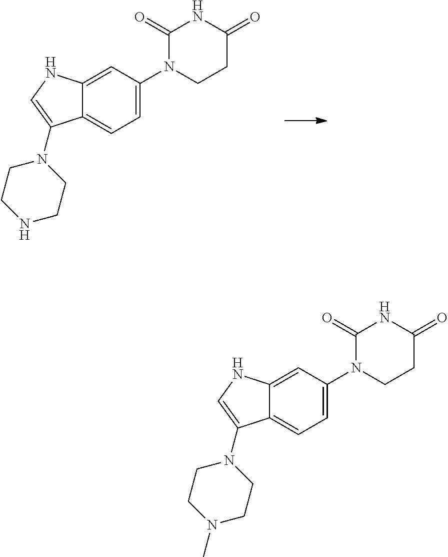 [SMILES: O=C1CCN(c2ccc3c(N4CCNCC4)c[nH]c3c2)C(=O)N1 ; CN1CCN(c2c[nH]c3cc(N4CCC(=O)NC4=O)ccc23)CC1]

Sodium triacetoxyborohydride (81.0 mg, 0.382 mmol) was added to a mixture of 1-(3-(piperazin-1-yl)-1H-indol-6-yl)dihydropyrimidine-2,4 (1H,3H)-dione (60.0 mg, 0.191 mmol), formaldehyde (16.0 μL, 0.581 mmol) and AcOH (22.0 μL, 0.384 mmol) in DCM (10 mL) at r.t. The resulting mixture was stirred at r.t. for 16 h. The solvent was removed under reduced pressure. Purification by preparative HPLC (Column Y, Eluent B, gradient: 15-20%) gave the title compound (18.0 mg, 29%) as a white solid. ¹H NMR: δ 2.22 (3H, s), 2.70 (2H, t), 2.91-2.97 (4H, m), 3.28-3.30 (4H, m), 3.76 (2H, t), 6.86 (2H, dd), 7.20 (1H, d), 7.45 (1H, d), 10.26 (1H, s), 10.57 (1H, s). m/z (ES⁺), [M+H]⁺=328.1.

### Example 73: 1-(3-(4-Acetylpiperazin-1-yl)-1H-indol-6-yl)dihydropyrimidine-2,4 (1H,3H)-dione

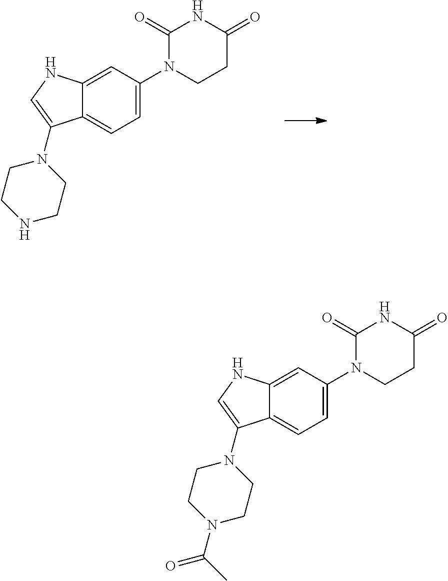 [SMILES: O=C1CCN(c2ccc3c(N4CCNCC4)c[nH]c3c2)C(=O)N1 ; CC(=O)N1CCN(c2c[nH]c3cc(N4CCC(=O)NC4=O)ccc23)CC1]

Ac₂O (30.0 μL, 0.318 mmol) was added to a mixture of 1-(3-(piperazin-1-yl)-1H-indol-6-yl)dihydropyrimidine-2,4 (1H,3H)-dione (50.0 mg, 0.160 mmol) and DIEA (84.0 μL, 0.481 mmol) in DCM (10 mL) at r.t. The resulting mixture was stirred at r.t. for 1 h. The solvent was removed under reduced pressure. Purification by C-18FC (gradient: 0-25% MeCN in water (10 mmol NH₄HCO₃)) gave the title compound (30.0 mg, 53%) as a white solid. ¹H NMR: δ 2.04 (3H, s), 2.72 (2H, t), 2.93 (4H, dt), 3.58-3.69 (4H, m), 3.78 (2H, t), 6.86-6.95 (2H, m), 7.24 (1H, d), 7.52 (1H, d), 10.28 (1H, s), 10.64 (1H, d). m/z (ES⁺), [M+H]⁺=356.3.

### Intermediate 74a: tert-Butyl 4-(6-bromo-1-methyl-1H-indol-3-yl)piperazine-1-carboxylate

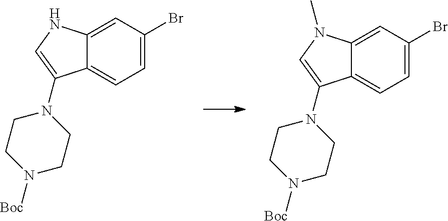 [SMILES: CC(C)(C)OC(=O)N1CCN(c2c[nH]c3cc(Br)ccc23)CC1 ; Cn1cc(N2CCN(C(=O)OC(C)(C)C)CC2)c2ccc(Br)cc21]

NaH (60% dispersion in mineral oil, 205 mg, 5.13 mmol) was added to a solution of tert-butyl 4-(6-bromo-1H-indol-3-yl)piperazine-1-carboxylate (1.30 g, 3.42 mmol) in DMF (20 mL) at 0° C. under N₂. The resulting mixture was stirred at 0° C. for 0.5 h before the addition of Mel (0.485 g, 3.42 mmol). The resulting mixture was stirred for 1 h at r.t. The reaction mixture was quenched with saturated NH₄Cl (50 mL) and extracted with EtOAc (3×100 mL). The combined organic solutions were washed with saturated brine (3×100 mL), dried (Na₂SO₄) and concentrated to give a dark gum. Purification by FSC (gradient: 0-30% EtOAc in petroleum ether) gave the title compound (1.02 g, 76%) as a purple gum. ¹H NMR: δ 1.41 (9H, s), 2.86 (4H, t), 3.49 (4H, t), 3.66 (3H, s), 6.88 (1H, s), 7.07 (1H, dd), 7.47 (1H, d), 7.61 (1H, d). m/z (ES⁺), [M+H]⁺=396.1.

### Intermediate 74b: tert-Butyl 4-(6-(2,4-dioxotetrahydropyrimidin-1 (2H)-yl)-1-methyl-1H-indol-3-yl)piperazine-1-carboxylate

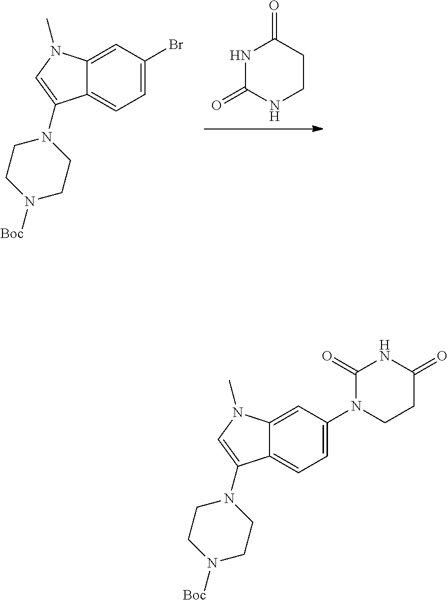 [SMILES: Cn1cc(N2CCN(C(=O)OC(C)(C)C)CC2)c2ccc(Br)cc21 ; O=C1CCNC(=O)N1 ; Cn1cc(N2CCN(C(=O)OC(C)(C)C)CC2)c2ccc(N3CCC(=O)NC3=O)cc21]

Cs₂CO₃ (1.653 g, 5.06 mmol) was added to a degassed mixture of tert-butyl 4-(6-bromo-1-methyl-1H-indol-3-yl)piperazine-1-carboxylate (1.00 g, 2.54 mmol), dihydropyrimidine-2,4 (1H,3H)-dione (1.16 g, 10.2 mmol), Ephos (68.0 mg, 0.127 mmol) and Ephos Pd G4 (116 mg, 0.126 mmol) in 1,4-dioxane (30 mL) at r.t. under N₂. The resulting mixture was stirred at 100° C. for 16 h. The solvent was removed under reduced pressure. Purification by FSC (gradient: 0-4% MeOH in DCM) gave the title compound (1.00 g, 92%) as a brown solid. ¹H NMR: δ 1.41 (9H, s), 2.71 (2H, t), 2.83-2.93 (4H, m), 3.46-3.55 (4H, m), 3.66 (3H, s), 3.78 (2H, t), 6.81-6.98 (2H, m), 7.31 (1H, d), 7.50 (1H, d), 10.27 (1H, s). m/z (ES⁺), [M+H]⁺=428.2.

### Example 74: 1-(1-Methyl-3-(piperazin-1-yl)-1H-indol-6-yl)dihydropyrimidine-2,4 (1H,3H)-dione

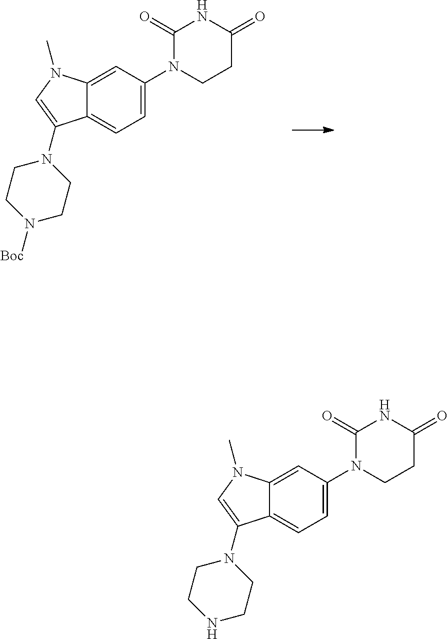 [SMILES: Cn1cc(N2CCN(C(=O)OC(C)(C)C)CC2)c2ccc(N3CCC(=O)NC3=O)cc21 ; Cn1cc(N2CCNCC2)c2ccc(N3CCC(=O)NC3=O)cc21]

tert-Butyldimethylsilyltrifluoromethanesulfonate (835 mg, 3.16 mmol) was added to a solution of tert-butyl 4-(6-(2,4-dioxotetrahydropyrimidin-1 (2H)-yl)-1-methyl-1H-indol-3-yl)piperazine-1-carboxylate (900 mg, 2.11 mmol) in MeCN (20 mL) at 0° C. The resulting mixture was stirred at r.t. for 1 h. The solvent was removed under reduced pressure. Purification by C-18FC (gradient: 0-25% MeCN in water (10 mmol NH₄HCO₃)) gave the title compound (570 mg, 83%) as a light brown solid. ¹H NMR: δ 2.74 (2H, t), 2.84-2.96 (8H, m), 3.68 (3H, s), 3.80 (2H, t), 6.85 (1H, s), 6.92 (1H, dd), 7.32 (1H, d), 7.50 (1H, d), 10.29 (1H, s). m/z (ES⁺), [M+H]⁺=328.2.

### Example 75: 1-(1-Methyl-3-(4-methylpiperazin-1-yl)-1H-indol-6-yl)dihydropyrimidine-2,4 (1H,3H)-dione

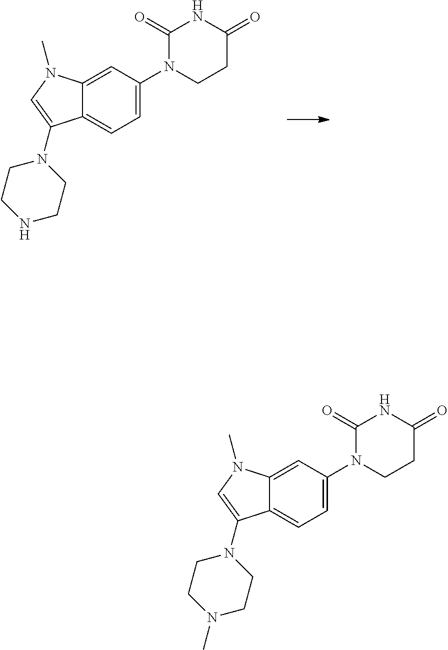 [SMILES: Cn1cc(N2CCNCC2)c2ccc(N3CCC(=O)NC3=O)cc21 ; CN1CCN(c2cn(C)c3cc(N4CCC(=O)NC4=O)ccc23)CC1]

Sodium triacetoxyborohydride (181 mg, 0.854 mmol) was added to a mixture of 1-(1-methyl-3-(piperazin-1-yl)-1H-indol-6-yl)dihydropyrimidine-2,4 (1H,3H)-dione (70.0 mg, 0.214 mmol), paraformaldehyde (19.3 mg, 0.643 mmol) and AcOH (36.7 μL, 0.641 mmol) in DCM (5 mL) at r.t. The resulting mixture was stirred at r.t. for 4 h. The solvent was removed under reduced pressure. Purification by C-18FC (gradient: 0-25% MeCN in water (10 mmol NH₄HCO₃)) gave material that was further purified by preparative HPLC (Column A, Eluent F, gradient: 13-25%) to give the title compound (20.0 mg, 27%) as a white solid. ¹H NMR: δ 2.25 (3H, s), 2.48-2.58 (4H, m), 2.73 (2H, t), 2.96 (4H, br s), 3.68 (3H, s), 3.80 (2H, t), 6.86 (1H, s), 6.92 (1H, dd), 7.33 (1H, d), 7.50 (1H, d), 10.31 (1H, s). m/z (ES⁺), [M+H]⁺=342.1.

### Intermediate 76a: 1-Acetyl-5-bromo-1H-indol-3-yl acetate

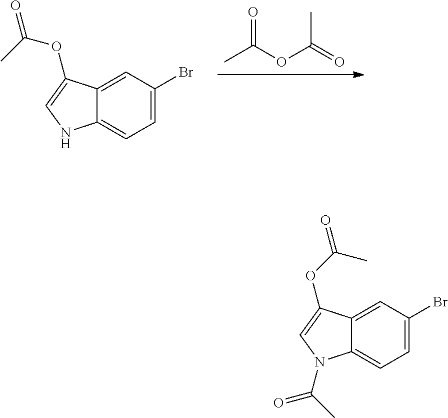

DMAP (481 mg, 3.94 mmol) was added to a solution of 5-bromo-1H-indol-3-yl acetate (5.00 g, 19.7 mmol), triethylamine (27.4 mL, 197 mmol) and Ac₂O (18.6 mL, 197 mmol) in THF (50 mL) at r.t. under air. The resulting solution was stirred at 80° C. for 2 h. The solvent was removed under reduced pressure. Purification by FSC (gradient: 0-30% EtOAc in petroleum ether) gave the title compound (5.20 g, 89%) as a white solid. ¹H NMR: δ 2.36 (3H, s), 2.60 (3H, s), 7.51 (1H, dd), 7.75 (1H, dd), 7.93 (1H, s), 8.27 (1H, dd). m/z (ES⁺), [M+H]⁺=295.9.

### Intermediate 76b: tert-Butyl 4-(5-bromo-1H-indol-3-yl)piperazine-1-carboxylate

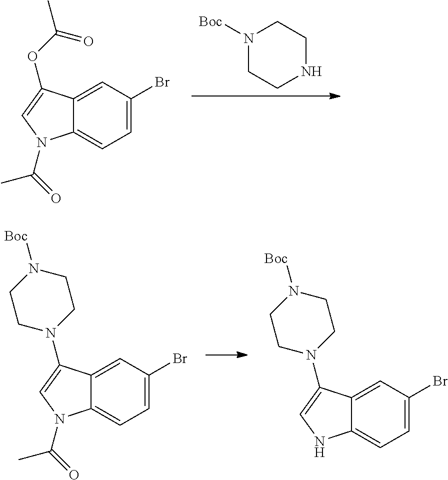 [SMILES: CC(=O)Oc1cn(C(C)=O)c2ccc(Br)cc12 ; CC(C)(C)OC(=O)N1CCNCC1 ; CC(=O)n1cc(N2CCN(C(=O)OC(C)(C)C)CC2)c2cc(Br)ccc21 ; CC(C)(C)OC(=O)N1CCN(c2c[nH]c3ccc(Br)cc23)CC1]

4-Methylbenzenesulfonic acid (593 mg, 3.44 mmol) was added to a mixture of 1-acetyl-5-bromo-1H-indol-3-yl acetate (5.10 g, 17.2 mmol) and tert-butyl piperazine-1-carboxylate (16.0 g, 85.9 mmol) in toluene (60 mL) at r.t. The resulting mixture was stirred at 120° C. for 16 h. The solvent was removed under reduced pressure. Purification by FSC (gradient: 0-50% EtOAc in petroleum ether) gave a 4:6 mixture of tert-butyl 4-(1-acetyl-5-bromo-1H-indol-3-yl)piperazine-1-carboxylate and tert-butyl 4-(5-bromo-1H-indol-3-yl)piperazine-1-carboxylate respectively (4.30 g) as a black solid which was carried over to the next step without any further purification. The mixture was dissolved in MeOH (40 mL) and triethylamine (7.10 ml, 50.9 mmol) was added to the solution at r.t. The resulting mixture was stirred at 60° C. for 2 h. The solvent was removed under reduced pressure. Purification by FSC (gradient: 0-100% EtOAc in petroleum ether) gave the title compound (3.50 g, 53% over two steps) as a purple solid. ¹H NMR: δ 1.42 (9H, s), 2.84-2.91 (4H, m), 3.47-3.54 (4H, m), 6.96 (1H, d), 7.16 (1H, dd), 7.28 (1H, d), 7.67 (1H, d), 10.80 (1H, d). m/z (ES⁺), [M+H]⁺=380.1.

### Intermediate 76c: tert-Butyl 5-bromo-3-(4-(tert-butoxycarbonyl)piperazin-1-yl)-1H-indole-1-carboxylate

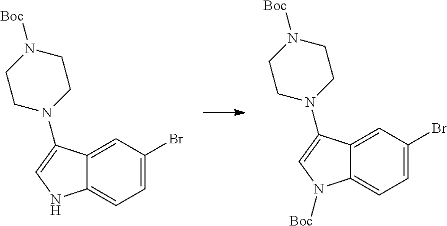 [SMILES: CC(C)(C)OC(=O)N1CCN(c2c[nH]c3ccc(Br)cc23)CC1 ; CC(C)(C)OC(=O)N1CCN(c2cn(C(=O)OC(C)(C)C)c3ccc(Br)cc23)CC1]

Triethylamine (3.85 mL, 27.6 mmol) was added to a mixture of tert-butyl 4-(5-bromo-1H-indol-3-yl)piperazine-1-carboxylate (3.50 g, 9.20 mmol), di-tert-butyl dicarbonate (3.21 mL, 13.8 mmol) and DMAP (0.112 g, 0.92 mmol) in DCM (30 mL) at r.t. under argon. The resulting mixture was stirred at r.t. for 16 h. The solvent was removed under reduced pressure. Purification by FSC (gradient: 0-50% EtOAc in petroleum ether) gave the title compound (3.50 g, 79%) as a yellow solid. ¹H NMR: δ 1.43 (9H, s), 1.61 (9H, s), 2.90-3.00 (4H, m), 3.48-3.57 (4H, m), 7.12 (1H, s), 7.49 (1H, dd), 7.83 (1H, d), 8.01 (1H, d). m/z (ES⁺), [M+H]⁺=482.1.

### Intermediate 76d: tert-Butyl 4-(5-bromo-1-methyl-1H-indol-3-yl)piperazine-1-carboxylate

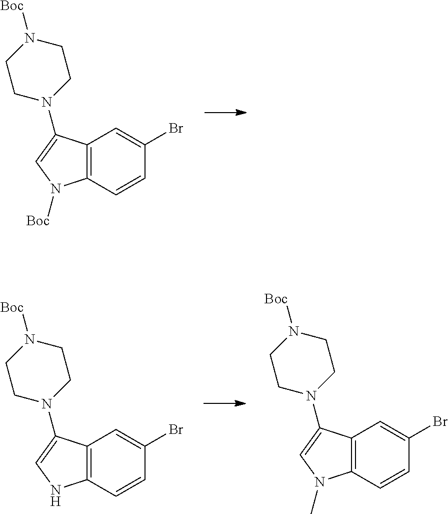 [SMILES: CC(C)(C)OC(=O)N1CCN(c2cn(C(=O)OC(C)(C)C)c3ccc(Br)cc23)CC1 ; CC(C)(C)OC(=O)N1CCN(c2c[nH]c3ccc(Br)cc23)CC1 ; Cn1cc(N2CCN(C(=O)OC(C)(C)C)CC2)c2cc(Br)ccc21]

tert-Butyl 5-bromo-3-(4-(tert-butoxycarbonyl)piperazin-1-yl)-1H-indole-1-carboxylate (900 mg, 1.87 mmol) were dissolved in 2,2,2-trifluoroethanol (8 mL) and sealed into a microwave tube. The reaction was heated to 120° C. for 1 h in a microwave reactor. The reaction was cooled to r.t. and the solvent was removed under reduced pressure to give the title compound as a black solid (800 mg) which was used in the next step without further purification. m/z (ES⁺), [M+H]⁺=380.1. The solid was then dissolved in DMF (3 mL) and cooled at 0° C. under N₂ before the addition of NaH (60% dispersion in mineral oil, 126 mg, 3.16 mmol). The resulting mixture was then stirred at r.t. for 0.5 h before the addition of Mel (132 μL, 2.10 mmol). The resulting mixture was stirred at r.t. for 1 h. The reaction mixture was quenched with saturated NH₄C1 (10 mL) and extracted with EtOAc (3×50 mL). The combined organic extracts were dried (Na₂SO₄) and evaporated to give the title compound as a purple solid (800 mg, 108% over two-steps [85% purity based on LCMS]). ¹H NMR: δ 1.43 (9H, s), 2.86-2.91 (4H, m), 3.48-3.55 (4H, m), 3.70 (3H, s), 6.96 (1H, s), 7.24 (1H, dd), 7.37 (1H, d), 7.69 (1H, d). m/z (ES⁺), [M+H]⁺=396.1.

### Intermediate 76e: tert-Butyl 4-(5-(2,4-dioxotetrahydropyrimidin-1 (2H)-yl)-1-methyl-1H-indol-3-yl)piperazine-1-carboxylate

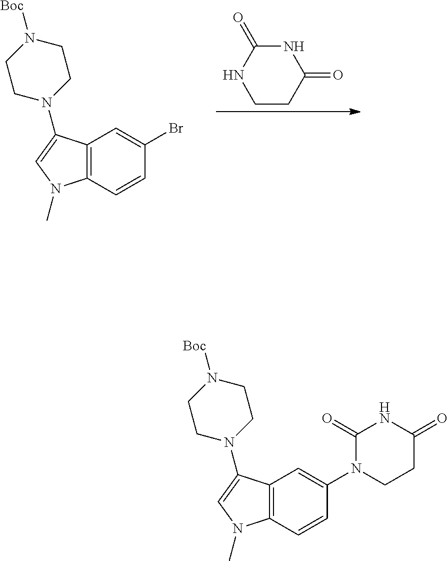 [SMILES: Cn1cc(N2CCN(C(=O)OC(C)(C)C)CC2)c2cc(Br)ccc21 ; O=C1CCNC(=O)N1 ; Cn1cc(N2CCN(C(=O)OC(C)(C)C)CC2)c2cc(N3CCC(=O)NC3=O)ccc21]

Ephos (24.4 mg, 0.0456 mmol) and Ephos Pd G4 (41.9 mg, 0.0456 mmol) were added to a degassed mixture of Cs₂CO₃ (595 mg, 1.83 mmol), tert-butyl 4-(5-bromo-1-methyl-1H-indol-3-yl)piperazine-1-carboxylate (360 mg, 0.913 mmol—assumed pure, however 85% purity later determined in the previous step based on LCMS) and dihydropyrimidine-2,4 (1H,3H)-dione (313 mg, 2.74 mmol) in DMF (20 mL) at r.t. under N₂. The resulting mixture was stirred at 100° C. for 16 h. The solvent was then removed under reduced pressure. Purification by C-18FC (gradient 5-50% MeCN in water (containing 0.1% FA)) gave the title compound in the form of a formate salt (90.0 mg, 21%) as a white solid. ¹H NMR: δ 1.43 (9H, s), 2.73 (2H, t), 2.85-2.93 (4H, m), 3.55-3.65 (4H, m), 3.70 (3H, s), 3.77 (2H, t), 6.93 (1H, s), 7.08 (1H, dd), 7.37 (1H, d), 7.47 (1H, d), 8.33 (1H, s), 10.26 (1H, s). m/z (ES⁺), [M+H]⁺=428.2.

### Example 76: 1-(1-Methyl-3-(piperazin-1-yl)-1H-indol-5-yl)dihydropyrimidine-2,4 (1H,3H)-dione

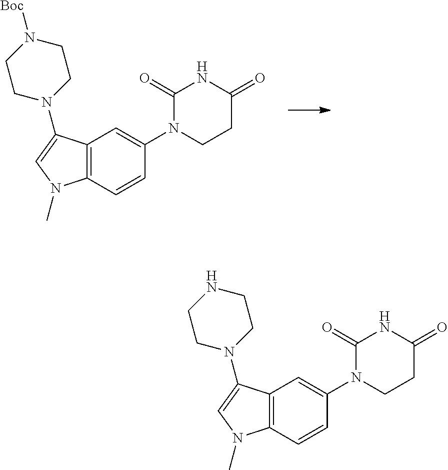 [SMILES: Cn1cc(N2CCN(C(=O)OC(C)(C)C)CC2)c2cc(N3CCC(=O)NC3=O)ccc21 ; Cn1cc(N2CCNCC2)c2cc(N3CCC(=O)NC3=O)ccc21]

A solution of HCl in 1,4-dioxane (4M, 845 μL, 3.38 mmol) was added to a solution of tert-butyl 4-(5-(2,4-dioxotetrahydropyrimidin-1 (2H)-yl)-1-methyl-1H-indol-3-yl)piperazine-1-carboxylate formate (80 mg, 0.169 mmol) in DCM (10 mL). The resulting mixture was stirred at r.t. for 2 h. The solvent was removed under reduced pressure. Purification by C-18FC (gradient: 5-15% MeCN in water (containing 0.05% conc. HCl)) gave the title compound in the form of a hydrochloride salt (40.0 mg, 65%) as an orange solid. ¹H NMR: δ 2.71 (2H, t), 3.12-3.18 (4H, m), 3.24-3.30 (4H, m), 3.70 (3H, s), 3.74 (2H, t), 7.03 (1H, s), 7.08 (1H, dd), 7.37 (1H, d), 7.49 (1H, d), 9.00 (2H, br s), 10.25 (1H, s). m/z (ES⁺), [M+H]⁺=328.2.

### Intermediate 77a: tert-Butyl 3-(4-(tert-butoxycarbonyl)piperazin-1-yl)-5-(2,4-dioxotetrahydropyrimidin-1 (2H)-yl)-1H-indole-1-carboxylate

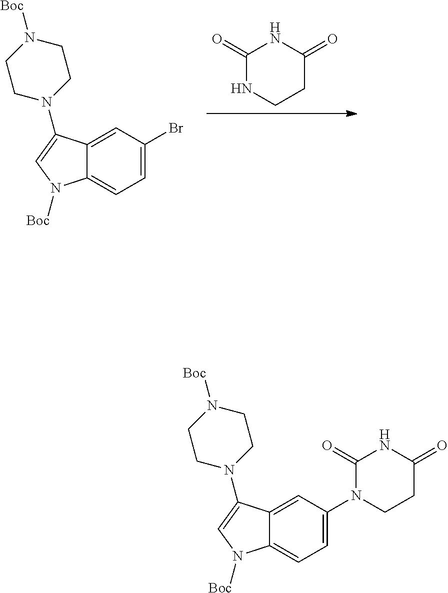 [SMILES: CC(C)(C)OC(=O)N1CCN(c2cn(C(=O)OC(C)(C)C)c3ccc(Br)cc23)CC1 ; O=C1CCNC(=O)N1 ; CC(C)(C)OC(=O)N1CCN(c2cn(C(=O)OC(C)(C)C)c3ccc(N4CCC(=O)NC4=O)cc23)CC1]

Ephos (83.0 mg, 0.155 mmol) and Ephos Pd G4 (143 mg, 0.156 mmol) were added to a degassed mixture of Cs₂CO₃ (3.05 g, 9.37 mmol), dihydropyrimidine-2,4 (1H,3H)-dione (1.07 g, 9.38 mmol) and tert-butyl 5-bromo-3-(4-(tert-butoxycarbonyl)piperazin-1-yl)-1H-indole-1-carboxylate (1.50 g, 3.12 mmol) in 1,4-dioxane (40 mL) at r.t. under N₂. The resulting mixture was stirred at 100° C. for 16 h. The solvent was then removed under reduced pressure. Purification by FSC (gradient: 0-80% EtOAc in petroleum ether) gave the title compound (1.00 g, 62%) as a white solid. ¹H NMR: δ 1.42 (9H, s), 1.62 (9H, s), 2.74 (2H, t), 2.94-2.98 (4H, m), 3.50-3.54 (4H, m), 3.81 (2H, t), 7.10 (1H, s), 7.29 (1H, dd), 7.59 (1H, d), 8.04 (1H, d), 10.36 (1H, s). m/z (ES⁺), [M+H]⁺=514.3.

### Example 77: 1-(3-(Piperazin-1-yl)-1H-indol-5-yl)dihydropyrimidine-2,4 (1H,3H)-dione

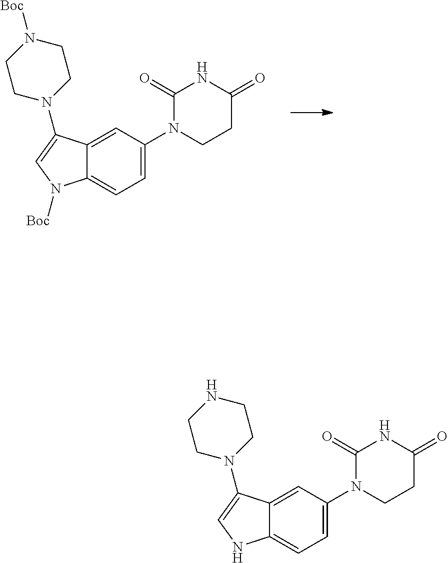 [SMILES: CC(C)(C)OC(=O)N1CCN(c2cn(C(=O)OC(C)(C)C)c3ccc(N4CCC(=O)NC4=O)cc23)CC1 ; O=C1CCN(c2ccc3[nH]cc(N4CCNCC4)c3c2)C(=O)N1]

tert-Butyl 3-(4-(tert-butoxycarbonyl)piperazin-1-yl)-5-(2,4-dioxotetrahydropyrimidin-1 (2H)-yl)-1H-indole-1-carboxylate (1.00 g, 1.95 mmol) was dissolved in 2,2,2-trifluoroethanol (20 mL) and sealed into a microwave tube. The reaction was heated to 150° C. for 6 h in a microwave reactor. The reaction was cooled to r.t. and the solvent was removed under reduced pressure. Purification by C-18FC (gradient: 3-40% MeCN in water (containing 0.1% NH₄HCO₃)) gave the title compound (420 mg, 69%) as a white solid. ¹H NMR: δ 2.72 (2H, t), 2.84-2.95 (8H, m), 3.75 (2H, t), 6.87 (1H, d), 6.98 (1H, dd), 7.28 (1H, d), 7.41 (1H, d), 10.23 (1H, s), 10.58 (1H, d). m/z (ES⁺), [M+H]⁺=314.2.

### Example 78: 1-(3-(4-Methylpiperazin-1-yl)-1H-indol-5-yl)dihydropyrimidine-2,4 (1H,3H)-dione

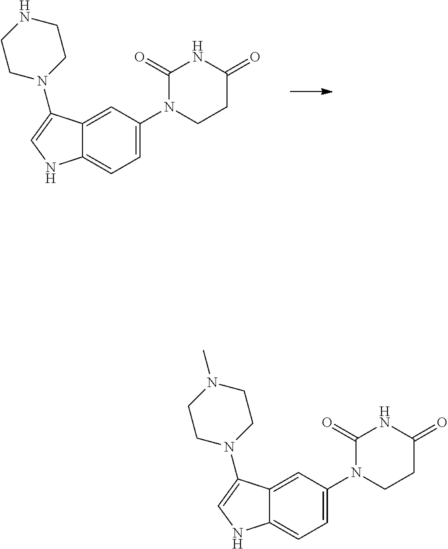 [SMILES: O=C1CCN(c2ccc3[nH]cc(N4CCNCC4)c3c2)C(=O)N1 ; CN1CCN(c2c[nH]c3ccc(N4CCC(=O)NC4=O)cc23)CC1]

Sodium triacetoxyborohydride (169 mg, 0.797 mmol) was added to a mixture of 1-(3-(piperazin-1-yl)-1H-indol-5-yl)dihydropyrimidine-2,4 (1H,3H)-dione (50.0 mg, 0.160 mmol), paraformaldehyde (9.6 mg, 0.32 mmol) in DCM (3 mL) at r.t. The resulting mixture was stirred at r.t. for 16 h. The reaction mixture was poured into water (10 mL), extracted with EtOAc (3×10 mL). The combined organic extracts were dried (Na₂SO₄) and concentrated to give a brown solid. Purification by C-18FC (gradient: 5-20% MeCN in water (containing 0.1% FA)) gave material that was further purified by preparative HPLC (Column C, Eluent B, gradient: 3-10%) to give the title compound (10.3 mg, 20%) as a white solid. ¹H NMR: δ 2.24 (3H, s), 2.72 (2H, t), 2.88-3.03 (4H, m), 3.27-3.32 (4H, m), 3.75 (2H, t), 6.88 (1H, d), 6.99 (1H, dd), 7.28 (1H, d), 7.41 (1H, d), 10.55-10.65 (1H, m). m/z (ES⁺), [M+H]⁺=328.2.

### Example 79: 1-(3-(4-Acetylpiperazin-1-yl)-1H-indol-5-yl)dihydropyrimidine-2,4 (1H,3H)-dione

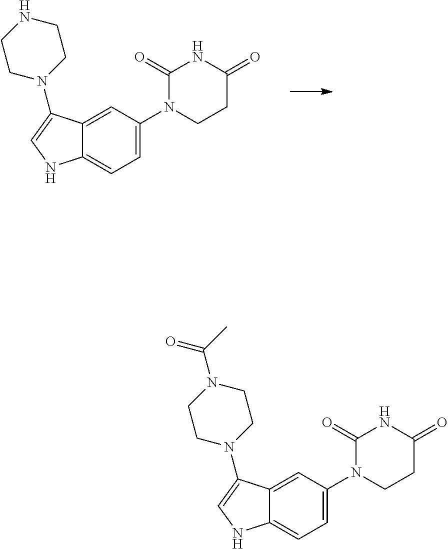 [SMILES: O=C1CCN(c2ccc3[nH]cc(N4CCNCC4)c3c2)C(=O)N1 ; CC(=O)N1CCN(c2c[nH]c3ccc(N4CCC(=O)NC4=O)cc23)CC1]

Ac₂O (19.6 mg, 0.192 mmol) was added to a solution of 1-(3-(piperazin-1-yl)-1H-indol-5-yl)dihydropyrimidine-2,4 (1H,3H)-dione (60.0 mg, 0.191 mmol) and triethylamine (80.0 μL, 0.574 mmol) in DCM (3 mL) at r.t. under air. The resulting mixture was stirred at r.t. for 1 h. The solvent was then removed under reduced pressure. Purification by C-18FC (gradient: 5-20% MeCN in water (containing 0.1% FA)) gave the title compound (52.0 mg, 76%) as a white powder. ¹H NMR: δ 2.04 (3H, s), 2.73 (2H, t), 2.88 (2H, t), 2.95 (2H, t), 3.57-3.67 (4H, m), 3.76 (2H, t), 6.93 (1H, d), 7.00 (1H, dd), 7.29 (1H, d), 7.46 (1H, d), 10.25 (1H, s), 10.66 (1H, s). m/z (ES⁺), [M+H]⁺=356.2.

### Example 80: 1-(1-Methyl-3-(4-methylpiperazin-1-yl)-1H-indol-5-yl)dihydropyrimidine-2,4 (1H,3H)-dione

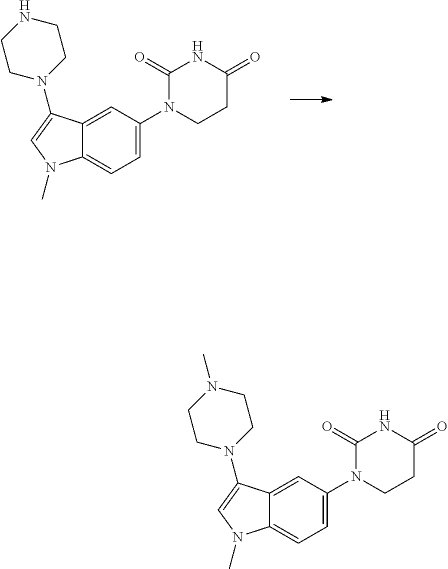 [SMILES: Cn1cc(N2CCNCC2)c2cc(N3CCC(=O)NC3=O)ccc21 ; CN1CCN(c2cn(C)c3ccc(N4CCC(=O)NC4=O)cc23)CC1]

NaOAc (33.8 mg, 0.412 mmol) was added to a mixture of 1-(1-methyl-3-(piperazin-1-yl)-1H-indol-5-yl)dihydropyrimidine-2,4 (1H,3H)-dione hydrochloride (50 mg, 0.137 mmol), sodium triacetoxyborohydride (87.0 mg, 0.410 mmol) and paraformaldehyde (12.4 mg, 0.413 mmol) in DCM (8 mL). The resulting mixture was stirred at r.t. for 1 h. The solvent was then removed under reduced pressure. The crude product was purified by C-18FC (gradient: 5-30% MeCN in water (containing 0.1% FA)) to provide material that was further purified by preparative HPLC (Column J, Eluent B, gradient: 9-17%) to give the title compound (12.0 mg, 26%) as a white solid. ¹H NMR: δ 2.26 (3H, s), 2.25-2.50 (4H, m), 2.72 (2H, t), 2.92-2.96 (4H, m), 3.69 (3H, s), 3.76 (2H, t), 6.88 (1H, s), 7.06 (1H, dd), 7.35 (1H, d), 7.43 (1H, d), 10.22 (1H, s). m/z (ES⁺), [M+H]⁺=342.2.

### Intermediate 81a: tert-Butyl 4-((2-amino-4-bromophenyl)ethynyl)piperidine-1-carboxylate

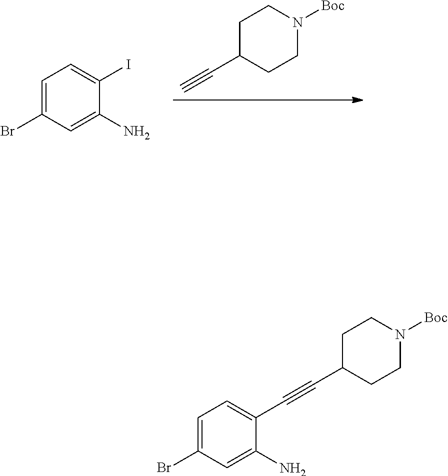 [SMILES: Nc1cc(Br)ccc1I ; C#CC1CCN(C(=O)OC(C)(C)C)CC1 ; CC(C)(C)OC(=O)N1CCC(C#Cc2ccc(Br)cc2N)CC1]

Pd(PPh₃)₄ (1.94 g, 1.68 mmol) was added to a mixture of 5-bromo-2-iodoaniline (5.00 g, 16.8 mmol), tert-butyl 4-ethynylpiperidine-1-carboxylate (3.51 g, 16.8 mmol) and copper(I) iodide (384 mg, 2.01 mmol) in triethylamine (200 mL) at r.t. under N₂. The resulting mixture was stirred at r.t. for 16 h. The solvent was then removed under reduced pressure. Purification by FSC (gradient: 0-22% EtOAc in petroleum ether) gave the title compound (5.80 g, 91%) as a brown gum. ¹H NMR: δ 1.40 (9H, s), 1.44-1.64 (2H, m), 1.75-1.90 (2H, m), 2.80-2.92 (1H, m), 3.02-3.13 (2H, m), 3.67 (2H, dt), 5.52 (2H, s), 6.61 (1H, dd), 6.88 (1H, d), 7.02 (1H, d). m/z (ES⁺), [M+H]⁺=379.2.

### Intermediate 81b: tert-Butyl 6-bromo-2-(1-(tert-butoxycarbonyl)piperidin-4-yl)-1H-indole-1-carboxylate

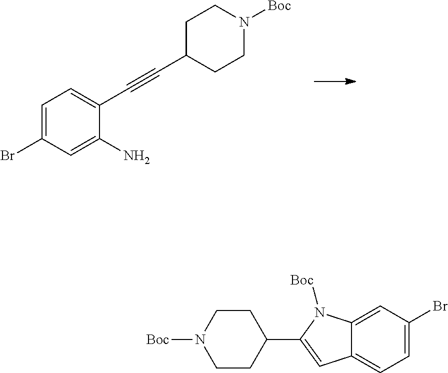 [SMILES: CC(C)(C)OC(=O)N1CCC(C#Cc2ccc(Br)cc2N)CC1 ; CC(C)(C)OC(=O)N1CCC(c2cc3ccc(Br)cc3n2C(=O)OC(C)(C)C)CC1]

Dichlorobis(acetonitrile)palladium(II) (780 mg, 3.01 mmol) was added to a solution of tert-butyl 4-((2-amino-4-bromophenyl)ethynyl)piperidine-1-carboxylate (5.70 g, 15.0 mmol) in DMF (80 mL) at r.t. under N₂. The resulting mixture was stirred at 80° C. for 16 h. The mixture was cooled to r.t. then DIEA (7.87 mL, 45.1 mmol), DMAP (184 mg, 1.51 mmol) and di-tert-butyl dicarbonate (5.23 mL, 22.53 mmol) were added. The mixture was stirred overnight at r.t. The solvent was then removed under reduced pressure. Purification by FSC (gradient: 0-12% EtOAc in petroleum ether) gave the title compound (5.50 g, 76%) as a pale yellow foam. ¹H NMR: (CDCl₃) δ 1.48 (9H, s), 1.49-1.64 (2H, m), 1.70 (9H, s), 2.00-2.10 (2H, m), 2.77-2.93 (2H, m), 3.43-3.57 (1H, m), 4.18-4.30 (2H, m), 6.34 (1H, d), 7.31 (1H, d), 7.31 (1H, s), 8.27 (1H, s). m/z (ES⁺), [M-tBu+2H]⁺=423.1.

### Intermediate 81c: tert-Butyl 2-(1-(tert-butoxycarbonyl)piperidin-4-yl)-6-(2,4-dioxotetrahydropyrimidin-1 (2H)-yl)-1H-indole-1-carboxylate

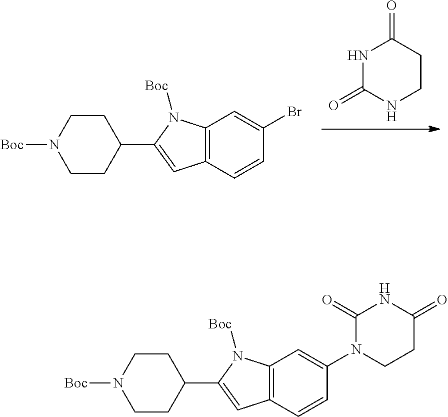

Ephos Pd G4 (96.0 mg, 0.105 mmol) was added to a degassed mixture of tert-butyl 6-bromo-2-(1-(tert-butoxycarbonyl)piperidin-4-yl)-1H-indole-1-carboxylate (1.00 g, 2.09 mmol), dihydropyrimidine-2,4 (1H,3H)-dione (714 mg, 6.26 mmol), Ephos (56.0 mg, 0.105 mmol) and Cs₂CO₃ (1.36 g, 4.17 mmol) in 1,4-dioxane (40 mL) at r.t. under N₂. The resulting mixture was stirred at 100° C. for 16 h. The solvent was then removed under reduced pressure. Purification by FSC (gradient: 0-60% EtOAc in petroleum ether) gave the title compound (960 mg, 90%) as a white solid. ¹H NMR: δ 1.30-1.51 (11H, m), 1.63 (9H, s), 1.99 (2H, d), 2.71 (2H, t), 2.79-2.85 (2H, m), 3.43 (1H, t), 3.79 (2H, t), 3.95-4.16 (2H, m), 6.54 (1H, s), 7.15 (1H, dd), 7.47 (1H, d), 7.98 (1H, d), 10.33 (1H, s). m/z (ES⁺), [M+Na]⁺=535.3.

### Example 81: 1-(2-(Piperidin-4-yl)-1H-indol-6-yl)dihydropyrimidine-2,4 (1H,3H)-dione

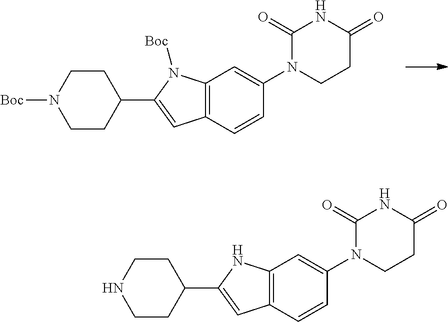 [SMILES: CC(C)(C)OC(=O)N1CCC(c2cc3ccc(N4CCC(=O)NC4=O)cc3n2C(=O)OC(C)(C)C)CC1 ; O=C1CCN(c2ccc3cc(C4CCNCC4)[nH]c3c2)C(=O)N1]

tert-Butyldimethylsilyl trifluoromethanesulfonate (1.96 g, 7.41 mmol) was added to a solution of tert-butyl 2-(1-(tert-butoxycarbonyl)piperidin-4-yl)-6-(2,4-dioxotetrahydropyrimidin-1 (2H)-yl)-1H-indole-1-carboxylate (950 mg, 1.85 mmol) in DCM (20 mL) at r.t. The resulting mixture was stirred at r.t. for 16 h. A further portion of tert-butyldimethylsilyl trifluoromethanesulfonate (1.96 g, 7.41 mmol) was added and stirred for a further 5 h. The solvent was then removed under reduced pressure. Purification by C-18FC (gradient: 0-40% MeCN in water (10 mmol NH₄HCO₃)) gave the title compound (330 mg, 57%) as a white solid. ¹H NMR: δ 1.54 (2H, qd), 1.85-1.95 (2H, m), 2.53-2.66 (2H, m), 2.66-2.83 (3H, m), 2.97-3.07 (2H, m), 3.15 (1H, s), 3.75 (2H, t), 6.10 (1H, s), 6.85 (1H, dd), 7.18 (1H, d), 7.37 (1H, d), 10.26 (1H, s), 10.98 (1H, s). m/z (ES⁺), [M+H]⁺=313.2.

### Example 82: 1-(2-(1-Methylpiperidin-4-yl)-1H-indol-6-yl)dihydropyrimidine-2,4 (1H,3H)-dione

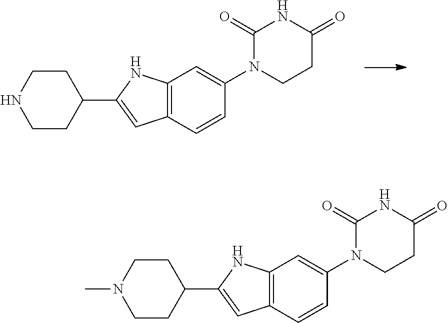 [SMILES: O=C1CCN(c2ccc3cc(C4CCNCC4)[nH]c3c2)C(=O)N1 ; CN1CCC(c2cc3ccc(N4CCC(=O)NC4=O)cc3[nH]2)CC1]

Methyl 4-methylbenzenesulfonate (59.6 mg, 0.320 mmol) was added to a solution of 1-(2-(piperidin-4-yl)-1H-indol-6-yl)dihydropyrimidine-2,4 (1H,3H)-dione (100 mg, 0.320 mmol) and DIEA (56.0 μL, 0.321 mmol) in DMF (3 mL) at r.t. The resulting mixture was stirred at r.t. for 3 h. The crude product was directly purified by preparative HPLC (Column A, Eluent F, gradient: 17-35%) to give the title compound (35.0 mg, 34%) as a white solid. ¹H NMR: δ 1.68 (2H, qd), 1.89-2.04 (4H, m), 2.18 (3H, s), 2.56-2.75 (3H, m), 2.84 (2H, d), 3.74 (2H, t), 6.12 (1H, s), 6.85 (1H, dd), 7.18 (1H, s), 7.37 (1H, d), 10.25 (1H, s), 10.97 (1H, s). m/z (ES⁺), [M+H]⁺=327.2.

### Intermediate 83a: tert-Butyl 4-(6-bromo-1H-indol-2-yl)piperidine-1-carboxylate

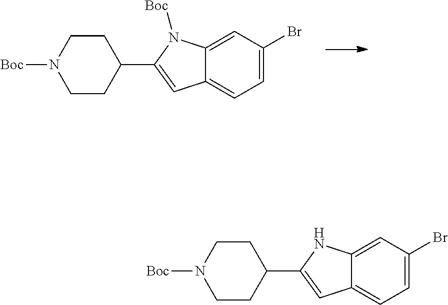 [SMILES: CC(C)(C)OC(=O)N1CCC(c2cc3ccc(Br)cc3n2C(=O)OC(C)(C)C)CC1 ; CC(C)(C)OC(=O)N1CCC(c2cc3ccc(Br)cc3[nH]2)CC1]

tert-Butyl 6-bromo-2-(1-(tert-butoxycarbonyl)piperidin-4-yl)-1H-indole-1-carboxylate (500 mg, 1.04 mmol) was dissolved in 2,2,2-trifluoroethanol (15 mL) and sealed into a microwave tube. The reaction was heated to 150° C. for 1 h in a microwave reactor and then cooled to r.t. The solvent was then removed under reduced pressure. Purification by FSC (gradient: 0-30% EtOAc in petroleum ether) gave the title compound (380 mg, 96%) as a pale yellow solid. ¹H NMR: δ 1.40 (9H, s), 1.51 (2H, qd), 1.88-2.00 (2H, m), 2.73-2.96 (3H, m), 3.97-4.07 (2H, m), 6.17 (1H, d), 7.03 (1H, dd), 7.36 (1H, d), 7.41 (1H, s), 11.12 (1H, s). m/z (ES⁺), [M+H]⁺=381.1.

### Intermediate 83b: tert-Butyl 4-(1-acetyl-6-bromo-1H-indol-2-yl)piperidine-1-carboxylate

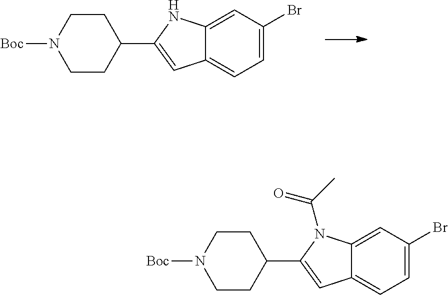 [SMILES: CC(C)(C)OC(=O)N1CCC(c2cc3ccc(Br)cc3[nH]2)CC1 ; CC(=O)n1c(C2CCN(C(=O)OC(C)(C)C)CC2)cc2ccc(Br)cc21]

Ac₂O (179 μL, 1.90 mmol) was added to a mixture of tert-butyl 4-(6-bromo-1H-indol-2-yl)piperidine-1-carboxylate (360 mg, 0.949 mmol), triethylamine (397 μL, 2.85 mmol) and DMAP (11.6 mg, 0.0949 mmol) in DCE (10 mL) at r.t. The resulting solution was stirred at 80° C. for 16 h. A second portion of Ac₂O (4 mL) and triethylamine (4 ml) was added and the reaction was heated to 80° C. for 4 days. The solvent was then removed under reduced pressure. Purification by FSC (gradient: 0-25% EtOAc in petroleum ether) gave the title compound (350 mg, 88%) as a yellow solid. ¹H NMR: δ 1.31-1.51 (11H, m), 1.92-2.04 (2H, m), 2.68-2.94 (5H, m), 3.32-3.49 (1H, m), 3.97-4.09 (2H, m), 6.61 (1H, s), 7.36 (1H, dd), 7.47 (1H, d), 8.00 (1H, d). m/z (ES⁺), [M+Na]⁺=445.1.

### Intermediate 83c: tert-Butyl 4-(6-(2,4-dioxotetrahydropyrimidin-1 (2H)-yl)-1H-indol-2-yl)piperidine-1-carboxylate

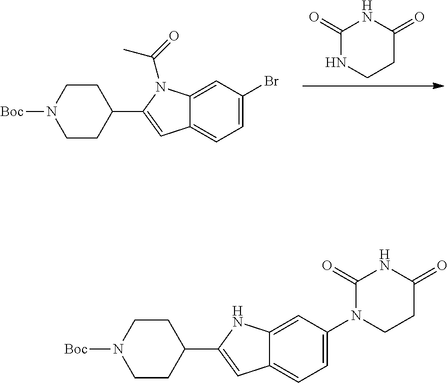 [SMILES: CC(=O)n1c(C2CCN(C(=O)OC(C)(C)C)CC2)cc2ccc(Br)cc21 ; O=C1CCNC(=O)N1 ; CC(C)(C)OC(=O)N1CCC(c2cc3ccc(N4CCC(=O)NC4=O)cc3[nH]2)CC1]

Ephos Pd G4 (41.9 mg, 0.0456 mmol) was added to a degassed mixture of tert-butyl 4-(1-acetyl-6-bromo-1H-indol-2-yl)piperidine-1-carboxylate (320 mg, 0.759 mmol), dihydropyrimidine-2,4 (1H,3H)-dione (260 mg, 2.28 mmol), Ephos (24.4 mg, 0.0456 mmol) and Cs₂CO₃ (742 mg, 2.28 mmol) in 1,4-dioxane (10 mL) at r.t. under N₂. The resulting mixture was stirred at 100° C. for 16 h. The solvent was then removed under reduced pressure. Purification by C-18FC (gradient: 0-40% MeCN in water (10 mmol NH₄HCO₃)) gave the title compound (210 mg, 67%) as a white solid. ¹H NMR: δ 1.42 (9H, s), 1.47-1.63 (2H, m), 1.91-2.02 (2H, m), 2.72 (2H, t), 2.79-2.99 (3H, m), 3.77 (2H, t), 3.99-4.12 (2H, m), 6.16 (1H, d), 6.88 (1H, dd), 7.21 (1H, d), 7.40 (1H, d), 10.27 (1H, s), 11.03 (1H, s). m/z (ES⁺), [M-tBu+2H]⁺=357.1.

### Example 83: 1-(2-(1-Acetylpiperidin-4-yl)-1H-indol-6-yl)dihydropyrimidine-2,4 (1H,3H)-dione

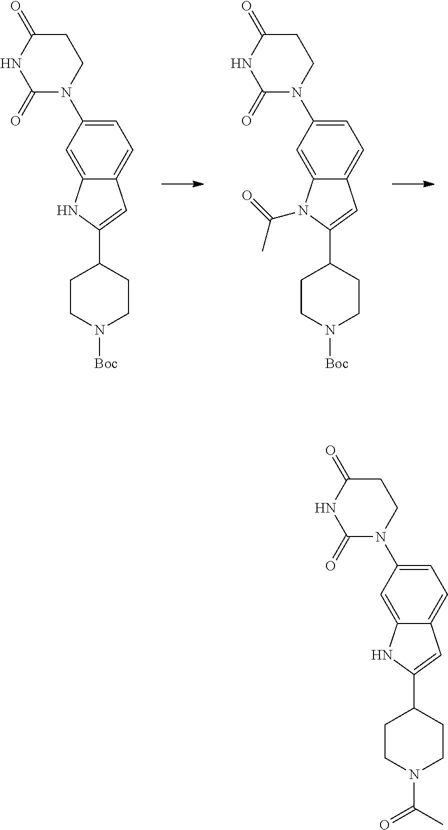 [SMILES: CC(C)(C)OC(=O)N1CCC(c2cc3ccc(N4CCC(=O)NC4=O)cc3[nH]2)CC1 ; CC(=O)n1c(C2CCN(C(=O)OC(C)(C)C)CC2)cc2ccc(N3CCC(=O)NC3=O)cc21 ; CC(=O)N1CCC(c2cc3ccc(N4CCC(=O)NC4=O)cc3[nH]2)CC1]

Ac₂O (1.00 mL, 10.6 mmol) was added to a solution of tert-butyl 4-(6-(2,4-dioxotetrahydropyrimidin-1 (2H)-yl)-1H-indol-2-yl)piperidine-1-carboxylate (200 mg, 0.485 mmol) and DIEA (2.00 mL, 11.5 mmol) in DCE (10 mL) at r.t. The resulting mixture was stirred at 80° C. for 3 days. The solvent was then removed under reduced pressure. The crude product was purified by FSC (gradient: 0-60% EtOAc in petroleum ether). Fractions containing the desired compound were concentrated to dryness to give an inseparable mixture of tert-butyl 4-(6-(2,4-dioxotetrahydropyrimidin-1 (2H)-yl)-1H-indol-2-yl)piperidine-1-carboxylate and tert-butyl 4-(1-acetyl-6-(2,4-dioxotetrahydropyrimidin-1 (2H)-yl)-1H-indol-2-yl)piperidine-1-carboxylate (150 mg, 1:1.7 ratio respectively) as a pale yellow solid. The crude mixture was carried over to the next step without any further purification. The ¹H NMR: (peaks of the desired compound): δ 1.42 (9H, s), 1.42-1.48 (1H, m), 1.56 (1H, dd), 1.93-2.04 (2H, m), 2.45 (3H, s), 2.83-2.97 (3H, m), 3.39-3.52 (2H, m), 3.84 (2H, t), 4.04-4.12 (2H, m), 6.19 (1H, d), 6.93 (1H, dd), 7.27 (1H, d), 7.43 (1H, d), 11.08 (1H, s). m/z (ES⁺), [M+Na]⁺=477.2. The inseparable mixture was then dissolved in 2,2,2-trifluoroethanol (5 mL) and sealed into a microwave tube. The reaction was heated to 150° C. for 6 h in a microwave reactor and then cooled to r.t. The solvent was then removed under reduced pressure. Purification by C-18FC (gradient: 0-30% MeCN in water (containing 0.05% TFA)) gave material that was further purified by preparative HPLC (Column E, Eluent E, gradient: 20-30%) to give the title compound (15.0 mg, 9% over two-steps) as a white solid (the acetyl migration from the indole to the piperidine occurred during the Boc deprotection step). ¹H NMR: δ 1.44-1.58 (1H, m), 1.58-1.70 (1H, m), 2.00 (2H, t), 2.04 (3H, s), 2.62-2.70 (1H, m), 2.72 (2H, t), 2.98 (1H, t), 3.18 (1H, t), 3.77 (2H, t), 3.92 (1H, d), 4.47 (1H, d), 6.16 (1H, d), 6.88 (1H, dd), 7.21 (1H, d), 7.41 (1H, d), 10.27 (1H, s), 11.04 (1H, s). m/z (ES⁺), [M+H]⁺=355.3.

### Intermediate 84a: tert-Butyl 4-(6-bromo-1-methyl-1H-indol-2-yl)piperidine-1-carboxylate

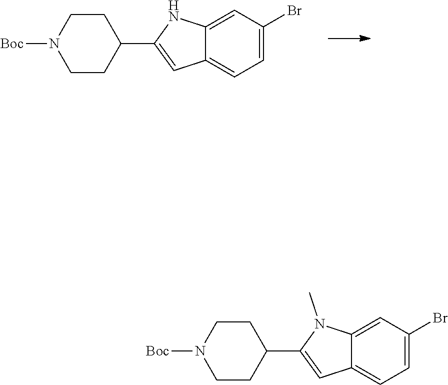 [SMILES: CC(C)(C)OC(=O)N1CCC(c2cc3ccc(Br)cc3[nH]2)CC1 ; Cn1c(C2CCN(C(=O)OC(C)(C)C)CC2)cc2ccc(Br)cc21]

NaH (60% dispersion in mineral oil, 79 mg, 1.98 mmol) was added to a solution of tert-butyl 4-(6-bromo-1H-indol-2-yl)piperidine-1-carboxylate (500 mg, 1.32 mmol) in THF (10 mL) at 0° C. under N₂. The resulting mixture was stirred at r.t. for 0.5 h before the addition of Mel (99.0 μL, 1.58 mmol). The resulting mixture was stirred at r.t. for 2 h. The reaction mixture was quenched with ice water (20 mL) and extracted with EtOAc (3×20 mL). The combined organic extracts were dried (Na₂SO₄) and concentrated to give a pale yellow gum. Purification by FSC (gradient: 0-60% EtOAc in petroleum ether) gave the title compound (360 mg, 69%) as a pale yellow solid. ¹H NMR: δ 1.40-1.54 (11H, m), 1.93 (2H, d), 2.87-3.04 (3H, m), 3.71 (3H, s), 4.02-4.13 (2H, m), 6.26 (1H, s), 7.10 (1H, dd), 7.40 (1H, d), 7.66 (1H, d). m/z (ES⁺), [M+H]⁺=393.2.

### Intermediate 84b: tert-Butyl 4-(6-(2,4-dioxotetrahydropyrimidin-1 (2H)-yl)-1-methyl-1H-indol-2-yl)piperidine-1-carboxylate

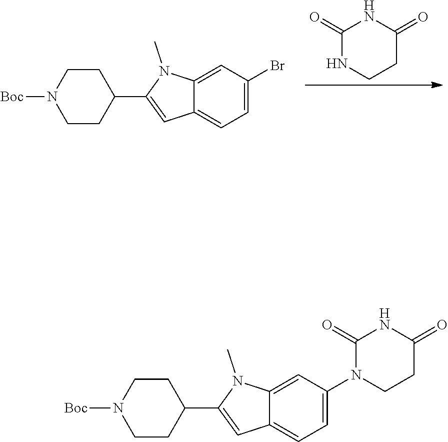 [SMILES: Cn1c(C2CCN(C(=O)OC(C)(C)C)CC2)cc2ccc(Br)cc21 ; O=C1CCNC(=O)N1 ; Cn1c(C2CCN(C(=O)OC(C)(C)C)CC2)cc2ccc(N3CCC(=O)NC3=O)cc21]

Cs₂CO₃ (497 mg, 1.53 mmol) was added to a degassed mixture of tert-butyl 4-(6-bromo-1-methyl-1H-indol-2-yl)piperidine-1-carboxylate (200 mg, 0.508 mmol), dihydropyrimidine-2,4 (1H,3H)-dione (232 mg, 2.03 mmol), Ephos Pd G4 (46.7 mg, 0.0508 mmol) and Ephos (27.2 mg, 0.0509 mmol) in 1,4-dioxane (15 mL) at r.t. under N₂. The resulting solution was stirred at 100° C. for 17 h. The solvent was then removed under reduced pressure. Purification by C-18FC (gradient: 0-50% MeCN in water (containing 0.1% FA)) gave the title compound (120 mg, 55%) as a pale yellow solid. ¹H NMR: (CDCl₃) δ 1.51 (9H, s), 1.58-1.78 (2H, m), 1.94-2.08 (2H, m), 2.83-2.97 (5H, m), 3.73 (3H, s), 3.93 (2H, t), 4.29 (2H, d), 6.28 (1H, s), 6.98 (1H, dd), 7.28 (1H, s), 7.54 (1H, s), 7.58 (1H, d). m/z (ES⁺), [M-tBu+2H]⁺=371.2.

### Example 84: 1-(1-Methyl-2-(piperidin-4-yl)-1H-indol-6-yl)dihydropyrimidine-2,4 (1H,3H)-dione

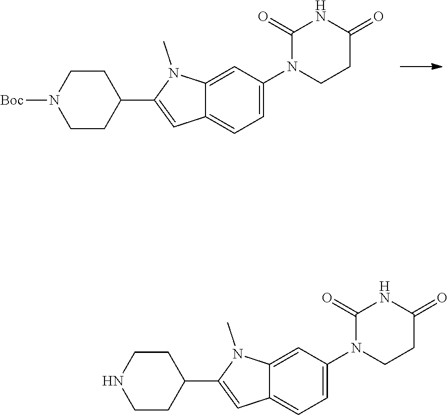 [SMILES: Cn1c(C2CCN(C(=O)OC(C)(C)C)CC2)cc2ccc(N3CCC(=O)NC3=O)cc21 ; Cn1c(C2CCNCC2)cc2ccc(N3CCC(=O)NC3=O)cc21]

tert-Butyldimethylsilyl trifluoromethanesulfonate (124 μL, 0.539 mmol) was added to tert-butyl 4-(6-(2,4-dioxotetrahydropyrimidin-1 (2H)-yl)-1-methyl-1H-indol-2-yl)piperidine-1-carboxylate (115 mg, 0.270 mmol) in MeCN (10 mL) at r.t. under air. The resulting solution was stirred at r.t. for 2 h. The solvent was then removed under reduced pressure. Purification by C-18FC (gradient: 0-20% MeCN in water (containing 0.1% FA)) gave the title compound in the form of a formate salt (40 mg, 37%) as a pale yellow solid. ¹H NMR: δ 1.71-1.85 (2H, m), 2.12 (2H, t), 2.74 (2H, t), 3.04-3.21 (3H, m), 3.41 (2H, d), 3.71 (3H, s), 3.79 (2H, t), 6.26 (1H, s), 6.96 (1H, dd), 7.39 (1H, d), 7.47 (1H, d), 8.43 (1H, br s), 10.29 (1H, s). m/z (ES⁺), [M+H]⁺=327.3.

### Intermediate 85a: 6-Bromo-1-methyl-2-(piperidin-4-yl)-1H-indole

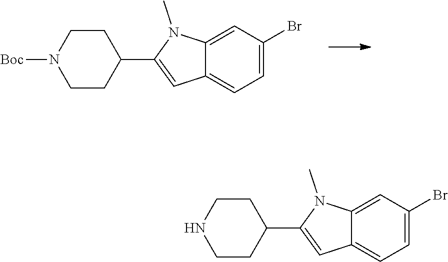 [SMILES: Cn1c(C2CCN(C(=O)OC(C)(C)C)CC2)cc2ccc(Br)cc21 ; Cn1c(C2CCNCC2)cc2ccc(Br)cc21]

tert-Butyl 4-(6-bromo-1-methyl-1H-indol-2-yl)piperidine-1-carboxylate (200 mg, 0.508 mmol) was dissolved in 2,2,2-trifluoroethanol (10 ml) and sealed into a microwave tube. The reaction was heated to 150° C. for 12 h in a microwave reactor and then cooled to r.t. The solvent was removed under reduced pressure to give the title compound (130 mg, 87%) as a pale yellow solid. ¹H NMR: (CDCl₃) δ 2.25 (4H, s), 3.06 (3H, d), 3.70 (5H, s), 6.35 (1H, s), 7.21 (1H, dd), 7.38-7.49 (2H, m). m/z (ES⁺), [M+H]⁺=295.2.

### Intermediate 85b: 6-Bromo-1-methyl-2-(1-methylpiperidin-4-yl)-1H-indole

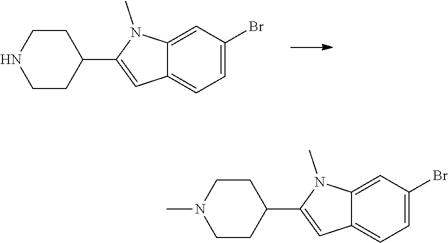 [SMILES: Cn1c(C2CCNCC2)cc2ccc(Br)cc21 ; CN1CCC(c2cc3ccc(Br)cc3n2C)CC1]

AcOH (19.5 μL, 0.341 mmol) was added to a mixture of NaOAc (56.0 mg, 0.683 mmol), sodium triacetoxyborohydride (723 mg, 3.41 mmol), formaldehyde (20.5 mg, 0.683 mmol) and 6-bromo-1-methyl-2-(piperidin-4-yl)-1H-indole (100 mg, 0.341 mmol) in DCM (10 mL) at r.t. under air. The resulting mixture was stirred at r.t. for 3 h. The solvent was removed under reduced pressure. Purification by C-18FC (gradient: 0-100% MeOH in water (containing 0.1% FA)) gave the title compound in the form of a formate salt (90.0 mg, 75%) as a pale yellow gum. ¹H NMR: (CDCl₃) δ 2.11 (4H, dt), 2.63 (3H, s), 2.80-2.90 (1H, m), 3.41 (2H, d), 3.68 (3H, s), 3.70-3.76 (2H, m), 6.31 (1H, s), 7.20 (1H, dd), 7.40-7.47 (2H, m), 8.51 (1H, s). m/z (ES⁺), [M+H]⁺=307.1.

### Example 85: 1-(1-Methyl-2-(1-methylpiperidin-4-yl)-1H-indol-6-yl)dihydropyrimidine-2,4 (1H,3H)-dione

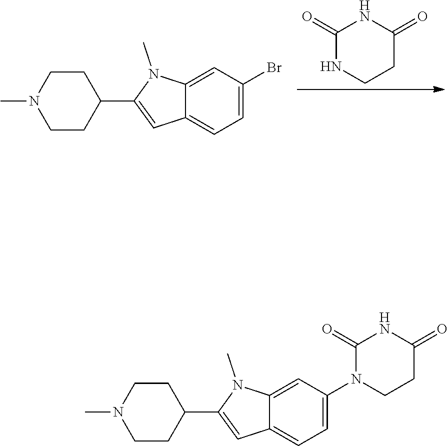 [SMILES: CN1CCC(c2cc3ccc(Br)cc3n2C)CC1 ; O=C1CCNC(=O)N1 ; CN1CCC(c2cc3ccc(N4CCC(=O)NC4=O)cc3n2C)CC1]

Ephos (7.6 mg, 0.014 mmol) and Ephos Pd G4 (13.0 mg, 0.0142 mmol) were added to a degassed mixture of Cs₂CO₃ (138 mg, 0.42 mmol), dihydropyrimidine-2,4 (1H,3H)-dione (48.4 mg, 0.42 mmol) and 6-bromo-1-methyl-2-(1-methylpiperidin-4-yl)-1H-indole formate (50.0 mg, 0.142 mmol) in 1,4-dioxane (5 mL) at r.t. under N₂. The resulting mixture was stirred at 100° C. for 17 h. The solvent was then removed under reduced pressure. Purification by C-18FC (gradient: 0-30% MeCN in water (containing 0.1% FA)) gave material that was further purified by preparative HPLC (Column K, Eluent E, gradient: 10-23%) to give the title compound in the form of a formate salt (8.0 mg, 14%) as a pale yellow solid. ¹H NMR: δ 1.62-1.78 (2H, m), 1.95 (2H, d), 2.20-2.34 (5H, m), 2.73 (2H, t), 2.76-2.86 (1H, m), 2.99 (2H, d), 3.68 (3H, s), 3.78 (2H, t), 6.22 (1H, s), 6.93 (1H, dd), 7.36 (1H, d), 7.43 (1H, d), 8.27 (1H, s), 10.30 (1H, s). m/z (ES⁺), [M+H]⁺=341.1.

### Intermediate 86a: tert-Butyl 4-((2-amino-6-bromophenyl)ethynyl)piperidine-1-carboxylate

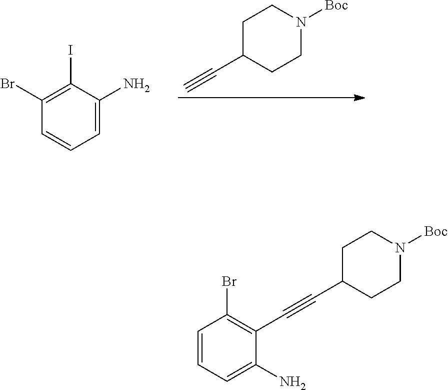 [SMILES: Nc1cccc(Br)c1I ; C#CC1CCN(C(=O)OC(C)(C)C)CC1 ; CC(C)(C)OC(=O)N1CCC(C#Cc2c(N)cccc2Br)CC1]

Pd(Ph₃P)₄ (3.10 g, 2.68 mmol) was added to a mixture of 3-bromo-2-iodoaniline (8.00 g, 26.9 mmol), tert-butyl 4-ethynylpiperidine-1-carboxylate (5.62 g, 26.9 mmol) and copper(I) iodide (614 mg, 3.22 mmol) in triethylamine (250 mL) at r.t. under N₂. The resulting mixture was stirred at r.t. for 16 h. The solvent was then removed under reduced pressure. Purification by FSC (gradient: 0-22% EtOAc in petroleum ether) gave the title compound (10.0 g, 98%) as a brown gum. ¹H NMR: (CDCl₃) δ 1.47 (9H, s), 1.68-1.80 (2H, m), 1.82-1.97 (2H, m), 2.91-3.01 (1H, m), 3.30-3.45 (2H, m), 3.66-3.80 (2H, m), 4.28 (2H, br s), 6.58-6.67 (1H, m), 6.86-6.97 (2H, m). m/z (ES⁺), [M+H]⁺=379.0.

### Intermediate 86b: tert-Butyl 4-(4-bromo-1H-indol-2-yl)piperidine-1-carboxylate

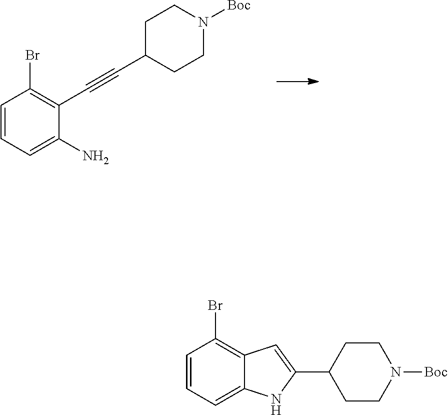 [SMILES: CC(C)(C)OC(=O)N1CCC(C#Cc2c(N)cccc2Br)CC1 ; CC(C)(C)OC(=O)N1CCC(c2cc3c(Br)cccc3[nH]2)CC1]

Dichlorobis(acetonitrile)palladium(II) (684 mg, 2.64 mmol) was added to a solution of tert-butyl 4-((2-amino-6-bromophenyl)ethynyl)piperidine-1-carboxylate (5.00 g, 13.2 mmol) in DMF (80 mL) at r.t. under N₂. The resulting mixture was stirred at 80° C. for 16 h. The reaction mixture was quenched with water (200 mL) and extracted with EtOAc (3×200 mL). The combined organic extracts were dried (Na₂SO₄) and concentrated to give a dark oil. Purification by FSC (gradient: 0-50% EtOAc in petroleum ether) gave the title compound (4.20 g, 84%) as a yellow solid. m/z (ES⁺), [M-tBu+2H]⁺=325.0.

### Intermediate 86c: tert-Butyl 4-bromo-2-(1-(tert-butoxycarbonyl)piperidin-4-yl)-1H-indole-1-carboxylate

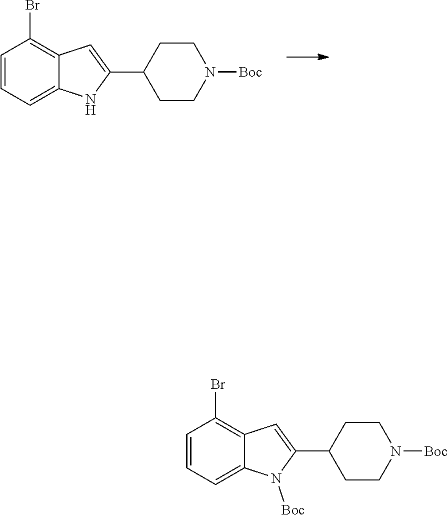 [SMILES: CC(C)(C)OC(=O)N1CCC(c2cc3c(Br)cccc3[nH]2)CC1 ; CC(C)(C)OC(=O)N1CCC(c2cc3c(Br)cccc3n2C(=O)OC(C)(C)C)CC1]

Di-tert-butyl dicarbonate (1.81 g, 8.29 mmol) was added to tert-butyl 4-(4-bromo-1H-indol-2-yl)piperidine-1-carboxylate (2.10 g, 5.54 mmol), triethylamine (1.12 g, 11.1 mmol) and DMAP (68.0 mg, 0.557 mmol) in DCM (50 mL) at r.t. under air. The resulting mixture was stirred at r.t. for 1 h. The solvent was then removed under reduced pressure. Purification by FSC (gradient: 0-25% EtOAc in petroleum ether) gave the title compound (1.45 g, 55%) as a white solid. ¹H NMR: (CDCl₃) δ 1.48 (9H, s), 1.58-1.65 (2H, m), 1.70 (9H, s), 2.05-2.12 (2H, m), 2.86 (2H, t), 3.53 (1H, t), 4.25 (2H, d), 6.46 (1H, s), 7.09 (1H, t), 7.35 (1H, d), 8.01 (1H, d). m/z (ES⁺), [M-tBu+2H]⁺=425.1.

### Intermediate 86d: tert-Butyl 2-(1-(tert-butoxycarbonyl)piperidin-4-yl)-4-(2,4-dioxotetrahydropyrimidin-1 (2H)-yl)-1H-indole-1-carboxylate

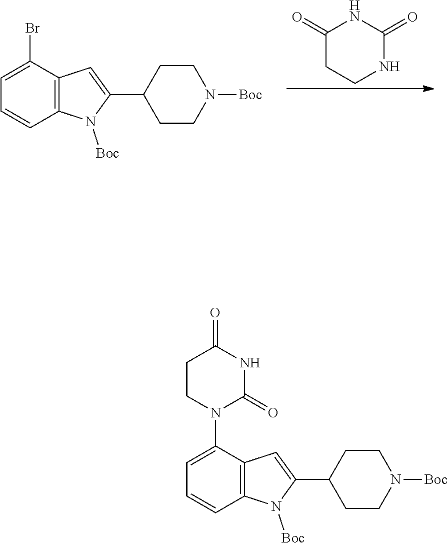 [SMILES: CC(C)(C)OC(=O)N1CCC(c2cc3c(Br)cccc3n2C(=O)OC(C)(C)C)CC1 ; O=C1CCNC(=O)N1 ; CC(C)(C)OC(=O)N1CCC(c2cc3c(N4CCC(=O)NC4=O)cccc3n2C(=O)OC(C)(C)C)CC1]

Ephos (156 mg, 0.292 mmol) and Ephos Pd G4 (268 mg, 0.292 mmol) were added to a degassed mixture of tert-butyl 4-bromo-2-(1-(tert-butoxycarbonyl)piperidin-4-yl)-1H-indole-1-carboxylate (1.40 g, 2.92 mmol), dihydropyrimidine-2,4 (1H,3H)-dione (1.00 g, 8.76 mmol) and Cs₂CO₃ (1.90 g, 5.83 mmol) in 1,4-dioxane (80 mL) at r.t. under N₂. The resulting mixture was stirred at 100° C. for 16 h. The solvent was then removed under reduced pressure. Purification by FSC (gradient: 0-80% EtOAc in petroleum ether) gave the title compound (490 mg, 33%) as a white solid. ¹H NMR: (CDCl₃) δ 1.47 (9H, s), 1.53-1.65 (2H, m), 1.69 (9H, s), 2.04-2.11 (2H, m), 2.78-2.95 (4H, m), 3.55 (1H, t), 3.88 (2H, t), 4.25 (2H, d), 6.24 (1H, t), 7.09 (1H, dd), 7.27-7.31 (1H, m), 7.52 (1H, br s), 8.06 (1H, dt). m/z (ES⁺), [M+H]⁺=513.4.

### Example 86: 1-(2-(Piperidin-4-yl)-1H-indol-4-yl)dihydropyrimidine-2,4 (1H,3H)-dione

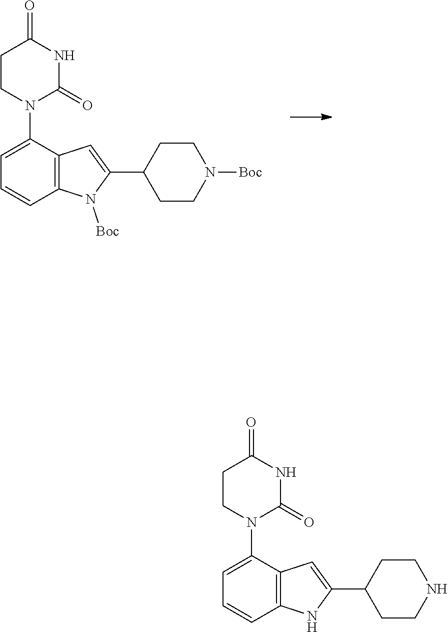 [SMILES: CC(C)(C)OC(=O)N1CCC(c2cc3c(N4CCC(=O)NC4=O)cccc3n2C(=O)OC(C)(C)C)CC1 ; O=C1CCN(c2cccc3[nH]c(C4CCNCC4)cc23)C(=O)N1]

tert-Butyl 2-(1-(tert-butoxycarbonyl)piperidin-4-yl)-4-(2,4-dioxotetrahydropyrimidin-1 (2H)-yl)-1H-indole-1-carboxylate (470 mg, 0.917 mmol) was dissolved in 2,2,2-trifluoroethanol (10 mL) and sealed into a microwave tube. The reaction was heated to 150° C. for 12 h in a microwave reactor and then cooled to r.t. The solvent was then removed under reduced pressure. Purification by C-18FC (gradient: 5-40% MeCN in water (10 mmol NH₄HCO₃)) gave the title compound (210 mg, 73%) as a white solid. ¹H NMR: δ 1.44-1.65 (2H, m), 1.90-1.94 (2H, m), 2.55-2.67 (1H, m), 2.70 (2H, t), 2.78-2.94 (1H, m), 3.00-3.31 (3H, m), 3.75 (2H, t), 3.90-4.28 (1H, m), 6.10 (1H, s), 6.86 (1H, dd), 7.01 (1H, t), 7.23 (1H, d), 10.27 (1H, s), 11.08 (1H, s). m/z (ES⁺), [M+H]⁺=313.1.

### Example 87: 1-(2-(1-Methylpiperidin-4-yl)-1H-indol-4-yl)dihydropyrimidine-2,4 (1H,3H)-dione

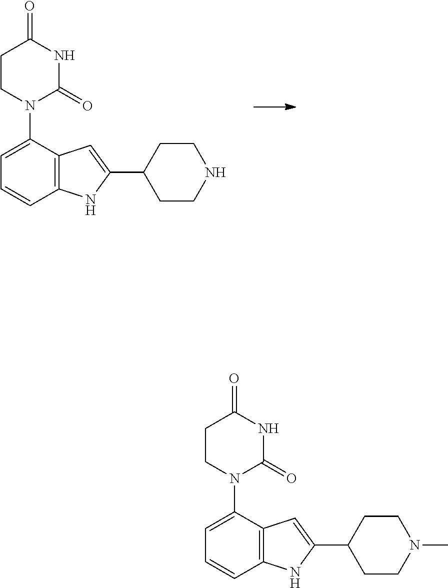 [SMILES: O=C1CCN(c2cccc3[nH]c(C4CCNCC4)cc23)C(=O)N1 ; CN1CCC(c2cc3c(N4CCC(=O)NC4=O)cccc3[nH]2)CC1]

Sodium triacetoxyborohydride (142 mg, 0.670 mmol) was added to a mixture of 1-(2-(piperidin-4-yl)-1H-indol-4-yl)dihydropyrimidine-2,4 (1H,3H)-dione (70.0 mg, 0.224 mmol), paraformaldehyde (33.6 mg, 1.12 mmol) in MeOH (0.5 mL) and DCM (5 mL) at r.t. under air. The resulting mixture was stirred at r.t. for 2 h. The solvent was then removed under reduced pressure. Purification by C-18FC (gradient: 5-35% MeCN in water (10 mmol NH₄HCO₃)) gave the title compound (50 mg, 68%) as a white solid. ¹H NMR: δ 1.60-1.82 (2H, m), 1.88-2.04 (4H, m), 2.19 (3H, s), 2.59-2.70 (1H, m), 2.75 (2H, t), 2.85 (2H, d), 3.75 (2H, t), 6.11 (1H, s), 6.86 (1H, d), 7.01 (1H, t), 7.23 (1H, d), 10.27 (1H, s), 11.07 (1H, s). m/z (ES⁺), [M+H]⁺=327.1.

### Intermediate 88a: tert-Butyl 4-(4-bromo-1-methyl-1H-indol-2-yl)piperidine-1-carboxylate

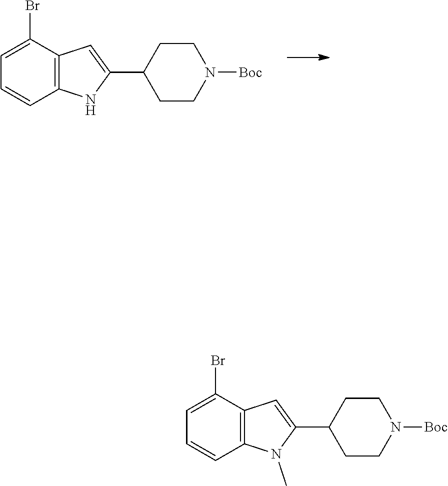 [SMILES: CC(C)(C)OC(=O)N1CCC(c2cc3c(Br)cccc3[nH]2)CC1 ; Cn1c(C2CCN(C(=O)OC(C)(C)C)CC2)cc2c(Br)cccc21]

NaH (60% dispersion in mineral oil, 316 mg, 7.91 mmol) was added to a solution of tert-butyl 4-(4-bromo-1H-indol-2-yl)piperidine-1-carboxylate (2.00 g, 5.27 mmol) in DMF (50 mL) at 0° C. under N₂. The mixture was stirred for 10 minutes at r.t. before the addition of Mel (328 μL, 5.27 mmol) at r.t. The resulting mixture was stirred at r.t. for 2 h. The reaction mixture was then quenched with water (100 mL) and extracted with EtOAc (3×100 mL). The combined organic extracts were dried (Na₂SO₄) and concentrated to give a yellow oil. Purification by C-18FC (gradient: 5-80% MeCN in water (containing 0.05% TFA)) gave the title compound (800 mg, 39%) as a yellow solid. ¹H NMR: (CDCl₃) δ 1.49 (9H, s), 1.62-1.78 (2H, m), 1.99 (2H, d), 2.80-2.96 (3H, m), 3.71 (3H, s), 4.27 (2H, br d), 6.30 (1H, s), 7.03 (1H, t), 7.21-7.26 (2H, m). m/z (ES⁺), [M+H]⁺=395.0.

### Intermediate 88b: tert-Butyl 4-(4-(2,4-dioxotetrahydropyrimidin-1 (2H)-yl)-1-methyl-1H-indol-2-yl)piperidine-1-carboxylate

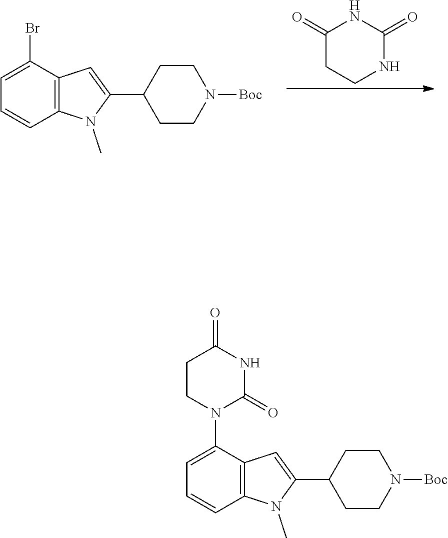 [SMILES: Cn1c(C2CCN(C(=O)OC(C)(C)C)CC2)cc2c(Br)cccc21 ; O=C1CCNC(=O)N1 ; Cn1c(C2CCN(C(=O)OC(C)(C)C)CC2)cc2c(N3CCC(=O)NC3=O)cccc21]

Ephos (109 mg, 0.204 mmol) and Ephos Pd G4 (187 mg, 0.204 mmol) were added to a mixture of tert-butyl 4-(4-bromo-1-methyl-1H-indol-2-yl)piperidine-1-carboxylate (800 mg, 2.03 mmol), dihydropyrimidine-2,4 (1H,3H)-dione (696 mg, 6.10 mmol) and Cs₂CO₃ (1.33 g, 4.08 mmol) in DMF (50 mL) at r.t. under N₂. The resulting mixture was stirred at 100° C. for 72 h. The reaction mixture was then quenched with water (100 mL), extracted with EtOAc (3×50 mL). The combined organic extracts were dried (Na₂SO₄) and concentrated to give a yellow oil. Purification by FSC (gradient: 0-100% EtOAc in petroleum ether) gave the title compound (80.0 mg, 9%) as a yellow solid. ¹H NMR: δ 1.42 (9H, s), 1.44-1.60 (2H, m), 1.87-1.96 (2H, m), 2.77 (2H, t), 2.82-3.09 (3H, m), 3.68-3.79 (5H, m), 4.05-4.16 (2H, m), 6.25 (1H, s), 6.91 (1H, d), 7.09 (1H, t), 7.36 (1H, d), 10.28 (1H, s). m/z (ES⁺), [M-tBu+2H]⁺=371.1.

### Example 88: 1-(1-Methyl-2-(piperidin-4-yl)-1H-indol-4-yl)dihydropyrimidine-2,4 (1H,3H)-dione

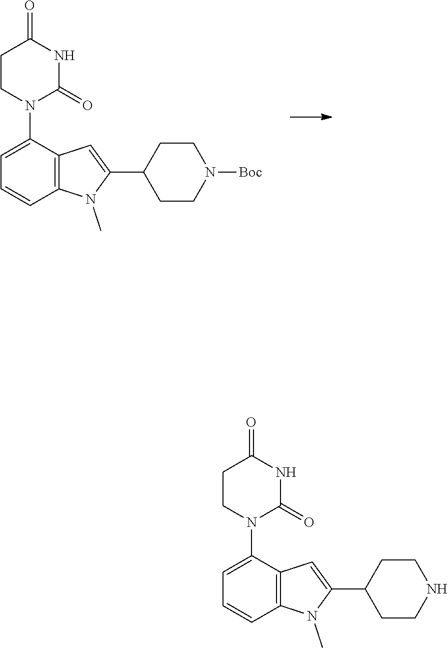 [SMILES: Cn1c(C2CCN(C(=O)OC(C)(C)C)CC2)cc2c(N3CCC(=O)NC3=O)cccc21 ; Cn1c(C2CCNCC2)cc2c(N3CCC(=O)NC3=O)cccc21]

tert-Butyl 4-(4-(2,4-dioxotetrahydropyrimidin-1 (2H)-yl)-1-methyl-1H-indol-2-yl)piperidine-1-carboxylate (180 mg, 0.422 mmol) was dissolved in 2,2,2-trifluoroethanol (8 mL) and sealed into a microwave tube. The reaction was heated to 150° C. for 7 h in a microwave reactor and then cooled to r.t. The solvent was removed under reduced pressure. Purification by C-18FC (gradient: 5-20% MeCN in water (containing 0.05% TFA)) gave the title compound in the form of a trifluoroacetate salt (150 mg, 81%) as a white solid. ¹H NMR: δ 1.67-1.94 (2H, m), 2.09-2.13 (2H, m), 2.77 (2H, t), 2.99-3.28 (3H, m), 3.38-3.42 (2H, m), 3.67-3.82 (5H, m), 6.20 (1H, s), 6.94 (1H, d), 7.13 (1H, t), 7.38 (1H, d), 8.38 (1H, s), 8.57-8.87 (1H, m), 10.31 (1H, s). ¹⁹F NMR (282 MHz 6-73.92. m/z (ES⁺), [M+H]⁺=327.1.

### Example 89: 1-(1-Methyl-2-(1-methylpiperidin-4-yl)-1H-indol-4-yl)dihydropyrimidine-2,4 (1H,3H)-dione

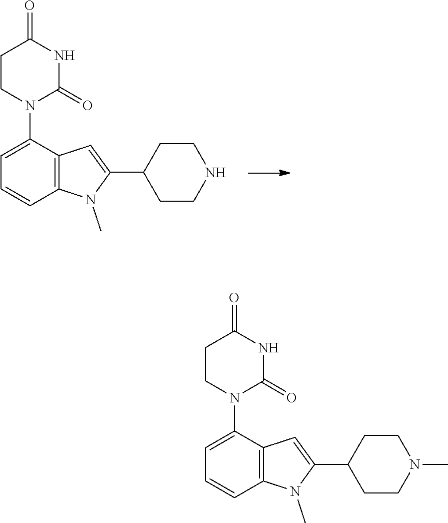 [SMILES: Cn1c(C2CCNCC2)cc2c(N3CCC(=O)NC3=O)cccc21 ; CN1CCC(c2cc3c(N4CCC(=O)NC4=O)cccc3n2C)CC1]

Sodium triacetoxyborohydride (130 mg, 0.613 mmol) was added to a mixture of 1-(1-methyl-2-(piperidin-4-yl)-1H-indol-4-yl)dihydropyrimidine-2,4 (1H,3H)-dione 2,2,2-trifluoroacetate (90.0 mg, 0.204 mmol), paraformaldehyde (30.7 mg, 1.02 mmol) and NaOAc (50.3 mg, 0.613 mmol) in MeOH (1 mL) and DCM (4 mL) at r.t. under air. The resulting mixture was stirred at r.t. for 16 h. The solvent was then removed under reduced pressure. The crude product was purified by C-18FC (gradient: 5-40% MeCN in water (10 mmol NH₄HCO₃)) then further purified by preparative HPLC (Column D, Eluent A, gradient: 8-30%) to give the title compound in the form of a trifluoroacetate salt (58 mg, 63%) as a white solid. ¹H NMR: (CD₃OD) δ 1.76-2.06 (2H, m), 2.23-2.27 (2H, m), 2.81-3.01 (5H, m), 3.08-3.23 (3H, m), 3.52-3.56 (2H, m), 3.77 (3H, s), 3.89 (2H, t), 6.23 (1H, s), 7.02 (1H, d), 7.21 (1H, t), 7.38 (1H, d). ¹⁹F NMR (376 MHz) δ −74.04. m/z (ES⁺), [M+H]⁺=341.1.

### Intermediate 90a: 6-(4-Benzylpiperazin-1-yl)-4-bromo-1H-indole

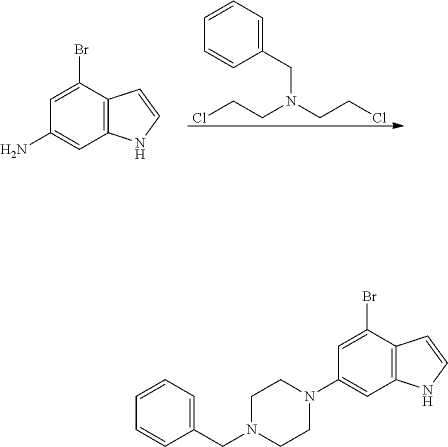

DIEA (12.4 ml, 71.0 mmol) was added to a solution of N-benzyl-2-chloro-N-(2-chloroethyl)ethan-1-amine (5.50 g, 23.7 mmol) and 4-bromo-1H-indol-6-amine (5.00 g, 23.7 mmol) in DMF (20 mL) at r.t. under air. The resulting solution was stirred at 100° C. for 2 h. The reaction mixture was poured into water (50 mL) and the resulting solid was filtered and dried to give the title compound (4.50 g, 51%) as a red solid. ¹H NMR: δ 3.12-3.19 (4H, m), 3.34-3.41 (2H, m), 3.72 (2H, d), 4.36-4.41 (2H, m), 6.26 (1H, s), 6.89 (1H, s), 7.03 (1H, s), 7.29 (1H, d), 7.45-7.51 (3H, m), 7.62-7.68 (2H, m), 11.23 (1H, s). m/z (ES⁺), [M+H]⁺=370.1.

### Intermediate 90b: 6-(4-Benzylpiperazin-1-yl)-4-bromo-1-methyl-1H-indole

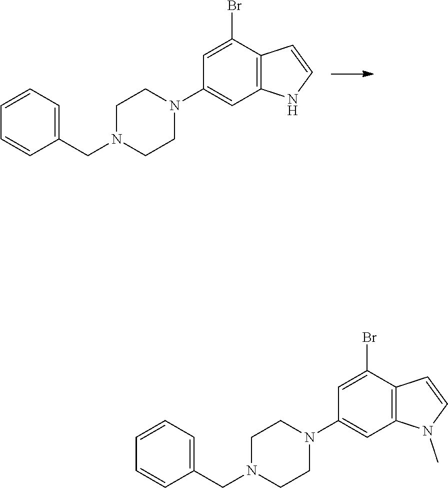 [SMILES: Brc1cc(N2CCN(Cc3ccccc3)CC2)cc2[nH]ccc12 ; Cn1ccc2c(Br)cc(N3CCN(Cc4ccccc4)CC3)cc21]

NaH (60% dispersion in mineral oil, 342 mg, 8.55 mmol) was added to a solution of 6-(4-benzylpiperazin-1-yl)-4-bromo-1H-indole (2.11 g, 5.70 mmol) in DMF (30 mL) at 0° C. under N₂. The resulting mixture was stirred at r.t. for 0.5 h before the addition of Mel (321 μl, 5.13 mmol). The resulting mixture was stirred at r.t. for 1 h. The reaction mixture was poured into water (50 mL) and extracted with EtOAc (3×50 mL). The combined organic extracts were dried (Na₂SO₄) and concentrated to give a brown solid. Purification by FSC (gradient: 0-50% EtOAc in petroleum ether) gave the title compound (2.00 g, 91%) as a white solid. ¹H NMR: δ 2.52-2.59 (4H, m), 3.11-3.20 (4H, m), 3.55 (2H, s), 3.72 (3H, s), 6.22 (1H, dd), 6.90 (1H, d), 7.00 (1H, d), 7.23 (1H, d), 7.32-7.37 (5H, m). m/z (ES⁺), [M+H]⁺=384.1.

### Example 90: 1-(1-Methyl-6-(piperazin-1-yl)-1H-indol-4-yl)dihydropyrimidine-2,4 (1H,3H)-dione

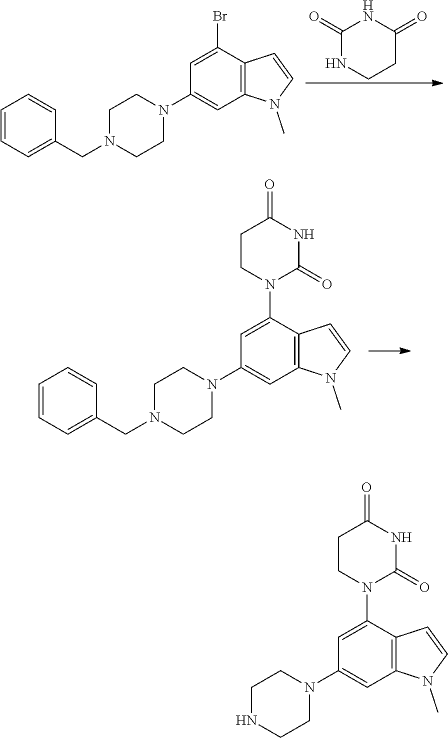 [SMILES: Cn1ccc2c(Br)cc(N3CCN(Cc4ccccc4)CC3)cc21 ; O=C1CCNC(=O)N1 ; Cn1ccc2c(N3CCC(=O)NC3=O)cc(N3CCN(Cc4ccccc4)CC3)cc21 ; Cn1ccc2c(N3CCC(=O)NC3=O)cc(N3CCNCC3)cc21]

Ephos (139 mg, 0.260 mmol) and Ephos Pd G4 (239 mg, 0.260 mmol) were added to a degassed mixture of Cs₂CO₃ (3.39 g, 10.4 mmol), 6-(4-benzylpiperazin-1-yl)-4-bromo-1-methyl-1H-indole (2.00 g, 5.20 mmol) and dihydropyrimidine-2,4 (1H,3H)-dione (1.78 g, 15.6 mmol) in 1,4-dioxane (40 mL) at r.t. under N₂. The resulting mixture was stirred at 100° C. for 16 h. The solvent was then removed under reduced pressure. Purification by C-18FC (gradient: 5-50% MeCN in water (containing 0.05% conc. HCl)) gave 1-(6-(4-benzylpiperazin-1-yl)-1-methyl-1H-indol-4-yl)dihydropyrimidine-2,4 (1H,3H)-dione (500 mg) as a black solid which was used in the next step without further purification. m/z (ES⁺), [M+H]⁺=418.2. The above solid was then dissolved in DMF (20 mL) and to the solution was added Pd/C (10% on activated carbon, 127 mg, 0.119 mmol). The reaction was stirred at r.t. under H₂ (1 atm) for 2 h. The reaction mixture was then filtered through a pad of celite. The solvent was then removed under reduced pressure. Purification by preparative HPLC (Column D, Eluent E, gradient: 5-13%) gave the title compound in the form of a formate salt (380 mg, 20% over two steps) as a white solid. ¹H NMR: δ 2.74 (2H, t), 2.91-2.98 (4H, m), 3.06-3.13 (4H, m), 3.73 (3H, s), 3.75 (2H, t), 6.23 (1H, d), 6.76 (1H, d), 6.83 (1H, d), 7.14 (1H, d), 8.24 (1H, s), 10.28 (1H, s). m/z (ES⁺), [M+H]⁺=328.3.

### Intermediate 91a: tert-Butyl 6-(4-benzylpiperazin-1-yl)-4-bromo-1H-indole-1-carboxylate

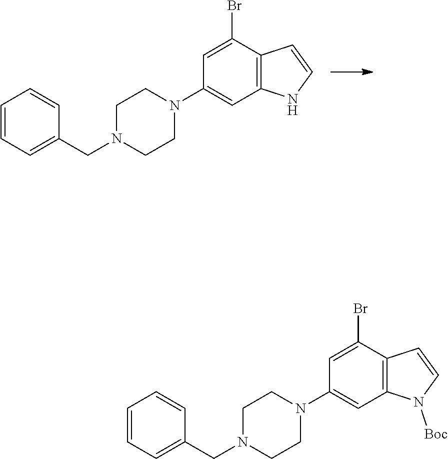 [SMILES: Brc1cc(N2CCN(Cc3ccccc3)CC2)cc2[nH]ccc12 ; CC(C)(C)OC(=O)n1ccc2c(Br)cc(N3CCN(Cc4ccccc4)CC3)cc21]

Triethylamine (2.26 mL, 16.2 mmol) was added to a mixture of DMAP (66.0 mg, 0.540 mmol), 6-(4-benzyl-piperazin-1-yl)-4-bromo-1H-indole (2.00 g, 5.40 mmol) and di-tert-butyl dicarbonate (2.36 g, 10.8 mmol) in DCM (20 mL) at r.t. The resulting mixture was stirred at r.t. for 2 h. The solvent was then removed under reduced pressure. Purification FSC (gradient: 0-50% EtOAc in petroleum ether) gave the title compound (1.70 g, 67%) as a purple solid. ¹H NMR: δ 1.61 (9H, s), 2.51-2.62 (4H, m), 3.14-3.23 (4H, m), 3.53 (2H, s), 6.46-6.52 (1H, m), 7.18 (1H, d), 7.23-7.30 (1H, m), 7.31-7.37 (4H, m), 7.51-7.60 (2H, m). m/z (ES⁺), [M+H]⁺=472.1.

### Example 91: 1-(6-(Piperazin-1-yl)-1H-indol-4-yl)dihydropyrimidine-2,4 (1H,3H)-dione

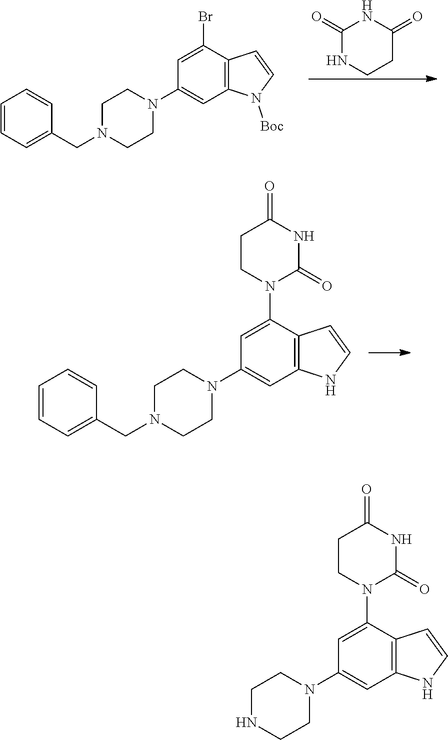 [SMILES: CC(C)(C)OC(=O)n1ccc2c(Br)cc(N3CCN(Cc4ccccc4)CC3)cc21 ; O=C1CCNC(=O)N1 ; O=C1CCN(c2cc(N3CCN(Cc4ccccc4)CC3)cc3[nH]ccc23)C(=O)N1 ; O=C1CCN(c2cc(N3CCNCC3)cc3[nH]ccc23)C(=O)N1]

Ephos (97.0 mg, 0.181 mmol) and Ephos Pd G4 (166 mg, 0.181 mmol) were added to Cs₂CO₃ (2.36 g, 7.24 mmol), tert-butyl 6-(4-benzylpiperazin-1-yl)-4-bromo-1H-indole-1-carboxylate (1.70 g, 3.61 mmol) and dihydropyrimidine-2,4 (1H,3H)-dione (1.24 g, 10.9 mmol) in DMF (40 mL) at r.t. under N₂. The resulting mixture was stirred at 100° C. for 16 h. The solvent was then removed under reduced pressure. Purification by C-18FC (gradient: 5-30% MeCN in water) gave 1-(6-(4-benzylpiperazin-1-yl)-1H-indol-4-yl)dihydropyrimidine-2,4 (1H,3H)-dione (300 mg) as a dark solid which was used in the next step without further purification. m/z (ES⁺), [M+H]⁺=404.2. The solid was then dissolved in DMF (15 mL) and to the solution was added Pd/C (10% on activated carbon, 79 mg, 0.074 mmol). The reaction was stirred at r.t. under H₂ (1 atm) for 2 h. The reaction mixture was then filtered through a pad of celite and washed with DMF (10 mL). The filtrate was concentrated to dryness. Purification by C-18FC (gradient: 5-30% MeCN in water (10 mmol NH₄HCO₃)) gave material that was further purified by preparative HPLC (Column D, Eluent A, gradient: 5-15%) to give the title compound in the form of a trifluoroacetate salt (120 mg, 8% over two steps) as a white solid. ¹H NMR: δ 2.75 (2H, t), 3.21-3.31 (8H, m), 3.77 (2H, t), 6.28 (1H, s), 6.78 (1H, s), 6.86 (1H, s), 7.22 (1H, d), 8.69 (2H, br s), 10.30 (1H, s), 10.99 (1H, s). m/z (ES⁺), [M+H]⁺=314.2.

### Intermediate 92a: tert-Butyl 4-(7-bromoimidazo[1,2-a]pyridin-2-yl)piperidine-1-carboxylate

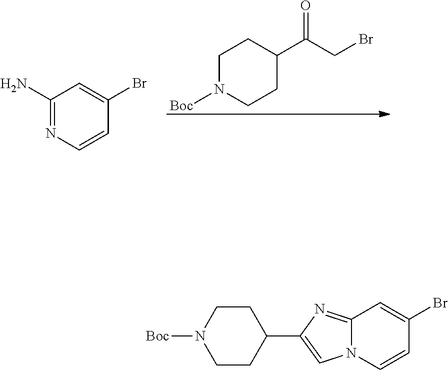 [SMILES: Nc1cc(Br)ccn1 ; CC(C)(C)OC(=O)N1CCC(C(=O)CBr)CC1 ; CC(C)(C)OC(=O)N1CCC(c2cn3ccc(Br)cc3n2)CC1]

Sodium bicarbonate (2.91 g, 34.7 mmol) was added to a solution of 4-bromopyridin-2-amine (2.00 g, 11.6 mmol) and tert-butyl 4-(2-bromoacetyl)piperidine-1-carboxylate (3.54 g, 11.6 mmol) in ethanol (50 mL) at r.t. The resulting mixture was stirred at 80° C. overnight. The solvent was then removed under reduced pressure. Purification FSC (gradient: 0-70% EtOAc in petroleum ether) gave the title compound (3.60 g, 82%) as a white solid. ¹H NMR: (CDCl₃) δ 1.49 (9H, s), 1.59-1.74 (2H, m), 2.04-2.13 (2H, m), 2.83-3.03 (3H, m), 4.21-4.28 (2H, m), 6.86 (1H, dd), 7.32 (1H, s), 7.71-7.76 (1H, m), 7.94 (1H, dd). m/z (ES⁺), [M+H]⁺=380.1.

### Intermediate 92b: tert-Butyl 4-(7-(2,4-dioxotetrahydropyrimidin-1 (2H)-yl)imidazo[1,2-a]pyridin-2-yl)piperidine-1-carboxylate

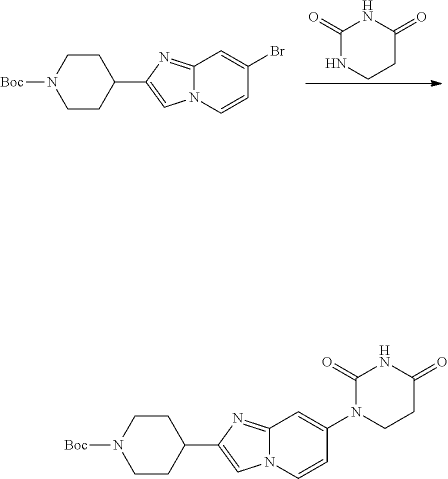 [SMILES: CC(C)(C)OC(=O)N1CCC(c2cn3ccc(Br)cc3n2)CC1 ; O=C1CCNC(=O)N1 ; CC(C)(C)OC(=O)N1CCC(c2cn3ccc(N4CCC(=O)NC4=O)cc3n2)CC1]

Cs₂CO₃ (1.29 g, 3.96 mmol) was added to a degassed mixture of Ephos (70.3 mg, 0.131 mmol), Ephos Pd G4 (121 mg, 0.132 mmol), dihydropyrimidine-2,4 (1H,3H)-dione (450 mg, 3.94 mmol) and tert-butyl 4-(7-bromoimidazo[1,2-a]pyridin-2-yl)piperidine-1-carboxylate (500 mg, 1.31 mmol) in 1,4-dioxane (30 mL) at r.t. under N₂. The resulting mixture was stirred at 100° C. overnight. The solvent was then removed under reduced pressure. Purification by C-18FC (gradient: 0-40% MeCN in water (containing 0.1% conc. HCl)) gave the title compound (320 mg, 59%) as a pale yellow solid. m/z (ES⁺), [M-tBu+2H]⁺=358.2.

### Example 92: 1-(2-(Piperidin-4-yl)imidazo[1,2-a]pyridin-7-yl)dihydropyrimidine-2,4 (1H,3H)-dione

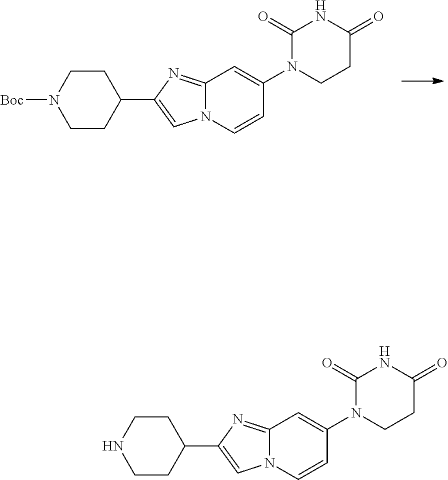 [SMILES: CC(C)(C)OC(=O)N1CCC(c2cn3ccc(N4CCC(=O)NC4=O)cc3n2)CC1 ; O=C1CCN(c2ccn3cc(C4CCNCC4)nc3c2)C(=O)N1]

A solution of HCl in 1,4-dioxane (4M, 5.00 mL, 20.0 mmol) was added to a solution of tert-butyl 4-(7-(2,4-dioxotetrahydropyrimidin-1 (2H)-yl)imidazo[1,2-a]pyridin-2-yl)piperidine-1-carboxylate (270 mg, 0.653 mmol) in DCM (5 mL) at r.t. under air. The resulting mixture was stirred at r.t. for 2 h. The solvent was then removed under reduced pressure. Purification by C-18FC (gradient: 0-2% MeCN in water (containing 0.1% conc. HCl)) gave the title compound in the form of a hydrochloride salt (120 mg, 53%) as a pale yellow solid. ¹H NMR: δ 1.74-1.90 (2H, m), 2.06-2.15 (2H, m), 2.74 (2H, t), 2.90-3.02 (3H, m), 3.24-3.33 (2H, m), 3.86 (2H, t), 6.95 (1H, dd), 7.38 (1H, d), 7.71 (1H, s), 8.34 (2H, s), 8.43 (1H, d), 10.50 (1H, s). m/z (ES⁺), [M+H]⁺=314.0.

### Intermediate 93a: tert-Butyl 4-(6-bromobenzo[d]isoxazol-3-yl)piperazine-1-carboxylate

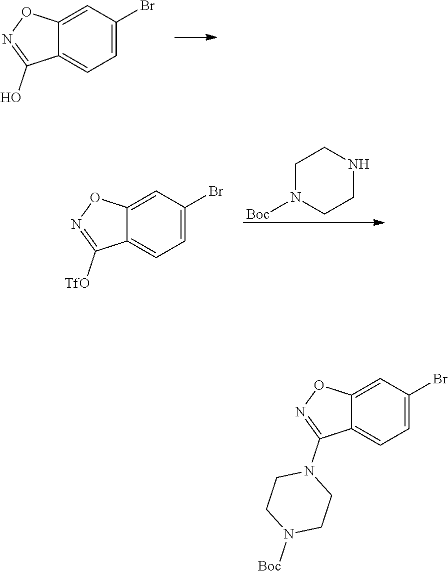 [SMILES: Oc1noc2cc(Br)ccc12 ; O=S(=O)(Oc1noc2cc(Br)ccc12)C(F)(F)F ; CC(C)(C)OC(=O)N1CCNCC1 ; CC(C)(C)OC(=O)N1CCN(c2noc3cc(Br)ccc23)CC1]

Trifluoromethanesulfonic anhydride (8.21 mL, 48.8 mmol) was added to a solution of 6-bromobenzo[d]-isoxazol-3-ol (9.50 g, 44.4 mmol) and pyridine (10.8 mL, 134 mmol) in DCM (200 mL) at 0° C. under air. The resulting mixture was stirred at r.t. for 4 h. The reaction mixture was then quenched with water (250 mL) and extracted with DCM (3×150 mL). The combined organic extracts were dried (Na₂SO₄) and concentrated to give 6-bromobenzo[d]isoxazol-3-yl trifluoromethanesulfonate as a yellow oil (15.0 g) which was used in the next step without further purification. The yellow oil was then dissolved in MeCN (300 mL) and to the solution was added sequentially tert-butyl piperazine-1-carboxylate (8.88 g, 47.7 mmol) and DIEA (15.1 mL, 86.5 mmol) at r.t. under air. The resulting mixture was stirred at 80° C. for 16 h. The solvent was then removed under reduced pressure. Purification FSC (gradient: 0-70% EtOAc in petroleum ether) gave the title compound (4.50 g, 27% over two-steps) as a yellow solid. ¹H NMR: δ 1.43 (9H, s), 3.44-3.48 (4H, m), 3.49-3.56 (4H, m), 7.49 (1H, dd), 7.96 (1H, s), 7.98 (1H, d). m/z (ES⁺), [M+H]⁺=382.0.

### Intermediate 93b: tert-Butyl 4-(6-(2,4-dioxotetrahydropyrimidin-1 (2H)-yl)benzo[d]isoxazol-3-yl)-piperazine-1-carboxylate

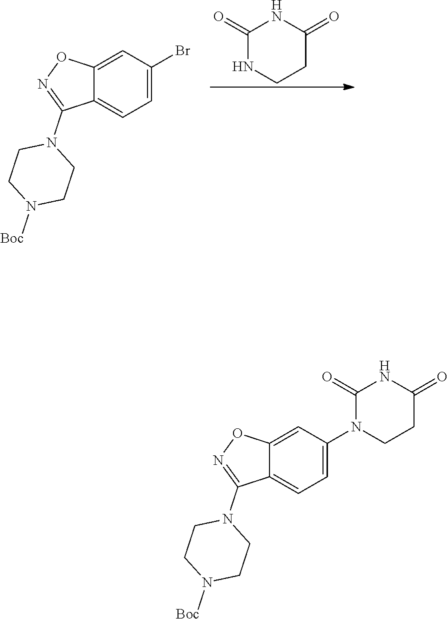 [SMILES: CC(C)(C)OC(=O)N1CCN(c2noc3cc(Br)ccc23)CC1 ; O=C1CCNC(=O)N1 ; CC(C)(C)OC(=O)N1CCN(c2noc3cc(N4CCC(=O)NC4=O)ccc23)CC1]

Ephos Pd G4 (541 mg, 0.589 mmol) was added to a degassed mixture of tert-butyl 4-(6-bromobenzo[d]isoxazol-3-yl)piperazine-1-carboxylate (4.50 g, 11.8 mmol), dihydropyrimidine-2,4 (1H,3H)-dione (4.03 g, 35.3 mmol), Ephos (315 mg, 0.589 mmol) and Cs₂CO₃ (7.67 g, 23.5 mmol) in 1,4-dioxane (200 mL) at r.t. under N₂. The resulting mixture was stirred at 100° C. for 16 h. The solvent was then removed under reduced pressure. Purification by FSC (gradient: 0-100% EtOAc in petroleum ether) gave the title compound (4.50 g, 92%) as a yellow solid. ¹H NMR: δ 1.43 (9H, s), 2.74 (2H, t), 3.41-3.59 (8H, m), 3.89 (2H, t), 7.32 (1H, dd), 7.56 (1H, d), 7.97 (1H, d), 10.50 (1H, s). m/z (ES⁺), [M+H]⁺=416.1.

### Example 93: 1-(3-(Piperazin-1-yl)benzo[d]isoxazol-6-yl)dihydropyrimidine-2,4 (1H,3H)-dione

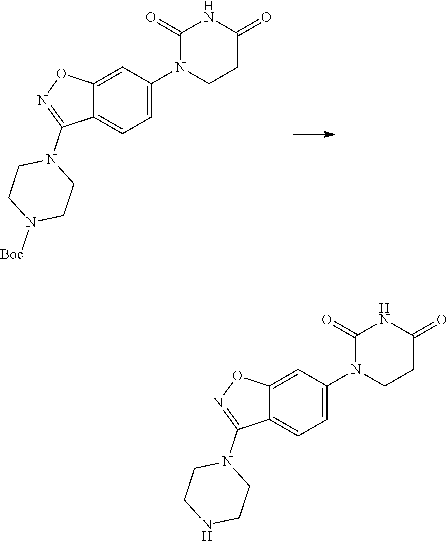 [SMILES: CC(C)(C)OC(=O)N1CCN(c2noc3cc(N4CCC(=O)NC4=O)ccc23)CC1 ; O=C1CCN(c2ccc3c(N4CCNCC4)noc3c2)C(=O)N1]

4-(6-(2,4-Dioxotetrahydropyrimidin-1 (2H)-yl)benzo[d]isoxazol-3-yl)piperazine-1-carboxylate (1.40 g, 3.37 mmol) was added to a solution of 1,4-dioxane (100 mL) and HCl in 1,4-dioxane (4M, 100 mL) at r.t. The resulting solution was stirred at r.t. for 2 h. The solvent was then removed under reduced pressure. Purification C-18FC (gradient: 5-40% MeCN in water) gave the title compound in the form of a hydrochloride salt (1.00 g, 84%) as a white solid. ¹H NMR: δ 2.73 (2H, t), 3.29 (4H, t), 3.68 (4H, t), 3.86 (2H, t), 7.33 (1H, dd), 7.57 (1H, d), 7.96 (1H, d). m/z (ES⁺), [M+H]⁺=316.2.

### Example 94: 1-(3-(4-Methylpiperazin-1-yl)benzo[d]isoxazol-6-yl)dihydropyrimidine-2,4 (1H,3H)-dione

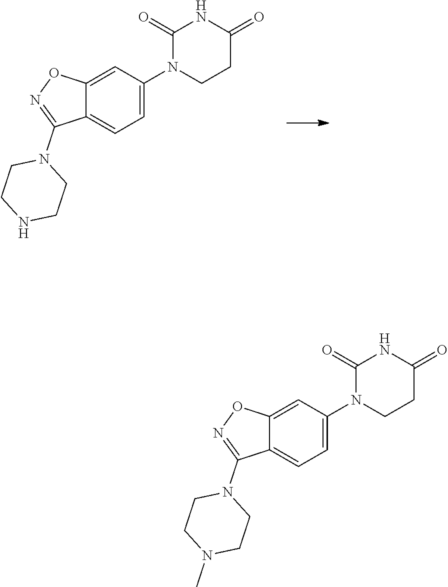 [SMILES: O=C1CCN(c2ccc3c(N4CCNCC4)noc3c2)C(=O)N1 ; CN1CCN(c2noc3cc(N4CCC(=O)NC4=O)ccc23)CC1]

Sodium triacetoxyborohydride (271 mg, 1.28 mmol) was added to a mixture of 1-(3-(piperazin-1-yl)benzo[d]-isoxazol-6-yl)dihydropyrimidine-2,4 (1H,3H)-dione hydrochloride (150 mg, 0.426 mmol), paraformaldehyde (25.6 mg, 0.853 mmol) and NaOAc (105 mg, 1.28 mmol) in DCM (10 mL) at r.t. The resulting solution was stirred at r.t. for 16 h. The solvent was then removed under reduced pressure. Purification by C-18FC (gradient: 0-40% MeCN in water) gave the title compound (40.0 mg, 28%) as a white solid. ¹H NMR: δ 2.24 (3H, s), 2.48 (4H, m), 2.74 (2H, t), 3.42-3.54 (4H, m), 3.88 (2H, t), 7.30 (1H, dd), 7.55 (1H, d), 7.96 (1H, d). m/z (ES⁺), [M+H]⁺=330.2.

### Example 95: 1-(3-(4-Acetylpiperazin-1-yl)benzo[d]isoxazol-6-yl)dihydropyrimidine-2,4 (1H,3H)-dione

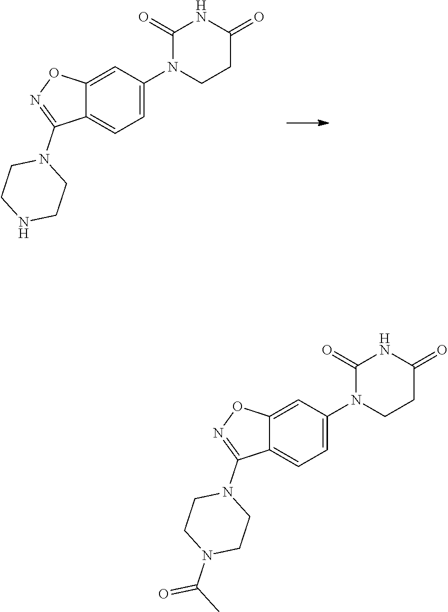 [SMILES: O=C1CCN(c2ccc3c(N4CCNCC4)noc3c2)C(=O)N1 ; CC(=O)N1CCN(c2noc3cc(N4CCC(=O)NC4=O)ccc23)CC1]

Ac₂O (52.3 μL, 0.554 mmol) was added to a solution of 1-(3-(piperazin-1-yl)benzo[d]isoxazol-6-yl)dihydro-pyrimidine-2,4 (1H,3H)-dione hydrochloride (150 mg, 0.426 mmol) and triethylamine (238 μL, 1.71 mmol) in DCM (10 mL) at r.t. The resulting solution was stirred at r.t. for 16 h. The solvent was then removed under reduced pressure. Purification by C-18FC (gradient: 0-40% MeCN in water) gave the title compound (108 mg, 71%) as a white solid. ¹H NMR: δ 2.05 (3H, s), 2.73 (2H, t), 3.39-3.52 (4H, m), 3.57-3.68 (4H, m), 3.87 (2H, t), 7.31 (1H, dd), 7.56 (1H, d), 7.97 (1H, d), 10.48 (1H, s). m/z (ES⁺), [M+H]⁺=358.2.

### Intermediate 96a: 6-Bromo-2-(piperidin-4-yl)benzo[d]oxazole

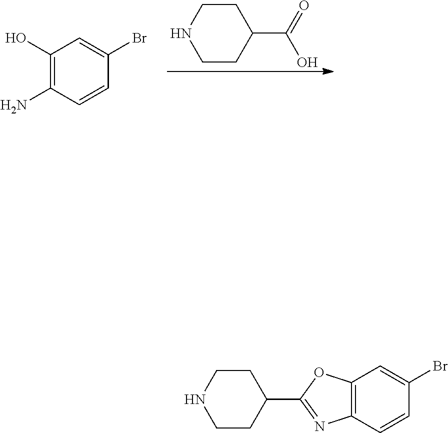 [SMILES: Nc1ccc(Br)cc1O ; O=C(O)C1CCNCC1 ; Brc1ccc2nc(C3CCNCC3)oc2c1]

Piperidine-4-carboxylic acid (687 mg, 5.32 mmol) was added to a solution of 2-amino-5-bromophenol (1.00 g, 5.32 mmol) in PPA (40 mL). The resulting mixture was stirred at 190° C. for 5 h. The reaction mixture was adjusted to pH=8 using a solution of NaOH (aq 10%). The mixture was then poured into water (500 mL) and extracted with EtOAc (6×500 mL). The combined organic extracts were dried (Na₂SO₄) and concentrated to give a dark solid (800 mg) which was used directly in the next step without further purification. m/z (ES⁺), [M+H]⁺=281.0.

### Intermediate 96b: tert-Butyl 4-(6-bromobenzo[d]oxazol-2-yl)piperidine-1-carboxylate

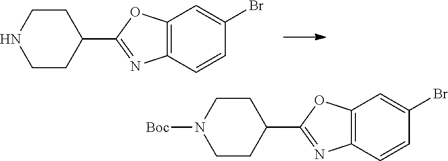

The crude product of the above reaction (Intermediate 96a) was dissolved in DCM (20 mL) and to the mixture were added sequentially di-tert-butyl dicarbonate (991 μL, 4.27 mmol) and DIEA (1.49 mL, 8.53 mmol) at r.t. The resulting mixture was stirred at r.t. for 2 h. The solvent was then removed under reduced pressure. Purification by FSC (gradient: 0-25% EtOAc in petroleum ether) gave the title compound (380 mg, 19% over two-steps) as a brown solid. ¹H NMR: δ 1.42 (9H, s), 1.60-1.74 (2H, m), 2.04-2.13 (2H, m), 2.99 (2H, br s), 3.19-3.31 (1H, m), 3.91-3.99 (2H, m), 7.53 (1H, dd), 7.68 (1H, d), 8.03 (1H, d). m/z (ES⁺), [M+H]⁺=381.2.

### Intermediate 96c: tert-Butyl 4-(6-(2,4-dioxotetrahydropyrimidin-1 (2H)-yl)benzo[d]oxazol-2-yl)piperidine-1-carboxylate

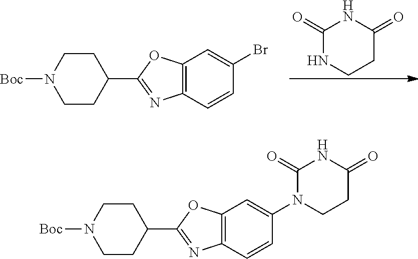 [SMILES: CC(C)(C)OC(=O)N1CCC(c2nc3ccc(Br)cc3o2)CC1 ; O=C1CCNC(=O)N1 ; CC(C)(C)OC(=O)N1CCC(c2nc3ccc(N4CCC(=O)NC4=O)cc3o2)CC1]

Ephos (49.1 mg, 0.0918 mmol) and Ephos Pd G4 (84.0 mg, 0.0914 mmol) were added to a mixture of Cs₂CO₃ (598 mg, 1.84 mmol), tert-butyl 4-(6-bromobenzo[d]oxazol-2-yl)piperidine-1-carboxylate (350 mg, 0.92 mmol) and dihydropyrimidine-2,4 (1H,3H)-dione (314 mg, 2.75 mmol) in DMF (15 mL) at r.t. under N₂. The resulting mixture was stirred at 100° C. for 16 h. The solvent was then removed under reduced pressure. Purification by FSC (gradient: 0-8% MeOH in DCM) gave the title compound (220 mg, 58%) as a brown solid. ¹H NMR: δ 1.42 (9H, s), 1.60-1.77 (2H, m), 2.09 (2H, dd), 2.94-3.08 (3H, m), 3.21-3.31 (2H, m), 3.83 (2H, t), 3.92-3.99 (2H, m), 7.32 (1H, dd), 7.70 (2H, dd), 10.42 (1H, s). m/z (ES⁺), [M-tBu+2H]⁺=359.2.

### Example 96: 1-(2-(Piperidin-4-yl)benzo[d]oxazol-6-yl)dihydropyrimidine-2,4 (1H,3H1)-dione

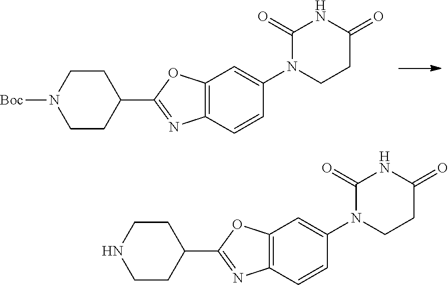 [SMILES: CC(C)(C)OC(=O)N1CCC(c2nc3ccc(N4CCC(=O)NC4=O)cc3o2)CC1 ; O=C1CCN(c2ccc3nc(C4CCNCC4)oc3c2)C(=O)N1]

tert-Butyl 4-(6-(2,4-dioxotetrahydropyrimidin-1 (2H)-yl)benzo[d]oxazol-2-yl)piperidine-1-carboxylate (230 mg, 0.555 mmol) was dissolved in 2,2,2-trifluoroethanol (6 mL) and sealed into a microwave tube. The reaction was heated to 140° C. for 10 h in a microwave reactor. The reaction was then cooled to r.t. and the solvent was removed under reduced pressure. Purification by C-18FC (gradient: 0-20% MeCN in water (containing 0.1% FA)) gave the title compound in the form of a formate salt (51.9 mg, 26%) as a white solid. ¹H NMR: δ 1.75-1.86 (2H, m), 2.07-2.16 (2H, m), 2.74 (2H, t), 2.87-2.96 (2H, m), 3.15-3.21 (3H, m), 3.82 (2H, t), 7.31 (1H, d), 7.66-7.73 (2H, m), 8.31 (1H, s), 10.40 (1H, s). m/z (ES⁺), [M+H]⁺=315.0.

### Example 97: 1-(2-(1-Methylpiperidin-4-yl)benzo[d]oxazol-6-yl)dihydropyrimidine-2,4 (1H,3H)-dione

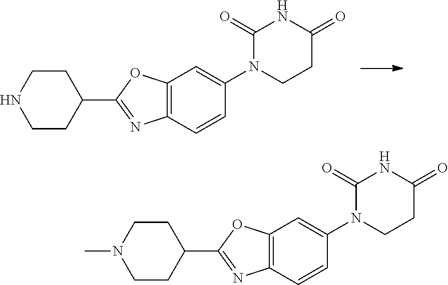 [SMILES: O=C1CCN(c2ccc3nc(C4CCNCC4)oc3c2)C(=O)N1 ; CN1CCC(c2nc3ccc(N4CCC(=O)NC4=O)cc3o2)CC1]

NaOAc (34.1 mg, 0.416 mmol) was added to a mixture of 1-(2-(piperidin-4-yl)benzo[d]oxazol-6-yl)dihydro-pyrimidine-2,4 (1H,3H)-dione formate (50.0 mg, 0.139 mmol), sodium triacetoxyborohydride (88.0 mg, 0.415 mmol) and paraformaldehyde (12.5 mg, 0.416 mmol) in DCM (15 mL). The resulting mixture was stirred at r.t. for 1 h. The solvent was then removed under reduced pressure. Purification by C-18FC (gradient: 0-15% MeCN in water (containing 0.1% FA)) gave the title compound in the form of a diformate salt (19.8 mg, 34%) as a white solid. ¹H NMR: δ 1.80-1.91 (2H, m), 2.07-2.16 (4H, m), 2.22 (3H, s), 2.74 (2H, t), 2.82 (2H, d), 2.97-3.01 (1H, m), 3.82 (2H, t), 7.31 (1H, d), 7.65-7.72 (2H, m), 8.17 (2H, s), 10.40 (1H, s). m/z (ES⁺), [M+H]⁺=329.2.

### Intermediate 98a: 6-Bromobenzo[d]oxazole-2 (3H)-thione

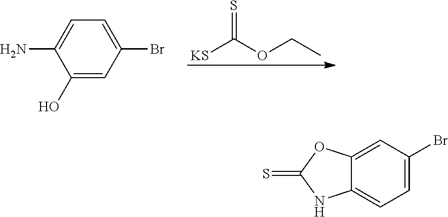

Potassium O-ethyl carbonodithioate (4.26 g, 26.6 mmol) was added to a solution of 2-amino-5-bromophenol (5.00 g, 26.6 mmol) in EtOH (60 mL) at r.t. under N₂. The resulting mixture was stirred at 80° C. for 16 h. The solvent was then removed under reduced pressure. The reaction mixture was diluted with EtOAc (300 mL) and washed sequentially with 2M HCl (50 mL), water (100 mL) and saturated brine (100 mL). The organic layer was dried (Na₂SO₄) and concentrated to give the title compound (4.60 g, 75%) as a brown solid which was used in the next step without further purification. ¹H NMR: δ 7.17 (1H, d), 7.45 (1H, dd), 7.83 (1H, d), 13.70 (1H, s). m/z (ES⁺), [M+H]⁺=230.2.

### Intermediate 98b: tert-Butyl 4-(6-bromobenzo[d]oxazol-2-yl)piperazine-1-carboxylate

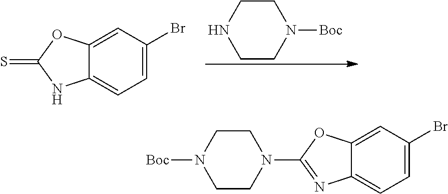 [SMILES: S=c1[nH]c2ccc(Br)cc2o1 ; CC(C)(C)OC(=O)N1CCNCC1 ; CC(C)(C)OC(=O)N1CCN(c2nc3ccc(Br)cc3o2)CC1]

Two of the following reactions were run in parallel: A mixture of 6-bromobenzo[d]oxazole-2 (3H)-thione (1.00 g, 4.35 mmol), tert-butyl piperazine-1-carboxylate (1.62 g, 8.70 mmol) and DIEA (2.28 mL, 13.1 mmol) in n-butanol (12 mL) was sealed into a microwave tube. The reaction was heated to 150° C. for 1 h in a microwave reactor and then cooled to r.t. The solvent was then removed under reduced pressure. The combined crude products of the two parallel reactions were purified by FSC (gradient: 0-23% EtOAc in petroleum ether) to give the title compound (2.70 g, 81% average of two reactions) as an off-white solid. ¹H NMR: δ 1.41 (9H, s), 3.41-3.51 (4H, m), 3.52-3.62 (4H, m), 7.23 (1H, d), 7.31 (1H, dd), 7.68 (1H, d). m/z (ES⁺), [M+H]⁺=382.1.

### Intermediate 98c: tert-Butyl 4-(6-(2,4-dioxotetrahydropyrimidin-1 (2H)-yl)benzo[d]oxazol-2-yl)piperazine-1-carboxylate

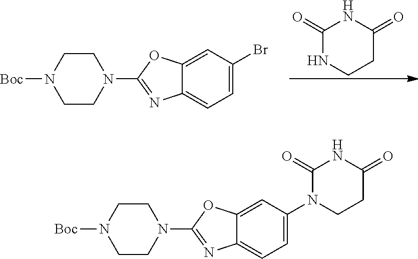 [SMILES: CC(C)(C)OC(=O)N1CCN(c2nc3ccc(Br)cc3o2)CC1 ; O=C1CCNC(=O)N1 ; CC(C)(C)OC(=O)N1CCN(c2nc3ccc(N4CCC(=O)NC4=O)cc3o2)CC1]

Ephos Pd G4 (231 mg, 0.251 mmol) was added to a degassed mixture of tert-butyl 4-(6-bromobenzo[d]oxazol-2-yl)piperazine-1-carboxylate (1.60 g, 4.19 mmol), dihydropyrimidine-2,4 (1H,3H)-dione (1.43 g, 12.5 mmol), Cs₂CO₃ (4.09 g, 12.6 mmol) and Ephos (134 mg, 0.251 mmol) in 1,4-dioxane (80 mL) at r.t. under N₂. The resulting mixture was stirred at 100° C. for 16 h. The solvent was then removed under reduced pressure. Purification by FSC (gradient: 0-100% EtOAc in petroleum ether, then maintained at 100% for 0.5 h) gave the title compound (1.50 g, 86%) as a white solid. ¹H NMR: δ 1.43 (9H, s), 2.71 (2H, t), 3.47 (4H, dd), 3.59 (4H, dd), 3.76 (2H, t), 7.12 (1H, dd), 7.28 (1H, d), 7.44 (1H, d), 10.35 (1H, s). m/z (ES⁺), [M+H]⁺=416.2.

### Example 98: 1-(2-(Piperazin-1-yl)benzo[d]oxazol-6-yl)dihydropyrimidine-2,4 (1H,3H)-dione

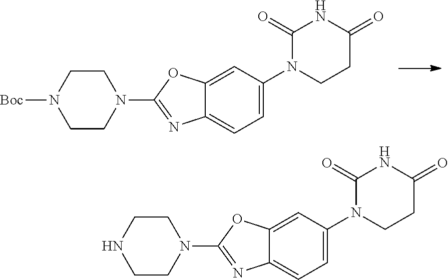 [SMILES: CC(C)(C)OC(=O)N1CCN(c2nc3ccc(N4CCC(=O)NC4=O)cc3o2)CC1 ; O=C1CCN(c2ccc3nc(N4CCNCC4)oc3c2)C(=O)N1]

Two of the following reactions were run in parallel: A mixture of tert-butyl 4-(6-(2,4-dioxotetrahydropyrimidin-1 (2H)-yl)benzo[d]oxazol-2-yl)piperazine-1-carboxylate (700 mg, 1.68 mmol) and 2,2,2-trifluoroethanol (15 mL) were sealed into a microwave tube. The reaction was heated to 150° C. for 8 h in a microwave reactor and then cooled to r.t. The solvent was then removed under reduced pressure. The combined crude products of the two parallel reactions were purified by C-18FC (gradient: 0-35% MeCN in water (10 mmol NH₄HCO₃)) to give the title compound (900 mg, 85% average of two reactions) as a white solid. ¹H NMR: δ 2.71 (2H, t), 2.75-2.82 (4H, m), 3.48-3.55 (4H, m), 3.76 (2H, t), 7.10 (1H, dd), 7.25 (1H, d), 7.41 (1H, d), 10.35 (1H, s). m/z (ES⁺), [M+H]⁺=316.3.

### Example 99: 1-(2-(4-Methylpiperazin-1-yl)benzo[d]oxazol-6-yl)dihydropyrimidine-2,4 (1H,3H)-dione

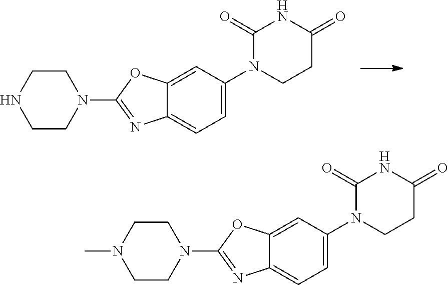 [SMILES: O=C1CCN(c2ccc3nc(N4CCNCC4)oc3c2)C(=O)N1 ; CN1CCN(c2nc3ccc(N4CCC(=O)NC4=O)cc3o2)CC1]

Sodium triacetoxyborohydride (101 mg, 0.477 mmol) was added to a mixture of 1-(2-(piperazin-1-yl)benzo[d]-oxazol-6-yl)dihydropyrimidine-2,4 (1H,3H)-dione (50.0 mg, 0.159 mmol), paraformaldehyde (14.3 mg, 0.476 mmol) and AcOH (27.3 μL, 0.477 mmol) in DCM (5 mL) at r.t. The resulting mixture was stirred at r.t. for 16 h. The solvent was then removed under reduced pressure. Purification by C-18FC (gradient: 0-5% MeCN in water (containing 0.1% TFA)) provided a residue which was further purified by preparative HPLC (Column B, Eluent G, gradient: 10-35%) to give the title compound (16.0 mg, 31%) as a white solid. ¹H NMR: δ 2.23 (3H, s), 2.40-2.50 (4H, m), 2.71 (2H, t), 3.60 (4H, t), 3.76 (2H, t), 7.11 (1H, dd), 7.26 (1H, d), 7.42 (1H, d), 10.34 (1H, s). m/z (ES⁺), [M+H]⁺=330.2.

### Intermediates 100a & 100b: tert-Butyl 4-(5-bromobenzo[d]thiazol-2-yl)piperazine-1-carboxylate & tert-butyl 4-(7-bromobenzo[d]thiazol-2-yl)piperazine-1-carboxylate

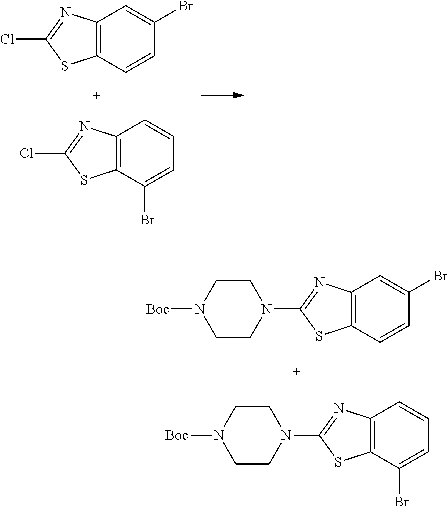 [SMILES: Clc1nc2cc(Br)ccc2s1 ; Clc1nc2cccc(Br)c2s1 ; CC(C)(C)OC(=O)N1CCN(c2nc3cc(Br)ccc3s2)CC1 ; CC(C)(C)OC(=O)N1CCN(c2nc3cccc(Br)c3s2)CC1]

K₂CO₃ (3.34 g, 24.2 mmol) was added to an isomeric 1.6:1 mixture of 5-bromo-2-chlorobenzo[d]thiazole and 7-bromo-2-chlorobenzo[d]thiazole (3.00 g, 12.1 mmol, commercial reagent—initially presumed pure & later discovered to be a mixture of isomers) and tert-butyl piperazine-1-carboxylate (2.25 g, 12.1 mmol) in DMF (30 mL) at r.t. under air. The resulting solution was stirred at 80° C. for 4 h. The reaction mixture was then poured into water (150 mL), extracted with EtOAc (3×100 mL). The combined organic extracts were dried (Na₂SO₄) and concentrated to give a pale yellow liquid. Purification by FSC (gradient: 0-50% EtOAc in petroleum ether) gave the title compounds as an inseparable isomeric mixture (1.6:1, 4.50 g, 94%) as a pale yellow solid. ¹H NMR peaks of the major isomer (Intermediate 100a): δ 1.41 (9H, s), 3.42-3.51 (4H, m), 3.51-3.62 (4H, m), 7.21 (1H, dd), 7.60 (1H, d), 7.73 (1H, d). m/z (ES⁺), [M+H]⁺=400.1.

### Intermediates 100c & 100d: tert-Butyl 4-(5-(2,4-dioxotetrahydropyrimidin-1 (2H)-yl)benzo[d]thiazol-2-yl)piperazine-1-carboxylate & tert-butyl 4-(7-(2,4-dioxotetrahydropyrimidin-1 (2H)-yl)benzo[d]thiazol-2-yl)piperazine-1-carboxylate

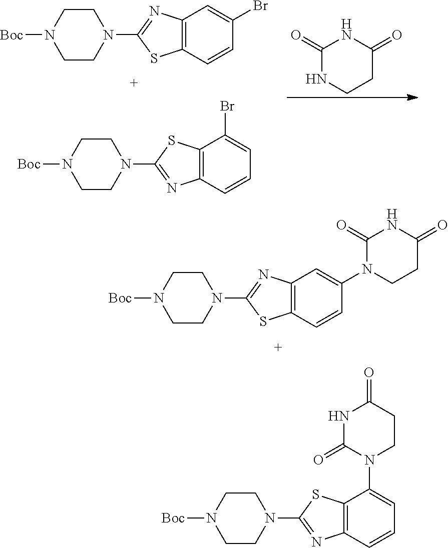 [SMILES: CC(C)(C)OC(=O)N1CCN(c2nc3cc(Br)ccc3s2)CC1 ; O=C1CCNC(=O)N1 ; CC(C)(C)OC(=O)N1CCN(c2nc3cccc(Br)c3s2)CC1 ; CC(C)(C)OC(=O)N1CCN(c2nc3cc(N4CCC(=O)NC4=O)ccc3s2)CC1 ; CC(C)(C)OC(=O)N1CCN(c2nc3cccc(N4CCC(=O)NC4=O)c3s2)CC1]

1^{st} experiment: Ephos Pd G4 (115 mg, 0.125 mmol) was added to a degassed mixture of an isomeric 1.6:1 mixture of tert-butyl 4-(5-bromobenzo[d]thiazol-2-yl)piperazine-1-carboxylate and tert-butyl 4-(7-bromobenzo-[d]thiazol-2-yl)piperazine-1-carboxylate (1.00 g, 2.51 mmol), dihydropyrimidine-2,4 (1H,3H)-dione (859 mg, 7.53 mmol), Ephos (67.0 mg, 0.125 mmol) and Cs₂CO₃ (1.636 g, 5.02 mmol) in 1,4-dioxane (10 mL) at r.t. under N₂. The resulting solution was stirred at 120° C. for 12 h. The reaction mixture was then concentrated to give the crude product.

2^{nd} experiment: The 1^{st} experiment was repeated and the crude products of both reactions were combined. Purification by C-18FC (gradient: 20-70% MeCN in water) gave the title compounds as an isomeric mixture (3:1, 1.100 g, 51% average yield for the two experiments) as a white solid. ¹H NMR:-peaks of the major isomer (Intermediate 100c): δ 1.41 (9H, s), 2.70 (2H, t), 3.42-3.51 (4H, m), 3.51-3.58 (4H, m), 3.78 (2H, t), 7.04 (1H, dd), 7.41 (1H, d), 7.75 (1H, d), 10.33 (1H, s). m/z (ES⁺), [M+H]⁺=432.3.

### Example 100: 1-(2-(Piperazin-1-yl)benzo[d]thiazol-5-yl)dihydropyrimidine-2,4 (1H,3H)-dione

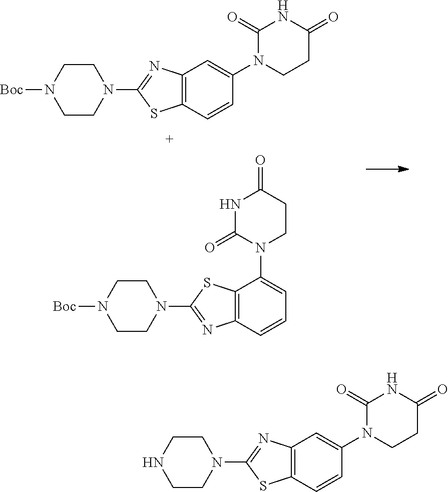 [SMILES: CC(C)(C)OC(=O)N1CCN(c2nc3cc(N4CCC(=O)NC4=O)ccc3s2)CC1 ; CC(C)(C)OC(=O)N1CCN(c2nc3cccc(N4CCC(=O)NC4=O)c3s2)CC1 ; O=C1CCN(c2ccc3sc(N4CCNCC4)nc3c2)C(=O)N1]

An isomeric mixture (3:1) of tert-butyl 4-(5-(2,4-dioxotetrahydropyrimidin-1 (2H)-yl)benzo[d]thiazol-2-yl)piperazine-1-carboxylate & tert-butyl 4-(7-(2,4-dioxotetrahydropyrimidin-1 (2H)-yl)benzo[d]thiazol-2-yl)piperazine-1-carboxylate (100 mg, 0.232 mmol) was added to a solution of HCl in 1,4-dioxane (4M, 2 mL, 8.00 mmol) at r.t. The resulting mixture was stirred at r.t. for 1 h. The solvent was then removed under reduced pressure. Purification by preparative SFC (Column: DAICEL DCpak P4VP, 20*250 mm, 5 μm; mobile phase A: scCO₂, mobile phase B: MeOH (8 mmol NH₃·MeOH)-HPLC; Flow rate: 50 mL/min; Gradient: 20% B; 254 nm; r.t.1:3.72; r.t.2:4.48; Injection Volume: 2 mL; Number Of Runs: 10) gave the title compound (33.2 mg, 43%) as a white solid. ¹H NMR: δ 10.33 (s, 1H), 7.74 (d, 1H), 7.40 (d, 1H), 7.03 (dd, 1H), 3.80 (t, 2H), 3.48 (t, 4H), 2.81 (t, 4H), 2.72 (t, 2H). m/z (ES⁺), [M+H]⁺=332.2.

### Example 101: 1-(2-(Piperazin-1-yl)benzo[d]thiazol-7-yl)dihydropyrimidine-2,4 (1H,3H)-dione

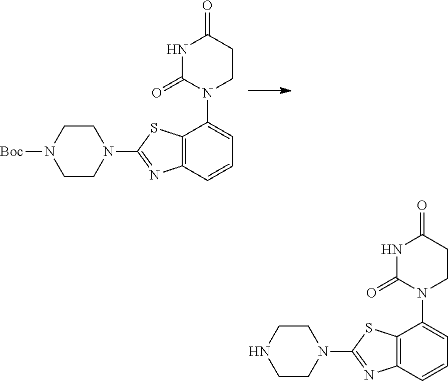 [SMILES: CC(C)(C)OC(=O)N1CCN(c2nc3cccc(N4CCC(=O)NC4=O)c3s2)CC1 ; O=C1CCN(c2cccc3nc(N4CCNCC4)sc23)C(=O)N1]

tert-Butyl 4-(7-(2,4-dioxotetrahydropyrimidin-1 (2H)-yl)benzo[d]thiazol-2-yl)piperazine-1-carboxylate (the stated compound was isolated via preparative TLC of the 3:1 isomeric mixture of intermediate 100c and 100d respectively) (30.0 mg, 0.070 mmol) was added to a solution of HCl in 1,4-dioxane (4 M, 1 mL, 4.00 mmol) at r.t. The resulting mixture was stirred at r.t. for 1 h. The precipitate was collected by filtration and washed with Et₂O to give the title compound in the form of a dihydrochloride salt (23.7 mg, 84%) as a white solid. ¹H NMR: δ 2.74 (2H, t), 3.18-3.30 (4H, m), 3.75-3.88 (6H, m), 7.14 (1H, dd), 7.38 (1H, t), 7.46 (1H, dd), 9.32 (2H, br s), 10.53 (1H, s). m/z (ES⁺), [M+H]⁺=332.1.

### Intermediate 102a: Ethyl 6-bromo-1-(cyanomethyl)-1H-indole-2-carboxylate

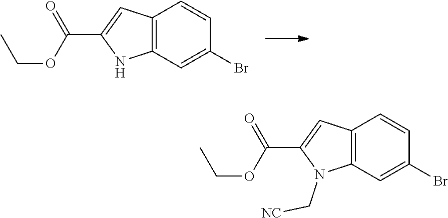 [SMILES: CCOC(=O)c1cc2ccc(Br)cc2[nH]1 ; CCOC(=O)c1cc2ccc(Br)cc2n1CC#N]

KOtBu (1.63 g, 14.5 mmol) was added to ethyl 6-bromo-1H-indole-2-carboxylate (2.60 g, 9.70 mmol) in DMF (20 mL) at r.t. The resulting mixture was stirred for 1 h before the addition of 2-chloroacetonitrile (879 mg, 11.6 mmol). The resulting mixture was stirred at r.t. for 3 h. The reaction mixture was then diluted with water. The precipitate was collected by filtration, washed with water and dried under vacuum to give the title compound (2.00 g, 67%) as a grey solid, which was used in the next step without further purification. ¹H NMR: δ 1.36 (3H, t), 4.37 (2H, q), 5.76 (2H, s), 7.38 (1H, dd), 7.43 (1H, d), 7.72 (1H, d), 8.09-8.20 (1H, m).

### Intermediate 102b: 7-Bromo-1,2,3,4-tetrahydropyrazino[1,2-a]indole

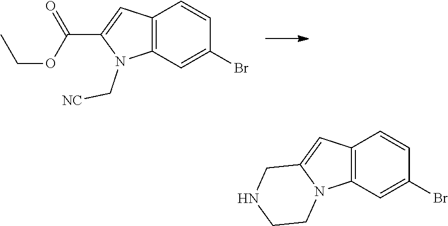 [SMILES: CCOC(=O)c1cc2ccc(Br)cc2n1CC#N ; Brc1ccc2cc3n(c2c1)CCNC3]

A solution of LiAlH₄ in THF (1M, 18.0 mL, 18.0 mmol) was added dropwise to a solution of ethyl 6-bromo-1-(cyanomethyl)-1H-indole-2-carboxylate (1.80 g, 5.86 mmol) in THF (30 mL) at r.t. under N₂. The resulting mixture was stirred at 60° C. for 2 h. The reaction mixture was then quenched with a saturated solution of Rochelle's salt (200 mL) and extracted with EtOAc (3×100 mL). The organic layer was dried (Na₂SO₄) and evaporated. Purification by C-18FC (gradient: 5-30% MeCN in water (containing 0.1% NH₄HCO₃)) gave the title compound (500 mg, 34%) as a yellow solid. ¹H NMR: δ 3.18 (2H, t), 3.96 (2H, t), 4.04 (2H, s), 6.17 (1H, s), 7.12 (1H, dd), 7.41 (1H, d), 7.60 (1H, d). m/z (ES⁺), [M+H]⁺=250.9.

### Intermediate 102c: tert-Butyl 7-bromo-3,4-dihydropyrazino[1,2-a]indole-2 (1H)-carboxylate

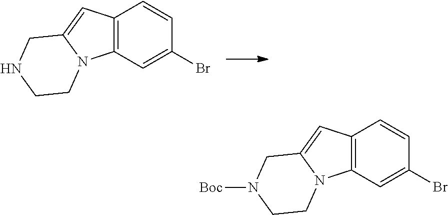 [SMILES: Brc1ccc2cc3n(c2c1)CCNC3 ; CC(C)(C)OC(=O)N1CCn2c(cc3ccc(Br)cc32)C1]

Di-tert-butyl dicarbonate (416 μL, 1.79 mmol) was added to a mixture of 7-bromo-1,2,3,4-tetrahydro-pyrazino[1,2-a]indole (300 mg, 1.19 mmol) in THF (2 mL) and an aqueous solution of saturated Na₂CO₃ (2 mL) at r.t. The resulting mixture was stirred at r.t. for 16 h. The reaction mixture was then diluted with EtOAc, and washed sequentially with water and saturated brine. The organic layer was dried (Na₂SO₄) and concentrated. Purification by FSC (gradient: 0-5% EtOAc in petroleum ether) gave the title compound (304 mg, 72%) as a yellow solid. ¹H NMR: δ 1.42 (9H, s), 3.82 (2H, t), 4.08 (2H, dd), 4.71 (2H, s), 6.31 (1H, s), 7.13 (1H, dd), 7.43 (1H, d), 7.64 (1H, d). m/z (ES⁺), [M+H]⁺=351.1.

### Intermediate 102d: tert-Butyl 7-(2,4-dioxotetrahydropyrimidin-1 (2H)-yl)-3,4-dihydropyrazino[1,2-a]-indole-2 (1H)-carboxylate

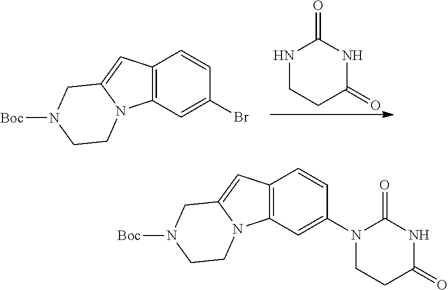 [SMILES: CC(C)(C)OC(=O)N1CCn2c(cc3ccc(Br)cc32)C1 ; O=C1CCNC(=O)N1 ; CC(C)(C)OC(=O)N1CCn2c(cc3ccc(N4CCC(=O)NC4=O)cc32)C1]

Ephos Pd G4 (68.0 mg, 0.0740 mmol) was added to a degassed mixture of tert-butyl 7-bromo-3,4-dihydro-pyrazino[1,2-a]indole-2 (1H)-carboxylate (260 mg, 0.740 mmol), dihydropyrimidine-2,4 (1H,3H)-dione (253 mg, 2.22 mmol), Ephos (39.5 mg, 0.0739 mmol) and Cs₂CO₃ (482 mg, 1.48 mmol) in 1,4-dioxane (7 mL) at r.t. under N₂. The resulting mixture was stirred at 120° C. for 16 h. The solvent was then removed under reduced pressure. Purification by FSC (gradient: 0-70% EtOAc in petroleum ether) gave the title compound (130 mg, 46%) as a yellow solid. ¹H NMR: δ 10.30 (s, 1H), 7.47 (d, 1H), 7.36 (d, 1H), 6.99 (dd, 1H), 6.31 (d, 1H), 4.74 (s, 2H), 4.06 (q, 2H), 3.85 (t, 2H), 3.79 (t, 2H), 2.73 (t, 2H), 1.44 (s, 9H). m/z (ES⁺), [M+H]⁺=385.2.

### Example 102: 1-(1,2,3,4-Tetrahydropyrazino[1,2-a]indol-7-yl)dihydropyrimidine-2,4 (1H,3H)-dione

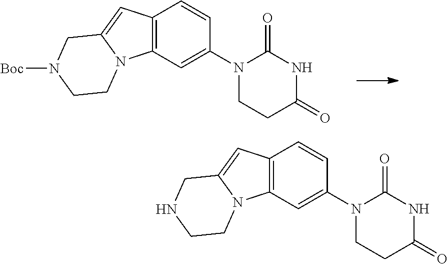

tert-Butyl 7-(2,4-dioxotetrahydropyrimidin-1 (2H)-yl)-3,4-dihydropyrazino[1,2-a]indole-2 (1H)-carboxylate (30.0 mg, 0.0780 mmol) was added to formic acid (1.00 mL, 26.1 mmol) at r.t. The resulting solution was stirred at r.t. for 1 h. The solvent was then removed under reduced pressure. The residue was diluted with DMF (0.4 mL) and then triturated with Et₂O (30 mL) to give a solid which was collected by filtration and dried under vacuum to give the title compound in the form of a formate salt (7.6 mg, 29%) as a yellow solid. ¹H NMR: δ 2.73 (2H, t), 3.22 (2H, d), 3.80 (2H, t), 3.96 (2H, t), 4.08 (2H, d), 6.16 (1H, s), 6.97 (1H, dd), 7.33 (1H, d), 7.44 (1H, d), 8.16 (1H, br s), 10.29 (1H, s). m/z (ES⁺), [M+H]⁺=285.2.

### Intermediate 103a: 7-Bromo-2-methyl-1,2,3,4-tetrahydropyrazino[1,2-a]indole

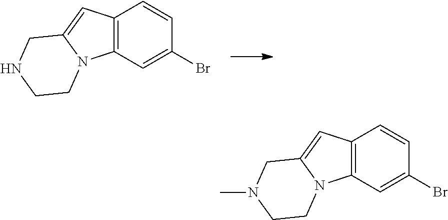

Paraformaldehyde (67.0 mg, 2.23 mmol) was added to a mixture of 7-bromo-1,2,3,4-tetrahydropyrazino[1,2-a]-indole (140 mg, 0.557 mmol) in DCM (5 mL) at r.t. The reaction was stirred for 16 h before the addition of sodium triacetoxyborohydride (295 mg, 1.39 mmol). The resulting mixture was stirred at r.t. for 2 h. The solvent was then removed under reduced pressure. Purification by C-18FC (gradient: 0-40% MeOH in water (containing 0.1% NH₄HCO₃)) gave the title compound (20.0 mg, 14%) as a yellow solid. m/z (ES⁺), [M+H]⁺=267.0.

### Example 103: 1-(2-Methyl-1,2,3,4-tetrahydropyrazino[1,2-a]indol-7-yl)dihydropyrimidine-2,4 (1H,3H)-dione

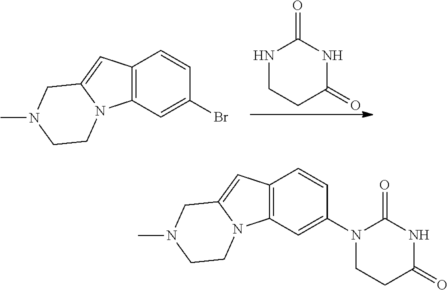 [SMILES: CN1CCn2c(cc3ccc(Br)cc32)C1 ; O=C1CCNC(=O)N1 ; CN1CCn2c(cc3ccc(N4CCC(=O)NC4=O)cc32)C1]

Ephos Pd G4 (13.9 mg, 0.0151 mmol) was added to a degassed mixture of 7-bromo-2-methyl-1,2,3,4-tetrahydro-pyrazino[1,2-a]indole (20.0 mg, 0.0754 mmol), dihydropyrimidine-2,4 (1H,3H)-dione (17.2 mg, 0.151 mmol), Ephos (8.1 mg, 0.0151 mmol) and Cs₂CO₃ (73.7 mg, 0.226 mmol) in 1,4-dioxane (1 mL) at r.t. under N₂. The resulting mixture was stirred at 120° C. for 16 h. The reaction mixture was diluted with DCM, and washed sequentially with 5% AcOH, water and saturated brine. The organic layer was dried (Na₂SO₄) and concentrated. Purification by C-18FC (gradient: 5-40% MeCN in water (containing 0.1% NH₄HCO₃)) gave the title compound (4.4 mg, 20%) as a yellow solid. ¹H NMR: δ 2.40 (3H, s), 2.73 (2H, t), 2.86 (2H, t), 3.69 (2H, s), 3.79 (2H, t), 4.03 (2H, t), 6.18 (1H, s), 6.97 (1H, dd), 7.32 (1H, s), 7.44 (1H, d), 10.27 (1H, s). m/z (ES⁺), [M+H]⁺=299.1.

### Intermediate 104a: Ethyl 5-bromo-1-(cyanomethyl)-1H-indole-2-carboxylate

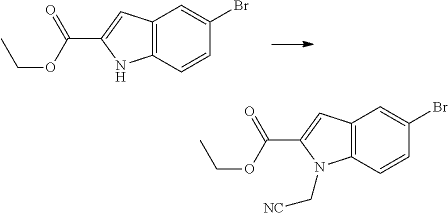 [SMILES: CCOC(=O)c1cc2cc(Br)ccc2[nH]1 ; CCOC(=O)c1cc2cc(Br)ccc2n1CC#N]

Potassium t-butoxide (6.28 g, 56.0 mmol) was added to a solution of ethyl 5-bromo-1H-indole-2-carboxylate (10.0 g, 37.3 mmol) in DMF (50 mL) at r.t. under N₂. The resulting solution was stirred at r.t. for 0.5 h before the addition of 2-chloroacetonitrile (3.38 g, 44.8 mmol). The resulting solution was stirred at r.t. for 10 h. The reaction mixture was then poured into water (150 mL), extracted with EtOAc (3×100 mL). The combined organic solutions were dried (Na₂SO₄) and concentrated to give the title compound (8.00 g, 70%) as a pale yellow solid which was used in the next step without further purification. ¹H NMR: δ 1.34 (3H, t), 4.36 (2H, q), 5.75 (2H, s), 7.36 (1H, d), 7.57 (1H, dd), 7.77 (1H, d), 7.97 (1H, d). m/z (ES⁺), [M+H]⁺=307.1.

### Intermediate 104b: 8-Bromo-1,2,3,4-tetrahydropyrazino[1,2-a]indole

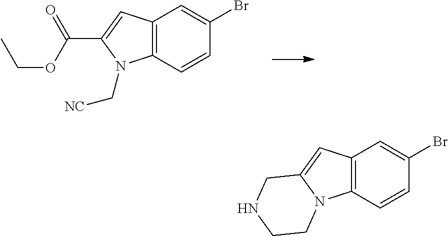 [SMILES: CCOC(=O)c1cc2cc(Br)ccc2n1CC#N ; Brc1ccc2c(c1)cc1n2CCNC1]

A solution of LiAlH₄ in THF (1M, 78.0 mL, 78.0 mmol) was added dropwise to a solution of ethyl 5-bromo-1-(cyanomethyl)-1H-indole-2-carboxylate (8.00 g, 26.0 mmol) in THF (40 mL) at r.t. under N₂. The resulting solution was stirred at 60° C. for 2 h. The reaction mixture was poured into a saturated aqueous solution of Rochelle's salt (25 mL) and extracted with EtOAc (3×30 ml). The combined organic extracts were dried (Na₂SO₄) and concentrated to give a yellow gum. Purification by C-18FC (gradient: 30-100% MeCN in water) gave the title compound (2.00 g, 31%) as a white solid. ¹H NMR: δ 3.14 (2H, t), 3.92 (2H, t), 4.01 (2H, d), 6.11 (1H, d), 7.14 (1H, dd), 7.31 (1H, d), 7.61 (1H, d). m/z (ES⁺), [M+H]⁺=253.1.

### Intermediate 104c: tert-Butyl 8-bromo-3,4-dihydropyrazino[1,2-a]indole-2 (1H)-carboxylate

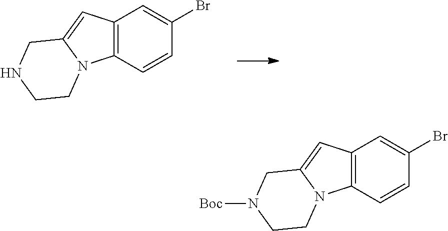 [SMILES: Brc1ccc2c(c1)cc1n2CCNC1 ; CC(C)(C)OC(=O)N1CCn2c(cc3cc(Br)ccc32)C1]

Di-tert-butyl dicarbonate (2.77 mL, 11.9 mmol) was added to a mixture of 8-bromo-1,2,3,4-tetrahydropyrazino-[1,2-a]indole (2.00 g, 7.96 mmol) and Na₂CO₃ (4.22 g, 39.8 mmol) in THF (5 mL) and water (5 mL) at r.t. The resulting solution was stirred at r.t. for 4 h. The reaction mixture was then concentrated. Purification by FSC (gradient: 0-80% EtOAc in petroleum ether) gave the title compound (1.30 g, 46%) as a yellow solid. ¹H NMR: δ 1.42 (9H, s), 3.83 (2H, t), 4.07 (2H, dd), 4.73 (2H, s), 6.26-6.31 (1H, m), 7.20 (1H, dd), 7.36 (1H, d), 7.66 (1H, d). m/z (ES⁺), [M+H]⁺=353.1.

### Intermediate 104d: tert-Butyl 8-(2,4-dioxotetrahydropyrimidin-1 (2H)-yl)-3,4-dihydropyrazino[1,2-a]-indole-2 (1H)-carboxylate

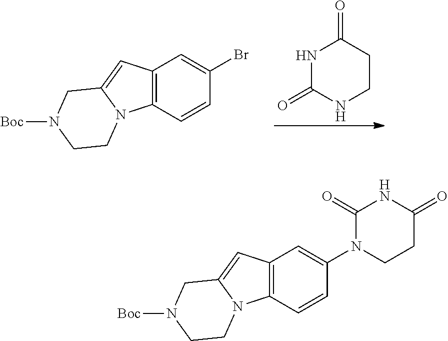 [SMILES: CC(C)(C)OC(=O)N1CCn2c(cc3cc(Br)ccc32)C1 ; O=C1CCNC(=O)N1 ; CC(C)(C)OC(=O)N1CCn2c(cc3cc(N4CCC(=O)NC4=O)ccc32)C1]

Ephos Pd G4 (78.0 mg, 0.0849 mmol) was added to a degassed mixture of tert-butyl 8-bromo-3,4-dihydro-pyrazino-[1,2-a]indole-2 (1H)-carboxylate (600 mg, 1.71 mmol), dihydropyrimidine-2,4 (1H,3H)-dione (585 mg, 5.12 mmol), Ephos (45.7 mg, 0.0855 mmol) and Cs₂CO₃ (1.11 g, 3.41 mmol) in 1,4-dioxane (10 mL) at r.t. under N₂. The resulting mixture was stirred at 120° C. for 16 h. The reaction mixture was then concentrated. Purification by C-18FC (gradient: 0-50% MeCN in water) gave the title compound (300 mg, 46%) as a pale yellow solid. ¹H NMR: δ 1.42 (9H, s), 2.70 (2H, t), 3.74 (2H, t), 3.83 (2H, t), 4.08 (2H, t), 4.73 (2H, s), 6.29 (1H, s), 7.04 (1H, dd), 7.37 (1H, d), 7.40 (1H, d), 10.24 (1H, s). m/z (ES⁺), [M+H]⁺=385.3.

### Example 104: 1-(1,2,3,4-Tetrahydropyrazino[1,2-a]indol-8-yl)dihydropyrimidine-2,4 (1H,3H)-dione

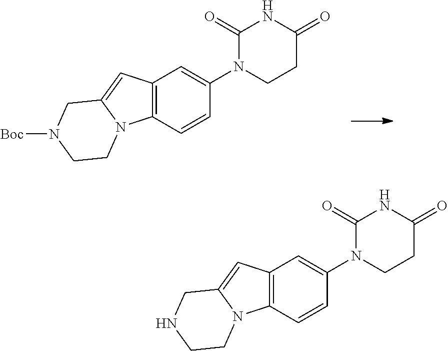 [SMILES: CC(C)(C)OC(=O)N1CCn2c(cc3cc(N4CCC(=O)NC4=O)ccc32)C1 ; O=C1CCN(c2ccc3c(c2)cc2n3CCNC2)C(=O)N1]

tert-Butyl 8-(2,4-dioxotetrahydropyrimidin-1(2H)-yl)-3,4-dihydropyrazino[1,2-a]indole-2 (1H)-carboxylate (40.0 mg, 0.104 mmol) was added to formic acid (1 mL) at r.t. The resulting solution was stirred at r.t. for 4 h. The solvent was then removed and the resulting gum was triturated with Et₂O (30 mL). A resulting solid was collected by filtration and dried under vacuum to give the title compound in the form of a formate salt (20.0 mg, 58%) as a yellow solid. ¹H NMR: δ 2.70 (2H, t), 3.18 (2H, t), 3.74 (2H, t), 3.95 (3H, t), 4.04 (2H, s), 6.13 (1H, d), 6.99 (1H, dd), 7.33 (1H, d), 7.36 (1H, d), 8.17 (1H, s), 10.22 (1H, s). m/z (ES⁺), [M+H]⁺=285.2.

### Example 105: 1-(2-Methyl-1,2,3,4-tetrahydropyrazino[1,2-a]indol-8-yl)dihydropyrimidine-2,4 (1H,3H)-dione

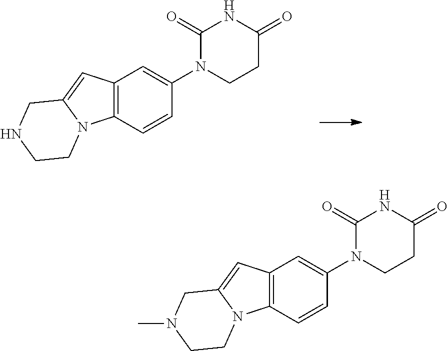 [SMILES: O=C1CCN(c2ccc3c(c2)cc2n3CCNC2)C(=O)N1 ; CN1CCn2c(cc3cc(N4CCC(=O)NC4=O)ccc32)C1]

1-(1,2,3,4-Tetrahydropyrazino[1,2-a]indol-8-yl)dihydropyrimidine-2,4 (1H,3H)-dione (90.0 mg, 0.317 mmol) was added to a mixture of paraformaldehyde (76.0 mg, 2.53 mmol) in DCM (1 mL) at r.t. under air. The resulting solution was stirred at r.t. for 1 h before the addition of sodium triacetoxyborohydride (168 mg, 0.793 mmol). The resulting solution was stirred at r.t. for 12 h and then purified directly by preparative TLC (DCM:MeOH=10:1), to give crude product which was further purified by preparative SFC (Column: Torus 2-PIC, 01083900811201; Mobile Phase A: scCO2, Mobile Phase B: MeOH (8 mmol NH₃·MeOH)-HPLC: 25; Flow rate: 50 mL/min; 254 nm; r.t.1:1.75). Pure fractions were evaporated to dryness to give the title compound (4.0 mg, 4%) as a yellow solid. ¹H NMR: δ 2.38 (3H, s), 2.70 (2H, t), 2.85 (2H, dd), 3.67 (2H, s), 3.74 (2H, t), 4.03 (2H, dd), 6.16 (1H, s), 7.00 (1H, dd), 7.33 (1H, d), 7.37 (1H, d), 10.23 (1H, s). m/z (ES⁺), [M+H]⁺=299.2.

### Intermediates 106a & 106b: tert-Butyl 4-(5-bromo-1H-indazol-1-yl)piperidine-1-carboxylate & tert-butyl 4-(5-bromo-2H-indazol-2-yl)piperidine-1-carboxylate

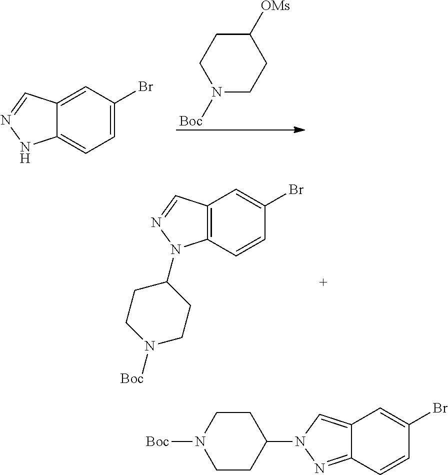 [SMILES: Brc1ccc2[nH]ncc2c1 ; CC(C)(C)OC(=O)N1CCC(OS(C)(=O)=O)CC1 ; CC(C)(C)OC(=O)N1CCC(n2ncc3cc(Br)ccc32)CC1 ; CC(C)(C)OC(=O)N1CCC(n2cc3cc(Br)ccc3n2)CC1]

A mixture of Cs₂CO₃ (5.21 g, 16.0 mmol), 5-bromo-1H-indazole (2.10 g, 10.7 mmol) and tert-butyl 4-(methylsulfonyl)oxy)piperidine-1-carboxylate (3.57 g, 12.8 mmol) in NMP (25 mL) was stirred at 100° C. overnight. The resulting mixture was filtered and the filtrate was directly purified by C-18FC (gradient: 0-100% MeCN in water) to give the title compounds as an inseparable regioisomeric mixture (2:1, 3.10 g, 76% combined yield) as a pale yellow solid. The mixture was used in the next step without further purification. m/z (ES⁺), [M+H]⁺=382.3.

### Intermediates 106c & 107a: tert-butyl 4-(5-(2,4-Dioxotetrahydropyrimidin-1 (2H)-yl)-1H-indazol-1-yl)piperidine-1-carboxylate & tert-butyl 4-(5-(2,4-dioxotetrahydropyrimidin-1 (2H)-yl)-2H-indazol-2-yl)piperidine-1-carboxylate

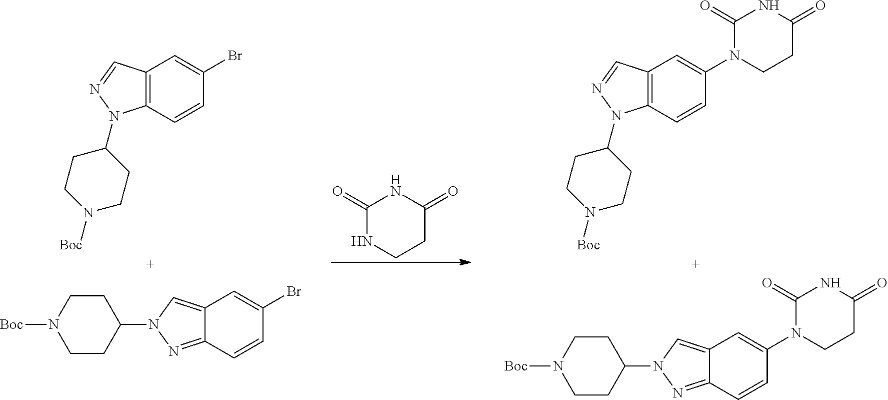 [SMILES: CC(C)(C)OC(=O)N1CCC(n2ncc3cc(Br)ccc32)CC1 ; O=C1CCNC(=O)N1 ; CC(C)(C)OC(=O)N1CCC(n2ncc3cc(N4CCC(=O)NC4=O)ccc32)CC1 ; CC(C)(C)OC(=O)N1CCC(n2cc3cc(Br)ccc3n2)CC1 ; CC(C)(C)OC(=O)N1CCC(n2cc3cc(N4CCC(=O)NC4=O)ccc3n2)CC1]

Ephos (14.1 mg, 0.0264 mmol) and Ephos Pd G4 (24.1 mg, 0.0262 mmol) were added to a degassed mixture of Cs₂CO₃ (514 mg, 1.58 mmol), dihydropyrimidine-2,4 (1H,3H)-dione (180 mg, 1.58 mmol) and a regioisomeric mixture (2:1) of tert-butyl 4-(5-bromo-1H-indazol-1-yl)piperidine-1-carboxylate and tert-butyl 4-(5-bromo-2H-indazol-2-yl)piperidine-1-carboxylate (200 mg, 0.526 mmol, combined molarity) in 1,4-dioxane (10 mL). The resulting mixture was stirred at 100° C. under N₂ for 15 h. The mixture was then filtered and washed with THF. The filtrate was then concentrated. Purification by C-18FC (gradient: 0-100% MeCN in water) gave the title compounds as an inseparable regioisomeric mixture (2:1, 180 mg, 83% combined yield) as a white solid. The mixture was used in the next step without further purification. m/z (ES⁺), [M+H]⁺=414.4.

### Example 106: 1-(1-(Piperidin-4-yl)-1H-indazol-5-yl)dihydropyrimidine-2,4 (1H,3H)-dione and Example 107: 1-(2-(Piperidin-4-yl)-2H-indazol-5-yl)dihydropyrimidine-2,4 (1H,3H)-dione

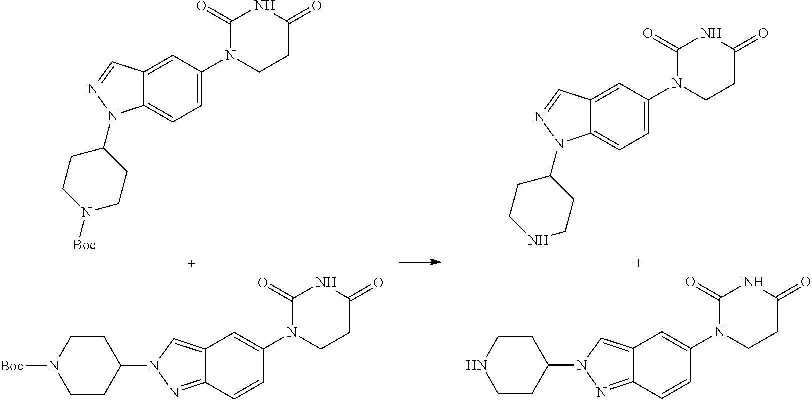 [SMILES: CC(C)(C)OC(=O)N1CCC(n2ncc3cc(N4CCC(=O)NC4=O)ccc32)CC1 ; O=C1CCN(c2ccc3c(cnn3C3CCNCC3)c2)C(=O)N1 ; CC(C)(C)OC(=O)N1CCC(n2cc3cc(N4CCC(=O)NC4=O)ccc3n2)CC1 ; O=C1CCN(c2ccc3nn(C4CCNCC4)cc3c2)C(=O)N1]

TFA (10 mL) was added to a regioisomeric mixture (2:1) of tert-butyl 4-(5-(2,4-dioxotetrahydropyrimidin-1 (2H)-yl)-1H-indazol-1-yl)piperidine-1-carboxylate and tert-butyl 4-(5-(2,4-dioxotetrahydropyrimidin-1 (2H)-yl)-2H-indazol-2-yl)piperidine-1-carboxylate (180 mg, 0.435 mmol, combined molarity) in DCM (10 mL) at r.t. The resulting solution was stirred at r.t. for 1 h. The solvents were then removed under reduced pressure. Purification by preparative HPLC (Column Z, Eluent B, gradient: 9-15%) gave the title compounds (Example 106: 38.3 mg, 28%, and Example 107: 13.9 mg, 10%) as white solids.

Example 106: ¹H NMR: δ 1.80-1.90 (m, 2H), 1.91-2.10 (m, 2H), 2.63-2.78 (m, 4H), 3.08 (d, 2H). 3.80 (t, 2H), 4.60-4.70 (m, 1H), 7.34 (dd, 1H), 7.67 (s, 1H), 7.73 (d, 1H), 8.06 (s, 1H), 10.34 (s, 1H). m/z (ES⁺), [M+H]⁺=314.3.

Example 107: ¹H NMR: δ 1.90-2.06 (m, 4H), 2.65 (t, 2H), 2.73 (t, 2H), 3.09 (d, 2H), 3.79 (t, 2H), 4.48-4.58 (m, 1H), 7.19 (dd, 1H), 7.58-7.59 (m, 2H), 8.41 (s, 1H), δ 10.32 (s, 1H). m/z (ES⁺), [M+H]⁺=314.2.

### Intermediates 108a & 108b: 5-Bromo-1-(piperidin-4-yl)-1H-indazole & 5-bromo-2-(piperidin-4-yl)-2H-indazole

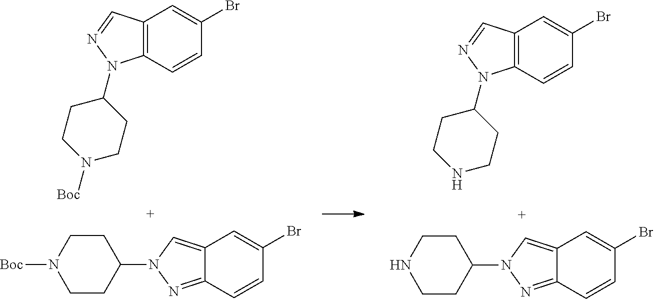

TFA (15 mL) was added to a solution of a regioisomeric mixture (2:1) of tert-butyl 4-(5-bromo-1H-indazol-1-yl)piperidine-1-carboxylate and tert-butyl 4-(5-bromo-2H-indazol-2-yl)piperidine-1-carboxylate (900 mg, 2.37 mmol, combined molarity) in DCM (15 mL). The resulting solution was stirred for 2 h. The solvent was then removed under reduced pressure to give the title compounds in the form of a trifluoroacetate salt as an isomeric mixture (2:1, 600 mg, 64% combined yield) as a white solid. The mixture was used in the next step without further purification. m/z (ES⁺), [M+H]⁺=280.1.

### Intermediate 108c: 5-Bromo-1-(1-methylpiperidin-4-yl)-1H-indazole

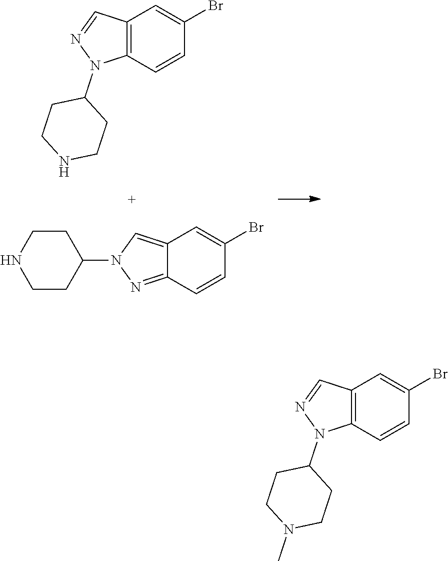 [SMILES: Brc1ccc2c(cnn2C2CCNCC2)c1 ; Brc1ccc2nn(C3CCNCC3)cc2c1 ; CN1CCC(n2ncc3cc(Br)ccc32)CC1]

Paraformaldehyde (171 mg, 5.71 mmol) was added to an isomeric mixture (2:1) of ≥5-bromo-1-(piperidin-4-yl)-1H-indazole 2,2,2-trifluoroacetate and 5-bromo-2-(piperidin-4-yl)-2H-indazole 2,2,2-trifluoroacetate (750 mg, 1.90 mmol, combined molarity) in MeOH (20 mL) at r.t. The resulting mixture was stirred at r.t. for 2 h before the addition of NaBH₃CN (359 mg, 5.71 mmol). The resulting mixture was stirred at r.t. overnight. The reaction mixture was quenched with saturated aqueous NaHCO₃ (150 mL) and extracted with EtOAc (2×150 mL). The combined organic extracts were dried (Na₂SO₄) and concentrated. Purification by C-18FC (gradient: 0-40% MeCN in water) gave the title compound (300 mg, 54%) as a white solid. ¹H NMR: δ 1.81-2.00 (m, 2H), 2.04-2.17 (m, 4H), 2.23 (s, 3H), 2.82-2.98 (m, 2H), 4.50-4.56 (m, 1H), 7.48 (dd, 1H), 7.73 (d, 1H), 7.99 (d, 1H), 8.06 (s, 1H). m/z (ES⁺), [M+H]⁺=294.0.

### Example 108: 1-(1-(1-Methylpiperidin-4-yl)-1H-indazol-5-yl)dihydropyrimidine-2,4 (1H,3H)-dione

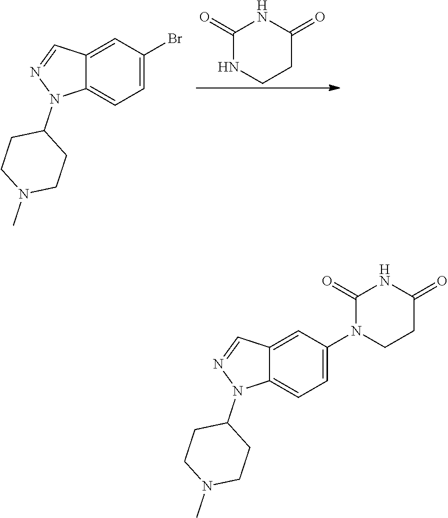

Ephos (9.1 mg, 0.017 mmol) and Ephos Pd G4 (15.6 mg, 0.0170 mmol) were added to a degassed mixture of Cs₂CO₃ (332 mg, 1.02 mmol), dihydropyrimidine-2,4 (1H,3H)-dione (116 mg, 1.02 mmol) and 5-bromo-1-(1-methylpiperidin-4-yl)-1H-indazole (100 mg, 0.340 mmol) in 1,4-dioxane (10 mL) at r.t. under N₂. The resulting mixture was stirred at 100° C. for 15 h. The mixture was then diluted with THF, filtered and washed with THF. The filtrates were concentrated. Purification C-18FC (gradient: 0-100% MeCN in water) gave material which was further purified by preparative HPLC (Column B, Eluent B, gradient: 13-20%) to give the title compound (10.3 mg, 9%) as a white solid. ¹H NMR: δ 10.40 (br s, 1H), 8.07 (s, 1H), 7.73 (d, 1H), 7.67 (s, 1H), 7.34 (d, 1H), 4.54-4.63 (m, 1H), 3.80 (t, 2H), 2.90 (d, 2H), 2.74 (t, 2H), 2.24 (s, 3H), 2.10-2.19 (m, 4H), 1.82-1.93 (br s, 2H). m/z (ES⁺), [M+H]⁺=328.1

### Intermediate 109a: tert-Butyl 4-(6-bromo-2H-indazol-2-yl)piperidine-1-carboxylate

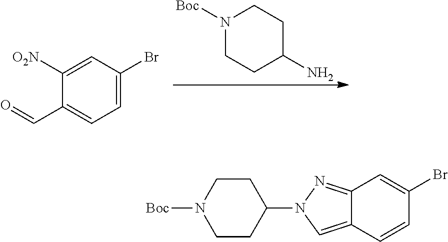 [SMILES: O=Cc1ccc(Br)cc1[N+](=O)[O-] ; CC(C)(C)OC(=O)N1CCC(N)CC1 ; CC(C)(C)OC(=O)N1CCC(n2cc3ccc(Br)cc3n2)CC1]

4-Bromo-2-nitrobenzaldehyde (2.00 g, 8.70 mmol) was added to a solution of tert-butyl 4-aminopiperidine-1-carboxylate (1.92 g, 9.59 mmol) in iPrOH (24 mL) at r.t. The resulting mixture was stirred at 80° C. for 4 h before the addition of tri-n-butylphosphine (6.44 mL, 26.1 mmol). The resulting mixture was stirred overnight at 80° C. The solvent was then removed under reduced pressure. Purification by FSC (gradient: 0-18% EtOAc in petroleum ether) gave the title compound (3.00 g, 91%) as a white solid. ¹H NMR: δ 1.43 (9H, s), 1.84-2.03 (2H, m), 2.05-2.16 (2H, m), 2.84-3.07 (2H, m), 4.04-4.15 (2H, m), 4.71 (1H, tt), 7.14 (1H, dd), 7.69 (1H, dd), 7.87 (1H, dt), 8.51 (1H, d). m/z (ES⁺), [M+H]⁺=382.1.

### Intermediate 109b: tert-Butyl 4-(6-(2,4-dioxotetrahydropyrimidin-1 (2H)-yl)-2H-indazol-2-yl)piperidine-1-carboxylate

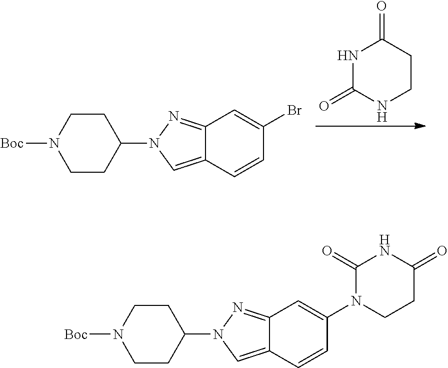

Ephos Pd G4 (60.4 mg, 0.0658 mmol) was added to a degassed mixture of tert-butyl 4-(6-bromo-2H-indazol-2-yl)piperidine-1-carboxylate (500 mg, 1.31 mmol), dihydropyrimidine-2,4 (1H,3H)-dione (450 mg, 3.94 mmol), Ephos (35.2 mg, 0.0658 mmol) and Cs₂CO₃ (857 mg, 2.63 mmol) in 1,4-dioxane (10 mL) at r.t. under N₂. The resulting mixture was stirred at 100° C. for 16 h. The solvent was then removed under reduced pressure. Purification by FSC (gradient: 0-100% EtOAc in petroleum ether, then maintained 100% EtOAc for 10 minutes) gave the title compound (500 mg, 92%) as a white solid. ¹H NMR: δ 1.41 (9H, s), 1.84-2.03 (2H, m), 2.03-2.16 (2H, m), 2.71 (2H, t), 2.86-3.05 (2H, m), 3.81 (2H, t), 3.96-4.20 (2H, m), 4.60-4.76 (1H, m), 7.01 (1H, dd), 7.48 (1H, s), 7.65 (1H, dd), 8.43 (1H, d), 10.35 (1H, s). m/z (ES⁺), [M+H]⁺=414.2.

### Example 109: 1-(2-(Piperidin-4-yl)-2H-indazol-6-yl)dihydropyrimidine-2,4 (1H,3H)-dione

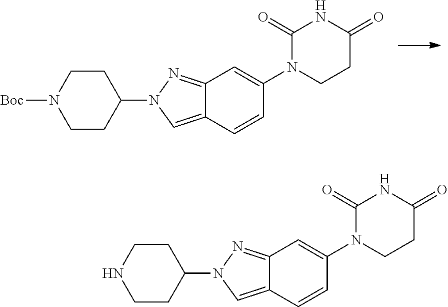 [SMILES: CC(C)(C)OC(=O)N1CCC(n2cc3ccc(N4CCC(=O)NC4=O)cc3n2)CC1 ; O=C1CCN(c2ccc3cn(C4CCNCC4)nc3c2)C(=O)N1]

A solution of HCl in 1,4-dioxane (4M, 10.0 mL, 40.0 mmol) was added to a solution of tert-butyl 4-(6-(2,4-dioxotetrahydropyrimidin-1 (2H)-yl)-2H-indazol-2-yl)piperidine-1-carboxylate (500 mg, 1.21 mmol) in DCM (10 mL) at r.t. The resulting mixture was stirred at r.t. for 2 h. The solvent was then removed under reduced pressure. Purification by C-18FC (gradient: 0-30% MeCN in water (10 mmol NH₄HCO₃)) gave the title compound in the form of a hydrochloride salt (400 mg, 95%) as a white solid. ¹H NMR: δ 1.87-2.12 (4H, m), 2.59-2.69 (2H, m), 2.74 (2H, t), 2.84-3.18 (2H, m), 3.83 (2H, t), 3.99-4.22 (1H, m), 4.51-4.79 (1H, m), 7.02 (1H, dd), 7.50 (1H, d), 7.67 (1H, d), 8.42 (1H, s), 10.37 (1H, s). m/z (ES⁺), [M+H]⁺=314.2.

### Example 110: 1-(2-(1-Methylpiperidin-4-yl)-2H-indazol-6-yl)dihydropyrimidine-2,4 (1H,3H)-dione

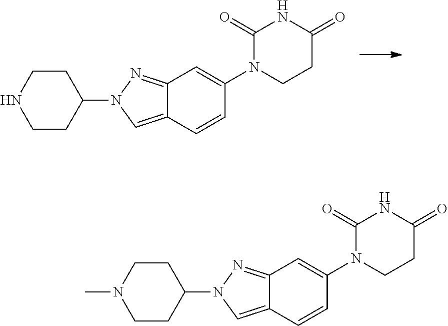 [SMILES: O=C1CCN(c2ccc3cn(C4CCNCC4)nc3c2)C(=O)N1 ; CN1CCC(n2cc3ccc(N4CCC(=O)NC4=O)cc3n2)CC1]

Sodium triacetoxyborohydride (127 mg, 0.599 mmol) was added to a mixture of 1-(2-(piperidin-4-yl)-2H-indazol-6-yl)dihydropyrimidine-2,4 (1H,3H)-dione hydrochloride (70.0 mg, 0.200 mmol), formaldehyde (11.0 μL, 0.399 mmol), AcOH (23.0 μL, 0.402 mmol) and NaOAc (49.2 mg, 0.600 mmol) in DCM (5 mL) at r.t. The resulting mixture was stirred at r.t. for 2 h. The solvent was then removed under reduced pressure. Purification by C-18FC (gradient: 0-40% MeCN in water (10 mmol NH₄HCO₃)) gave the title compound (40.0 mg, 61%) as a white solid. ¹H NMR: δ 2.03-2.19 (6H, m), 2.23 (3H, s), 2.74 (2H, t), 2.83-2.95 (2H, m), 3.83 (2H, t), 4.42-4.50 (1H, m), 7.02 (1H, dd), 7.51 (1H, d), 7.67 (1H, d), 8.43 (1H, d), 10.37 (1H, s). m/z (ES⁺), [M+H]⁺=328.1.

Intermediate 111a: tert-Butyl 4-(4-bromo-1H-indol-1-yl)piperidine-1-carboxylate

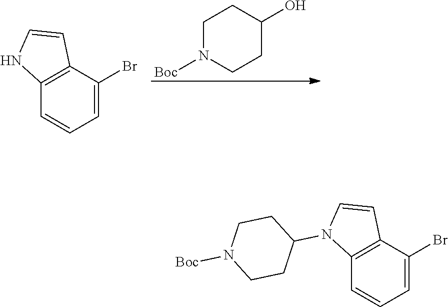 [SMILES: Brc1cccc2[nH]ccc12 ; CC(C)(C)OC(=O)N1CCC(O)CC1 ; CC(C)(C)OC(=O)N1CCC(n2ccc3c(Br)cccc32)CC1]

(Cyanomethylene)tri-n-butylphosphorane (92.0 g, 382.56 mmol) was added to a stirred solution of tert-butyl 4-hydroxypiperidine-1-carboxylate (77.0 g, 382.56 mmol) and 4-bromo-1H-indole (50.0 g, 255.04 mmol) in toluene (500 mL) under N₂ at r.t. The resulting mixture was stirred at 100° C. for 18 h. The solvent was then removed under reduced pressure. Purification by C-18FC (gradient: 0-70% MeCN in water (containing 0.4% NH₄HCO₃)) gave the title compound (20.0 g, 21%) as a yellow solid. ¹H NMR: δ 1.44 (9H, s), 1.76-2.00 (4H, m), 2.98 (2H, m), 4.13 (2H, br d), 4.60 (1H, ddt), 6.42 (1H, d), 7.08 (1H, t), 7.23-7.28 (1H, m), 7.58-7.74 (2H, m). m/z: (ES⁺) [M+H]⁺=379.1.

### Intermediate 11b: tert-Butyl 4-(4-(2,4-dioxotetrahydropyrimidin-1 (2H)-yl)-1H-indol-1-yl)piperidine-1-carboxylate

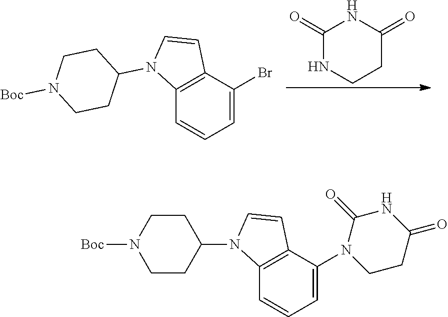 [SMILES: CC(C)(C)OC(=O)N1CCC(n2ccc3c(Br)cccc32)CC1 ; O=C1CCNC(=O)N1 ; CC(C)(C)OC(=O)N1CCC(n2ccc3c(N4CCC(=O)NC4=O)cccc32)CC1]

Ephos Pd G4 (240 mg, 0.261 mmol) and Ephos (140 mg, 0.262 mmol) were added in one portion to a degassed mixture of tert-butyl 4-(4-bromo-1H-indol-1-yl)piperidine-1-carboxylate (1.25 g, 2.64 mmol), dihydro-pyrimidine-2,4 (1H,3H)-dione (1.20 g, 10.5 mmol) and Cs₂CO₃ (2.58 g, 7.92 mmol) in 1,4-dioxane (40 mL) at r.t. under N₂. The resulting mixture was stirred at 100° C. for 17 h. The solvent was then removed under reduced pressure. Purification by FSC (gradient: 0-60% EtOAc in petroleum ether) gave the title compound (650 mg, 60%) as a pale yellow solid. ¹H NMR: δ 1.44 (9H, s), 1.77-1.97 (4H, m), 2.76 (2H, t), 2.87-3.07 (2H, m), 3.77 (2H, t), 4.14 (2H, d), 4.53-4.65 (1H, m), 6.43 (1H, d), 6.97 (1H, d), 7.16 (1H, t), 7.50-7.59 (2H, m), 10.33 (1H, s). m/z: (ES⁺) [M+Na]⁺=435.2.

### Example 111: 1-(1-(Piperidin-4-yl)-1H-indol-4-yl)dihydropyrimidine-2,4 (1H,3H)-dione

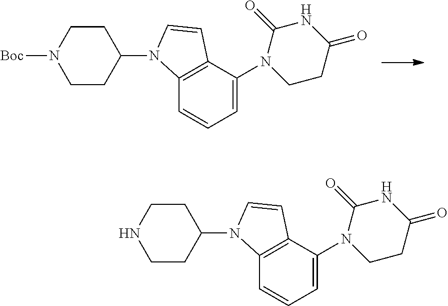 [SMILES: CC(C)(C)OC(=O)N1CCC(n2ccc3c(N4CCC(=O)NC4=O)cccc32)CC1 ; O=C1CCN(c2cccc3c2ccn3C2CCNCC2)C(=O)N1]

tert-Butyl 4-(4-(2,4-dioxotetrahydropyrimidin-1 (2H)-yl)-1H-indol-1-yl)piperidine-1-carboxylate (600 mg, 1.45 mmol) was added to AcOH (20 ml) at r.t. under air. The resulting solution was stirred at 100° C. for 2 days. The solvent was then removed under reduced pressure. Purification by C-18FC (gradient 0-30% MeCN in water (containing 0.1% FA)) gave the title compound in the form of a formate salt (340 mg, 66%) as a pale yellow solid. ¹H NMR: δ 2.01-2.15 (4H, m), 2.77 (2H, t), 2.94 (2H, t), 3.28 (2H, d), 3.79 (2H, t), 4.59-4.64 (1H, m), 6.45 (1H, d), 6.98 (1H, d), 7.17 (1H, t), 7.47 (1H, d), 7.56 (1H, d), 8.36 (1H, s), 10.34 (1H, s). m/z (ES⁺), [M+H]⁺=313.2.

### Example 112: 1-(1-(1-Methylpiperidin-4-yl)-1H-indol-4-yl)dihydropyrimidine-2,4 (1H,3H)-dione

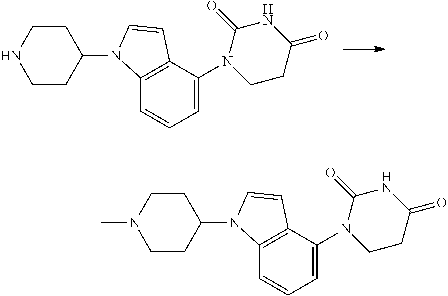 [SMILES: O=C1CCN(c2cccc3c2ccn3C2CCNCC2)C(=O)N1 ; CN1CCC(n2ccc3c(N4CCC(=O)NC4=O)cccc32)CC1]

AcOH (16.0 μL, 0.279 mmol) was added to a mixture of NaOAc (45.8 mg, 0.558 mmol), sodium triacetoxyborohydride (177 mg, 0.835 mmol), formaldehyde (84.0 mg, 2.80 mmol) and 1-(1-(piperidin-4-yl)-1H-indol-4-yl)dihydropyrimidine-2,4 (1H3H)-dione formate (100 mg, 0.279 mmol) in DCM (10 mL) and MeOH (1.0 mL) at r.t. The resulting mixture was stirred at r.t. for 3 h. The solvent was then removed under reduced pressure. Purification by C-18FC (gradient: 0-30% MeCN in water (containing 0.1% FA)) gave the title compound in the form of a formate salt (76.0 mg, 72%) as a pale yellow solid. ¹H NMR: δ 1.90-1.98 (2H, m), 1.99-2.12 (2H, m), 2.24-2.36 (5H, m), 2.76 (2H, t), 2.95-3.03 (2H, m), 3.78 (2H, t), 4.34-4.45 (1H, m), 6.42 (1H, d), 6.96 (1H, d), 7.15 (1H, t), 7.48-7.56 (2H, m), 8.20 (s, 1H), 10.31 (1H, s). m/z (ES⁺), [M+H]⁺=327.1.

### Intermediate 113a: tert-Butyl 4-(5-(2,4-dioxotetrahydropyrimidin-1 (2H)-yl)-1H-indol-1-yl)piperidine-1-carboxylate

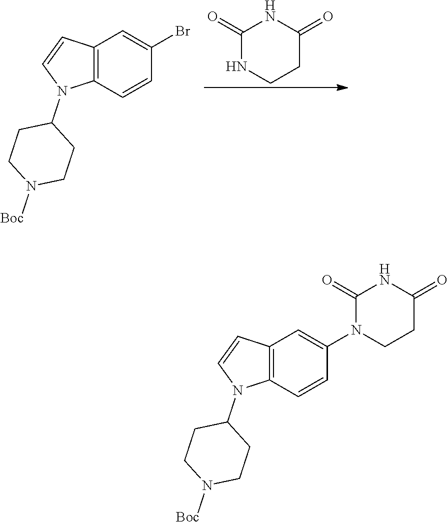

Ephos Pd G4 (1.09 g, 1.19 mmol) and Ephos (635 mg, 1.19 mmol) were added in one portion to a degassed mixture of tert-butyl 4-(5-bromo-1H-indol-1-yl)piperidine-1-carboxylate (synthesised by the method described in WO2006038006, 9.00 g, 23.7 mmol), dihydropyrimidine-2,4 (1H,3H)-dione (10.83 g, 94.91 mmol), and Cs₂CO₃ (15.46 g, 47.46 mmol) in 1,4-dioxane (450 mL) at r.t. under N₂. The resulting mixture was stirred at 100° C. for 16 h. The solvent was then removed under reduced pressure. Purification by FSC (gradient: 0-10% MeOH in DCM) gave the title compound (8.20 g, 84%) as a pale yellow solid. m/z: (ES⁺) [M−tBu+2H]⁺357.2.

### Example 113: 1-(1-(Piperidin-4-yl)-1H-indol-5-yl)dihydropyrimidine-2,4 (1H,3H)-dione

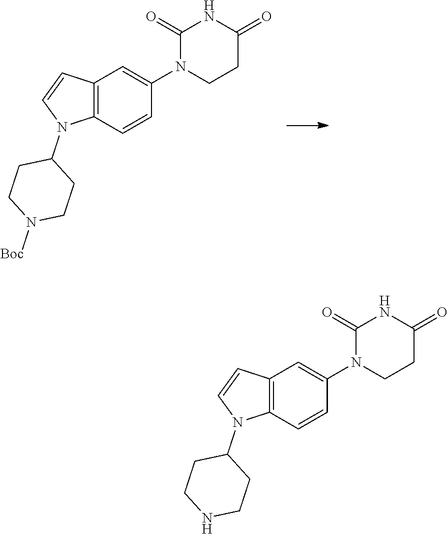 [SMILES: CC(C)(C)OC(=O)N1CCC(n2ccc3cc(N4CCC(=O)NC4=O)ccc32)CC1 ; O=C1CCN(c2ccc3c(ccn3C3CCNCC3)c2)C(=O)N1]

tert-Butyl 4-(5-(2,4-dioxotetrahydropyrimidin-1 (2H)-yl)-1H-indol-1-yl)piperidine-1-carboxylate (2.10 g, 5.09 mmol) was added to formic acid (50 mL) at r.t. The resulting mixture was stirred at 50° C. for 1 h. The solvent was then removed under reduced pressure. The reaction mixture was basified using saturated NaHCO₃ solution. The precipitate was collected by filtration, washed with water (20 mL) and dried under vacuum to give the title compound (1.57 g, 99%) as a pink solid. ¹H NMR: δ 1.80-1.93 (4H, m), 2.69-2.87 (3H, m), 2.87-3.21 (2H, m), 3.77 (2H, t), 3.92-4.32 (1H, m), 4.32-4.71 (1H, m), 6.47 (1H, d), 7.08 (1H, dd), 7.47 (1H, d), 7.53 (1H, br s), 7.57 (1H, d), 10.28 (1H, s). m/z (ES⁺), [M+H]⁺=313.1.

### Example 114: 1-(1-(1-Methylpiperidin-4-yl)-1H-indol-5-yl)dihydropyrimidine-2,4 (1H,3H)-dione

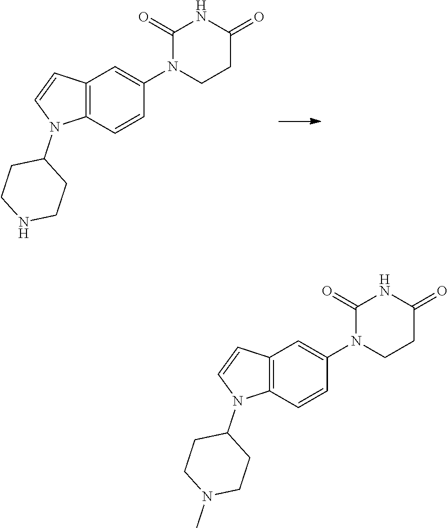 [SMILES: O=C1CCN(c2ccc3c(ccn3C3CCNCC3)c2)C(=O)N1 ; CN1CCC(n2ccc3cc(N4CCC(=O)NC4=O)ccc32)CC1]

Formaldehyde (33.6 mg, 1.12 mmol) was added to a mixture of 1-(1-(piperidin-4-yl)-1H-indol-5-yl)dihydro-pyrimidine-2,4 (1H,3H)-dione (70.0 mg, 0.224 mmol), and sodium triacetoxyborohydride (237 mg, 1.12 mmol) in DCM (10 mL) and MeOH (4 mL) at r.t. The resulting mixture was stirred at r.t. for 3 days. The solvent was then removed under reduced pressure. Purification by C-18FC (gradient: 0-30% MeCN in water (containing 0.1% FA)) gave material which was further purified by preparative HPLC (Column D, Eluent C, gradient: 15-25%) to give the title compound in the form of a trifluoroacetate salt (40.0 mg, 42%) as a white solid. ¹H NMR: (CD₃OD) δ 2.18-2.42 (4H, m), 2.86 (2H, t), 3.00 (3H, s), 3.36-3.43 (2H, m), 3.73 (2H, d), 3.90 (2H, t), 4.68-4.83 (1H, m), 6.59 (1H, d), 7.19 (1H, dd), 7.40 (1H, d), 7.51-7.65 (2H, m). m/z (ES⁺), [M+H]⁺=327.3.

### Example 115: 1-(1-(1-Acetylpiperidin-4-yl)-1H-indol-5-yl)dihydropyrimidine-2,4 (1H,3H)-dione

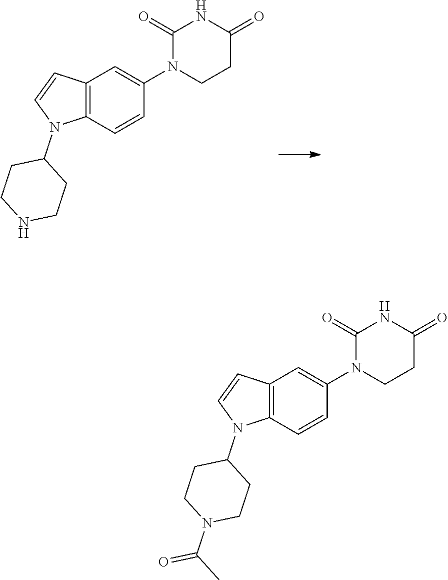 [SMILES: O=C1CCN(c2ccc3c(ccn3C3CCNCC3)c2)C(=O)N1 ; CC(=O)N1CCC(n2ccc3cc(N4CCC(=O)NC4=O)ccc32)CC1]

Ac₂O (49.0 mg, 0.480 mmol) was added to a mixture of 1-(1-(piperidin-4-yl)-1H-indol-5-yl)dihydropyrimidine-2,4 (1H,3H)-dione (75.0 mg, 0.240 mmol) and triethylamine (100 μL, 0.717 mmol) in DCM (4 mL) at r.t. under air. The resulting mixture was stirred at r.t. for 3 h. The solvent was then removed under reduced pressure. Purification by C-18FC (gradient: 0-60% MeCN in water (containing 0.05% TFA)) gave the title compound (53.0 mg, 62%) as a light pink solid. ¹H NMR: δ 1.80 (1H, qd), 1.93-1.98 (3H, m), 2.07 (3H, s), 2.70-2.79 (3H, m), 3.29 (1H, ddd), 3.78 (2H, t), 3.98 (1H, dd), 4.54-4.72 (2H, m), 6.47 (1H, d), 7.10 (1H, dd), 7.47 (1H, d), 7.54 (1H, d), 7.60 (1H, d), 10.26 (1H, s). m/z (ES⁺), [M+H]⁺=355.2.

### Intermediate 116a: tert-Butyl 6-bromo-3-(1-(tert-butoxycarbonyl)piperidin-4-yl)-1H-indole-1-carboxylate

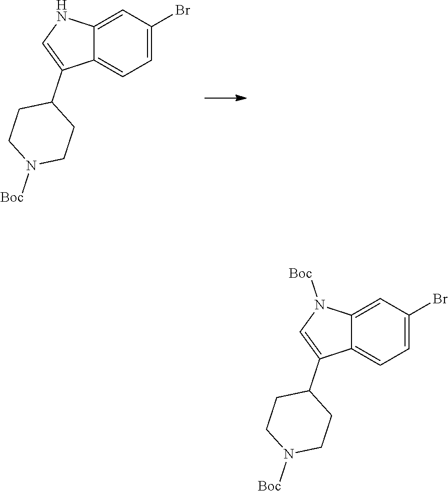 [SMILES: CC(C)(C)OC(=O)N1CCC(c2c[nH]c3cc(Br)ccc23)CC1 ; CC(C)(C)OC(=O)N1CCC(c2cn(C(=O)OC(C)(C)C)c3cc(Br)ccc23)CC1]

Di-tert-butyl dicarbonate (918 μL, 3.95 mmol) was added to a mixture of tert-butyl 4-(6-bromo-1H-indol-3-yl)piperidine-1-carboxylate (prepared by the method described in WO2011128455, 1.00 g, 2.64 mmol), DIEA (1.38 ml, 7.90 mmol) and DMAP (32.0 mg, 0.262 mmol) in DCM (30 mL) at r.t. The resulting mixture was stirred at r.t. for 16 h. The solvent was then removed under reduced pressure. Purification by FSC (gradient: 0-15% EtOAc in petroleum ether) gave the title compound (1.13 g, 89%) as a white oil which solidified on standing. ¹H NMR: (CDCl₃) 1.51 (9H, s), 1.59-1.69 (2H, m), 1.69 (9H, s), 1.97-2.09 (2H, m), 2.90 (3H, t), 4.27 (2H, d), 7.30 (1H, s), 7.37 (1H, dd), 7.42 (1H, d), 8.37 (1H, s) m z: (ES⁺), [M+Na]⁺ 501.0.

### Intermediate 116b: tert-Butyl 3-(1-(tert-butoxycarbonyl)piperidin-4-yl)-6-(2,4-dioxotetrahydro-pyrimidin-1 (2H)-yl)-1H-indole-1-carboxylate

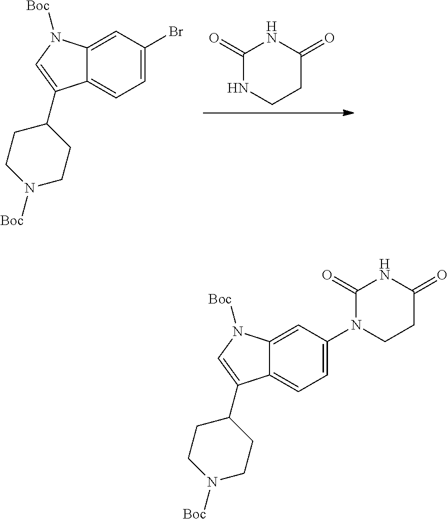

Ephos Pd G4 (105 mg, 0.114 mmol) and Ephos (61.0 mg, 0.114 mmol) were added in one portion to a degassed mixture of tert-butyl 6-bromo-3-(1-(tert-butoxycarbonyl)piperidin-4-yl)-1H-indole-1-carboxylate (1.10 g, 2.29 mmol), dihydropyrimidine-2,4 (1H,31H)-dione (1.05 g, 9.20 mmol), and Cs₂CO₃ (1.50 g, 4.60 mmol) in 1,4-dioxane (30 mL) at r.t. under N₂. The resulting mixture was stirred at 100° C. for 16 h. The reaction was then cooled to r.t. and the solvent was removed under reduced pressure. Purification by FSC (gradient: 0-5% MeOH in DCM) gave the title compound (1.03 g, 88%) as a pale yellow solid. ¹H NMR: 1.20-1.37 (1H, m), 1.40 (9H, s), 1.42-1.56 (2H, m), 1.60 (9H, s), 1.86-1.97 (2H, m), 2.72 (2H, t), 2.84-3.01 (2H, m), 3.81 (2H, t), 4.02-4.14 (2H, m), 7.20 (1H, dd), 7.42 (1H, d), 7.65 (1H, d), 8.01 (1H, d), 10.34 (1H, s). m z: (ES⁺), [M+Na]⁺ 535.3.

### Example 116: 1-(3-(Piperidin-4-yl)-1H-indol-6-yl)dihydropyrimidine-2,4 (1H,3H)-dione

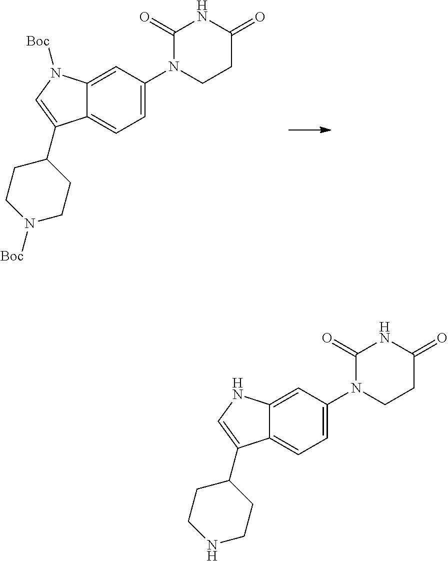 [SMILES: CC(C)(C)OC(=O)N1CCC(c2cn(C(=O)OC(C)(C)C)c3cc(N4CCC(=O)NC4=O)ccc23)CC1 ; O=C1CCN(c2ccc3c(C4CCNCC4)c[nH]c3c2)C(=O)N1]

tert-Butyl 3-(1-(tert-butoxycarbonyl)piperidin-4-yl)-6-(2,4-dioxotetrahydropyrimidin-1 (2H)-yl)-1H-indole-1-carboxylate (1.00 g, 1.95 mmol) was added to 2,2,2-trifluoroethanol (8 mL). The resulting mixture was heated in a microwave reactor at 150° C. for 10 h and then cooled to r.t. The solvent was then removed under reduced pressure. Purification by C-18FC (gradient: 5-15% MeCN in water (containing 0.1% FA)) gave the title compound in the form of a formate salt (480 mg, 69%) as a white solid. ¹H NMR: 1.83 (2H, q), 2.03 (2H, d), 2.72 (2H, t), 2.90-3.05 (3H, m), 3.28 (2H, d), 3.78 (2H, t), 6.93 (1H, dd), 7.16 (1H, d), 7.28 (1H, d), 7.60 (1H, d), 8.37 (1H, s), 10.26 (1H, s), 10.94 (1H, s). m/z: (ES⁺), [M+H]⁺ 313.1.

### Example 117: 1-(3-(1-Methylpiperidin-4-yl)-1H-indol-6-yl)dihydropyrimidine-2,4 (1H,3H)-dione

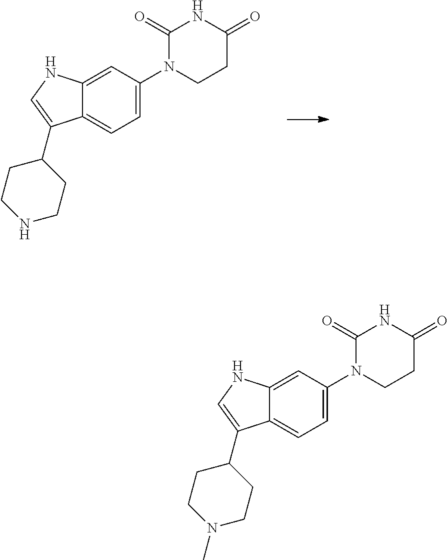 [SMILES: O=C1CCN(c2ccc3c(C4CCNCC4)c[nH]c3c2)C(=O)N1 ; CN1CCC(c2c[nH]c3cc(N4CCC(=O)NC4=O)ccc23)CC1]

NaOAc (68.7 mg, 0.837 mmol) was added to a mixture of 1-(3-(piperidin-4-yl)-1H-indol-6-yl)dihydropyrimidine-2,4 (1H,3H)-dione formate (100 mg, 0.279 mmol), sodium triacetoxyborohydride (177 mg, 0.835 mmol) and paraformaldehyde (25.1 mg, 0.836 mmol) in DCM (10 mL). The resulting mixture was stirred at r.t. for 4 h. The solvent was then removed under reduced pressure. Purification by C-18FC (gradient: 0-8% MeCN in water (containing 0.1% FA)) gave the title compound in the form of a formate salt (23.0 mg, 22%) as a white solid. ¹H NMR: δ 1.75 (2H, qd), 1.95 (2H, d), 2.18-2.29 (2H, m), 2.32 (3H, s), 2.68-2.82 (3H, m), 2.97 (2H, d), 3.78 (2H, t), 6.91 (1H, dd), 7.13 (1H, d), 7.27 (1H, d), 7.54 (1H, d), 8.24 (1H, s), 10.25 (1H, s), 10.87 (1H, s). m/z (ES⁺), [M+H]⁺=327.1.

### Example 118: 1-(3-(1-Acetylpiperidin-4-yl)-1H-indol-6-yl)dihydropyrimidine-2,4 (1H,3H)-dione

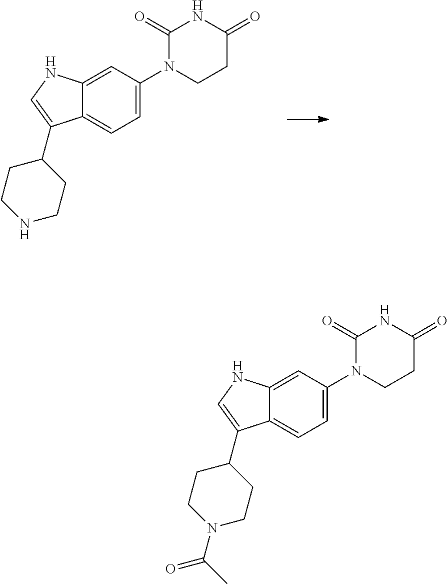 [SMILES: O=C1CCN(c2ccc3c(C4CCNCC4)c[nH]c3c2)C(=O)N1 ; CC(=O)N1CCC(c2c[nH]c3cc(N4CCC(=O)NC4=O)ccc23)CC1]

Triethylamine (117 μL, 0.839 mmol) was added to a solution of Ac₂O (23.7 μL, 0.251 mmol) and 1-(3-(piperidin-4-yl)-1H-indol-6-yl)dihydropyrimidine-2,4 (1H,3H)-dione formate (100 mg, 0.279 mmol) in DCM (5 mL). The resulting mixture was stirred at r.t. for 1 h. The solvent was then removed under reduced pressure. Purification by C-18FC (gradient: 5-30% MeCN in water (containing 0.1% FA)) gave the title compound (38.7 mg, 39%) as a white solid. ¹H NMR: δ 1.42-1.53 (1H, m), 1.55-1.66 (1H, m), 1.97 (2H, t), 2.03 (3H, s), 2.62-2.76 (3H, m), 2.98-3.09 (1H, m), 3.20 (1H, t), 3.78 (2H, t), 3.91 (1H, d), 4.50 (1H, d), 6.92 (1H, dd), 7.14 (1H, d), 7.27 (1H, d), 7.56 (1H, d), 10.25 (1H, s), 10.88 (1H, s). m/z (ES⁺), [M+H]⁺=355.1.

### Intermediate 119a: tert-Butyl 4-(6-bromo-1-methyl-1H-indol-3-yl)piperidine-1-carboxylate

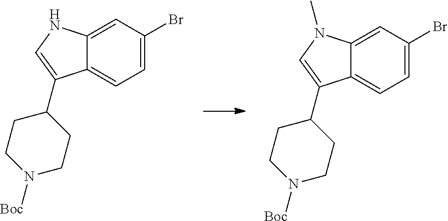 [SMILES: CC(C)(C)OC(=O)N1CCC(c2c[nH]c3cc(Br)ccc23)CC1 ; Cn1cc(C2CCN(C(=O)OC(C)(C)C)CC2)c2ccc(Br)cc21]

NaH (60% dispersion in mineral oil, 47.0 mg, 1.18 mmol) was added to a solution of tert-butyl 4-(6-bromo-1H-indol-3-yl)piperidine-1-carboxylate (prepared by the method described in WO2011128455, 300 mg, 0.790 mmol) in DMF (10 mL) at 0° C. under N₂. The resulting mixture was stirred at 0° C. for 0.5 h. Mel (52.0 μl, 0.832 mmol) was added and the mixture was stirred at r.t. for 1 h. The reaction was quenched with saturated NH₄Cl (10 mL) and extracted with EtOAc (3×10 mL). The combined organic extracts were washed with saturated brine (3×2 mL). The organic layer was dried (MgSO₄) and concentrated to give the title compound (305 mg, 98%) as pale yellow gum which was used in the next step without further purification. ¹H NMR: (CDCl₃) δ 1.51 (9H, s), 1.57-1.72 (2H, m), 1.97-2.05 (2H, m), 2.84-2.97 (3H, m), 3.73 (3H, s), 4.20-4.31 (2H, m), 6.80 (1H, s), 7.21 (1H, dd), 7.47 (1H, d), 7.49 (1H, d). m/z (ES⁺), [M-tBu+2H]⁺=337.0.

### Intermediate 119b: tert-Butyl 4-(6-(2,4-dioxotetrahydropyrimidin-1 (2H)-yl)-1-methyl-1H-indol-3-yl)piperidine-1-carboxylate

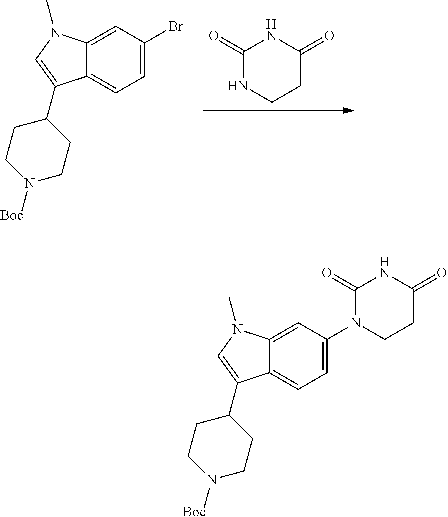

Ephos Pd G4 (35.0 mg, 0.0381 mmol) was added to a degassed mixture of tert-butyl 4-(6-bromo-1-methyl-1H-indol-3-yl)piperidine-1-carboxylate (300 mg, 0.763 mmol), dihydropyrimidine-2,4 (1H,3H)-dione (348 mg, 3.05 mmol), Ephos (20.4 mg, 0.0381 mmol) and Cs₂CO₃ (497 mg, 1.53 mmol) in 1,4-dioxane (10 mL) at r.t. under N₂. The resulting mixture was stirred at 100° C. for 16 h. The solvent was then removed under reduced pressure. Purification by FSC (gradient: 0-5% MeOH in DCM) gave the title compound (300 mg, 92%) as a pale yellow solid. ¹H NMR: δ 1.40 (9H, s), 1.31-1.57 (2H, m), 1.84-1.97 (2H, m), 2.66-2.77 (2H, m), 2.79-3.05 (3H, m), 3.69 (3H, s), 3.78 (2H, t), 4.03 (2H, d), 6.94 (1H, dd), 7.13 (1H, s), 7.33 (1H, d), 7.54 (1H, d), 10.28 (1H, s). m/z (ES⁺), [M+Na]=449.3.

### Intermediate 119c: 1-(1-Methyl-3-(piperidin-4-yl)-1H-indol-6-yl)dihydropyrimidine-2,4 (1H,3H)-dione

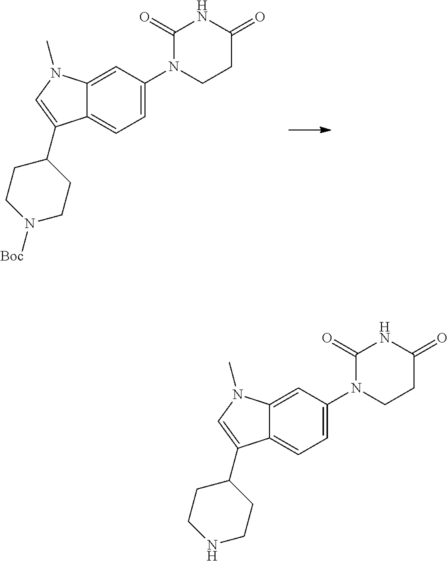 [SMILES: Cn1cc(C2CCN(C(=O)OC(C)(C)C)CC2)c2ccc(N3CCC(=O)NC3=O)cc21 ; Cn1cc(C2CCNCC2)c2ccc(N3CCC(=O)NC3=O)cc21]

tert-Butyldimethylsilyltrifluoromethanesulfonate (347 mg, 1.31 mmol) was added to a solution of tert-butyl 4-(6-(2,4-dioxotetrahydropyrimidin-1 (2H)-yl)-1-methyl-1H-indol-3-yl)piperidine-1-carboxylate (280 mg, 0.656 mmol) in MeCN (10 mL) at 0° C. under N₂. The resulting mixture was stirred at r.t. for 1 h. The solvent was then removed under reduced pressure. Purification by C-18FC (gradient: 0-25% MeCN in water (10 mmol NH₄HCO₃)) gave the title compound (190 mg, 89%) as a white solid. ¹H NMR: δ 1.40-1.64 (2H, m), 1.81-2.00 (2H, m), 2.59-2.71 (1H, m), 2.74 (2H, t), 2.78-2.98 (2H, m), 3.04 (1H, br d), 3.72 (3H, s), 3.80 (2H, t), 3.91-4.17 (1H, m), 6.96 (1H, d), 7.06-7.18 (1H, m), 7.35 (1H, s), 7.56 (1H, d), 10.30 (1H, s). m/z (ES⁺), [M+H]⁺=327.3.

### Example 119: 1-(1-Methyl-3-(1-methylpiperidin-4-yl)-1H-indol-6-yl)dihydropyrimidine-2,4 (1H,3H)-dione

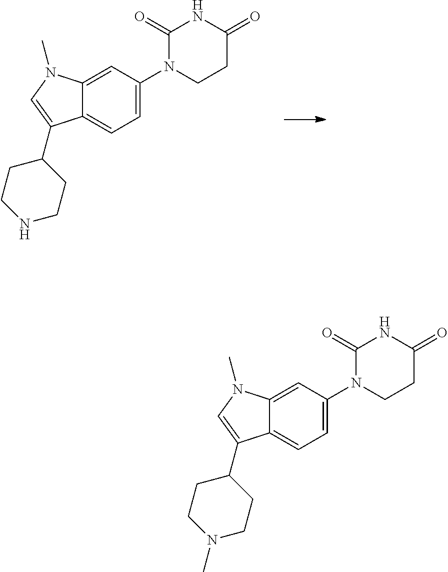 [SMILES: Cn1cc(C2CCNCC2)c2ccc(N3CCC(=O)NC3=O)cc21 ; CN1CCC(c2cn(C)c3cc(N4CCC(=O)NC4=O)ccc23)CC1]

Sodium triacetoxyborohydride (156 mg, 0.736 mmol) was added to a mixture of 1-(1-methyl-3-(piperidin-4-yl)-1H-indol-6-yl)dihydropyrimidine-2,4 (1H,3H)-dione (80.0 mg, 0.245 mmol) and paraformaldehyde (36.8 mg, 1.23 mmol) in DCM (3 mL) and MeOH (3 mL) at r.t. The resulting mixture was stirred at r.t. for 16 h. The solvent was then removed under reduced pressure. Purification by C-18FC (gradient: 0-30% MeCN in water (10 mmol NH₄HCO₃)) gave the title compound (62.0 mg, 74%) as a white solid. ¹H NMR: δ 1.68 (2H, qd), 1.86-1.95 (2H, m), 2.04 (2H, td), 2.21 (3H, s), 2.64-2.77 (3H, m), 2.86 (2H, dt), 3.71 (3H, s), 3.80 (2H, t), 6.95 (1H, dd), 7.12 (1H, s), 7.34 (1H, d), 7.54 (1H, d), 10.29 (1H, s). m/z (ES⁺), [M+H]⁺=341.2.

### Intermediate 120a: tert-Butyl 4-(6-bromo-1H-indol-1-yl)piperidine-1-carboxylate

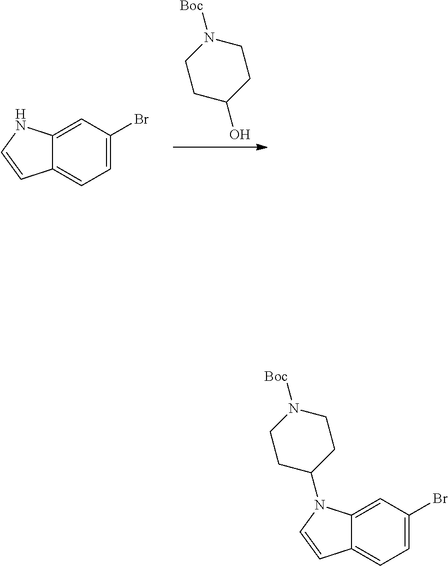 [SMILES: Brc1ccc2cc[nH]c2c1 ; CC(C)(C)OC(=O)N1CCC(O)CC1 ; CC(C)(C)OC(=O)N1CCC(n2ccc3ccc(Br)cc32)CC1]

(Cyanomethylene)tri-n-butylphosphorane (2.95 g, 12.2 mmol) was added to a stirred solution of tert-butyl 4-hydroxypiperidine-1-carboxylate (3.08 g, 15.3 mmol) and 6-bromo-1H-indole (2.00 g, 10.2 mmol) in 1,4-dioxane (20 mL) at r.t. under N₂. The resulting mixture was stirred at 100° C. for 16 h then concentrated under reduced pressure. Purification by FSC (gradient: 0-5% EtOAc in petroleum ether) gave the title compound (1.30 g, 34%) as a yellow solid. ¹H NMR: 1.41 (9H, s), 1.69-1.94 (4H, m), 2.95 (2H, br s), 4.09 (2H, br d), 4.53-4.67 (1H, m), 6.46 (1H, d), 7.12-7.14 (1H, m), 7.48 (1H, d), 7.54 (1H, d), 7.84-7.85 (1H, m). m/z: (ES⁺) [M+H]⁺ 379.1.

### Intermediate 120b: tert-Butyl 4-(6-(2,4-dioxotetrahydropyrimidin-1 (2H)-yl)-1H-indol-1-yl)piperidine-1-carboxylate

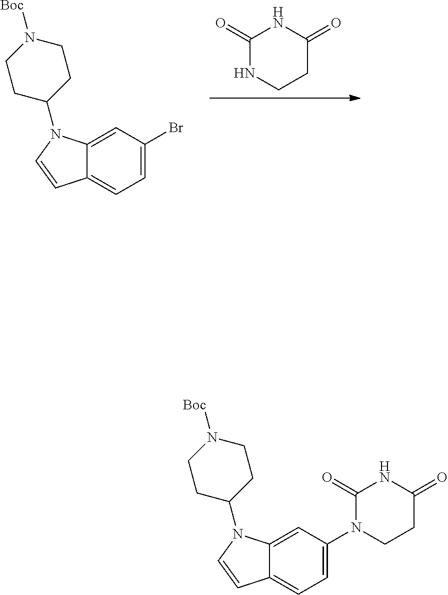 [SMILES: CC(C)(C)OC(=O)N1CCC(n2ccc3ccc(Br)cc32)CC1 ; O=C1CCNC(=O)N1 ; CC(C)(C)OC(=O)N1CCC(n2ccc3ccc(N4CCC(=O)NC4=O)cc32)CC1]

Ephos Pd G4 (157 mg, 0.171 mmol) and Ephos (92.0 mg, 0.172 mmol) were added in one portion to a degassed mixture of tert-butyl 4-(6-bromo-1H-indol-1-yl)piperidine-1-carboxylate (1.30 g, 3.43 mmol), dihydro-pyrimidine-2,4 (1H,3H)-dione (1.17 g, 10.3 mmol), and Cs₂CO₃ (2.23 g, 6.84 mmol) in 1,4-dioxane (40 mL) at r.t. under N₂. The resulting mixture was stirred at 100° C. for 16 h. The reaction was cooled to r.t. and concentrated. Purification by FSC (gradient: 0-70% EtOAc in petroleum ether) gave the title compound (580 mg, 41%) as a white solid. ¹H NMR: 1.44 (9H, s), 1.78-1.93 (3H, m), 2.74 (2H, t), 2.97 (3H, br s), 3.81 (2H, t), 4.07-4.18 (2H, m), 4.49-4.60 (1H, m), 6.47 (1H, d), 6.98-7.11 (1H, m), 7.49-7.59 (3H, m), 10.31 (1H, s). m/z: (ES⁺) [M+H]⁺ 413.1.

### Example 120: 1-(1-(Piperidin-4-yl)-1H-indol-6-yl)dihydropyrimidine-2,4 (1H,3H)-dione

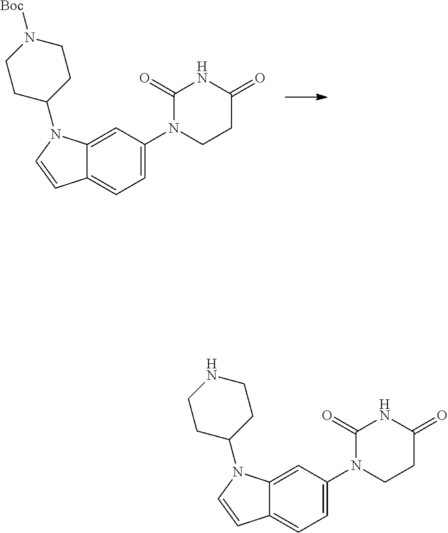 [SMILES: CC(C)(C)OC(=O)N1CCC(n2ccc3ccc(N4CCC(=O)NC4=O)cc32)CC1 ; O=C1CCN(c2ccc3ccn(C4CCNCC4)c3c2)C(=O)N1]

tert-Butyl 4-(6-(2,4-dioxotetrahydropyrimidin-1 (2H)-yl)-1H-indol-1-yl)piperidine-1-carboxylate (500 mg, 1.21 mmol) was added to AcOH (10 mL) at r.t. The resulting mixture was stirred at 100° C. for 16 h. The solvent was then removed under reduced pressure. Purification by C-18FC (gradient: 0-30% MeCN in water (containing 0.05% conc. HCl)) gave the title compound in the form of a hydrochloride salt (380 mg, 90%) as a pale yellow solid. ¹H NMR: 1.92-2.15 (4H, m), 2.74 (2H, t), 2.91 (2H, td), 3.26 (2H, d), 3.80 (2H, t), 4.54 (1H, dt), 6.48 (1H, d), 6.99 (1H, dd), 7.47 (1H, d), 7.53 (2H, d), 8.35 (1H, s), 10.33 (1H, s). m/z: (ES⁺) [M+H]⁺ 313.1.

### Example 121: 1-(1-(1-Methylpiperidin-4-yl)-1H-indol-6-yl)dihydropyrimidine-2,4 (1H,3H)-dione

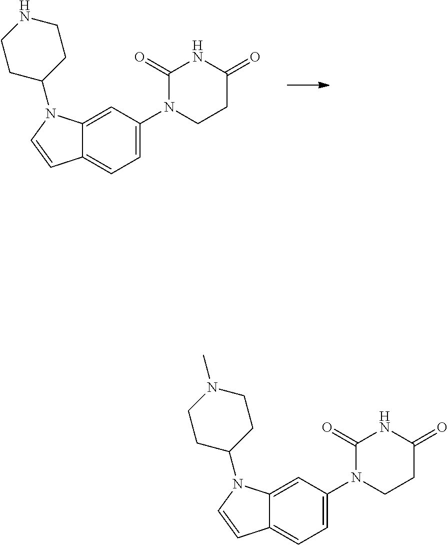 [SMILES: O=C1CCN(c2ccc3ccn(C4CCNCC4)c3c2)C(=O)N1 ; CN1CCC(n2ccc3ccc(N4CCC(=O)NC4=O)cc32)CC1]

AcOH (12.3 μL, 0.215 mmol) was added to a mixture of 1-(1-(piperidin-4-yl)-1H-indol-6-yl)dihydro-pyrimidine-2,4 (1H,3H)-dione hydrochloride (75.0 mg, 0.215 mmol), formaldehyde (19.4 mg, 0.646 mmol) in DCM (3 mL) at r.t. under air. The reaction mixture was stirred for 0.5 h before the addition of sodium triacetoxyborohydride (137 mg, 0.646 mmol). The resulting mixture was stirred at r.t. for 1 h. The solvent was then removed under reduced pressure. Purification by C-18FC (gradient: 5-50% MeCN in water (containing 0.05% conc. HCl)) gave material that was further purified by preparative HPLC (Column F, Eluent A, gradient: 20-30%) and further purified by preparative HPLC (Column A, Eluent F, gradient: 16-30%) to give the title compound (13.0 mg, 19%) as a white solid. ¹H NMR: δ 1.86-1.92 (2H, m), 1.94-2.10 (2H, m), 2.15 (2H, t), 2.24 (3H, s), 2.73 (2H, t), 2.91 (2H, d), 3.80 (2H, t), 4.24-4.35 (1H, m), 6.46 (1H, d), 6.97 (1H, dd), 7.48-7.56 (3H, m), 10.30 (1H, s). m/z (ES⁺), [M+H]⁺=327.1.

### Example 122: 1-(1-(1-Acetylpiperidin-4-yl)-1H-indol-6-yl)dihydropyrimidine-2,4 (1H,3H)-dione

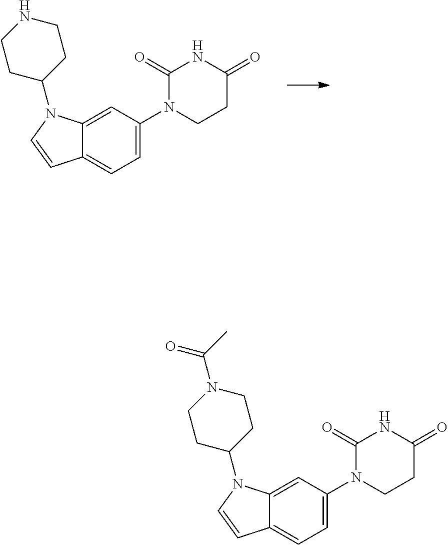 [SMILES: O=C1CCN(c2ccc3ccn(C4CCNCC4)c3c2)C(=O)N1 ; CC(=O)N1CCC(n2ccc3ccc(N4CCC(=O)NC4=O)cc32)CC1]

Ac₂O (41.0 mg, 0.402 mmol) was added to a solution of 1-(1-(piperidin-4-yl)-1H-indol-6-yl)dihydropyrimidine-2,4 (1H,3H)-dione hydrochloride (70 mg, 0.201 mmol) and triethylamine (84.0 μL, 0.603 mmol) in DCM (3 mL) at r.t. under air. The resulting mixture was stirred at r.t. for 1 h. The solvent was then removed under reduced pressure. Purification by C-18FC (gradient: 5-50% MeCN in water (containing 0.05% conc. HCl)) gave material that was further purified by preparative HPLC (Column F, Eluent A, gradient: 30-35%) to give the title compound (30.0 mg, 42%) as a white solid. ¹H NMR: δ 1.70-1.85 (1H, m), 1.89-1.99 (3H, m), 2.06 (3H, s), 2.67-2.79 (3H, m), 3.21-3.33 (1H, m), 3.81 (2H, t), 3.93-4.01 (1H, m), 4.53-4.68 (2H, m), 6.47 (1H, d), 6.98 (1H, dd), 7.49-7.58 (3H, m), 10.31 (1H, s). m/z (ES⁺), [M+H]⁺=355.3.

### Intermediate 123a: tert-Butyl 4-(4-bromo-1H-indol-7-yl)piperazine-1-carboxylate

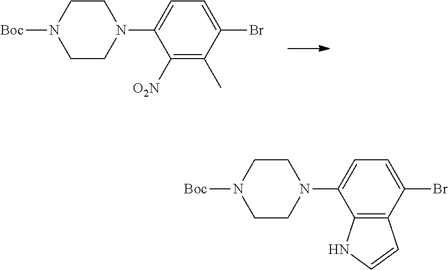 [SMILES: Cc1c(Br)ccc(N2CCN(C(=O)OC(C)(C)C)CC2)c1[N+](=O)[O-] ; CC(C)(C)OC(=O)N1CCN(c2ccc(Br)c3cc[nH]c23)CC1]

1-tert-Butoxy-N,N,N′,N′-tetramethylmethanediamine (50.0 mL, 242 mmol) was added to tert-butyl 4-(4-bromo-3-methyl-2-nitrophenyl)piperazine-1-carboxylate (prepared by the method described in WO2016049524, 7.00 g, 17.5 mmol) in DMF (50 mL) at r.t. under air. The resulting solution was stirred at 100° C. for 17 h. The reaction mixture was poured into water (150 mL) and extracted with EtOAc (3×100 mL) and the combined organic extracts were dried (Na₂SO₄) and concentrated. Iron (19.5 g, 349 mmol) and saturated aq. NH₄Cl (30 mL, 17.49 mmol) were added to the resulting residue dissolved in ethanol (150 mL) and the mixture was stirred at 80° C. for 16 h. The reaction mixture was then filtered and the organics removed under reduced pressure. The concentrated mixture was diluted with water (50 mL) and extracted with EtOAc (3×100 mL). The combined organic extracts were dried (Na₂SO₄) and concentrated. Purification by FSC (gradient: 0-30% EtOAc in petroleum ether) gave the title compound (3.00 g, 45%) as a pale yellow solid. ¹H NMR: 1.44 (9H, s), 2.92-2.99 (4H, m), 3.54-3.61 (4H, m), 6.39 (1H, dd), 6.61 (1H, d), 7.11 (1H, d), 7.41 (1H, t), 11.30 (1H, s). m/z: (ES⁺) [M+H]⁺ 382.1.

### Intermediate 123b: tert-Butyl 4-bromo-7-(4-(tert-butoxycarbonyl)piperazin-1-yl)-1H-indole-1-carboxylate

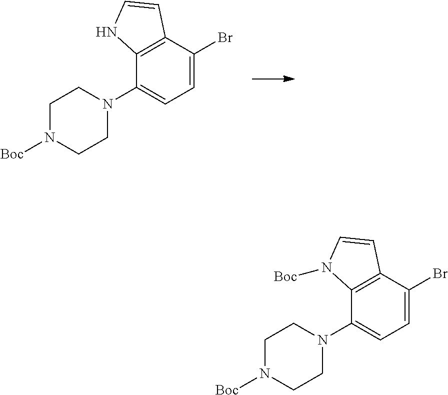 [SMILES: CC(C)(C)OC(=O)N1CCN(c2ccc(Br)c3cc[nH]c23)CC1 ; CC(C)(C)OC(=O)N1CCN(c2ccc(Br)c3ccn(C(=O)OC(C)(C)C)c23)CC1]

DMAP (48.0 mg, 0.393 mmol) was added to a mixture of DIEA (1.38 mL, 7.90 mmol), di-tert-butyl dicarbonate (1.37 mL, 5.90 mmol) and tert-butyl 4-(4-bromo-1H-indol-7-yl)piperazine-1-carboxylate (1.50 g, 3.94 mmol) in DCM (20 mL) at r.t. under air. The resulting mixture was stirred at r.t. for 2 h. The solvent was then removed under reduced pressure. Purification by FSC (gradient: 0-20% EtOAc in petroleum ether) gave the title compound (1.70 g, 90%) as a pale yellow solid. ¹H NMR: (CDCl₃) 1.51 (9H, s), 1.66 (9H, s), 2.97 (4H, br s), 3.64 (4H, br s), 6.64 (1H, d), 6.80 (1H, d), 7.34 (1H, d), 7.53 (1H, d). m/z: (ES⁺) [M-Boc+2H]⁺=380.2.

### Intermediate 123c: tert-Butyl 7-(4-(tert-butoxycarbonyl)piperazin-1-yl)-4-(2,4-dioxotetrahydro-pyrimidin-1 (2H)-yl)-1H-indole-1-carboxylate

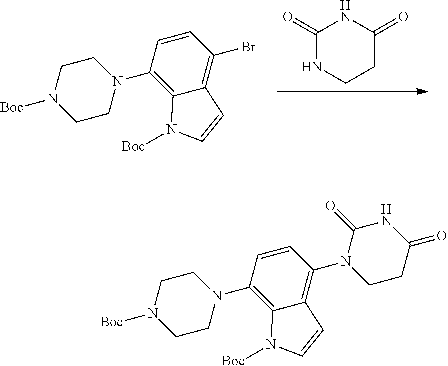 [SMILES: CC(C)(C)OC(=O)N1CCN(c2ccc(Br)c3ccn(C(=O)OC(C)(C)C)c23)CC1 ; O=C1CCNC(=O)N1 ; CC(C)(C)OC(=O)N1CCN(c2ccc(N3CCC(=O)NC3=O)c3ccn(C(=O)OC(C)(C)C)c23)CC1]

Ephos Pd G4 (325 mg, 0.354 mmol) and Ephos (189 mg, 0.353 mmol) were added in one portion to a degassed mixture of tert-butyl 4-bromo-7-(4-(tert-butoxycarbonyl)piperazin-1-yl)-1H-indole-1-carboxylate (1.70 g, 3.54 mmol), dihydropyrimidine-2,4 (1H,31H)-dione (1.21 g, 10.6 mmol), and Cs₂CO₃ (3.46 g, 10.6 mmol) in DMF (100 mL) at r.t. under N₂. The resulting mixture was stirred at 100° C. for 17 h. The reaction mixture was then poured into ice water (200 mL) and extracted with EtOAc (3×200 mL). The combined organic extracts were dried (Na₂SO₄) and concentrated to give a red gum. Purification FSC (gradient: 0-100% EtOAc in petroleum ether) gave the title compound (300 mg, 17%) as a pale yellow solid. m/z: (ES⁺) [M+H]⁺ 514.3.

### Example 123: 1-(7-(Piperazin-1-yl)-1H-indol-4-yl)dihydropyrimidine-2,4 (1H,3H)-dione

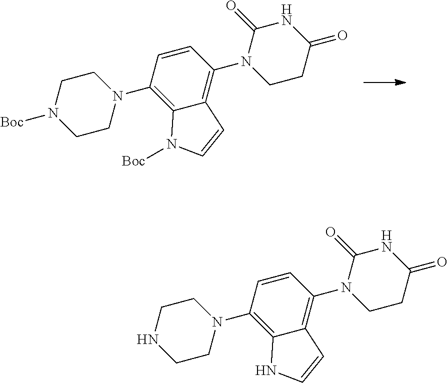 [SMILES: CC(C)(C)OC(=O)N1CCN(c2ccc(N3CCC(=O)NC3=O)c3ccn(C(=O)OC(C)(C)C)c23)CC1 ; O=C1CCN(c2ccc(N3CCNCC3)c3[nH]ccc23)C(=O)N1]

tert-Butyldimethylsilyl trifluoromethanesulfonate (288 mg, 1.09 mmol) was added to a stirred solution of tert-butyl 7-(4-(tert-butoxycarbonyl)piperazin-1-yl)-4-(2,4-dioxotetrahydropyrimidin-1 (2H)-yl)-1H-indole-1-carboxylate (280 mg, 0.545 mmol) in MeCN (100 mL) at r.t. under air. The resulting mixture was stirred at r.t. for 1 h. The solvent was then removed under reduced pressure. Purification by C-18FC (gradient: 0-20% MeCN in water (containing 0.1% conc. HCl)) gave the title compound in the form of a hydrochloride salt (180 mg, 94%) as a pale yellow solid. ¹H NMR: 2.76 (2H, t), 3.25 (4H, t), 3.34 (4H, d), 3.74 (2H, t), 6.41 (1H, dd), 6.71 (1H, d), 6.88 (1H, d), 7.36 (1H, t), 9.22 (2H, s), 10.29 (1H, s), 11.23 (1H, s). m/z: (ES⁺) [M+H]⁺=314.2.

### Example 124: 1-(7-(4-Methylpiperazin-1-yl)-1H-indol-4-yl)dihydropyrimidine-2,4 (1H,3H)-dione

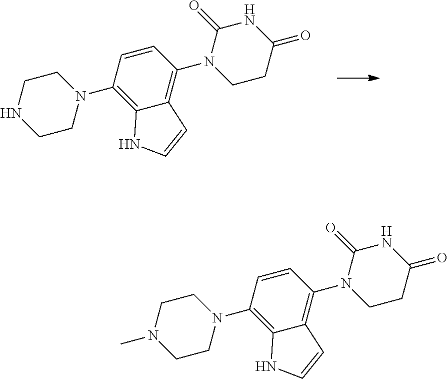 [SMILES: O=C1CCN(c2ccc(N3CCNCC3)c3[nH]ccc23)C(=O)N1 ; CN1CCN(c2ccc(N3CCC(=O)NC3=O)c3cc[nH]c23)CC1]

AcOH (13.1 μL, 0.229 mmol) was added to a mixture of NaOAc (75.0 mg, 0.914 mmol), sodium triacetoxyborohydride (242 mg, 1.14 mmol), formaldehyde (20.6 mg, 0.686 mmol) and 1-(7-(piperazin-1-yl)-1H-indol-4-yl)dihydropyrimidine-2,4 (1H,3H)-dione hydrochloride (80.0 mg, 0.229 mmol) in DCM (10 mL) and MeOH (1.0 mL) at r.t. under air. The resulting mixture was stirred at r.t. for 17 h. The solvent was then removed under reduced pressure. Purification by C-18FC (gradient: 0-20% MeCN in water (containing 0.1% FA)) gave the title compound in the form of a formate salt (45.0 mg, 53%) as a pale yellow solid. ¹H NMR: δ 2.36 (3H, s), 2.70 (4H, s), 2.75 (2H, t), 3.08 (4H, s), 3.73 (2H, t), 6.37 (1H, t), 6.66 (1H, d), 6.84 (1H, d), 7.29 (1H, t), 8.17 (1H, s), 10.26 (1H, s), 10.98 (1H, s). m/z (ES⁺), [M+H]⁺=328.2.

### Intermediate 125a: tert-Butyl 4-(4-(2,4-dioxotetrahydropyrimidin-1 (2H)-yl)-1-methyl-1H-indol-7-yl)piperazine-1-carboxylate

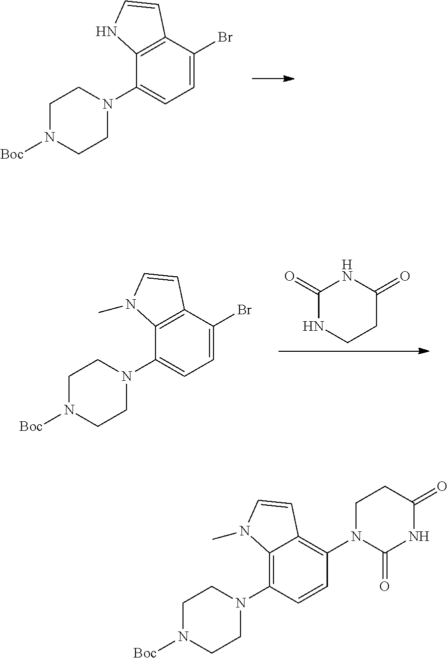 [SMILES: CC(C)(C)OC(=O)N1CCN(c2ccc(Br)c3cc[nH]c23)CC1 ; Cn1ccc2c(Br)ccc(N3CCN(C(=O)OC(C)(C)C)CC3)c21 ; O=C1CCNC(=O)N1 ; Cn1ccc2c(N3CCC(=O)NC3=O)ccc(N3CCN(C(=O)OC(C)(C)C)CC3)c21]

NaH (60% dispersion in mineral oil, 268 mg, 6.71 mmol) was added to a solution of tert-butyl 4-(4-bromo-1H-indol-7-yl)piperazine-1-carboxylate (1.70 g, 4.47 mmol) in THF (20 mL) at 0° C. under N₂ and stirred at r.t. for 0.5 h. Mel (334 μL, 5.36 mmol) was then added at 0° C. and the reaction was stirred at r.t. for 2 h. The reaction mixture was quenched with saturated aq. NH₄C1 (20 mL) and extracted with EtOAc (3×50 mL). The combined organic extracts were dried (Na₂SO₄) and concentrated to give a pale yellow gum (1.70 g) which was used in the next step without further purification. m/z (ES⁺), [M+H]⁺=396.2. The gum was then dissolved in DMF (100 mL) and to the solution was added Cs₂CO₃ (4.21 g, 12.9 mmol) and dihydropyrimidine-2,4 (1H,3H)-dione (1.48 g, 13.0 mmol) before the mixture was degassed. Ephos Pd G4 (396 mg, 0.431 mmol) and Ephos (231 mg, 0.432 mmol) were added in one portion at r.t. under N₂. The resulting mixture was stirred at 100° C. for 17 h. The reaction was cooled to r.t., poured into ice water (200 mL) and extracted with EtOAc (3×200 mL). The combined organic extracts were dried (Na₂SO₄) and concentrated to give a red oil. Purification by FSC (gradient: 0-100% EtOAc in petroleum ether) gave the title compound (300 mg, 16% over two-steps) as a pale yellow solid. ¹H NMR: (CDCl₃) δ 1.52 (9H, s), 2.84-2.95 (4H, m), 3.05-3.25 (4H, m), 3.91 (2H, t), 4.06-4.26 (5H, m), 6.33 (1H, d), 6.96 (2H, d), 7.03 (1H, d), 7.49 (1H, s). m/z (ES⁺), [M+H]⁺=428.1.

### Example 125: 1-(1-Methyl-7-(piperazin-1-yl)-1H-indol-4-yl)dihydropyrimidine-2,4 (1H,3H)-dione

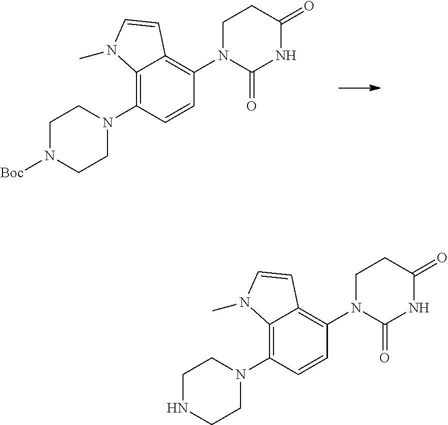 [SMILES: Cn1ccc2c(N3CCC(=O)NC3=O)ccc(N3CCN(C(=O)OC(C)(C)C)CC3)c21 ; Cn1ccc2c(N3CCC(=O)NC3=O)ccc(N3CCNCC3)c21]

tert-Butyldimethylsilyl trifluoromethanesulfonate (346 mg, 1.31 mmol) was added to a stirred solution of tert-butyl 4-(4-(2,4-dioxotetrahydropyrimidin-1 (2H)-yl)-1-methyl-1H-indol-7-yl)piperazine-1-carboxylate (280 mg, 0.655 mmol) in MeCN (100 mL) at r.t. under air. The resulting mixture was stirred at r.t. for 1 h. The solvent was then removed under reduced pressure. Purification by C-18FC (gradient: 0-20% MeCN in water (containing 0.1% conc. HCl)) gave the title compound in the form of a hydrochloride salt (200 mg, 84%) as a pale yellow solid. ¹H NMR: 2.76 (2H, t), 3.05-3.16 (2H, m), 3.21-3.29 (4H, m), 3.33-3.41 (2H, m) 3.68-3.76 (2H, m), 4.10 (3H, s), 6.38 (1H, d), 6.91 (2H, s), 7.28 (1H, d), 9.08-9.12 (1H, m), 9.36 (1H, br s), 10.31 (1H, s). m/z: (ES⁺) [M+H]⁺ 328.2.

### Example 126: 1-(1-Methyl-7-(4-methylpiperazin-1-yl)-1H-indol-4-yl)dihydropyrimidine-2,4 (1H,3H)-dione

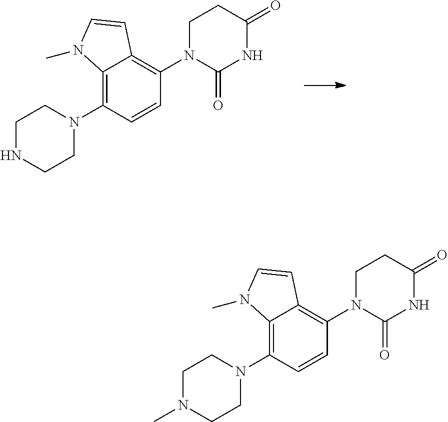 [SMILES: Cn1ccc2c(N3CCC(=O)NC3=O)ccc(N3CCNCC3)c21 ; CN1CCN(c2ccc(N3CCC(=O)NC3=O)c3ccn(C)c23)CC1]

AcOH (12.6 μL, 0.218 mmol) was added to a mixture of NaOAc (72.1 mg, 0.879 mmol), sodium triacetoxyborohydride (140 mg, 0.661 mmol), formaldehyde (19.8 mg, 0.659 mmol) and 1-(1-methyl-7-(piperazin-1-yl)-1H-indol-4-yl)dihydropyrimidine-2,4 (1H,3H)-dione hydrochloride (80 mg, 0.220 mmol) in DCM (1 mL) and MeOH (0.1 mL) at r.t. under air. The resulting mixture was stirred at r.t. for 17 h. The solvent was then removed under reduced pressure. Purification by C-18FC (gradient: 0-20% MeCN in water (containing 0.1% FA)) gave the title compound in the form of a formate salt (45.0 mg, 53%) as a pale yellow solid. ¹H NMR: δ 2.38 (3H, s), 2.58-2.63 (2H, m), 2.75 (2H, t), 2.87-2.98 (4H, m), 3.06-3.13 (2H, m), 3.71 (2H, t), 4.09 (3H, s), 6.35 (1H, d), 6.84-6.93 (2H, m), 7.23 (1H, d), 8.17 (1H, s), 10.28 (1H, s). m/z (ES⁺), [M+H]⁺=342.3.

### Intermediate 127a: tert-Butyl (S)-(2-(2-(4-(4-chlorophenyl)-2,3,9-trimethyl-6H-thieno[3,2-f][1,2,4]triazolo-[4,3-a][1,4]diazepin-6-yl)acetamido)ethyl)carbamate

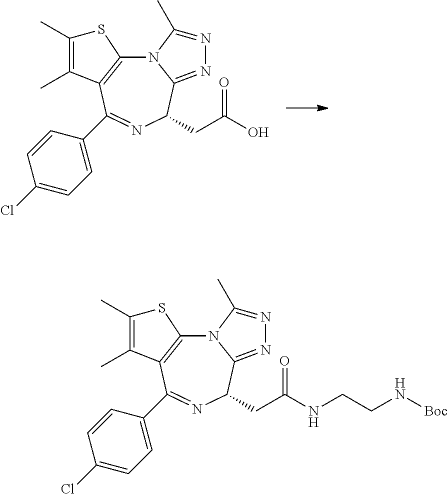 [SMILES: Cc1sc2c(c1C)C(c1ccc(Cl)cc1)=N[C@@H](CC(=O)O)c1nnc(C)n1-2 ; Cc1sc2c(c1C)C(c1ccc(Cl)cc1)=N[C@@H](CC(=O)NCCNC(=O)OC(C)(C)C)c1nnc(C)n1-2]

tert-Butyl (2-aminoethyl)carbamate (719 mg, 4.49 mmol) was added to a mixture of DIEA (1.31 mL, 7.50 mmol), O-(7-azabenzotriazol-1-yl)-N,N′,N′-tetramethyluronium hexafluorophosphate (1.71 g, 4.50 mmol) and (S)-2-(4-(4-chlorophenyl)-2,3,9-trimethyl-6H-thieno[3,2-f][1,2,4]triazolo[4,3-a][1,4]diazepin-6-yl)acetic acid (1.50 g, 3.74 mmol) in MeCN (30 mL). The resulting solution was stirred at r.t. for 2 h. The solvent was then removed under reduced pressure. Purification by C-18FC (gradient 0-30% MeCN in water (containing 0.1% TFA)) gave the title compound (1.90 g, 93%) as a yellow solid. ¹H NMR: δ 1.38 (9H, s), 1.60-1.66 (3H, m), 2.08 (2H, s), 2.42 (3H, s), 2.60 (3H, s), 2.98-3.27 (4H, m), 4.51 (1H, t), 6.80 (1H, t), 7.39-7.53 (4H, m), 8.23 (1H, t). m/z (ES⁺), [M+H]⁺=543.20.

### Intermediate 127b: (S)—N-(2-Aminoethyl)-2-(4-(4-chlorophenyl)-2,3,9-trimethyl-6H-thieno[3,2-f][1,2,4]triazolo[4,3-a][1,4]diazepin-6-yl)acetamide

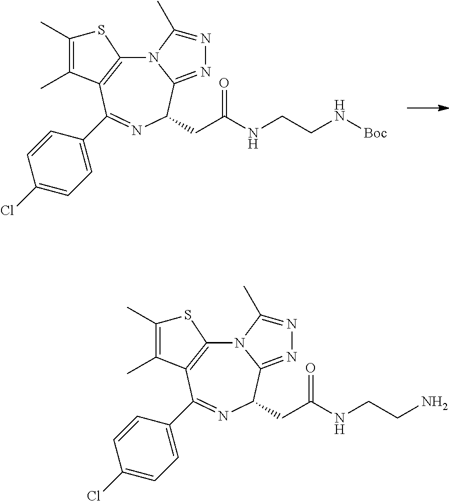 [SMILES: Cc1sc2c(c1C)C(c1ccc(Cl)cc1)=N[C@@H](CC(=O)NCCNC(=O)OC(C)(C)C)c1nnc(C)n1-2 ; Cc1sc2c(c1C)C(c1ccc(Cl)cc1)=N[C@@H](CC(=O)NCCN)c1nnc(C)n1-2]

tert-Butyl (S)-(2-(2-(4-(4-chlorophenyl)-2,3,9-trimethyl-6H-thieno[3,2-f][1,2,4]triazolo[4,3-a][1,4]diazepin-6-yl)acetamido)ethyl)carbamate (1.85 g, 3.41 mmol) was added to a solution of TFA (15 mL) and DCM (15 mL). The resulting solution was stirred at r.t. for 2 h. The solvent was then removed under reduced pressure. Purification by C-18FC (gradient: 0-50% MeOH in water (containing 0.1% conc. HCl)) gave the title compound in the form of a hydrochloride salt (1.46 g, 89%) as a yellow solid. ¹H NMR: δ 1.62 (3H, s), 2.41 (3H, s), 2.62 (3H, s), 2.83-2.95 (2H, m), 3.30 (2H, d), 3.38 (2H, q), 4.55 (1H, t), 7.43 (2H, d), 7.50 (2H, d), 8.12 (3H, br s), 8.53 (1H, t). m/z (ES⁺), [M+H]⁺=443.2.

### Example 127: (S)-2-(4-(4-Chlorophenyl)-2,3,9-trimethyl-6H-thieno[3,2-f][1,2,4]triazolo[4,3-a][1,4]-diazepin-6-yl)-N-(2-(2-(6-(2,4-dioxotetrahydropyrimidin-1 (2H)-yl)-1H-indol-3-yl)acetamido)ethyl)-acetamide

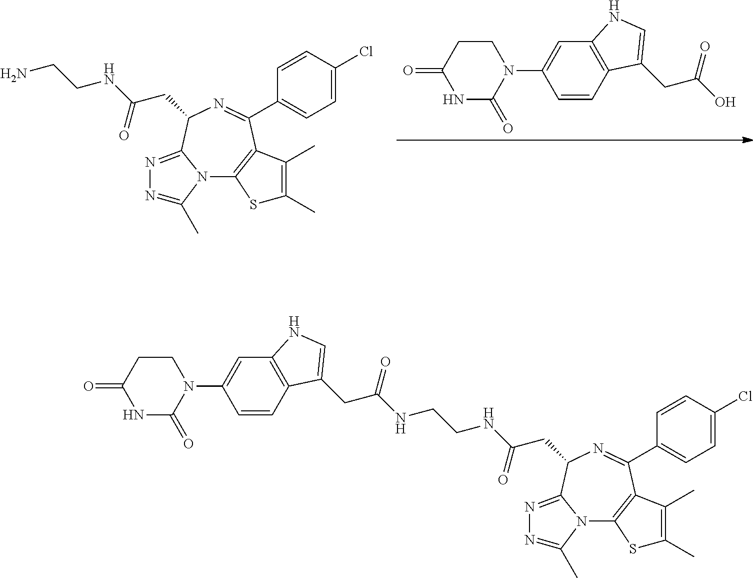 [SMILES: Cc1sc2c(c1C)C(c1ccc(Cl)cc1)=N[C@@H](CC(=O)NCCN)c1nnc(C)n1-2 ; O=C(O)Cc1c[nH]c2cc(N3CCC(=O)NC3=O)ccc12 ; Cc1sc2c(c1C)C(c1ccc(Cl)cc1)=N[C@@H](CC(=O)NCCNC(=O)Cc1c[nH]c3cc(N4CCC(=O)NC4=O)ccc13)c1nnc(C)n1-2]

2-(6-(2,4-Dioxotetrahydropyrimidin-1 (2H)-yl)-1H-indol-3-yl)acetic acid (50.0 mg, 0.174 mmol) was added to a mixture of (S)—N-(2-aminoethyl)-2-(4-(4-chlorophenyl)-2,3,9-trimethyl-6H-thieno[3,2-f][1,2,4]triazolo[4,3-a][1,4]diazepin-6-yl)acetamide hydrochloride (83.0 mg, 0.173 mmol), HOBt (32.0 mg, 0.209 mmol), EDC (40.0 mg, 0.209 mmol) and DIEA (60.8 μL, 0.348 mmol) in DMF (2 mL). The resulting mixture was stirred at r.t. for 16 h and then purified directly by preparative HPLC (Column G, Eluent E, gradient: 33-35%) to give the title compound (52.0 mg, 42%) as a brown solid. ¹H NMR: δ 1.62 (3H, s), 2.41 (3H, s), 2.59 (3H, s), 2.71 (2H, t), 3.12-3.19 (4H, m), 3.22 (2H, dd), 3.50 (2H, s), 3.77 (2H, t), 4.51 (1H, t), 6.92 (1H, dd), 7.23 (1H, d), 7.27 (1H, d), 7.38-7.47 (2H, m), 7.44-7.55 (3H, m), 7.90-7.96 (1H, m), 8.22-8.30 (1H, m), 10.27 (1H, s), 10.95 (1H, d). m/z (ES⁺), M⁺=712.3.

### Intermediate 128a: tert-Butyl (S)-(2-(2-(2-(2-(4-(4-chlorophenyl)-2,3,9-trimethyl-6H-thieno[3,2-f]-[1,2,4]triazolo[4,3-a][1,4]diazepin-6-yl)acetamido)ethoxy)ethoxy)ethyl)carbamate

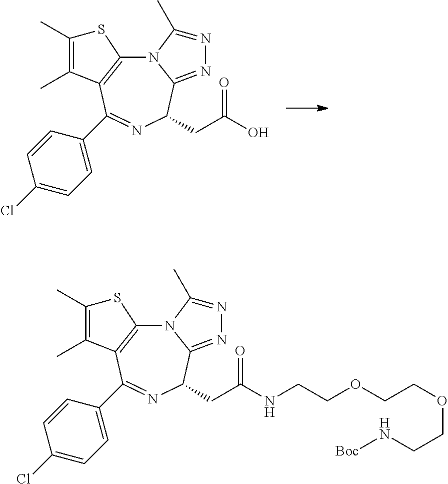 [SMILES: Cc1sc2c(c1C)C(c1ccc(Cl)cc1)=N[C@@H](CC(=O)O)c1nnc(C)n1-2 ; Cc1sc2c(c1C)C(c1ccc(Cl)cc1)=N[C@@H](CC(=O)NCCOCCOCCNC(=O)OC(C)(C)C)c1nnc(C)n1-2]

tert-Butyl (2-(2-(2-aminoethoxy)ethoxy)ethyl)carbamate (1.115 g, 4.490 mmol) was added to a mixture of DIEA (1.046 mL, 5.989 mmol), O-(7-azabenzotriazol-1-yl)-N,N,N′,N′-tetramethyluronium hexafluorophosphate (1.366 g, 3.593 mmol) and (S)-2-(4-(4-chlorophenyl)-2,3,9-trimethyl-6H-thieno[3,2-f][1,2,4]triazolo[4,3-a][1,4]-diazepin-6-yl)acetic acid (1.200 g, 2.993 mmol) in MeCN (30 mL). The resulting solution was stirred at r.t. for 2 h. The solvent was then removed under reduced pressure. Purification by C-18FC (gradient: 0-30% MeCN in water (containing 0.1% TFA)) gave the title compound (1.800 g, 95%) as a yellow solid. ¹H NMR: δ 1.37 (9H, s), 1.63 (3H, s), 2.42 (3H, s), 2.60 (3H, s), 3.07 (2H, q), 3.25 (2H, t), 3.34-3.55 (8H, m), 3.55-3.71 (2H, m), 4.33-4.67 (1H, m), 6.77 (1H, t), 7.38-7.54 (4H, m), 8.28 (1H, t). m/z (ES⁺), [M+H]⁺=631.3

### Intermediate 128b: (S)—N-(2-(2-(2-Aminoethoxy)ethoxy)ethyl)-2-(4-(4-chlorophenyl)-2,3,9-trimethyl-6H-thieno[3,2-f][1,2,4]triazolo[4,3-a][1,4]diazepin-6-yl)acetamide

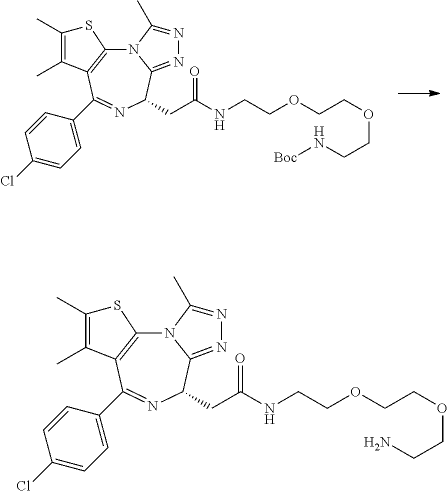 [SMILES: Cc1sc2c(c1C)C(c1ccc(Cl)cc1)=N[C@@H](CC(=O)NCCOCCOCCNC(=O)OC(C)(C)C)c1nnc(C)n1-2 ; Cc1sc2c(c1C)C(c1ccc(Cl)cc1)=N[C@@H](CC(=O)NCCOCCOCCN)c1nnc(C)n1-2]

tert-Butyl (S)-(2-(2-(2-(2-(4-(4-chlorophenyl)-2,3,9-trimethyl-6H-thieno[3,2-f][1,2,4]triazolo[4,3-a][1,4]-diazepin-6-yl)acetamido)ethoxy)ethoxy)ethyl)carbamate (1.75 g, 2.77 mmol) was added to a solution of TFA (15 mL) and DCM (15 mL). The resulting solution was stirred at r.t. for 2 h. The solvent was then removed under reduced pressure. Purification by C-18FC (gradient: 0-50% MeOH in water (containing 0.1% conc. HCl)) gave the title compound in the form of a hydrochloride salt (1.40 g, 89%) as a yellow solid. ¹H NMR: δ 1.62 (3H, s), 2.42 (3H, s), 2.64 (3H, s), 2.95 (2H, q), 3.22-3.34 (4H, m), 3.47 (2H, t), 3.58 (4H, br s), 3.63 (2H, t), 4.56 (1H, t), 7.38-7.55 (4H, m), 8.08 (3H, br s), 8.35 (1H, t). m/z (ES⁺), [M+H]⁺=531.3.

### Example 128: (S)-2-(4-(4-Chlorophenyl)-2,3,9-trimethyl-6H-thieno[3,2-f][1,2,4]triazolo[4,3-a][1,4]-diazepin-6-yl)-N-(2-(2-(2-(2-(6-(2,4-dioxotetrahydropyrimidin-1 (2H)-yl)-1H-indol-3-yl)acetamido)-ethoxy)ethoxy)ethyl)acetamide

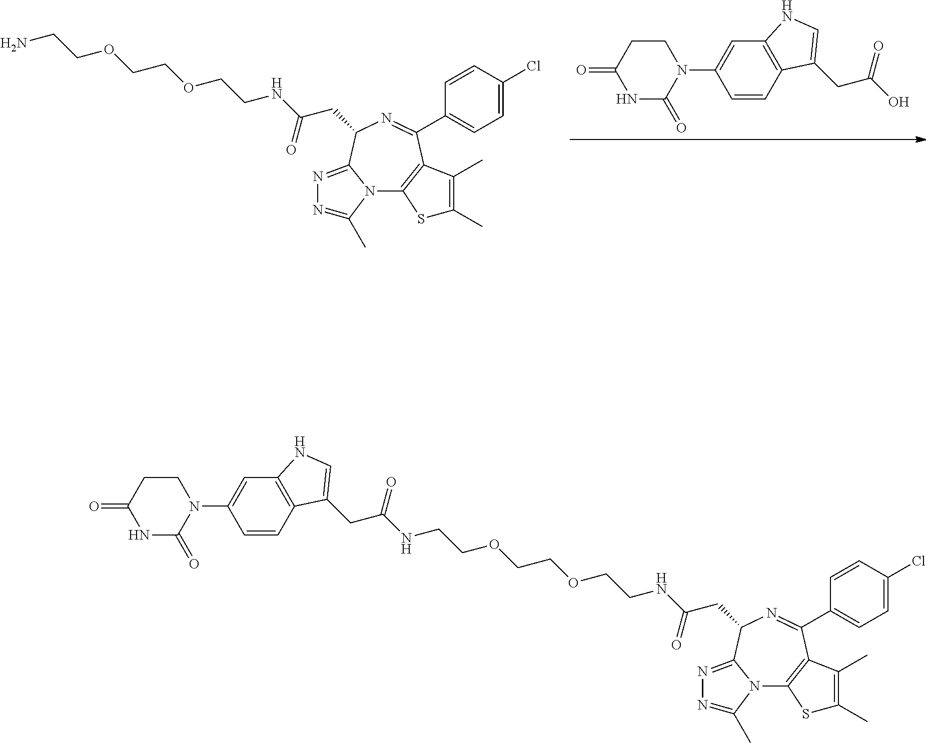 [SMILES: Cc1sc2c(c1C)C(c1ccc(Cl)cc1)=N[C@@H](CC(=O)NCCOCCOCCN)c1nnc(C)n1-2 ; O=C(O)Cc1c[nH]c2cc(N3CCC(=O)NC3=O)ccc12 ; Cc1sc2c(c1C)C(c1ccc(Cl)cc1)=N[C@@H](CC(=O)NCCOCCOCCNC(=O)Cc1c[nH]c3cc(N4CCC(=O)NC4=O)ccc13)c1nnc(C)n1-2]

2-(6-(2,4-Dioxotetrahydropyrimidin-1 (26)-yl)-1H-indol-3-yl)acetic acid (30.0 mg, 0.104 mmol) was added to a mixture of(S)—N-(2-(2-(2-aminoethoxy)ethoxy)ethyl)-2-(4-(4-chlorophenyl)-2,3,9-trimethyl-6H-thieno[3,2-f][1,2,4]triazolo[4,3-a][1,4]diazepin-6-yl)acetamide hydrochloride (59.3 mg, 0.104 mmol), HOBt (19.2 mg, 0.125 mmol), EDC (24.0 mg, 0.125 mmol) and DIEA (36.5 μL, 0.209 mmol) in DMF (2 me). The resulting mixture was stirred at r.t. for 16 h and then purified directly by preparative HPLC (Column G, Eluent E, 28-42%) gave the title compound (14.0 mg, 17%) as a white solid. H NMR: δ 1.61 (3H, s), 2.40 (3H, s), 2.59 (3H5, s), 2.71 (2H, t), 3.16-3.34 (6H, s), 3.37-3.53 (4H, m), 3.51 (6H, m), 3.77 (2H, t), 4.51 (1H, t), 6.91 (1H, dd), 7.22 (1H, d), 7.27 (1H, d), 7.38-7.46 (2H, n), 7.46-7.55 (3H, n), 7.95 (1H, t), 8.29 (1, t), 10.27 (1, s), 10.94 (1H, d). m/z (ES⁺), [M+H]⁺=800.4.

### Intermediate 129a: tert-Butyl (S)-(1-(4-(4-chlorophenyl)-2,3,9-trimethyl-6H-thieno[3,2-f][1,2,4]triazolo-[4,3-a][1,4]diazepin-6-yl)-2-oxo-6,9,12,15,18,21-hexaoxa-3-azatricosan-23-yl)carbamate

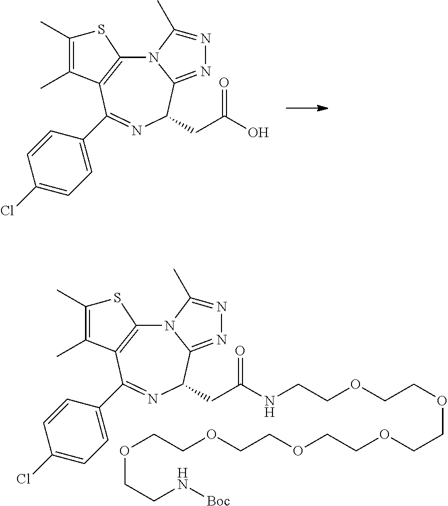 [SMILES: Cc1sc2c(c1C)C(c1ccc(Cl)cc1)=N[C@@H](CC(=O)O)c1nnc(C)n1-2 ; Cc1sc2c(c1C)C(c1ccc(Cl)cc1)=N[C@@H](CC(=O)NCCOCCOCCOCCOCCOCCOCCNC(=O)OC(C)(C)C)c1nnc(C)n1-2]

tert-Butyl (20-amino-3,6,9,12,15,18-hexaoxaicosyl)carbamate (1.38 g, 3.25 mmol) was added to a mixture of DIEA (0.871 mL, 4.99 mmol), O-(7-azabenzotriazol-1-yl)-N,N,N′,N′-tetramethyluronium hexafluorophosphate (1.14 g, 3.00 mmol) and (S)-2-(4-(4-chlorophenyl)-2,3,9-trimethyl-6H-thieno[3,2-][1,2,4]triazolo[4,3-a][1,4]-diazepin-6-yl)acetic acid (1.00 g, 2.49 mmol) in MeCN (30 mL). The resulting solution was stirred at r.t. for 2 h. The solvent was then removed under reduced pressure. Purification by C-18FC (gradient: 0-30% MeCN in water (containing 0.1% TFA)) gave the title compound (1.90 g, 94%) as a yellow solid. ¹H NMR: δ 1.37 (9H, s), 1.63 (3H, s), 2.42 (3H, s), 2.60 (3H, s), 2.91 (2H, br s), 3.00-3.31 (6H, m), 3.37 (2H, t), 3.46-3.58 (20H, in), 4.51 (1H, dd), 6.75 (1H, t), 7.43 (2H, d), 7.50 (2H, d), 8.28 (1H, t). m/z (ES⁺), [M+H]⁺=807.4.

### Intermediate 129b: (S)—N-(20-Amino-3,6,9,12,15,18-hexaoxaicosyl)-2-(4-(4-chlorophenyl)-2,3,9-trimethyl-6H-thieno[3,2-f][1,2,4]triazolo[4,3-a][1,4]diazepin-6-yl)acetamide

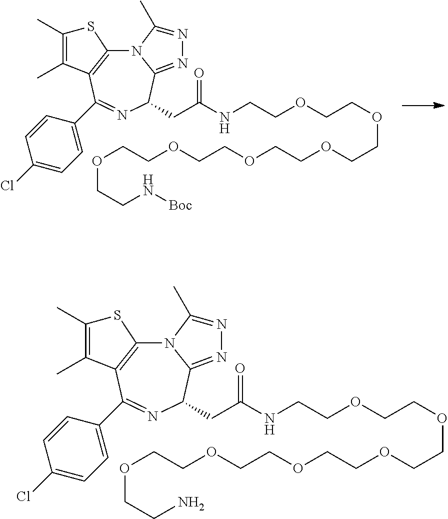 [SMILES: Cc1sc2c(c1C)C(c1ccc(Cl)cc1)=N[C@@H](CC(=O)NCCOCCOCCOCCOCCOCCOCCNC(=O)OC(C)(C)C)c1nnc(C)n1-2 ; Cc1sc2c(c1C)C(c1ccc(Cl)cc1)=N[C@@H](CC(=O)NCCOCCOCCOCCOCCOCCOCCN)c1nnc(C)n1-2]

tert-Butyl (S)-(1-(4-(4-chlorophenyl)-2,3,9-trimethyl-6H-thieno[3,2-f][1,2,4]triazolo[4,3-a][1,4]diazepin-6-yl)-2-oxo-6,9,12,15,18,21-hexaoxa-3-azatricosan-23-yl)carbamate (1.80 g, 2.23 mmol) was added to a solution of HCl in 1,4-dioxane (4M, 30 mL). The resulting solution was stirred at r.t. for 2 h. The solvent was then removed under reduced pressure. Purification by C-18FC (gradient: 0-50% MeOH in water (containing 0.1% conc. HCl)) gave the title compound in the form of a hydrochloride salt (1.50 g, 90%) as a yellow solid. ¹H NMR: δ 1.61 (3H, s), 2.40 (3H, s), 2.62 (3H, s), 2.95 (2H, t), 3.15-3.36 (4H, m), 3.40-3.64 (24H, m), 4.54 (1H, t), 7.42 (2H, d), 7.49 (2H, d). m/z (ES⁺), [M+H]⁺=707.3.

### Example 129: (S)-2-(4-(4-Chlorophenyl)-2,3,9-trimethyl-6H-thieno[3,2-f][1,2,4]triazolo[4,3-a][1,4]-diazepin-6-yl)-N-(1-(6-(2,4-dioxotetrahydropyrimidin-1 (2H)-yl)-1H-indol-3-yl)-2-oxo-6,9,12,15,18,21-hexaoxa-3-azatricosan-23-yl)acetamide

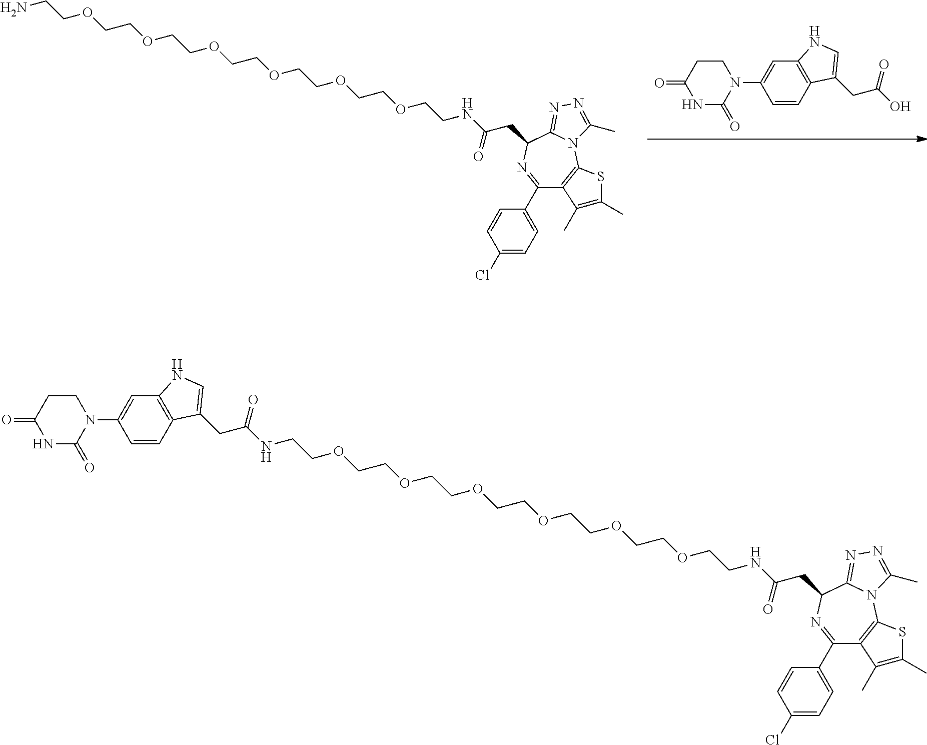 [SMILES: Cc1sc2c(c1C)C(c1ccc(Cl)cc1)=N[C@@H](CC(=O)NCCOCCOCCOCCOCCOCCOCCN)c1nnc(C)n1-2 ; O=C(O)Cc1c[nH]c2cc(N3CCC(=O)NC3=O)ccc12 ; Cc1sc2c(c1C)C(c1ccc(Cl)cc1)=N[C@@H](CC(=O)NCCOCCOCCOCCOCCOCCOCCNC(=O)Cc1c[nH]c3cc(N4CCC(=O)NC4=O)ccc13)c1nnc(C)n1-2]

2-(6-(2,4-Dioxotetrahydropyrimidin-1 (2H)-yl)-1H-indol-3-yl)acetic acid (30.0 mg, 0.104 mmol) was added to a mixture of (S)—N-(20-amino-3,6,9,12,15,18-hexaoxaicosyl)-2-(4-(4-chlorophenyl)-2,3,9-trimethyl-6H-thieno[3,2-f][1,2,4]triazolo[4,3-a][1,4]diazepin-6-yl)acetamide hydrochloride (78.0 mg, 0.105 mmol), HOBt (19.2 mg, 0.125 mmol), EDC (24.0 mg, 0.125 mmol) and DIEA (36.5 μL, 0.209 mmol) in DMF (2 mL). The resulting mixture was stirred at r.t. for 16 h and then purified directly by preparative HPLC (Column L, eluting with decreasingly polar mixtures of water (with 0.1% FA) and MeOH:gradient: 55-65%) to give the title compound (40.0 mg, 39%) as a tan solid. ¹H NMR: δ 1.62 (3H, s), 2.40 (3H, s), 2.59 (3H, s), 2.72 (2H, t), 3.15-3.33 (6H, m), 3.39 (2H, t), 3.42-3.55 (24H, m), 3.77 (2H, t), 4.50 (1H, t), 6.92 (1H, dd), 7.21 (1H, d), 7.27 (1H, d), 7.39-7.45 (2H, m), 7.46-7.54 (3H, m), 7.94 (1H, t), 8.28 (1H, t), 10.27 (1H, s), 10.93 (1H, d). m/z (ES⁺), [M+H]⁺=976.5.

### Example 130: 1-(Benzo[b]thiophen-5-yl)dihydropyrimidine-2,4 (1H,3H)-dione

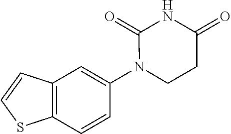 [SMILES: O=C1CCN(c2ccc3sccc3c2)C(=O)N1]

The title compound is commercially available and may be prepared using similar cross-coupling chemistry described hereinabove.

### Intermediate 131a: tert-Butyl 4-(6-bromo-2H-indazol-2-yl)piperidine-1-carboxylate

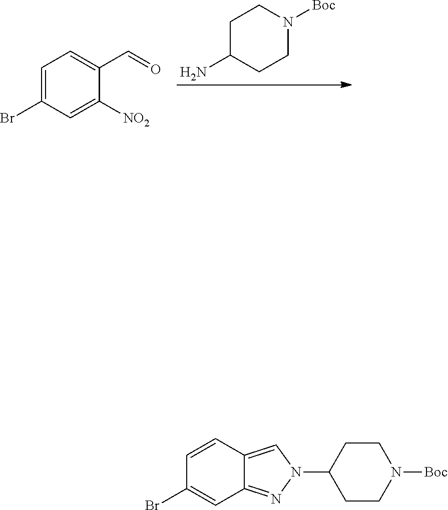

4-Bromo-2-nitrobenzaldehyde (2.00 g, 8.70 mmol) was added to a solution of tert-butyl 4-aminopiperidine-1-carboxylate (1.92 g, 9.59 mmol) in iPrOH (24 mL) at r.t. The resulting mixture was stirred at 80° C. for 4 h before the addition of tri-n-butylphosphine (6.44 mL, 26.1 mmol). The mixture was then stirred overnight at 80° C. The solvent was removed under reduced pressure. Purification by FSC (gradient: 0-18% EtOAc in petroleum ether) gave the title compound (3.00 g, 91%) as a white solid. ¹H NMR: δ 1.43 (9H, s), 1.84-2.03 (2H, m), 2.05-2.16 (2H, m), 2.84-3.07 (2H, m), 4.04-4.15 (2H, m), 4.71 (1H, tt), 7.14 (1H, dd), 7.69 (1H, dd), 7.87 (1H, dt), 8.51 (1H, d). m/z (ES⁺), [M+H]⁺=382.1.

### Intermediate 131b: tert-Butyl 4-(6-amino-2H-indazol-2-yl)piperidine-1-carboxylate

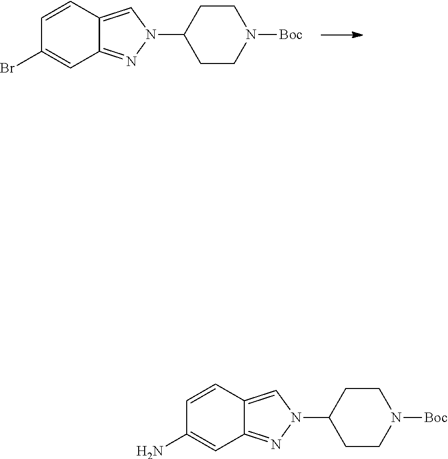 [SMILES: CC(C)(C)OC(=O)N1CCC(n2cc3ccc(Br)cc3n2)CC1 ; CC(C)(C)OC(=O)N1CCC(n2cc3ccc(N)cc3n2)CC1]

Aqueous NH₄OH (28%, 3.66 mL, 26.3 mmol) was added to a mixture of tert-butyl 4-(6-bromo-2H-indazol-2-yl)piperidine-1-carboxylate (1.00 g, 2.63 mmol), copper(I) iodide (50.0 mg, 0.263 mmol), L-proline (30.0 mg, 0.261 mmol) and K₂CO₃ (727 mg, 5.26 mmol) in DMSO (10 mL) at r.t. under N₂. The resulting mixture was stirred at 90° C. for 3 h. The crude product was directly purified by C-18FC (gradient: 0-30% MeCN in water (10 mmol NH₄HCO₃)) to give the title compound (650 mg, 78%) as a pale yellow solid. ¹H NMR: δ 1.41 (9H, s), 1.85 (2H, qd), 1.97-2.08 (2H, m), 2.90 (2H, br s), 4.05 (2H, d), 4.46 (1H, tt), 5.01 (2H, s), 6.41-6.54 (2H, m), 7.32 (1H, dd), 8.07 (1H, d). m/z (ES⁺), [M+H]⁺=317.2.

### Intermediate 131c: tert-Butyl (E)-4-(6-(3-(3-ethoxyacryloyl)ureido)-2H-indazol-2-yl)piperidine-1-carboxylate

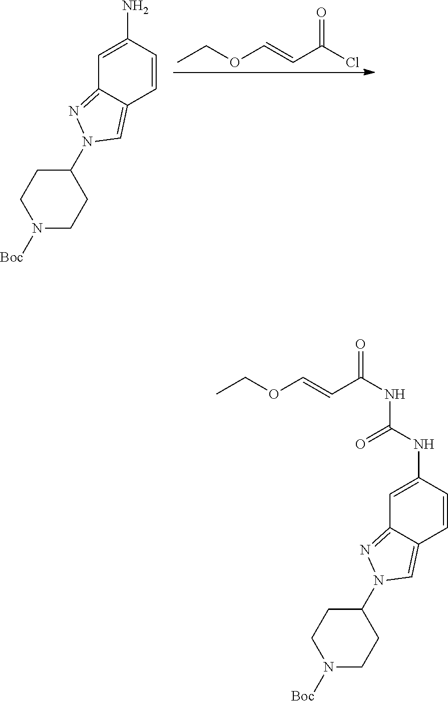

(E)-3-Ethoxyacryloyl chloride (510 mg, 3.79 mmol) was added to a mixture of silver cyanate (947 mg, 6.32 mmol) in toluene (5 mL) at r.t. under N₂. The resulting slurry was stirred at 120° C. for 1 h before being cooled to 0° C. The slurry was then added to a solution of tert-butyl 4-(6-amino-2H-indazol-2-yl)piperidine-1-carboxylate (400 mg, 1.26 mmol) in DMF (5 mL) at 0° C. The resulting mixture was stirred for 1 h at 0° C. The reaction mixture was diluted with EtOAc (100 mL), and washed sequentially with water (1×50 mL) and saturated brine (3×50 mL). The organic layer was dried (Na₂SO₄) and concentrated to afford title compound (580 mg, 100%) which was used directly in the next step without any further purification. m/z (ES⁺), [M+H]⁺=458.2.

### Example 131: 1-(2-(Piperidin-4-yl)-2H-indazol-6-yl)pyrimidine-2,4 (1H,3H)-dione

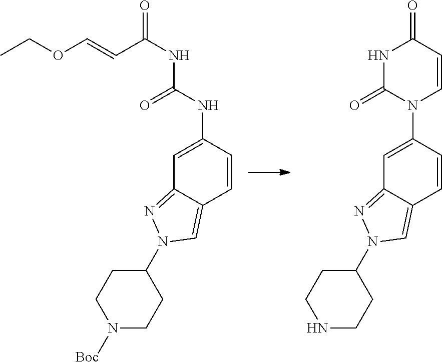 [SMILES: CCO/C=C/C(=O)NC(=O)Nc1ccc2cn(C3CCN(C(=O)OC(C)(C)C)CC3)nc2c1 ; O=c1ccn(-c2ccc3cn(C4CCNCC4)nc3c2)c(=O)[nH]1]

Benzenesulfonic acid (387 mg, 2.45 mmol) was added to a solution of tert-butyl (E)-4-(6-(3-(3-ethoxy-acryloyl)ureido)-2H-indazol-2-yl)piperidine-1-carboxylate (560 mg, 1.22 mmol) in MeCN (1 mL) at r.t. The resulting mixture was stirred at 80° C. for 1 h. The solvent was removed under reduced pressure. Purification by C-18 FC (gradient: 0-30% MeCN in water (10 mmol NH₄HCO₃)) gave the title compound (260 mg, 68%) as a red solid. ¹H NMR: δ 1.89-2.07 (4H, m), 2.65 (2H, t), 3.08 (2H, d), 4.50-4.63 (1H, br s), 5.65 (1H, d), 7.01 (1H, dd), 7.65 (1H, t), 7.74 (2H, dd), 8.49 (1H, s). m/z (ES⁺), [M+H]⁺=312.2.

### Example 132: 1-(2-(1-Methylpiperidin-4-yl)-2H-indazol-6-yl)pyrimidine-2,4 (1H,3H)-dione

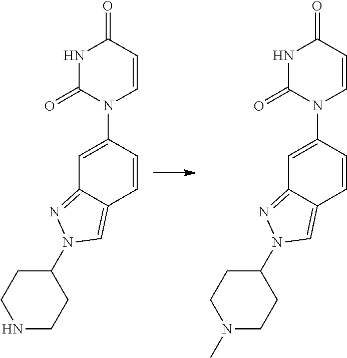 [SMILES: O=c1ccn(-c2ccc3cn(C4CCNCC4)nc3c2)c(=O)[nH]1 ; CN1CCC(n2cc3ccc(-n4ccc(=O)[nH]c4=O)cc3n2)CC1]

Sodium triacetoxyborohydride (102 mg, 0.481 mmol) was added to a mixture of 1-(2-(piperidin-4-yl)-2H-indazol-6-yl)pyrimidine-2,4 (1H, 3H)-dione (50.0 mg, 0.161 mmol), paraformaldehyde (14.5 mg, 0.483 mmol) and AcOH (46.0 μL, 0.804 mmol) in DCM (2 mL) at r.t. The resulting mixture was stirred at r.t. for 16 h. The reaction monitoring showed an incomplete reaction and thus further paraformaldehyde (14.5 mg, 0.483 mmol) and sodium triacetoxyborohydride (102 mg, 0.481 mmol) were added to the mixture and stirred at r.t. for a further 24 h. The solvent was then removed under reduced pressure. Purification by preparative HPLC (Column A, Eluent F, gradient: 7-22%) gave the title compound (29.0 mg, 56%) as a pink solid. ¹H NMR: δ 2.03-2.18 (6H, m), 2.23 (3H, s), 2.90 (2H, q), 4.49 (1H, dt), 5.67 (1H, d), 7.03 (1H, dd), 7.65-7.70 (1H, m), 7.76 (2H, t), 8.52 (1H, d), 11.41 (1H, s). m/z (ES⁺), [M+H]⁺=326.0.

### Intermediate 133a: tert-Butyl 6-bromo-4-nitro-1H-indole-1-carboxylate

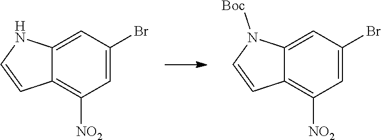 [SMILES: O=[N+]([O-])c1cc(Br)cc2[nH]ccc12 ; CC(C)(C)OC(=O)n1ccc2c([N+](=O)[O-])cc(Br)cc21]

DMAP (51.0 mg, 0.417 mmol) was added to a mixture of DIEA (1.45 ml, 8.30 mmol), di-tert-butyl dicarbonate (1.45 ml, 6.25 mmol) and 6-bromo-4-nitro-1H-indole (1.00 g, 4.15 mmol) in DCM (20 mL) at r.t. under air. The resulting solution was stirred at r.t. for 2 h. The solvent was removed under reduced pressure. Purification by FSC (gradient: 0-5% EtOAc in petroleum ether) gave the title compound (1.20 g, 85%) as a yellow solid. ¹H NMR: δ 1.63 (9H, s), 7.18 (1H, dd), 8.01 (1H, d), 8.26 (1H, d), 8.58 (1H, dd).

### Intermediate 133b: tert-Butyl 6-(2,4-dioxotetrahydropyrimidin-1 (2H)-yl)-4-nitro-1H-indole-1-carboxylate

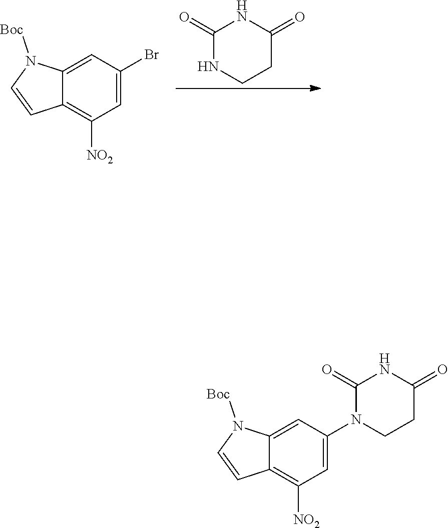 [SMILES: CC(C)(C)OC(=O)n1ccc2c([N+](=O)[O-])cc(Br)cc21 ; O=C1CCNC(=O)N1 ; CC(C)(C)OC(=O)n1ccc2c([N+](=O)[O-])cc(N3CCC(=O)NC3=O)cc21]

Ephos (141 mg, 0.264 mmol) and Ephos Pd G4 (242 mg, 0.263 mmol) were added to a degassed mixture of Cs₂CO₃ (1.72 g, 5.28 mmol), dihydropyrimidine-2,4 (1H,3H)-dione (903 mg, 7.91 mmol) and tert-butyl 6-bromo-4-nitro-1H-indole-1-carboxylate (900 mg, 2.64 mmol) in 1,4-dioxane (20 mL) at r.t. under N₂. The resulting mixture was stirred at 100° C. for 17 h. The reaction mixture was then poured into water (20 mL) and extracted with EtOAc (3×20 mL). The combined organic extracts were dried (Na₂SO₄) and concentrated to afford a yellow solid. Purification by C-18FC (gradient: 40-70% MeCN in water (containing 0.1% FA)) gave the title compound (580 mg, 59%) as a yellow solid. ¹H NMR: δ 1.65 (9H, s), 2.79 (2H, t), 3.95 (2H, t), 7.25 (1H, dd), 8.06 (1H, d), 8.25 (1H, d), 8.51-8.58 (1H, m), 10.54 (1H, s). m/z (ES⁺), [M+H]⁺=375.2.

### Intermediate 133c: tert-Butyl 4-amino-6-(2,4-dioxotetrahydropyrimidin-1 (2H)-yl)-1H-indole-1-carboxylate

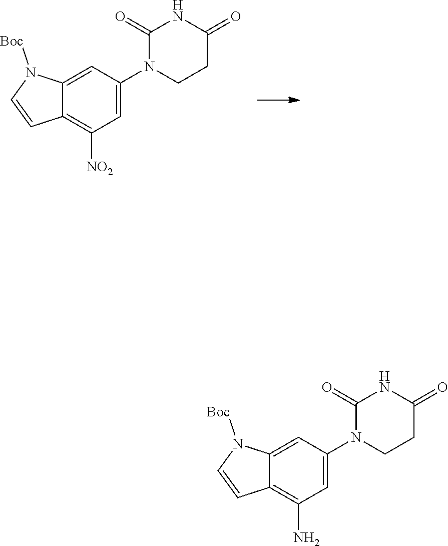 [SMILES: CC(C)(C)OC(=O)n1ccc2c([N+](=O)[O-])cc(N3CCC(=O)NC3=O)cc21 ; CC(C)(C)OC(=O)n1ccc2c(N)cc(N3CCC(=O)NC3=O)cc21]

AcOH (17.7 μL, 0.309 mmol) was added to a mixture of iron (1.73 g, 31.0 mmol) and tert-butyl 6-(2,4-dioxotetrahydropyrimidin-1 (2H)-yl)-4-nitro-1H-indole-1-carboxylate (580 mg, 1.55 mmol) in EtOH (20 mL). The resulting mixture was stirred at 60° C. for 2 h before being adjusted to pH 8 using saturated aqueous NaHCO₃. The reaction mixture was then filtered and poured into water (20 mL) and extracted with EtOAc (3×20 mL). The combined organic extracts were dried (Na₂SO₄) and concentrated to afford a brown solid. Purification by FSC (gradient: 60-100% EtOAc in petroleum ether) gave the title compound (200 mg, 38%) as a brown solid. ¹H NMR: δ 1.65 (9H, s), 2.79 (2H, t), 3.31 (2H, s), 3.95 (2H, t), 7.25 (1H, d), 8.06 (1H, d), 8.25 (1H, d), 8.55 (1H, d), 10.54 (1H, s). m/z (ES⁺), [M+H]⁺=345.3.

### Intermediate 133d: tert-Butyl 4-(dimethylamino)-6-(2,4-dioxotetrahydropyrimidin-1 (2H)-yl)-1H-indole-1-carboxylate

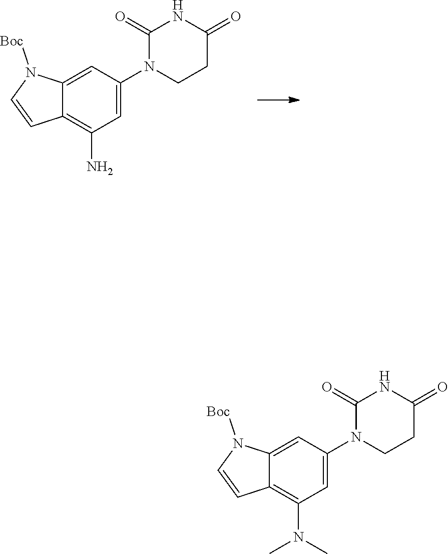 [SMILES: CC(C)(C)OC(=O)n1ccc2c(N)cc(N3CCC(=O)NC3=O)cc21 ; CN(C)c1cc(N2CCC(=O)NC2=O)cc2c1ccn2C(=O)OC(C)(C)C]

Sodium triacetoxyborohydride (369 mg, 1.74 mmol) was added to a mixture of tert-butyl 4-amino-6-(2,4-dioxotetrahydropyrimidin-1 (2H)-yl)-1H-indole-1-carboxylate (200 mg, 0.581 mmol), paraformaldehyde (52.3 mg, 1.74 mmol) in DCM (10 mL) at r.t. The resulting mixture was stirred at r.t. for 16 h. The mixture was poured into water (20 mL) and extracted with DCM (3×20 mL). The combined organic extracts were dried (Na₂SO₄) and concentrated to give a brown solid. Purification by FSC (gradient: 10-30% EtOAc in petroleum ether) gave the title compound (130 mg, 60%) as a brown solid. ¹H NMR: δ 1.62 (9H, s), 2.73 (2H, t), 2.93 (6H, s), 3.81 (2H, t), 6.60 (1H, s), 6.79 (1H, d), 7.53-7.65 (2H, m), 10.32 (1H, s). m/z (ES⁺), [M+H]⁺=373.2.

### Example 133: 1-(4-(Dimethylamino)-1H-indol-6-yl)dihydropyrimidine-2,4 (1H,3H)-dione AZ14229497

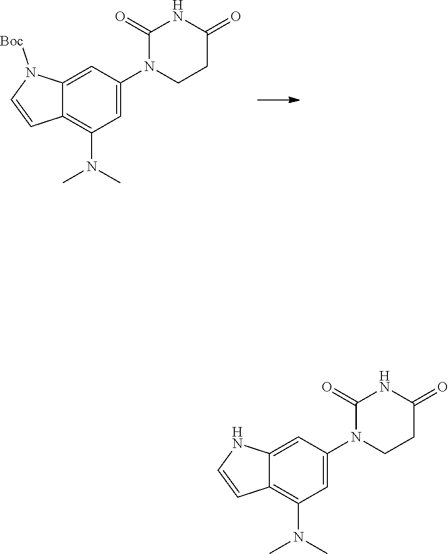 [SMILES: CN(C)c1cc(N2CCC(=O)NC2=O)cc2c1ccn2C(=O)OC(C)(C)C ; CN(C)c1cc(N2CCC(=O)NC2=O)cc2[nH]ccc12]

tert-Butyl 4-(dimethylamino)-6-(2,4-dioxotetrahydropyrimidin-1 (2H)-yl)-1H-indole-1-carboxylate (120 mg, 0.322 mmol) was dissolved in formic acid (10 mL) and the resulting mixture was stirred at r.t. for 2 h. The solvent was removed under reduced pressure. Purification by preparative HPLC (Column J, Eluent D, gradient: 17-22%) gave the title compound (46.0 mg, 52%) as a white solid. ¹H NMR: (CD₃OD) δ 2.84 (2H, t), 2.97 (6H, s), 3.90 (2H, t), 6.46 (1H, d), 6.57 (1H, dd), 6.99-7.01 (1H, m), 7.22 (1H, d). m/z (ES⁺), [M+H]⁺=273.1.

### Intermediate 134a: tert-Butyl 6-bromo-4-cyano-1H-indole-1-carboxylate

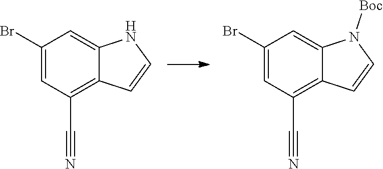 [SMILES: N#Cc1cc(Br)cc2[nH]ccc12 ; CC(C)(C)OC(=O)n1ccc2c(C#N)cc(Br)cc21]

DMAP (16.6 mg, 0.136 mmol) was added to a mixture of DIEA (474 μL, 2.71 mmol), di-tert-butyl dicarbonate (473 μL, 2.04 mmol) and 6-bromo-1H-indole-4-carbonitrile (300 mg, 1.36 mmol) in DCM (20 mL). The resulting solution was stirred at r.t. for 2 h. The solvent was removed under reduced pressure. Purification by FSC (gradient: 0-5% EtOAc in petroleum ether) gave the title compound (430 mg, 99%) as a white solid. ¹H NMR: δ 1.62 (9H, s), 6.80-6.88 (1H, m), 7.94 (1H, d), 8.03 (1H, d), 8.42-8.49 (1H, m).

### Intermediate 134b: tert-Butyl 4-cyano-6-(2,4-dioxotetrahydropyrimidin-1 (2H)-yl)-1H-indole-1-carboxylate

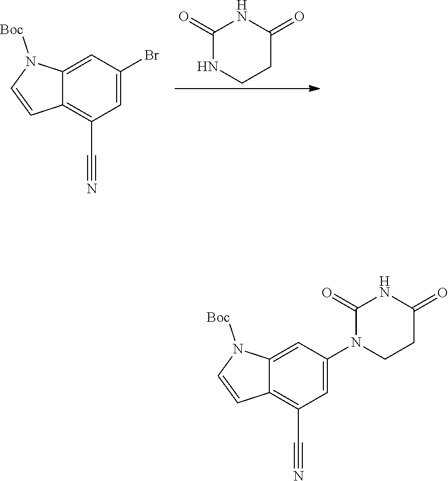 [SMILES: CC(C)(C)OC(=O)n1ccc2c(C#N)cc(Br)cc21 ; O=C1CCNC(=O)N1 ; CC(C)(C)OC(=O)n1ccc2c(C#N)cc(N3CCC(=O)NC3=O)cc21]

Ephos (66.6 mg, 0.125 mmol) and Ephos Pd G4 (114 mg, 0.124 mmol) were added to a degassed mixture of Cs₂CO₃ (812 mg, 2.49 mmol), dihydropyrimidine-2,4 (1H,3H)-dione (426 mg, 3.73 mmol) and tert-butyl 6-bromo-4-cyano-1H-indole-1-carboxylate (400 mg, 1.25 mmol) in 1,4-dioxane (16 mL) at r.t. under N₂. The resulting mixture was stirred at 100° C. for 17 h. The solvent was then removed under reduced pressure. Purification by FSC (gradient: 0-10% MeOH in DCM) gave the title compound (180 mg, 41%) as a brown solid. ¹H NMR: δ 1.62 (9H, s), 2.74 (2H, t), 3.87 (2H, t), 6.80-6.88 (1H, m), 7.80 (1H, d), 7.94 (1H, d), 8.33-8.40 (1H, m), 10.49 (1H, s). m/z (ES⁺), [M+Na]=377.1.

### Example 134: 6-(2,4-Dioxotetrahydropyrimidin-1 (2H)-yl)-1H-indole-4-carbonitrile

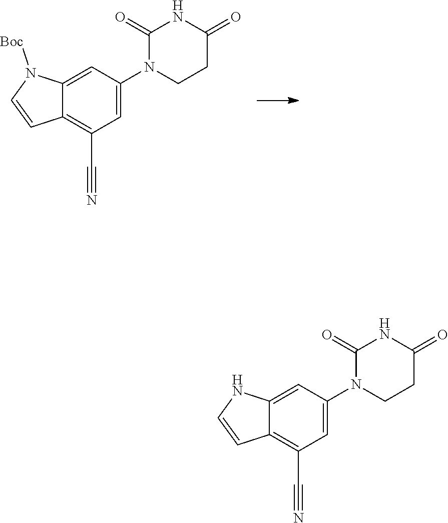 [SMILES: CC(C)(C)OC(=O)n1ccc2c(C#N)cc(N3CCC(=O)NC3=O)cc21 ; N#Cc1cc(N2CCC(=O)NC2=O)cc2[nH]ccc12]

tert-Butyl 4-cyano-6-(2,4-dioxotetrahydropyrimidin-1 (2H)-yl)-1H-indole-1-carboxylate (180 mg, 0.508 mmol) was added to 2,2,2-trifluoroethanol (3 mL). The resulting mixture was heated in a microwave reactor at 120° C. for 1 h and then cooled to r.t. The solvent was then removed under reduced pressure. Purification by C-18FC (gradient: 0-20% MeCN in water (containing 0.1% FA) gave the title compound (114 mg, 88%) as a white solid. ¹H NMR: δ 2.75 (2H, t), 3.83 (2H, t), 6.57-6.62 (1H, m), 7.56 (1H, d), 7.69-7.75 (2H, m), 10.42 (1H, s), 11.81 (1H, s). m/z (ES⁺), [M+H]⁺=255.2.

### Intermediate 135a: tert-Butyl 4-bromo-7-cyano-1H-pyrrolo[2,3-c]pyridine-1-carboxylate

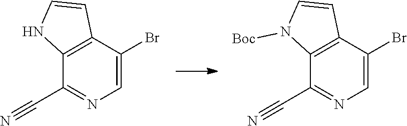 [SMILES: N#Cc1ncc(Br)c2cc[nH]c12 ; CC(C)(C)OC(=O)n1ccc2c(Br)cnc(C#N)c21]

Di-tert-butyl dicarbonate (544 μL, 2.34 mmol) was added to a mixture of 4-bromo-1H-pyrrolo[2,3-c]pyridine-7-carbonitrile (prepared by the method described in WO2014210255, 400 mg, 1.80 mmol), DMAP (44.0 mg, 0.36 mmol) and triethylamine (753 μL, 5.40 mmol) in DCM (8 mL). The resulting mixture was stirred at r.t. for 14 h. The solvent was removed under reduced pressure. Purification by FSC (gradient: 0-20% EtOAc in petroleum ether) gave the title compound (220 mg, 38%) as a white solid. ¹H NMR: δ 1.66 (9H, s), 6.91 (1H, d), 8.18 (1H, d), 8.71 (1H, s). m/z (ES⁺), [M+H]⁺=324.1.

### Example 135: 4-(2,4-dioxotetrahydropyrimidin-1 (2H)-yl)-1H-pyrrolo[2,3-c]pyridine-7-carbonitrile

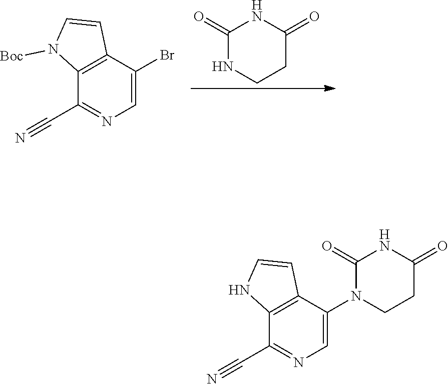 [SMILES: CC(C)(C)OC(=O)n1ccc2c(Br)cnc(C#N)c21 ; O=C1CCNC(=O)N1 ; N#Cc1ncc(N2CCC(=O)NC2=O)c2cc[nH]c12]

Ephos (29.9 mg, 0.0559 mmol) and Ephos Pd G4 (51.3 mg, 0.0558 mmol) were added to a degassed mixture of Cs₂CO₃ (546 mg, 1.68 mmol), dihydropyrimidine-2,4 (1H,3H)-dione (191 mg, 1.67 mmol) and tert-butyl 4-bromo-7-cyano-1H-pyrrolo[2,3-c]pyridine-1-carboxylate (180 mg, 0.559 mmol) in 1,4-dioxane (10 mL) at r.t. under N₂. The resulting mixture was stirred at 100° C. for 24 h. The solvent was then removed under reduced pressure. Purification by C-18FC (gradient: 0-1% MeCN in water (containing 0.1% FA) gave material that was further purified by preparative HPLC (Column G, Eluent E) to give the title compound (35.0 mg, 25%) as a pale yellow solid. ¹H NMR: δ 2.80 (2H, m), 3.94 (2H, m), 6.71 (1H, m), 7.83 (1H, d), 8.28 (1H, d), 10.59 (1H, s), 12.66 (1H, br s). m/z (ES⁺), [M+H]⁺=256.0.

### Biological Assays

The following assays and biological procedures were used to investigate and measure the effects of the compounds of the present specification.

### Protein Preparation—CBRN/DDB1 Complex

CBRN: A codon optimized DNA sequence (for Baculovirus mediated expression in insect cells) encoding amino acid residues 1 to 442 (Uniprot Q96SW2) of human DDB1 was synthesized by Genscript USA Inc (Piscataway, New Jersey, USA) and subcloned into pFastBac™ 1. The synthesized sequence was designed to encode an N-terminal hexahistidine tag, a thrombin cleavage site, an AviTag™ and a tobacco etch virus protease (TEV) cleavage site followed by the CBRN sequence. The resulting protein sequence is listed below.

MHHHHHHLVPRGSGLNDIFEAQKIEWHEENLYFQG
MAGEGDQQDAAHNMGNHLPLLPAESEEEDEMEVED
QDSKEAKKPNIINFDTSLPTSHTYLGADMEEFHGR
TLHDDDSCQVIPVLPQVMMILIPGQTLPLQLFHPQ
EVSMVRNLIQKDRTFAVLAYSNVQEREAQFGTTAE
IYAYREEQDFGIEIVKVKAIGRQRFKVLELRTQSD
GIQQAKVQILPECVLPSTMSAVQLESLNKCQIFPS
KPVSREDQCSYKWWQKYQKRKFHCANLTSWPRWLY
SLYDAETLMDRIKKQLREWDENLKDDSLPSNPIDF
SYRVAACLPIDDVLRIQLLKIGSAIQRLRCELDIM
NKCTSLCCKQCQETEITTKNEIFSLSLCGPMAAYV
NPHGYVHETLTVYKACNLNLIGRPSTEHSWFPGYA
WTVAQCKICASHIGWKFTATKKDMSPQKFWGLTRS
ALLPTIPDTEDEISPDKVILCL

DDB1: A codon optimized DNA sequence (for Baculovirus mediated expression in insect cells) encoding amino acid residues 1 to 395 and 706 to 1140 (Uniprot Q16531) of human DDB1 was synthesized by Genscript USA Inc (Piscataway, New Jersey, USA) and subcloned into pFastBac™ 1. The synthesized sequence was designed to encode an N-terminal hexahistidine tag, a tobacco etch virus protease (TEV) cleavage site followed by the DDB1 sequence. The DDB1 sequence was mutated to replace an internal domain (amino acid residues 396 to 705) with linker amino acid residues resulting protein sequence is listed below. The linker amino acid residues are underlined and shown in italics.

MHHHHHHVDEENLYFQGGGRMSYNYVVTAQKPTAV
NGCVTGHFTSAEDLNLLIAKNTRLEIYVVTAEGLR
PVKEVGMYGKIAVMELFRPKGESKDLLFILTAKYN
ACILEYKQSGESIDIITRAHGNVQDRIGRPSETGI
IGIIDPECRMIGLRLYDGLFKVIPLDRDNKELKAF
NIRLEELHVIDVKFLYGCQAPTICFVYQDPQGRHV
KTYEVSLREKEFNKGPWKQENVEAEASMVIA VPE
PFGGAIIIGQESITYHNGDKYLAIAPPIIKQSTIV
CHNRVDPNGSRYLLGDMEGRLFMLLLEKEEQMDGT
VTLKDLRVELLGETSIAECLTYLDNGVVFVGSRLG
DSQLVKLNVDSNEQGSYVVAMETFTNLGPIVDMCV
VDLERQGQGQLVTCSGAFKEGSLRIIRNGIGGNGN
SGEIQKLHIRTVPLYESPRKICYQEVSQCFGVLSS
RIEVQDTSGGTTALRPSASTQALSSSVSSSKLFSS
STAPHETSFGEEVEVHNLLIIDQHTFEVLHAHQFL
QNEYALSLVSCKLGKDPNTYFIVGTAMVYPEEAEP
KQGRIVVFQYSDGKLQTVAEKEVKGAVYSMVEFNG
KLLASINSTVRLYEWTTEKELRTECNHYNNIMALY
LKTKGDFILVGDLMRSVLLLAYKPMEGNFEEIARD
FNPNWMSAVEILDDDNFLGAENAFNLFVCQKDSAA
TTDEERQHLQEVGLFHLGEFVNVFCHGSLVMQNLG
ETSTPTQGSVLFGTVNGMIGLVTSLSESWYNLLLD
MQNRLNKVIKSVGKIEHSFWRSFHTERKTEPATGF
IDGDLIESFLDISRPKMQEVVANLQYDDGSGMKRE
ATADDLIKVVEELTRIH

Baculovirus production of CBRN and DDB1: Recombinant bacmid and P2 virus of CBRN and DDB1 was generated according to the Bac-to-Bac Baculovirus Expression System manual (ThermoFisher Scientific).

Protein Expression: To produce recombinant CBRN/DDB1 complex, 2.5 L of Sf9 (clonal isolate of Spodoptera frugiperda Sf21) cells at a cell density of 2.5×10⁶ cells/mL in Optimum Growth™ Flasks (Thomson Instrument Company) was inoculated with 75 mL of CBRN P2 virus and 25 mL of DDB1 P2 virus and incubated for 48 h at 27° C. Cells were harvested by centrifugation at 3000×g for 20 minutes and was resuspended in 5 mL Lysis buffer (50 mM Tris-Cl, 20 mM imidazole, 10% Glycerol, 1 mM TCEP, complete protease inhibitor tablets EDTA-free (Roche)) per 1 g of cells before freezing at −80° C.

Protein Purification: Protein purification was initiated by thawing cells (resuspended in Lysis buffer) at r.t. Once thawed, NaCl was added to a final concentration of 200 mM, followed by 0.01 μL/mL Benzonase™ and the lysate was incubated with slow stirring for 20 minutes. After the freeze-thaw lysis, the whole cell lysate was centrifuged at 48 000 g for 45 minutes and all following purification steps were done on ice or in cold-room. Supernatant (cell lysate) was mixed with 1 mL/50 mL lysate Ni Sepharose™ 6 Fast Flow (Cytiva) pre-equilibrated with Wash buffer (50 mM Tris-Cl pH 8, 20 mM Imidazole, 500 mM NaCl, 10% Glycerol, 1 mM TCEP) and incubated with slow stirring for 1 h. The mix was loaded in a 70 mL open gravity flow column, washed with 10 bed volumes of Wash buffer, and stepwise eluted with 300 mM imidazole in Wash buffer. CRBN/DDB1 was subsequently biotinylated by adding 50 mM Bicine pH 8.3, 10 mM Mg(OAc)₂, 10 mM ATP, 50 μM Biotin and BirA enzyme at a molar ratio of 1/10 (BirA/CRBN) and incubated with slow stirring at 4° C. overnight. After biotinylation, the CBRN/DDB1 complex was purified by Size Exclusion Chromatography on a HiLoad™ 26/60 Superdex™ 200 column equilibrated in SEC buffer (50 mM HEPES pH 7.5, 200 mM NaCl, 5% glycerol, 1 mM TCEP) before flash-freezing in liquid nitrogen and storage at −80° C.

### Cereblon HTRF Binding Assay

To determine the binding affinity of the test compounds to cereblon (CRBN)—DNA damage protein 1 (DDB1) complex, the biochemical assay was conducted using the homogeneous time resolved fluorescence (HTRF) format at r.t. Compounds dissolved in DMSO were delivered into the 384-well assay plate in a 40 nL volume, using a 3-fold serial dilutions scheme from top concentration of 10 mM. Each dose response curve includes 10 concentrations of compound from 100 μM to 3 nM in the final assay mixture. The assay mixture in a total volume of 4 μL consists of 50 mM Tris-HCl at pH 7.5, 100 mM NaCl, 0.01% Pluronic F127, 1 mM TCEP, 500 μM EDTA, 0.2 nM CRBN-DDB1, 0.2 nM terbium cryptate labeled streptavidin, and 2 nM Cyanine 5-labeled lenalidomide as the probe. The positive control (DMSO) and negative control (10 μM pomalidomide, final concentration) were included on the same plate using same amount of DMSO as compound treated wells. Reagents were dispensed into the 384-well assay microplate using a Certus Flex liquid dispenser. After incubation for 2 h at r.t., the assay plates were read on a PHERAstar FSX plate reader (BMG Labtech) in the HTRF mode. The raw data from PHERAstar was directly imported into Genedata Screener for analysis. All three layers are imported: Channel A (665 nm), Channel B (620 nm) and ratio (Channel 10000×B/A). The ratio was normalized to positive and negative controls on the same plates to calculate percent inhibition: I %=(I−NC)/(PC−NC), where I are readings from the compound treated well, NC and PC are average readings from negative control wells and positive control wells, respectively. IC₅₀ values were obtained by fitting the I % vs. compound concentration [I] to I %=S₀+(S_{Inf}−S₀)/(1+((IC₅₀/[I])\hat{ }n)), where S₀ is the fitted activity level at zero concentration of test compound, S_{Inf} is the fitted activity level at zero infinite concentration of test compound, and n is the hill coefficient of the curve.

The examples were tested in the HTRF assay and the following data was observed. The IC₅₀ values reported below are the calculated mean result of one or more experiments. For the negative control, pomalidomide, IC₅₀ was 101 nm.

### Cereblon HTRF Binding Assay Data for the Examples:

| | Cereblon HTRF |
|---|---|
| Example No. | Binding IC₅₀ (μM) |
| 1 | 2.098 |
| 2 | 1.997 |
| 3 | 3.284 |
| 4 | 3.876 |
| 5 | 6.744 |
| 6 | 3.931 |
| 7 | 3.003 |
| 8 | 4.573 |
| 9 | 5.348 |
| 10 | 7.880 |
| 11 | 26.924 |
| 12 | 10.953 |
| 13 | 0.765 |
| 14 | 0.516 |
| 15 | 10.586 |
| 16 | 0.211 |
| 17 | 0.206 |
| 18 | 0.347 |
| 19 | 0.430 |
| 20 | 0.357 |
| 21 | 0.784 |
| 22 | 0.487 |
| 23 | 1.058 |
| 24 | 8.977 |
| 25 | 2.961 |
| 26 | 1.490 |
| 27 | 1.036 |
| 28 | 17.032 |
| 29 | 8.012 |
| 30 | 8.675 |
| 31 | 4.103 |
| 32 | 1.227 |
| 33 | 0.762 |
| 34 | 0.627 |
| 35 | 2.973 |
| 36 | 0.115 |
| 37 | 3.097 |
| 38 | 0.566 |
| 39 | 0.082 |
| 40 | 0.186 |
| 41 | 1.170 |
| 42 | 2.115 |
| 43 | 3.626 |
| 44 | 8.473 |
| 45 | 0.967 |
| 46 | 3.748 |
| 47 | 3.063 |
| 48 | 1.970 |
| 49 | 0.124 |
| 50 | 0.226 |
| 51 | 0.511 |
| 52 | 0.445 |
| 53 | 0.244 |
| 54 | 0.188 |
| 55 | 0.220 |
| 56 | 0.341 |
| 57 | 0.096 |
| 58 | 0.288 |
| 59 | 0.329 |
| 60 | 0.444 |
| 61 | 1.508 |
| 62 | 2.137 |
| 63 | 0.235 |
| 64 | 0.078 |
| 65 | 0.755 |
| 66 | 0.264 |
| 67 | 0.974 |
| 68 | 2.214 |
| 69 | 3.541 |
| 70 | 1.013 |
| 71 | 1.125 |
| 72 | 0.926 |
| 73 | 0.963 |
| 74 | 3.814 |
| 75 | 4.042 |
| 76 | 1.922 |
| 77 | 0.562 |
| 78 | 1.867 |
| 79 | 1.976 |
| 80 | 3.153 |
| 81 | 0.065 |
| 82 | 0.168 |
| 83 | 0.113 |
| 84 | 0.357 |
| 85 | 0.195 |
| 86 | 0.291 |
| 87 | 0.379 |
| 88 | 0.679 |
| 89 | 1.210 |
| 90 | 0.771 |
| 91 | 0.762 |
| 92 | 5.240 |
| 93 | 12.785 |
| 94 | 4.201 |
| 95 | 6.579 |
| 96 | 3.736 |
| 97 | 2.423 |
| 98 | 3.121 |
| 99 | 4.557 |
| 100 | 1.071 |
| 101 | 0.161 |
| 102 | 0.274 |
| 103 | 0.682 |
| 104 | 0.283 |
| 105 | 0.460 |
| 106 | 4.044 |
| 107 | 3.781 |
| 108 | 3.808 |
| 109 | 1.034 |
| 110 | 0.655 |
| 111 | 0.050 |
| 112 | 0.089 |
| 113 | 1.078 |
| 114 | 1.053 |
| 115 | 0.933 |
| 116 | 0.368 |
| 117 | 2.164 |
| 118 | 0.389 |
| 119 | 1.447 |
| 120 | 2.205 |
| 121 | 3.841 |
| 122 | 2.122 |
| 123 | 0.597 |
| 124 | 0.527 |
| 125 | 0.458 |
| 126 | 0.577 |
| 127 | 0.014 |
| 128 | 0.083 |
| 129 | 0.094 |
| 130 | 0.706 |
| 131 | 2.130 |
| 132 | 2.156 |
| 133 | 2.824 |
| 134 | 1.665 |
| 135 | 0.594 |

### HiBiT Degradation Assay:

Determination of PROTAC potency was determined in HEK 293 cells expressing BRD4 tagged to a HiBiT tag. To generate those cells, HiBiT tag (aminoacid sequence: VSGWRLFKKIS) was inserted, via CRISPR CAS9 knock-in, at the N-terminus of endogenous BRD4.

The DNA sequences used were:

1.
gRNA:
TGGGATCACTAGCATGTCTGC,
SSODN:
2.
TGGGATCACTAGCATGGTGAGCGGCTGGCGGCT
GTTCAAGAAGATTAGCTCTGCAG

To measure success of knock-in the cells were then tested for their level of luminescence. Finally a stable pool was generated from them. In a typical experiment, 14×10³ cells are seed in the wells of a 384 well plate (white flat bottom, tissue culture ready, corning 781080) in 40 μL of culture medium (DMEM, 10% FBS and 2 mM Gln). Immediately after seeding, PROTAC compounds, diluted in DMSO solution, are added to the wells via ECHO 555 acoustic dispenser at the appropriate concentration. DMSO is used as a not active control and as normalization. In each plate a series of compounds of known DC₅₀ are included as quality control. The 384 well plate is placed in an incubator at 37° C. and 5% CO₂ for 18 h. At the end of the incubation period plates are removed from the incubator and the medium is replaced with 10 μL of PBS and 10 μL of complete NanoGlo HiBiT solution (Promega N3040, consisting of 9.7 μL of Lysis buffer, with 0.1 μL of LgBiT protein and 0.2 μL of nanoluciferase substrate). After 15 minutes of incubation at r.t., the luciferase activity of the plate is recorded in a luminometer. dBET6 (MCE® Cat. No.: HY-112588) was used for normalization when calculating Dₘₐₓ.

### BRD4 Surface Plasmon Resonance (SPR) Assay:

Protein Construct: A codon-optimized DNA sequence for E. coli expression system encoding amino acid residues 42 to 169 (Uniprot 060885) of human BRD4 was synthesized by Genscript USA Inc (Piscataway, New Jersey, USA) and subcloned into a modified pET28a. The synthesized sequence was designed to encode an N-terminal hexahistidine tag, a tobacco etch virus protease (TEV) cleavage site followed by the BRD4 sequence. The resulting protein sequence is listed below:

MHHHHHHSSGVDLGTENLYFQSMNPPPPETSNPNKPKR
QTNQLQYLLRVVLKTLWKHQFAWPFQQPVDAVKLNLPD
YYKIIKTPMDMGTIKKRLENNYYWNAQECIQDFNTMFT
NCYTYNKPGDDIVLMAEALEKLFLQKINELPTEE

Protein Expression: The plasmid was transformed into E. coli BL21 Gold (DE3) cells (Agilent). 6 liters of bacterial culture were grown at 37° C. in TB (Terrific Broth) supplemented with 100 mg/l Kanamycin to an A₆₀₀ of approximately 0.6 and induced overnight with 0.2 mM isopropyl ß-D-thiogalactopyranoside (Melford). Harvested cells are frozen and stored at −80° C. until purification.

Protein Purification: Protein purification was initiated by thawing cells and resuspended in lysis buffer (50 mM HEPES pH7.5, 0.5 M NaCl, 5% glycerol, 30 mM imidazole, 1 mg/ml lysozyme, Complete Roche protease inhibitor-EDTA free and 0.01 μL/mL Benzonase™) at 4° C. Once thawed, and the lysate was incubated with slow stirring for 20 minutes. The sample was then lysed by passing the lysate through a french press. After lysis, the whole cell lysate was centrifuged at 48 000 g for 45 minutes and all following purification steps were done on ice or in a cold room. An 8 ml Ni NTA superflow column was packed in an XK26 and washed and equilibrated with the lysis buffer. The lysate supernatant was loaded at 1 ml/min, collecting the flow-through as a single pool. Once loaded the column was washed for 10 CV with lysis buffer supplemented with 40 mM imidazole and 2 ml fractions were collected. A final elution step was performed collecting 2 ml fractions over 10 CV with elution buffer (lysis buffer supplemented with 500 mM imidazole). As the next step, the his-tag was cleaved using TEV protease in the ratio of 1 mg to 100 mg of protein. Dialysis of the protein was performed simultaneously to remove high imidazole concentration overnight. As a final step of purification, the protein was loaded onto a HiLoad™ 26/60 Superdex™ 200 column pre-equilibrated in size exclusion buffer containing 50 mM HEPES pH 7.5, 200 mM NaCl, 5% glycerol, 1 mM TCEP. SDS-PAGE was run to check the quality of the protein after each step of purification. Final quality control included analytical size exclusion and whole intact mass spectroscopy to check on the protein size.

Chemical Biotinylation: To make the protein suitable for SPR, it was decided to perform chemical biotinylation of the protein. The protein was then incubated on ice with 2 mM of EZ-Link NHS-PEG4-Biotin (75 μl) on ice. To reduce chemical over labeling of the protein the incubation time was set at 2 h. To remove excess NHS-PEG4-Biotin, the reaction mixture was dialysed overnight against the size exclusion buffer (50 mM HEPES pH 7.5, 200 mM NaCl, 5% glycerol, 1 mM TCEP). The protein was aliquoted and flash-frozen in liquid nitrogen and stored at −80° C.

Surface plasmon resonance: Biotinylated BRD4 protein was used for surface plasmon resonance (SPR) experiments. Prior to running each experiment, a Desorb method was run and a series S streptavidin Biacore chip (#BR100531, Cytiva) was docked onto a T200 Biacore instrument (Cytiva) and primed with buffer consisting of 20 mM Tris (pH 7.5), 150 mM NaCl, 50 mM EDTA (Ethylenediaminetetraacetic acid), 1 mM TCEP (tris(2 carboxyethyl)phosphine), 0.05% (v/v) Tween-20 (polyoxyethylene(20)sorbitan monolaurate) and 0.3% (v/v) DMSO (dimethyl sulfoxide) three times. All immobilization and binding experiments were equilibrated to 25° C. BRD4 protein was immobilized at a density of 150 to 250 RU. Unbound streptavidin sites were blocked with 50 μM amino-PEG biotin (#21346, ThermoFisher). Compounds were prepared in assay buffer in a 384-well polypropylene microplate (#781201, Greiner) and care was taken to ensure a final concentration of 0.3% (v/v) DMSO. Compounds were tested at concentrations from 3 nM to 30 mM using the ‘High Performance’ cycle type to generate binding sensorgams and a 50% DMSO wash was used between samples. All data were double referenced, solvent (DMSO) corrected and affinities were determined by globally fitting to a 1:1 binding model, or fitted to steady state binding model using the Biacore T200 Evaluation software (Cytiva).

### BRD4 Degradation and Affinity (SPR) Data for PROTAC Examples:

| | | | BRD4 Affinity K_{d} |
|---|---|---|---|
| Example No. | DC50 (μM) | Dₘₐₓ (%) | (μM) |
| 127 | 0.198 | 97.2 | 0.013 |
| 128 | 1.14 | 73.1 | 0.019 |
| 129 | 0.970 | 111 | 0.030 |

### Endogenous SALL4 Degradation Assay:

The neuroblastoma cell line, Kelly, was used to monitor the degradation of the endogenous SALL4 protein using a SDS-PAGE assay. Kelly cells were grown in RPMI 1640 medium supplemented with 10% fetal calf serum. Cells were splitted every 4 days at 1/10 ratio. Cells with less than 10 passages were used for the experiments.

For the experiment, the cells were seeded in 12 well plates at 500,000 cells per well in a final volume of 1 mL of medium. The next day, the cells were dosed at 10 μM with the different compounds. After 20 h of treatment, the cells were lysed in RIPA buffer plus protease cocktail inhibitors (100 μL/well) for 20 minutes on ice.

Lysates were collected and spinned at 10000 g for 5 min. The supernatant was collected and mix with sample buffer 4× containing a sample reducing agent (25 μL), then boil 5 minutes at 85° C. Lysates was stored at −20° C.

For the SDS-PAGE analysis, samples were loaded on precast gel (4-12%), runned at 150V constant on ice and transferred into membranes using P0 program from the fast transfer iBlot2 device (Thermofischer). Membranes were then incubated in LI-COR blocking solution (Intercept) for 30 min, then with primary antibodies overnight at 4° C. The membranes were then washed for a minimum of 30 minutes with TBS-tween 0.1% and incubated with secondary antibodies for LI-COR detection (1 h diluted at 1/10000). After several washes in PBS-Tween 0.1%, the membranes were analyzed by the Li-COR Odyssey CLx imaging system for protein level detection. GAPDH was used as loading control.

### List of Reagents

| Reagent | Source | Catalogue number |
|---|---|---|
| Kelly cells | Sigma-Aldrich | 92110411 |
| RPMI 1640 with Glutamine | Thermofischer | 11875093 |
| RIPA Lysis and Extraction Buffer | Thermofischer | 89900 |
| Halt ™ Protease Inhibitor Cocktail (100X) | Thermofischer | 78430 |
| NuPAGE ™ LDS Sample Buffer (4X) | Thermofischer | NP0007 |
| NuPAGE ™ Sample Reducing Agent (10X) | Thermofischer | NP0004 |
| NuPAGE ™ 4-12% Bis-Tris Protein Gels, 1.5 mm, 15-well | Thermofischer | NP0336BOX |
| iBlot ™ 2 Transfer Stacks, nitrocellulose, regular size | Thermofischer | IB23001 |
| Intercept ™ Blocking Buffer | LI-COR | 927-60001 |
| SALL4 antibody | Proteintech | 24500-1-AP |
| GAPDH antibody | Proteintech | 60004-1-Ig |
| IRDye ™ 800CW Donkey anti-Rabbit IgG Secondary Antibody | LI-COR | 926-32213 |
| IRDye ™ 800CW Donkey anti-Mouse IgG Secondary Antibody | LI-COR | 926-32212 |

### Example of SALL4 Degradation Data:

After 20 h of treatment, thalidomide at 10 μM induced the degradation of SALL4 to an undetected level whereas for the compounds of Examples 33, 55 and 59, no observable/meaningful SALL4 degradation was seen.

### Endogenous Ikaros 1 (IKZF1) Degradation Assay:

The leukemia cell line, NB4, was used to monitor the degradation of the endogenous IKZF1 protein using a SDS-PAGE assay. NB4 cells were grown in RPMI 1640 medium supplemented with 10% fetal calf serum. Cells were split every 3 days at 1/10 ratio. Cells with less than 10 passages were used for the experiments. For the experiment, the cells were seeded in 12 well plates at 1,000,000 cells per well in a final volume of 1 mL of medium. The next day, the cells were dosed at 1 μM and 10 μM with the different compounds. After 20 h of treatment, the cells were lysed in RIPA buffer plus protease cocktail inhibitors (100 μL/well) for 20 minutes on ice. Lysates were collected and spun at 10000 g for 5 minutes. The supernatant was collected and mixed with sample buffer 4× containing a sample reducing agent (25 μL), then boiled for 5 minutes at 85° C. Lysates were stored at −20° C. For the SDS-PAGE analysis, samples were loaded on precast gel (4-12%), run at 150V constant on ice and transferred into membranes using P0 program from the fast transfer iBlot2 device (Thermofischer). Membranes were then incubated in LI-COR blocking solution (Intercept) for 30 minutes, then with primary antibodies overnight at 4° C. The membranes were then washed for a minimum of 30 minutes with TBS-tween 0.1% and incubated with secondary antibodies for LI-COR detection (1 h diluted at 1/10000). After several washes in PBS-Tween 0.1%, the membranes were analyzed by the Li-COR Odyssey CLx imaging system for protein level detection. GAPDH was used as loading control.

### List of Reagents

| Reagent | Source | Catalogue number |
|---|---|---|
| NB4 cells | DSMZ | ACC-207 |
| RPMI 1640 with Glutamine | Thermofischer | 11875093 |
| RIPA Lysis and Extraction Buffer | Thermofischer | 89900 |
| Halt ™ Protease Inhibitor Cocktail (100X) | Thermofischer | 78430 |
| NuPAGE ™ LDS Sample Buffer (4X) | Thermofischer | NP0007 |
| NuPAGE ™ Sample Reducing Agent (10X) | Thermofischer | NP0004 |
| NuPAGE ™ 4-12% Bis-Tris Protein Gels, 1.5 mm, 15-well | Thermofischer | NP0336BOX |
| iBlot ™ 2 Transfer Stacks, nitrocellulose, regular size | Thermofischer | IB23001 |
| Intercept ™ Blocking Buffer | LI-COR | 927-60001 |
| IKZF1 antibody | Cell Signaling | 14859 |
| GAPDH antibody | Proteintech | 60004-1-Ig |
| IRDye ™ 800CW Donkey anti-Rabbit IgG Secondary Antibody | LI-COR | 926-32213 |
| IRDye ™ 800CW Donkey anti-Mouse IgG Secondary Antibody | LI-COR | 926-32212 |

### Example of IKZF1 Degradation Data

After 20 h of treatment, lenalidomide at 1 μM and 10 μM induced the degradation of IKZF1 to an undetected level whereas for the compounds of Examples 17, 33, 39, 40 and 55, at 1 μM and 10 μM, no observable/meaningful IKZF1 degradation was seen.

1. A compound of Formula (I): 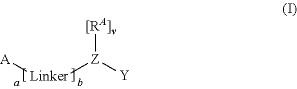
or a pharmaceutically acceptable salt thereof,
wherein:
A is a protein binder unit;
Z is Z^{A} or Z^{B}: 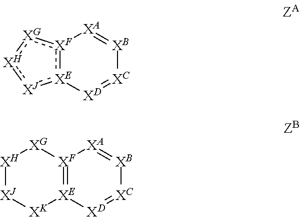
wherein: [symbol]
represents a single covalent bond or a double covalent bond;
1 of X^{A}, X^{B}, X^{C} & X^{D} is CY;
0, 1 or 2 of X^{A}, X^{B}, X^{C}, X^{D}, X^{E} & X^{F} is/are N where X^{E} & X^{F} are not both N, and are otherwise C;
and when Z is Z^{A}:
2 of X^{G}, X^{H} & X^{J} are independently selected from C and N; and
1 of X^{G}, X^{H} & X^{J} is C, N, S or O;
where at least one of X^{G}, X^{H}& X^{J} is N, S or O; and
where any one C of X^{G}, X^{H}& X^{J} is optionally substituted by oxo, or when both of X^{G} & X^{J} are C, they both may be optionally substituted by oxo; and
and when Z is Z^{B}
1 of X^{G}, X^{H}, X^{J} & X^{K} is N and are otherwise C; or alternatively
X^{G} & X^{K} are both N and X^{H} & X^{J} are both C;
Linker is a saturated or a partially or fully unsaturated framework comprising C and H atoms and at least one heteroatom, wherein said framework has end points of attachment ‘a’ and ‘b’ (and where ‘b’ may involve two attachment points ‘b1’ and ‘b2’ in cases where there are two points of attachment to Z at the ‘b’ end of the Linker) and a minimum length of from 6 to 26 atoms between ‘a’ and ‘b’; wherein said framework may include one or more straight and/or branched chains and/or rings and is optionally substituted on any available C atom(s) by one or more F; wherein said Linker is attached either:
once to Z: at any available C or N atom of Z; or
twice to Z: at any two adjacent available C atom(s) and/or N atom(s) at X^{H}, X^{G} & X^{J} (& X^{K} when present) such that a to 7-membered ring is formed by the attachment of the Linker at the two adjacent atoms of Z;
each R^{A} is a substituent on any available C or N atom of Z—in each case independently selected from R^{A1} optionally substituted by one or more R^{A2}; where R^{A} is further selected from R^{A2} when R^{A} is a substituent on an available C atom of Z;
each R^{A1} is independently C₁₋₄alkyl, C₂₋₃alkenyl, C₂₋₃alkynyl, C₁₋₃alkoxyC₁₋₃alkyl, carboxyC₁₋₃alkyl, C₅₋₇carbocyclyl or a 4-6 membered heterocyclyl;
each R^{A2} is independently selected from F, Cl, Br, CN, NH₂, C₁₋₃alkyl, O(C₁₋₃alkyl), NH(C₁₋₃alkyl) and N(C₁₋₃alkyl)₂;
wherein said C₁₋₃alkyls are optionally substituted by one or more F;
v is 0, 1, 2 or 3;
Y is: 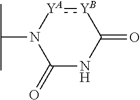
wherein:
Y^{A} & Y^{B} together represent CH—CH or C═C wherein Y^{A} & Y^{B} are each independently substituted by H, F, CN or Me.

2. The compound of Formula (I), as claimed in claim 1, or a pharmaceutically acceptable salt thereof, wherein the framework of the Linker is a saturated or partially unsaturated framework.

3. The compound of Formula (I), as claimed in claim 1 or claim 2, or a pharmaceutically acceptable salt thereof, wherein the framework of the Linker comprises C and H atoms and at least two heteroatoms selected from N & O.

4. The compound of Formula (I), as claimed in any one of claims 1 to 3, or a pharmaceutically acceptable salt thereof, wherein the framework of the Linker includes from 2 to 10 heteroatoms.

5. The compound of Formula (I), as claimed in any one of claims 1 to 4, or a pharmaceutically acceptable salt thereof, wherein the total number of C and hetero atoms in the Linker framework is from 8 to 30.

6. The compound of Formula (I), as claimed in any one of claims 1 to 5, or a pharmaceutically acceptable salt thereof, wherein Y and Linker are not attached at adjacent positions of Z.

7. A PROTAC compound containing an E3 ubiquitin ligase binding unit of Formula (Ia): 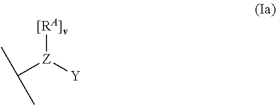
or a pharmaceutically acceptable salt thereof,
wherein the values of Z, Y, R^{A} and v are as defined in claim 1.

8. The PROTAC compound containing an E3 ubiquitin ligase binding unit of Formula (Ia), as claimed in claim 7, or a pharmaceutically acceptable salt thereof, where said PROTAC compound contains a unit of Formula (Ib): 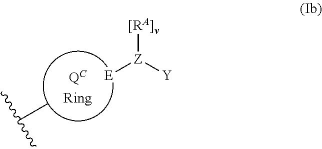
wherein:
Q^{C} Ring is a 4-11 membered saturated heterocyclic group;
E is linked to an available C or available N atom of Z, where
when E is linked to an available C atom of Z, E is C or N, and
when E is linked to an available N atom of Z, E is C.

9. The PROTAC compound containing an E3 ubiquitin ligase binding unit of Formula (Ia), as claimed in claim 7, or a pharmaceutically acceptable salt thereof, where said PROTAC compound contains a unit of Formula (Ic): 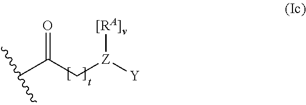
wherein t is 1 or 2.

10. The compound or pharmaceutically acceptable salt thereof according to any preceding claim wherein 0 or 1 of X^{A}, X^{B}, X^{C}, X^{D}, X^{E} & X^{F} is N, and are otherwise C.

11. The compound or pharmaceutically acceptable salt thereof according to any preceding claim wherein when Z is Z^{A}; X^{G}, X^{H} & X^{J} are collectively selected from (N, C, C), (O, N, C), (N, C, S), (N, N, N), (S, C, C), (N, N, C), (N, C, N), (O, C, C), (O, C, N), (C, N, C) and (N, N, C) respectively.

12. The compound or pharmaceutically acceptable salt thereof according to any preceding claim wherein when Z is Z^{A}; the (X^{G}—X^{H}-X^{J}) group together is selected from (N—C═C), (N—C—C), (N═C—C), (O—N═C), (N═C—S), (N—N═N), (S—C═C), (N—N═C), (N—C═N), (O—C═C), (O—C═N), (O—C—N), (C—N—C) and (N—N—C).

13. The compound or pharmaceutically acceptable salt thereof according to any preceding claim wherein Z is selected from indole, benzisoxazole, 1H-pyrrolo[2,3-c]pyridine, benzothiazole, 1H-pyrrolo[3,2-b]pyridine, indoline, benzotriazole, indazole, benzothiophene, 2H-indazole, benzimidazole, benzofuran, benzoxazole, 3H-1,3-benzoxazol-2-one, pyrazolo[1,5-a]pyridine, isoindolin-1-one, imidazo[1,2-a]pyridine, isoindoline, isoxazo[4,5-b]pyridine, furo[3,2-b]pyridine, 1H-pyrrolo[2,3-b]pyridine, 1,2,3,4-tetrahydroquinoline and 1,2,3,4-tetrahydroisoquinoline.

14. The compound or pharmaceutically acceptable salt thereof according to any preceding claim wherein Z, Y, R^{A} and v are collectively represented by any one or more of formulae 1 to 54 as shown in the description.

15. The compound or pharmaceutically acceptable salt thereof according to any preceding claim wherein R^{A} is a substituent on any available C or N atom of Z—in each case independently selected from C₁₋₃alkyl, N(C₁₋₃alkyl)₂ and C₁₋₃alkoxyC₁₋₃alkyl and R^{A} is further selected from F, Cl, CN and C₁₋₃alkoxy when said R^{A} is a substituent on an available C of Z.

16. The compound or pharmaceutically acceptable salt thereof according to any preceding claim wherein Y is selected from 6-fluoro-2,4-dioxohexahydropyrimidin-1-yl, 6-fluoro-2,4-dioxo-pyrimidin-1-yl, 2,4-dioxopyrimidin-1-yl, 6-methyl-2,4-dioxo-pyrimidin-1-yl and 2,6-dioxohexahydropyrimidin-1-yl.

17. The compound or pharmaceutically acceptable salt thereof according to any preceding claim wherein Y is 2,6-dioxohexahydropyrimidin-1-yl.

18. The compound or pharmaceutically acceptable salt thereof according to any preceding claim wherein Z, Y, R^{A} and v are collectively represented by any one or more of formulae 1 to 107 as shown in the description.

19. A compound of Formula (II): 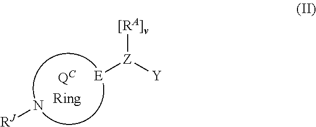
or a salt thereof, wherein:
R^{J} is H or a N-protecting group;
Q^{C} Ring is a 4-11 membered saturated heterocyclic group;
E is linked to an available C or available N atom of Z, where
when E is linked to an available C atom of Z, E is C or N, and
when E is linked to an available N atom of Z, E is C;
and the values of Z, V, R^{A} and v are as defined in any preceding claim.

20. The compound of Formula (II) as claimed in claim 19, or a salt thereof, wherein the N-protecting group is tert-butoxycarbonyl.

21. A compound of Formula (III): 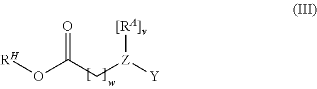
or a salt thereof, wherein:
w is 1 or 2;
R^{H} is H or C₁₋₈hydrocarbyl;
and the values of Z, Y, R^{A} and v are as defined in any preceding claim and where the compound of Formula (III) is other than 3-[6-(2,4-dioxohexahydropyrimidin-1-yl)-2-oxo-1,3-benzoxazol-3-yl]-propanoic acid.

22. A pharmaceutical composition, which comprises a compound of Formula (I), or a pharmaceutically acceptable salt thereof, as claimed in any one of claims 1-6 or 10-18, in association with a pharmaceutically acceptable excipient.

23. A pharmaceutical composition which comprises a PROTAC compound containing an E3 ubiquitin ligase binding unit of Formula (Ia), or a pharmaceutically acceptable salt thereof, as claimed in any one of claims 7 to 18, in association with a pharmaceutically acceptable excipient.

24. A pharmaceutical composition, which comprises a compound of Formula (I), or a pharmaceutically acceptable salt thereof, as claimed in any one of claims 1-6 or 10-18, for use in the treatment of cancer.

25. A pharmaceutical composition, which comprises a PROTAC compound containing an E3 ubiquitin ligase binding unit of Formula (Ia), or a pharmaceutically acceptable salt thereof, as claimed in any one of claims 7 to 18, for use in the treatment of cancer.

26. A pharmaceutical composition, which comprises a compound of Formula (I), or a pharmaceutically acceptable salt thereof, as claimed in any one of claims 1-6 or 10-18, for use in the treatment of a solid tumour.

27. A pharmaceutical composition, which comprises a PROTAC compound containing an E3 ubiquitin ligase binding unit of Formula (Ia), or a pharmaceutically acceptable salt thereof, as claimed in any one of claims 7 to 18, for use in the treatment of a solid tumour.

28. A pharmaceutical composition, which comprises a compound of Formula (I), or a pharmaceutically acceptable salt thereof, as claimed in any one of claims 1-6 or 10-18, for use in the treatment of a BRD4-sensitive tumour type.

29. The compound of Formula (I), or a pharmaceutically acceptable salt thereof, as claimed in any one of claims 1-6 or 10-18, for use as a medicament.

30. The PROTAC compound containing an E3 ubiquitin ligase binding unit of Formula (Ia), or a pharmaceutically acceptable salt thereof, as claimed in any one of claims 7 to 18, for use as a medicament.

31. The compound of Formula (I), or a pharmaceutically acceptable salt thereof, as claimed in any one of claims 1-6 or 10-18, for use in therapy.

32. The PROTAC compound containing an E3 ubiquitin ligase binding unit of Formula (Ia), or a pharmaceutically acceptable salt thereof, as claimed in any one of claims 7 to 18, for use in therapy.

33. The compound of Formula (I), or a pharmaceutically acceptable salt thereof, as claimed in any one of claims 1-6 or 10-18, for use in a method of treatment of the human or animal body by therapy.

34. The PROTAC compound containing an E3 ubiquitin ligase binding unit of Formula (Ia), or a pharmaceutically acceptable salt thereof, as claimed in any one of claims 7 to 18, for use in a method of treatment of the human or animal body by therapy.

35. The compound of Formula (I), or a pharmaceutically acceptable salt thereof, as claimed in any one of claims 1-6 or 10-18 for use in the production of an anti-proliferative effect in a warm-blooded animal such as man.

36. The PROTAC compound containing an E3 ubiquitin ligase binding unit of Formula (Ia), or a pharmaceutically acceptable salt thereof, as claimed in any one of claims 7 to 18, for use in the production of an anti-proliferative effect in a warm-blooded animal such as man.

37. The compound of Formula (I), or a pharmaceutically acceptable salt thereof, as claimed in any one of claims 1-6 or 10-18 for use in the production of a protein degrading effect in a warm-blooded animal such as man.

38. The PROTAC compound containing an E3 ubiquitin ligase binding unit of Formula (Ia), or a pharmaceutically acceptable salt thereof, as claimed in any one of claims 7 to 18, for use in the production of a protein degrading effect in a warm-blooded animal such as man.

39. Use of a compound of Formula (I), or a pharmaceutically acceptable salt thereof, as claimed in any one of claims 1-6 or 10-18, for the manufacture of a medicament for the production of an anti-proliferative effect (for example, in a warm-blooded animal such as man).

40. Use of a PROTAC compound containing an E3 ubiquitin ligase binding unit of Formula (Ia), or a pharmaceutically acceptable salt thereof, as claimed in any one of claims 7 to 18, for the manufacture of a medicament for the production of an anti-proliferative effect in a warm-blooded animal such as man.

41. Use of a PROTAC compound containing an E3 ubiquitin ligase binding unit of Formula (Ia), or a pharmaceutically acceptable salt thereof, as claimed in any one of claims 7 to 18, for the manufacture of a medicament for the production of a protein degrading effect in a warm-blooded animal such as man.

42. A method for producing an anti-proliferative effect in a warm-blooded animal, such as man, in need of such effect, which comprises administering to said animal an effective amount of a compound of Formula (I), or a pharmaceutically acceptable salt thereof, as claimed in any one of claims 1-6 or 10-18.

43. A method for producing an anti-proliferative effect in a warm-blooded animal, such as man, in need of such effect, which comprises administering to said animal an effective amount of a PROTAC compound containing an E3 ubiquitin ligase binding unit of Formula (Ia), or a pharmaceutically acceptable salt thereof, as claimed in any one of claims 7 to 18.

44. A method for producing a protein degrading effect in a warm-blooded animal, such as man, in need of such effect, which comprises administering to said animal an effective amount of a PROTAC compound containing an E3 ubiquitin ligase binding unit of Formula (Ia), or a pharmaceutically acceptable salt thereof.

45. The compound of Formula (I), or a pharmaceutically acceptable salt thereof, as claimed in any one of claims 1-6 or 10-18, for use as an anti-invasive agent in the containment and/or treatment of solid tumour disease.

46. The PROTAC compound containing an E3 ubiquitin ligase binding unit of Formula (Ia), or a pharmaceutically acceptable salt thereof, as claimed in any one of claims 7 to 18, for use as an anti-invasive agent in the containment and/or treatment of solid tumour disease.

47. Use of a compound of Formula (I), or a pharmaceutically acceptable salt thereof, as claimed in any one of claims 1-6 or 10-18, for the manufacture of a medicament for use as an anti-invasive agent in the containment and/or treatment of solid tumour disease.

48. Use of a PROTAC compound containing an E3 ubiquitin ligase binding unit of Formula (Ia), or a pharmaceutically acceptable salt thereof, as claimed in any one of claims 7 to 18, for the manufacture of a medicament for use as an anti-invasive agent in the containment and/or treatment of solid tumour disease.

49. A method for producing an anti-invasive effect by the containment and/or treatment of solid tumour disease, in a warm-blooded animal, such as man, in need of such effect, which comprises administering to said animal an effective amount of a compound of Formula (I), or a pharmaceutically acceptable salt thereof, as claimed in any one of claims 1-6 or 10-18.

50. A method for producing an anti-invasive effect by the containment and/or treatment of solid tumour disease, in a warm-blooded animal, such as man, in need of such effect, which comprises administering to said animal an effective amount of a PROTAC compound containing an E3 ubiquitin ligase binding unit of Formula (Ia), or a pharmaceutically acceptable salt thereof, as claimed in any one of claims 7 to 18.

51. The compound of Formula (I), or a pharmaceutically acceptable salt thereof, as claimed in any one of claims 1-6 or 10-18, for use in the prevention or treatment of cancer.

52. The PROTAC compound containing an E3 ubiquitin ligase binding unit of Formula (Ia), or a pharmaceutically acceptable salt thereof, as claimed in any one of claims 7 to 18, for use in the prevention or treatment of cancer.

53. Use of a compound of Formula (I), or a pharmaceutically acceptable salt thereof, as claimed in any one of claims 1-6 or 10-18, for the manufacture of a medicament for the prevention or treatment of cancer.

54. Use of a PROTAC compound containing an E3 ubiquitin ligase binding unit of Formula (Ia), or a pharmaceutically acceptable salt thereof, as claimed in any one of claims 7 to 18, for the manufacture of a medicament for the prevention or treatment of cancer.

55. A method for the prevention or treatment of cancer in a warm-blooded animal, such as man, in need of such treatment, which comprises administering to said animal an effective amount of a compound of Formula (I), or a pharmaceutically acceptable salt thereof, as claimed in any one of claims 1-6 or 10-18.

56. A method for the prevention or treatment of cancer in a warm-blooded animal, such as man, in need of such treatment, which comprises administering to said animal an effective amount of a PROTAC compound containing an E3 ubiquitin ligase binding unit of Formula (Ia), or a pharmaceutically acceptable salt thereof, as claimed in any one of claims 7 to 18.

57. The compound of Formula (I), or a pharmaceutically acceptable salt thereof, as claimed in any one of claims 1-6 or 10-18, for use in the prevention or treatment of solid tumour(s).

58. The PROTAC compound containing an E3 ubiquitin ligase binding unit of Formula (Ia), or a pharmaceutically acceptable salt thereof, as claimed in any one of claims 7 to 18, for use in the prevention or treatment of solid tumour(s).

59. Use of a compound of Formula (I), or a pharmaceutically acceptable salt thereof, as claimed in any one of claims 1-6 or 10-18, for the manufacture of a medicament for the prevention or treatment of solid tumour(s).

60. Use of a PROTAC compound containing an E3 ubiquitin ligase binding unit of Formula (Ia), or a pharmaceutically acceptable salt thereof, as claimed in any one of claims 7 to 18, for the manufacture of a medicament for the prevention or treatment of solid tumour(s).

61. A method for the prevention or treatment of solid tumour(s) in a warm-blooded animal, such as man, in need of such treatment, which comprises administering to said animal an effective amount of a compound of Formula (I), or a pharmaceutically acceptable salt thereof, as claimed in any one of claims 1-6 or 10-18.

62. A method for the prevention or treatment of solid tumour(s) in a warm-blooded animal, such as man, in need of such treatment, which comprises administering to said animal an effective amount of a PROTAC compound containing an E3 ubiquitin ligase binding unit of Formula (Ia), or a pharmaceutically acceptable salt thereof, as claimed in any one of claims 7 to 18.

63. The compound of Formula (I), or a pharmaceutically acceptable salt thereof, as claimed in any one of claims 1-6 or 10-18, for use in the prevention or treatment of tumour types that are sensitive to inhibition and/or degradation of BRD4.

64. Use of a compound of Formula (I), or a pharmaceutically acceptable salt thereof, as claimed in any one of claims 1-6 or 10-18, for the manufacture of a medicament for the prevention or treatment of those tumour types that are sensitive to inhibition and/or degradation of BRD4.

65. A method for the prevention or treatment of those tumour types that are sensitive to inhibition and/or degradation of BRD4, in a warm-blooded animal, such as man, in need of such treatment, which comprises administering to said animal an effective amount of a compound of Formula (I), or a pharmaceutically acceptable salt thereof, as claimed in any one of claims 1-6 or 10-18.

66. The compound of Formula (I), or a pharmaceutically acceptable salt thereof, as claimed in any one of claims 1-6 or 10-18, for use in providing an inhibitory and/or degrading effect on BRD4.

67. Use of a compound of Formula (I), or a pharmaceutically acceptable salt thereof, as claimed in any one of claims 1-6 or 10-18, for the manufacture of a medicament for providing an inhibitory and/or degrading effect on BRD4.

68. A method for providing an inhibitory and/degrading effect on BRD4 in a warm-blooded animal, such as man, in need of such effect, which comprises administering to said animal an effective amount of a compound of Formula (I), or a pharmaceutically acceptable salt thereof, as claimed in any one of claims 1-6 or 10-18.

69. The compound of Formula (I), or a pharmaceutically acceptable salt thereof, as claimed in any one of claims 1-6 or 10-18, for use in providing a selective inhibitory and/degrading effect on BRD4.

70. Use of a compound of Formula (I), or a pharmaceutically acceptable salt thereof, as claimed in any one of claims 1-6 or 10-18, for the manufacture of a medicament for providing a selective inhibitory and/or degrading effect on BRD4.

71. A method for providing a selective inhibitory and/degrading effect on BRD4 in a warm-blooded animal, such as man, in need of such effect, which comprises administering an effective amount of a compound of Formula (I), or a pharmaceutically acceptable salt thereof, as claimed in any one of claims 1-6 or 10-18.
